SEQUENCE LISTING (174 sequences)

SEQ ID NO: 1 - protein, 29 aa, synthetic construct
MHRPRRRGTRPPPLALLAALLLAARGADA

SEQ ID NO: 2 - DNA, 87 bp, synthetic construct
  source 1..87: mol_type = other DNA
atgcacagacctagacgtcgtggaactcgtccacctccactggcactgctcgctgctctcctcctggctgcacgtggtgctgatgca

SEQ ID NO: 3 - protein, 119 aa, synthetic construct
EVKLVESGGGLVQPGGSLKLSCATSGFTFSDYYMSWVRQTPEKRLEWVAYISSSGGSTYYSDTVKGQFTISRDNAKNTLYLQMSRLKSEDTAMYYCARREDYDGRLTYWGQGTLVTISA

SEQ ID NO: 4 - DNA, 357 bp, synthetic construct
  source 1..357: mol_type = other DNA
gaagtgaagctggtggagtctgggggaggcttagtgcagcctggagggtccctgaaactctcctgtgcaacctctggattcactttcagtgactattacatgtcttgggttcgccagactccagagaagaggctggagtgggtcgcatatattagtagtagtggtggtagcacctattattcagacactgtaaagggccaattcaccatctccagagacaatgccaagaacaccctgtacctgcaaatgagccgtctgaagtctgaggacacagccatgtattactgtgcaagacgagaagattacgacggaagacttacttactggggccaagggactctggtcaccatctctgca

SEQ ID NO: 5 - protein, 7 aa, synthetic construct
SGFTFSD

SEQ ID NO: 6 - protein, 17 aa, synthetic construct
LEWVAYISSSGGSTYYS

SEQ ID NO: 7 - protein, 10 aa, synthetic construct
REDYDGRLTY

SEQ ID NO: 8 - protein, 111 aa, synthetic construct
DIVLTQSPASLAVSLGQRATISCRASKSVSTSGYSYMNWYQQKPGQPPKVLIYLASKLESGVPARFSGSGSGTDFTLNIHPVEEEDAATYYCQHSRELPYTFGGGTKLEIK

SEQ ID NO: 9 - DNA, 333 bp, synthetic construct
  source 1..333: mol_type = other DNA
gacattgtgctgacacagtctcctgcttccttagctgtatctctggggcagagggccaccatctcctgcagggccagcaaaagtgtcagtacatctggttatagttatatgaactggtaccaacagaaaccaggacagccacccaaagtcctcatctatcttgcatccaaactagaatctggggtccctgccaggttcagtggcagtgggtcagggacagacttcaccctcaacatccatcctgtggaggaggaggatgctgcaacctattactgtcagcacagtagggagcttccgtacacgttcggaggggggaccaaactggaaataaaa

SEQ ID NO: 10 - protein, 9 aa, synthetic construct
KSVSTSGYS

SEQ ID NO: 11 - protein, 12 aa, synthetic construct
PKVLIYLASKLE

SEQ ID NO: 12 - protein, 6 aa, synthetic construct
SRELPY

SEQ ID NO: 13 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 14 - DNA, 981 bp, Homo sapiens
  source 1..981: mol_type = genomic DNA
gcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaa

SEQ ID NO: 15 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 16 - DNA, 978 bp, Homo sapiens
  source 1..978: mol_type = genomic DNA
gcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctccggtggcgggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaa

SEQ ID NO: 17 - protein, 107 aa, Homo sapiens [sequence omitted]

SEQ ID NO: 18 - DNA, 324 bp, Homo sapiens
  source 1..324: mol_type = genomic DNA
cgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 19 - protein, 12 aa, synthetic construct
GGGSGGGSGGGS

SEQ ID NO: 20 - DNA, 36 bp, synthetic construct
  source 1..36: mol_type = other DNA
ggaggcggctccggcggcggatccggcggcggctct

SEQ ID NO: 21 - protein, 361 aa, Homo sapiens
LSTSKTIDMELVKRKRIEAIRGQILSKLRLASPPSQGEVPPGPLPEAVLALYNSTRDRVAGESAEPEPEPEADYYAKEVTRVLMVETHNEIYDKFKQSTHSIYMFFNTSELREAVPEPVLLSRAELRLLRLKLKVEQHVELYQKYSNNSWRYLSNRLLAPSDSPEWLSFDVTGVVRQWLSRGGEIEGFRLSAHCSCDSRDNTLQVDINGFTTGRRGDLATIHGMNRPFLLLMATPLERAQHLQSSRHRRALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 22 - DNA, 1083 bp, Homo sapiens
  source 1..1083: mol_type = genomic DNA
ctatccaccagcaagactatcgacatggagctggtgaagcggaagcgcatcgaggccatccgcggccagatcctgtccaagctgcggctcgccagccccccgagccagggggaggtgccgcccggcccgctgcccgaggccgtgctcgccctgtacaacagcacccgcgaccgggtggccggggagagtgcagaaccggagcccgagcctgaggccgactactacgccaaggaggtcacccgcgtgctaatggtggaaacccacaacgaaatctatgacaagttcaagcagagtacacacagcatatatatgttcttcaacacatcagagctccgagaagcggtacctgaacccgtgttgctctcccgggcagagctgcgtctgctgaggctcaagttaaaagtggagcagcacgtggagctgtaccagaaatacagcaacaattcctggcgatacctcagcaaccggctgctggcacccagcgactcgccagagtggttatcttttgatgtcaccggagttgtgcggcagtggttgagccgtggaggggaaattgagggctttcgccttagcgcccactgctcctgtgacagcagggataacacactgcaagtggacatcaacgggttcactaccggccgccgaggtgacctggccaccattcatggcatgaaccggcctttcctgcttctcatggccaccccgctggagagggcccagcatctgcaaagctcccggcaccgccgagccctggacaccaactattgcttcagctccacggagaagaactgctgcgtgcggcagctgtacattgacttccgcaaggacctcggctggaagtggatccacgagcccaagggctaccatgccaacttctgcctcgggccctgcccctacatttggagcctggacacgcagtacagcaaggtcctggccctgtacaaccagcataacccgggcgcctcggcggcgccgtgctgcgtgccgcaggcgctggagccgctgcccatcgtgtactacgtgggccgcaagcccaaggtggagcagctgtccaacatgatcgtgcgctcctgcaagtgcagc

SEQ ID NO: 23 - protein, 112 aa, Homo sapiens
ALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 24 - DNA, 336 bp, Homo sapiens
  source 1..336: mol_type = genomic DNA
gccctggacaccaactattgcttcagctccacggagaagaactgctgcgtgcggcagctgtacattgacttccgcaaggacctcggctggaagtggatccacgagcccaagggctaccatgccaacttctgcctcgggccctgcccctacatttggagcctggacacgcagtacagcaaggtcctggccctgtacaaccagcataacccgggcgcctcggcggcgccgtgctgcgtgccgcaggcgctggagccgctgcccatcgtgtactacgtgggccgcaagcccaaggtggagcagctgtccaacatgatcgtgcgctcctgcaagtgcagc

SEQ ID NO: 25 - protein, 394 aa, Homo sapiens
LSTSSTLDMDQFMRKRIEAIRGQILSKLKLTSPPEDYPEPEEVPPEVISIYNSTRDLLQEKASRRAAACERERSDEEYYAKEVYKIDMPPFFPSENAIPPTFYRPYFRIVRFDVSAMEKNASNLVKAEFRVFRLQNPKARVPEQRIELYQILKSKDLTSPTQRYIDSKVVKTRAEGEWLSFDVTDAVHEWLHHKDRNLGFKISLHCPCCTFVPSNNYIIPNKSEELEARFAGIDGTSTYTSGDQKTIKSTRKKNSGKTPHLLLMLLPSYRLESQQTNRRKKRALDAAYCFRNVQDNCCLRPLYIDFKRDLGWKWIHEPKGYNANFCAGACPYLWSSDTQHSRVLSLYNTINPEASASPCCVSQDLEPLTILYYIGKTPKIEQLSNMIVKSCKCS

SEQ ID NO: 26 - DNA, 1182 bp, Homo sapiens
  source 1..1182: mol_type = genomic DNA
ttgtccacctcctctacactggatatggaccagttcatgcggaagagaatcgaggctatccggggccaaatcctgagcaagctcaaacttacctctccacctgaggactaccctgagcctgaggaagtccctcctgaggtgatctccatctacaactctactcgggacctgctgcaagagaaggcttctcgcagagccgctgcttgcgagcgggaacgctccgacgaggaatactacgccaaagaggtgtacaagatcgacatgcctcctttcttcccctctgagaatgccatccctcctaccttctacagaccttacttcagaatcgtccggttcgacgtgtctgccatggaaaagaacgcctccaatctcgtgaaggccgagttccgggtgttcagactgcagaaccccaaggccagggtgcccgaacagcggatcgagctgtaccagatcctgaagtccaaggacctgaccagccccacccagagatacatcgattctaaagttgtgaagacccgggccgagggcgagtggctgtccttcgacgtgaccgacgccgtgcatgagtggctgcaccacaaggaccggaacctgggcttcaagatctccctgcactgcccctgttgcaccttcgtgccttcaaacaactacatcatacctaacaagtccgaggaactggaggccagattcgccggaatcgatggcacctccacctacacctctggcgaccagaagaccatcaagtctacccggaaaaagaactctggcaagacaccccacctgctgctgatgctgctaccttcctaccggctggaatctcagcagaccaaccgaagaaagaagagagccctggacgccgcttattgcttccggaatgtgcaggacaactgctgcctgagacctctctacatcgacttcaagagagacctcggctggaagtggatccacgagcccaagggctacaacgctaacttctgcgcgggcgcctgcccttatctgtggtcctcagatacccagcattccagagtgttgtccctgtacaacaccatcaaccctgaagcctctgcctccccctgctgtgtctcccaagatctggagcccctgaccatcctgtactacatcggcaagacacctaagattgaacagctgtccaacatgatcgtgaagagctgcaagtgttcc

SEQ ID NO: 27 - protein, 112 aa, Homo sapiens
ALDAAYCFRNVQDNCCLRPLYIDFKRDLGWKWIHEPKGYNANFCAGACPYLWSSDTQHSRVLSLYNTINPEASASPCCVSQDLEPLTILYYIGKTPKIEQLSNMIVKSCKCS

SEQ ID NO: 28 - DNA, 336 bp, Homo sapiens
  source 1..336: mol_type = genomic DNA
gccctggacgccgcttattgcttccggaatgtgcaggacaactgctgcctgagacctctctacatcgacttcaagagagacctcggctggaagtggatccacgagcccaagggctacaacgctaacttctgcgcgggcgcctgcccttatctgtggtcctcagatacccagcattccagagtgttgtccctgtacaacaccatcaaccctgaagcctctgcctccccctgctgtgtctcccaagatctggagcccctgaccatcctgtactacatcggcaagacacctaagattgaacagctgtccaacatgatcgtgaagagctgcaagtgttcc

SEQ ID NO: 29 - protein, 245 aa, Homo sapiens
LSTCKTIDMELVKRKRIEAIRGQILSKLRLASPPSQGEVPPGPLPEAVLALYNSTRDRVAGESAEPEPEPEADYYAKEVTRVLMVETHNEIYDKFKQSTHSIYMFFNTSELREAVPEPVLLSRAELRLLRLKLKVEQHVELYQKYSNNSWRYLSNRLLAPSDSPEWLSFDVTGVVRQWLSRGGEIEGFRLSAHCSCDSRDNTLQVDINGFTTGRRGDLATIHGMNRPFLLLMATPLERAQHLQSS

SEQ ID NO: 30 - DNA, 735 bp, Homo sapiens
  source 1..735: mol_type = genomic DNA
ctatccacctgcaagactatcgacatggagctggtgaagcggaagcgcatcgaggccatccgcggccagatcctgtccaagctgcggctcgccagccccccgagccagggggaggtgccgcccggcccgctgcccgaggccgtgctcgccctgtacaacagcacccgcgaccgggtggccggggagagtgcagaaccggagcccgagcctgaggccgactactacgccaaggaggtcacccgcgtgctaatggtggaaacccacaacgaaatctatgacaagttcaagcagagtacacacagcatatatatgttcttcaacacatcagagctccgagaagcggtacctgaacccgtgttgctctcccgggcagagctgcgtctgctgaggctcaagttaaaagtggagcagcacgtggagctgtaccagaaatacagcaacaattcctggcgatacctcagcaaccggctgctggcacccagcgactcgccagagtggttatcttttgatgtcaccggagttgtgcggcagtggttgagccgtggaggggaaattgagggctttcgccttagcgcccactgctcctgtgacagcagggataacacactgcaagtggacatcaacgggttcactaccggccgccgaggtgacctggccaccattcatggcatgaaccggcctttcctgcttctcatggccaccccgctggagagggcccagcatctgcaaagctcc

SEQ ID NO: 31 - protein, 245 aa, Homo sapiens
LSTSKTIDMELVKRKRIEAIRGQILSKLRLASPPSQGEVPPGPLPEAVLALYNSTRDRVAGESAEPEPEPEADYYAKEVTRVLMVETHNEIYDKFKQSTHSIYMFFNTSELREAVPEPVLLSRAELRLLRLKLKVEQHVELYQKYSNNSWRYLSNRLLAPSDSPEWLSFDVTGVVRQWLSRGGEIEGFRLSAHCSCDSRDNTLQVDINGFTTGRRGDLATIHGMNRPFLLLMATPLERAQHLQSS

SEQ ID NO: 32 - DNA, 735 bp, Homo sapiens
  source 1..735: mol_type = genomic DNA
ctatccaccagcaagactatcgacatggagctggtgaagcggaagcgcatcgaggccatccgcggccagatcctgtccaagctgcggctcgccagccccccgagccagggggaggtgccgcccggcccgctgcccgaggccgtgctcgccctgtacaacagcacccgcgaccgggtggccggggagagtgcagaaccggagcccgagcctgaggccgactactacgccaaggaggtcacccgcgtgctaatggtggaaacccacaacgaaatctatgacaagttcaagcagagtacacacagcatatatatgttcttcaacacatcagagctccgagaagcggtacctgaacccgtgttgctctcccgggcagagctgcgtctgctgaggctcaagttaaaagtggagcagcacgtggagctgtaccagaaatacagcaacaattcctggcgatacctcagcaaccggctgctggcacccagcgactcgccagagtggttatcttttgatgtcaccggagttgtgcggcagtggttgagccgtggaggggaaattgagggctttcgccttagcgcccactgctcctgtgacagcagggataacacactgcaagtggacatcaacgggttcactaccggccgccgaggtgacctggccaccattcatggcatgaaccggcctttcctgcttctcatggccaccccgctggagagggcccagcatctgcaaagctcc

SEQ ID NO: 33-35 - skipped (3 entries)

SEQ ID NO: 36 - protein, 848 aa, synthetic construct
MHRPRRRGTRPPPLALLAALLLAARGADAEVKLVESGGGLVQPGGSLKLSCATSGFTFSDYYMSWVRQTPEKRLEWVAYISSSGGSTYYSDTVKGQFTISRDNAKNTLYLQMSRLKSEDTAMYYCARREDYDGRLTYWGQGTLVTISAASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGSGGGSGGGSLSTSKTIDMELVKRKRIEAIRGQILSKLRLASPPSQGEVPPGPLPEAVLALYNSTRDRVAGESAEPEPEPEADYYAKEVTRVLMVETHNEIYDKFKQSTHSIYMFFNTSELREAVPEPVLLSRAELRLLRLKLKVEQHVELYQKYSNNSWRYLSNRLLAPSDSPEWLSFDVTGVVRQWLSRGGEIEGFRLSAHCSCDSRDNTLQVDINGFTTGRRGDLATIHGMNRPFLLLMATPLERAQHLQSSRHRRALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 37 - DNA, 2544 bp, synthetic construct
  source 1..2544: mol_type = other DNA
atgcacagacctagacgtcgtggaactcgtccacctccactggcactgctcgctgctctcctcctggctgcacgtggtgctgatgcagaagtgaagctggtggagtctgggggaggcttagtgcagcctggagggtccctgaaactctcctgtgcaacctctggattcactttcagtgactattacatgtcttgggttcgccagactccagagaagaggctggagtgggtcgcatatattagtagtagtggtggtagcacctattattcagacactgtaaagggccaattcaccatctccagagacaatgccaagaacaccctgtacctgcaaatgagccgtctgaagtctgaggacacagccatgtattactgtgcaagacgagaagattacgacggaagacttacttactggggccaagggactctggtcaccatctctgcagcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaaggaggcggctccggcggcggatccggcggcggctctctatccaccagcaagactatcgacatggagctggtgaagcggaagcgcatcgaggccatccgcggccagatcctgtccaagctgcggctcgccagccccccgagccagggggaggtgccgcccggcccgctgcccgaggccgtgctcgccctgtacaacagcacccgcgaccgggtggccggggagagtgcagaaccggagcccgagcctgaggccgactactacgccaaggaggtcacccgcgtgctaatggtggaaacccacaacgaaatctatgacaagttcaagcagagtacacacagcatatatatgttcttcaacacatcagagctccgagaagcggtacctgaacccgtgttgctctcccgggcagagctgcgtctgctgaggctcaagttaaaagtggagcagcacgtggagctgtaccagaaatacagcaacaattcctggcgatacctcagcaaccggctgctggcacccagcgactcgccagagtggttatcttttgatgtcaccggagttgtgcggcagtggttgagccgtggaggggaaattgagggctttcgccttagcgcccactgctcctgtgacagcagggataacacactgcaagtggacatcaacgggttcactaccggccgccgaggtgacctggccaccattcatggcatgaaccggcctttcctgcttctcatggccaccccgctggagagggcccagcatctgcaaagctcccggcaccgccgagccctggacaccaactattgcttcagctccacggagaagaactgctgcgtgcggcagctgtacattgacttccgcaaggacctcggctggaagtggatccacgagcccaagggctaccatgccaacttctgcctcgggccctgcccctacatttggagcctggacacgcagtacagcaaggtcctggccctgtacaaccagcataacccgggcgcctcggcggcgccgtgctgcgtgccgcaggcgctggagccgctgcccatcgtgtactacgtgggccgcaagcccaaggtggagcagctgtccaacatgatcgtgcgctcctgcaagtgcagc

SEQ ID NO: 38 - protein, 881 aa, synthetic construct
MHRPRRRGTRPPPLALLAALLLAARGADAEVKLVESGGGLVQPGGSLKLSCATSGFTFSDYYMSWVRQTPEKRLEWVAYISSSGGSTYYSDTVKGQFTISRDNAKNTLYLQMSRLKSEDTAMYYCARREDYDGRLTYWGQGTLVTISAASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGSGGGSGGGSLSTSSTLDMDQFMRKRIEAIRGQILSKLKLTSPPEDYPEPEEVPPEVISIYNSTRDLLQEKASRRAAACERERSDEEYYAKEVYKIDMPPFFPSENAIPPTFYRPYFRIVRFDVSAMEKNASNLVKAEFRVFRLQNPKARVPEQRIELYQILKSKDLTSPTQRYIDSKVVKTRAEGEWLSFDVTDAVHEWLHHKDRNLGFKISLHCPCCTFVPSNNYIIPNKSEELEARFAGIDGTSTYTSGDQKTIKSTRKKNSGKTPHLLLMLLPSYRLESQQTNRRKKRALDAAYCFRNVQDNCCLRPLYIDFKRDLGWKWIHEPKGYNANFCAGACPYLWSSDTQHSRVLSLYNTINPEASASPCCVSQDLEPLTILYYIGKTPKIEQLSNMIVKSCKCS

SEQ ID NO: 39 - DNA, 2643 bp, synthetic construct
  source 1..2643: mol_type = other DNA
atgcacagacctagacgtcgtggaactcgtccacctccactggcactgctcgctgctctcctcctggctgcacgtggtgctgatgcagaagtgaagctggtggagtctgggggaggcttagtgcagcctggagggtccctgaaactctcctgtgcaacctctggattcactttcagtgactattacatgtcttgggttcgccagactccagagaagaggctggagtgggtcgcatatattagtagtagtggtggtagcacctattattcagacactgtaaagggccaattcaccatctccagagacaatgccaagaacaccctgtacctgcaaatgagccgtctgaagtctgaggacacagccatgtattactgtgcaagacgagaagattacgacggaagacttacttactggggccaagggactctggtcaccatctctgcagcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaaggaggcggctccggcggcggatccggcggcggctctttgtccacctcctctacactggatatggaccagttcatgcggaagagaatcgaggctatccggggccaaatcctgagcaagctcaaacttacctctccacctgaggactaccctgagcctgaggaagtccctcctgaggtgatctccatctacaactctactcgggacctgctgcaagagaaggcttctcgcagagccgctgcttgcgagcgggaacgctccgacgaggaatactacgccaaagaggtgtacaagatcgacatgcctcctttcttcccctctgagaatgccatccctcctaccttctacagaccttacttcagaatcgtccggttcgacgtgtctgccatggaaaagaacgcctccaatctcgtgaaggccgagttccgggtgttcagactgcagaaccccaaggccagggtgcccgaacagcggatcgagctgtaccagatcctgaagtccaaggacctgaccagccccacccagagatacatcgattctaaagttgtgaagacccgggccgagggcgagtggctgtccttcgacgtgaccgacgccgtgcatgagtggctgcaccacaaggaccggaacctgggcttcaagatctccctgcactgcccctgttgcaccttcgtgccttcaaacaactacatcatacctaacaagtccgaggaactggaggccagattcgccggaatcgatggcacctccacctacacctctggcgaccagaagaccatcaagtctacccggaaaaagaactctggcaagacaccccacctgctgctgatgctgctaccttcctaccggctggaatctcagcagaccaaccgaagaaagaagagagccctggacgccgcttattgcttccggaatgtgcaggacaactgctgcctgagacctctctacatcgacttcaagagagacctcggctggaagtggatccacgagcccaagggctacaacgctaacttctgcgcgggcgcctgcccttatctgtggtcctcagatacccagcattccagagtgttgtccctgtacaacaccatcaaccctgaagcctctgcctccccctgctgtgtctcccaagatctggagcccctgaccatcctgtactacatcggcaagacacctaagattgaacagctgtccaacatgatcgtgaagagctgcaagtgttcc

SEQ ID NO: 40 - protein, 247 aa, synthetic construct
MHRPRRRGTRPPPLALLAALLLAARGADADIVLTQSPASLAVSLGQRATISCRASKSVSTSGYSYMNWYQQKPGQPPKVLIYLASKLESGVPARFSGSGSGTDFTLNIHPVEEEDAATYYCQHSRELPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 41 - DNA, 741 bp, synthetic construct
  source 1..741: mol_type = other DNA
atgcacagacctagacgtcgtggaactcgtccacctccactggcactgctcgctgctctcctcctggctgcacgtggtgctgatgcagacattgtgctgacacagtctcctgcttccttagctgtatctctggggcagagggccaccatctcctgcagggccagcaaaagtgtcagtacatctggttatagttatatgaactggtaccaacagaaaccaggacagccacccaaagtcctcatctatcttgcatccaaactagaatctggggtccctgccaggttcagtggcagtgggtcagggacagacttcaccctcaacatccatcctgtggaggaggaggatgctgcaacctattactgtcagcacagtagggagcttccgtacacgttcggaggggggaccaaactggaaataaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 42 - protein, 847 aa, synthetic construct
MHRPRRRGTRPPPLALLAALLLAARGADAEVKLVESGGGLVQPGGSLKLSCATSGFTFSDYYMSWVRQTPEKRLEWVAYISSSGGSTYYSDTVKGQFTISRDNAKNTLYLQMSRLKSEDTAMYYCARREDYDGRLTYWGQGTLVTISAASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGSGGGSGGGSLSTSKTIDMELVKRKRIEAIRGQILSKLRLASPPSQGEVPPGPLPEAVLALYNSTRDRVAGESAEPEPEPEADYYAKEVTRVLMVETHNEIYDKFKQSTHSIYMFFNTSELREAVPEPVLLSRAELRLLRLKLKVEQHVELYQKYSNNSWRYLSNRLLAPSDSPEWLSFDVTGVVRQWLSRGGEIEGFRLSAHCSCDSRDNTLQVDINGFTTGRRGDLATIHGMNRPFLLLMATPLERAQHLQSSRHRRALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 43 - DNA, 2541 bp, synthetic construct
  source 1..2541: mol_type = other DNA
atgcatcgaccacgacgacgaggtactcgacctccccctctggctctgctggctgctctgcttctggccgcccggggggccgacgccgaggtgaagctggtggaatctggaggcggcctcgtgcagcctggcggcagcctgaaactgagttgtgctaccagcggcttcaccttctccgactactacatgtcttgggtcagacagaccccagaaaagagactggaatgggtggcctacatctctagctccggcggatctacctattactccgataccgtgaaaggccagtttaccatttctcgggataatgccaagaacaccctgtacctgcagatgtcccggctcaagtctgaggacaccgccatgtactactgcgccagaagagaggactacgacggcagactgacctactggggccagggcaccctggttacaatcagcgccgcttctaccaaggggccttctgtttttcctctggctccttgctctagatccacctccgagtctacagctgctctgggctgcctggtgaaggactacttccccgagcctgtgaccgtgtcctggaactctggcgccctgacctctggcgtgcacaccttccctgccgtgctgcagtcttccggcctctacagcctgagctctgtggtgactgtgccttcctctagcctgggaaccaagacctacacatgcaacgtggaccacaagcccagcaataccaaggtggacaagcgggtcgaatccaagtacggccctccttgcccaccttgtcctgctcctcctgtggctggcccgtcggtgttcctgttccctccaaagcccaaggataccctgatgatctcccggactcctgaggtgacctgcgtggtcgtggacgtgtctcaagaggatcctgaagtgcagttcaactggtatgtcgatggcgtcgaagtgcataatgccaaaacaaaacctcgggaggaacagttcaactccacctacagagtggtgtctgtgctgaccgtgctgcaccaggactggctgaacggcaaagagtacaagtgcaaggtgtccaacaagggcctgccctcctccatcgagaagaccatctctaaggccaagggtcagcctagagagccccaagtgtacaccctgcccccctcccaagaagagatgaccaagaaccaggtgtccctgacatgtctggtcaaaggcttctacccctccgacatcgccgtggaatgggagtccaatggccagcccgagaacaactacaagaccacccctcctgtactggactctgacggctctttctttctgtattcccggctgacggtggataagtccagatggcaggagggcaacgtgttttcctgctccgtgatgcacgaggctctgcataaccactacacccagaagtctttgagcctgtccctgggcaagggaggcggctccggcggcggatccggcggcggctctctgtccacctccaaaacaatcgacatggaactggtgaagagaaagcggatcgaggctatcagaggacagatcctgtctaaactgcggctggcctctcccccttcccagggcgaggtgcctcccggccccctgcctgaagctgtgctggccctgtacaactcgaccagagacagagtggccggcgagtccgccgagccagagccagaacctgaagctgactattatgctaaagaagtgaccagagtgctgatggtggagacacacaacgagatctacgacaagttcaagcagagcacccactccatctacatgttcttcaacacctccgaactgcgggaggccgtgcctgagcctgtgctgctgtctagagctgagctgcgcctgctgagactgaagctgaaggtcgagcagcacgtggagctgtaccagaagtactccaacaacagctggcggtacctgtccaaccgcctgctggctccatcagactcccctgagtggctgtccttcgacgtgaccggcgtggtgcggcagtggctgtctagaggcggagagatcgagggattcagactgtctgcccactgctcctgcgactctagggacaacaccttgcaagtcgatatcaacggctttactaccggcagacggggcgacctggccaccattcacggcatgaacagacctttcttactgctgatggccacacctctggagagagcccagcacctgcagtctagcagacaccggagagccctggataccaactactgcttctctagcaccgagaagaattgctgtgtcagacagctgtacatcgacttccggaaggatctgggctggaagtggatccacgagcctaagggctaccacgccaacttctgcctgggcccttgtccttacatctggtccctggacacccagtactctaaggtgctcgccctgtacaaccagcataaccctggcgctagtgctgccccatgctgcgtgcctcaggccctggaacctctgcctatcgtgtactacgtgggccggaagcctaaagtcgagcagctgtccaacatgatcgtgcggtcctgcaagtgttcc

SEQ ID NO: 44 - protein, 247 aa, synthetic construct; identical to SEQ ID NO: 40

SEQ ID NO: 45 - DNA, 741 bp, synthetic construct
  source 1..741: mol_type = other DNA
atgcatcgaccacgacgacgaggtactcgacccccccctctggccctgctggctgctctgctgctggctgctagaggcgctgatgccgatatcgtgctgacccagagccctgcatccctggccgtgtccctcggccagcgggccaccatctcctgcagagcctctaaaagcgtgtctacctccggctactcctacatgaactggtaccagcagaagcctggccagcctcctaaggtgctgatctacctggcttctaaactggaatctggcgtgcctgctcggttctccggctccggctctggcaccgatttcaccctgaacatccaccccgtggaagaagaggacgccgctacctactactgccagcattctagagagctgccttacaccttcggcggaggtacaaagctggagatcaagagaaccgtggccgccccttctgtgttcatctttcctccatccgacgagcagctgaagtctggcacagcctccgtggtgtgcctgctgaataacttctatcctcgcgaagccaaggtgcagtggaaggtggacaacgccctgcagtccggcaactcccaagagtccgtcactgaacaagactccaaggacagtacctacagcctgtcttctaccctcacactgagcaaggccgactacgagaagcacaaagtgtacgcctgtgaggtgacccaccagggactgtcctctccagtcaccaagtccttcaaccggggcgagtgc

SEQ ID NO: 46 - protein, 50 aa, synthetic construct
  VARIANT 1..50: note = This sequence may encompass 1-10 GGGGS repeating units
GGGGSGGGGSGGGGSGGGGSGGGGSGGGGSGGGGSGGGGSGGGGSGGGGS

SEQ ID NO: 47 - protein, 5 aa, synthetic construct
GGGGS

SEQ ID NO: 48 - protein, 10 aa, synthetic construct
GGGGSGGGGS

SEQ ID NO: 49 - protein, 15 aa, synthetic construct
GGGGSGGGGSGGGGS

SEQ ID NO: 50 - protein, 20 aa, synthetic construct
GGGGSGGGGSGGGGSGGGGS

SEQ ID NO: 51 - protein, 40 aa, synthetic construct
  VARIANT 1..40: note = This sequence may encompass 1-10 GGGS repeating units
GGGSGGGSGGGSGGGSGGGSGGGSGGGSGGGSGGGSGGGS

SEQ ID NO: 52 - protein, 4 aa, synthetic construct
GSGS

SEQ ID NO: 53 - protein, 8 aa, synthetic construct
GGGSGGGS

SEQ ID NO: 54 - protein, 16 aa, synthetic construct
GGGSGGGSGGGSGGGS

SEQ ID NO: 55 - protein, 20 aa, synthetic construct
GGGSGGGSGGGSGGGSGGGS

SEQ ID NO: 56 - protein, 25 aa, synthetic construct
GGGGSGGGGSGGGGSGGGGSGGGGS

SEQ ID NO: 57 - protein, 20 aa, synthetic construct
GGSEGKSSGSGSESKSTGGS

SEQ ID NO: 58 - protein, 14 aa, synthetic construct
IRPRAIGGSKPRVA

SEQ ID NO: 59 - protein, 15 aa, synthetic construct
GKGGSGKGGSGKGGS

SEQ ID NO: 60 - protein, 15 aa, synthetic construct
GGKGSGGKGSGGKGS

SEQ ID NO: 61 - protein, 15 aa, synthetic construct
GGGKSGGGKSGGGKS

SEQ ID NO: 62 - protein, 15 aa, synthetic construct
GKGKSGKGKSGKGKS

SEQ ID NO: 63 - protein, 15 aa, synthetic construct
GGGKSGGKGSGKGGS

SEQ ID NO: 64 - protein, 15 aa, synthetic construct
GKPGSGKPGSGKPGS

SEQ ID NO: 65 - protein, 20 aa, synthetic construct
GKGKSGKGKSGKGKSGKGKS

SEQ ID NO: 66 - protein, 14 aa, synthetic construct
STAGDTHLGGEDFD

SEQ ID NO: 67 - protein, 15 aa, synthetic construct
GEGGSGEGGSGEGGS

SEQ ID NO: 68 - protein, 15 aa, synthetic construct
GGEGSGGEGSGGEGS

SEQ ID NO: 69 - protein, 15 aa, synthetic construct
GEGESGEGESGEGES

SEQ ID NO: 70 - protein, 15 aa, synthetic construct
GGGESGGEGSGEGGS

SEQ ID NO: 71 - protein, 20 aa, synthetic construct
GEGESGEGESGEGESGEGES

SEQ ID NO: 72 - protein, 18 aa, synthetic construct
GSTSGSGKPGSGEGSTKG

SEQ ID NO: 73 - protein, 19 aa, synthetic construct
PRGASKSGSASQTGSAPGS

SEQ ID NO: 74 - protein, 19 aa, synthetic construct
GTAAAGAGAAGGAAAGAAG

SEQ ID NO: 75 - protein, 19 aa, synthetic construct
GTSGSSGSGSGGSGSGGGG

SEQ ID NO: 76 - protein, 20 aa, synthetic construct
GKPGSGKPGSGKPGSGKPGS

SEQ ID NO: 77 - protein, 10 aa, synthetic construct
APAPAPAPAP

SEQ ID NO: 78 - protein, 20 aa, synthetic construct
APAPAPAPAPAPAPAPAPAP

SEQ ID NO: 79 - protein, 32 aa, synthetic construct
AEAAAKEAAAKEAAAAKEAAAAKEAAAAKAAA

SEQ ID NO: 80 - protein, 245 aa, Homo sapiens
LSTSKTIDMELVKRKRIEAIRGQILSKLRLASPPSQGEVPPGPLPEAVLALYNSTRDRVAGESAEPEPEPEADYYAKEVTRVLMVETHNEIYDKFKQSTHSIYMFFNTSELREAVPEPVLLSRAELRLLRLKLKVEQHVELYQKYSNNSWRYLSNRLLAPSDSPEWLSFDVTGVVRQWLSRGGEIEGFRLSAHSSSDSRDNTLQVDINGFTTGRRGDLATIHGMNRPFLLLMATPLERAQHLQSS

SEQ ID NO: 81 - DNA, 735 bp, Homo sapiens
  source 1..735: mol_type = genomic DNA
ctatccaccagcaagactatcgacatggagctggtgaagcggaagcgcatcgaggccatccgcggccagatcctgtccaagctgcggctcgccagccccccgagccagggggaggtgccgcccggcccgctgcccgaggccgtgctcgccctgtacaacagcacccgcgaccgggtggccggggagagtgcagaaccggagcccgagcctgaggccgactactacgccaaggaggtcacccgcgtgctaatggtggaaacccacaacgaaatctatgacaagttcaagcagagtacacacagcatatatatgttcttcaacacatcagagctccgagaagcggtacctgaacccgtgttgctctcccgggcagagctgcgtctgctgaggctcaagttaaaagtggagcagcacgtggagctgtaccagaaatacagcaacaattcctggcgatacctcagcaaccggctgctggcacccagcgactcgccagagtggttatcttttgatgtcaccggagttgtgcggcagtggttgagccgtggaggggaaattgagggctttcgccttagcgcccacagctccagcgacagcagggataacacactgcaagtggacatcaacgggttcactaccggccgccgaggtgacctggccaccattcatggcatgaaccggcctttcctgcttctcatggccaccccgctggagagggcccagcatctgcaaagctcc

SEQ ID NO: 82 - protein, 274 aa, Homo sapiens
MPPSGLRLLPLLLPLLWLLVLTPGRPAAGLSTCKTIDMELVKRKRIEAIRGQILSKLRLASPPSQGEVPPGPLPEAVLALYNSTRDRVAGESAEPEPEPEADYYAKEVTRVLMVETHNEIYDKFKQSTHSIYMFFNTSELREAVPEPVLLSRAELRLLRLKLKVEQHVELYQKYSNNSWRYLSNRLLAPSDSPEWLSFDVTGVVRQWLSRGGEIEGFRLSAHCSCDSRDNTLQVDINGFTTGRRGDLATIHGMNRPFLLLMATPLERAQHLQSS

SEQ ID NO: 83 - DNA, 822 bp, Homo sapiens
  source 1..822: mol_type = genomic DNA
atgccgccctccgggctgcggctgctgccgctgctgctaccgctgctgtggctactggtgctgacgcctggccggccggccgcgggactatccacctgcaagactatcgacatggagctggtgaagcggaagcgcatcgaggccatccgcggccagatcctgtccaagctgcggctcgccagccccccgagccagggggaggtgccgcccggcccgctgcccgaggccgtgctcgccctgtacaacagcacccgcgaccgggtggccggggagagtgcagaaccggagcccgagcctgaggccgactactacgccaaggaggtcacccgcgtgctaatggtggaaacccacaacgaaatctatgacaagttcaagcagagtacacacagcatatatatgttcttcaacacatcagagctccgagaagcggtacctgaacccgtgttgctctcccgggcagagctgcgtctgctgaggctcaagttaaaagtggagcagcacgtggagctgtaccagaaatacagcaacaattcctggcgatacctcagcaaccggctgctggcacccagcgactcgccagagtggttatcttttgatgtcaccggagttgtgcggcagtggttgagccgtggaggggaaattgagggctttcgccttagcgcccactgctcctgtgacagcagggataacacactgcaagtggacatcaacgggttcactaccggccgccgaggtgacctggccaccattcatggcatgaaccggcctttcctgcttctcatggccaccccgctggagagggcccagcatctgcaaagctcc

SEQ ID NO: 84 - protein, 91 aa, Homo sapiens
EVPQLTDLSFVDITDSSIGLRWTPLNSSTIIGYRITVVAAGEGIPIFEDFVDSSVGYYTVTGLEPGIDYDISVITLINGGESAPTTLTQQT

SEQ ID NO: 85 - protein, 4 aa, unidentified
RHRR

SEQ ID NO: 86 - protein, 4 aa, unidentified
RRKR

SEQ ID NO: 87 - protein, 4 aa, unidentified
RKKR

SEQ ID NO: 88 - protein, 245 aa, Homo sapiens; identical to SEQ ID NO: 31

SEQ ID NO: 89 - DNA, 735 bp, Homo sapiens; identical to SEQ ID NO: 32
  source 1..735: mol_type = genomic DNA

SEQ ID NO: 90 - protein, 112 aa, synthetic construct
SLDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 91 - DNA, 339 bp, synthetic construct
  source 1..339: mol_type = other DNA
tccctggacaccaactattgcttcagctccacggagaagaactgctgcgtgcggcagctgtacattgacttccgcaaggacctcggctggaagtggatccacgagcccaagggctaccatgccaacttctgcctcgggccctgcccctacatttggagcctggacacgcagtacagcaaggtcctggccctgtacaaccagcataacccgggcgcctcggcggcgccgtgctgcgtgccgcaggcgctggagccgctgcccatcgtgtactacgtgggccgcaagcccaaggtggagcagctgtccaacatgatcgtgcgctcctgcaagtgcagctga

SEQ ID NO: 92 - protein, 361 aa, synthetic construct
LSTSKTIDMELVKRKRIEAIRGQILSKLRLASPPSQGEVPPGPLPEAVLALYNSTRDRVAGESAEPEPEPEADYYAKEVTRVLMVETHNEIYDKFKQSTHSIYMFFNTSELREAVPEPVLLSRAELRLLRLKLKVEQHVELYQKYSNNSWRYLSNRLLAPSDSPEWLSFDVTGVVRQWLSRGGEIEGFRLSAHCSCDSRDNTLQVDINGFTTGRRGDLATIHGMNRPFLLLMATPLERAQHLQSSRRKRSLDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 93 - DNA, 1086 bp, synthetic construct
  source 1..1086: mol_type = other DNA
ctatccaccagcaagactatcgacatggagctggtgaagcggaagcgcatcgaggccatccgcggccagatcctgtccaagctgcggctcgccagccccccgagccagggggaggtgccgcccggcccgctgcccgaggccgtgctcgccctgtacaacagcacccgcgaccgggtggccggggagagtgcagaaccggagcccgagcctgaggccgactactacgccaaggaggtcacccgcgtgctaatggtggaaacccacaacgaaatctatgacaagttcaagcagagtacacacagcatatatatgttcttcaacacatcagagctccgagaagcggtacctgaacccgtgttgctctcccgggcagagctgcgtctgctgaggctcaagttaaaagtggagcagcacgtggagctgtaccagaaatacagcaacaattcctggcgatacctcagcaaccggctgctggcacccagcgactcgccagagtggttatcttttgatgtcaccggagttgtgcggcagtggttgagccgtggaggggaaattgagggctttcgccttagcgcccactgctcctgtgacagcagggataacacactgcaagtggacatcaacgggttcactaccggccgccgaggtgacctggccaccattcatggcatgaaccggcctttcctgcttctcatggccaccccgctggagagggcccagcatctgcaaagctcccggaggaagcgatccctggacaccaactattgcttcagctccacggagaagaactgctgcgtgcggcagctgtacattgacttccgcaaggacctcggctggaagtggatccacgagcccaagggctaccatgccaacttctgcctcgggccctgcccctacatttggagcctggacacgcagtacagcaaggtcctggccctgtacaaccagcataacccgggcgcctcggcggcgccgtgctgcgtgccgcaggcgctggagccgctgcccatcgtgtactacgtgggccgcaagcccaaggtggagcagctgtccaacatgatcgtgcgctcctgcaagtgcagctga

SEQ ID NO: 94 - protein, 278 aa, synthetic construct
LSTSSTLDMDQFMRKRIEAIRGQILSKLKLTSPPEDYPEPEEVPPEVISIYNSTRDLLQEKASRRAAACERERSDEEYYAKEVYKIDMPPFFPSENAIPPTFYRPYFRIVRFDVSAMEKNASNLVKAEFRVFRLQNPKARVPEQRIELYQILKSKDLTSPTQRYIDSKVVKTRAEGEWLSFDVTDAVHEWLHHKDRNLGFKISLHCPCCTFVPSNNYIIPNKSEELEARFAGIDGTSTYTSGDQKTIKSTRKKNSGKTPHLLLMLLPSYRLESQQTNR

SEQ ID NO: 95 - DNA, 834 bp, synthetic construct
  source 1..834: mol_type = other DNA
ctgtctaccagcagcacactcgatatggaccagttcatgcgcaagaggatcgaggcgatccgcgggcagatcctgagcaagctgaagctcaccagtcccccagaagactatcctgagcccgaggaagtccccccggaggtgatttccatctacaacagcaccagggacttgctccaggagaaggcgagccggagggcggccgcctgcgagcgcgagaggagcgacgaagagtactacgccaaggaggtttacaaaatagacatgccgcccttcttcccctccgaaaatgccatcccgcccactttctacagaccctacttcagaattgttcgatttgacgtctcagcaatggagaagaatgcttccaatttggtgaaagcagagttcagagtctttcgtttgcagaacccaaaagccagagtgcctgaacaacggattgagctatatcagattctcaagtccaaagatttaacatctccaacccagcgctacatcgacagcaaagttgtgaaaacaagagcagaaggcgaatggctctccttcgatgtaactgatgctgttcatgaatggcttcaccataaagacaggaacctgggatttaaaataagcttacactgtccctgctgcacttttgtaccatctaataattacatcatcccaaataaaagtgaagaactagaagcaagatttgcaggtattgatggcacctccacatataccagtggtgatcagaaaactataaagtccactaggaaaaaaaacagtgggaagaccccacatctcctgctaatgttattgccctcctacagacttgagtcacaacagaccaaccgg

SEQ ID NO: 96 - protein, 10 aa, synthetic construct
GSGESGGGSG

SEQ ID NO: 97 - DNA, 30 bp, synthetic construct
  source 1..30: mol_type = other DNA
ggaagcggcgaaagcggcggtggcagcgga

SEQ ID NO: 98 - protein, 326 aa, synthetic construct; identical to SEQ ID NO: 15

SEQ ID NO: 99 - DNA, 978 bp, synthetic construct
  source 1..978: mol_type = other DNA
gcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccgtgcccagcaccacctgtggcaggaccatcagtcttcctgttccccccaaaacccaaggacactctcatgatatcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaa

SEQ ID NO: 100 - protein, 116 aa, synthetic construct [sequence omitted]

SEQ ID NO: 101 - DNA, 348 bp, synthetic construct
  source 1..348: mol_type = other DNA
gaggtgcagctgttggagtctgggggaggcttggtacagcctggggggtccctgagactctcctgtgcagcctctggattcacctttagcagtttttcgatgagctgggtccgccaggctccagggaaggggctggagtgggtctcatctattagtggtagttcgggtaccacatactacgcagactccgtgaagggccggttcaccatctccagagacaattccaagaacacgctgtatctgcaaatgaacagcctgagagccgaagacacggccgtatattactgtgcgaaaccgtttccgtattttgactactggggccagggaaccctggtcaccgtctccagt

SEQ ID NO: 102 - protein, 5 aa, synthetic construct
SFSMS

SEQ ID NO: 103 - protein, 17 aa, synthetic construct
SISGSSGTTYYADSVKG

SEQ ID NO: 104 - protein, 7 aa, synthetic construct
PFPYFDY

SEQ ID NO: 105 - protein, 108 aa, synthetic construct [sequence omitted]

SEQ ID NO: 106 - DNA, 324 bp, synthetic construct
  source 1..324: mol_type = other DNA
gaaattgtgttgacgcagtctccaggcaccctgtctttgtctccaggggaaagagccaccctctcctgcagggccagtcagagtgttagcagcagctttttagcctggtaccagcagaaacctggccaggctcccaggctcctcatctattatgcatccagcagggccactggcatcccagacaggttcagtggcagtgggtctgggacagacttcactctcaccatcagcagactggagcctgaagattttgcagtgtattactgtcagcagacgggtcgtattccgccgacgttcggccaagggaccaaggtggaaatcaaa

SEQ ID NO: 107 - protein, 12 aa, synthetic construct
RASQSVSSSFLA

SEQ ID NO: 108 - protein, 7 aa, synthetic construct
YASSRAT

SEQ ID NO: 109 - protein, 9 aa, synthetic construct
QQTGRIPPT

SEQ ID NO: 110 - protein, 842 aa, synthetic construct
MHRPRRRGTRPPPLALLAALLLAARGADAEVQLLESGGGLVQPGGSLRLSCAASGFTFSSFSMSWVRQAPGKGLEWVSSISGSSGTTYYADSVKGRFTISRDNSKNTLYLQMNSLRAEDTAVYYCAKPFPYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPGGGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGSGESGGGSGLSTSKTIDMELVKRKRIEAIRGQILSKLRLASPPSQGEVPPGPLPEAVLALYNSTRDRVAGESAEPEPEPEADYYAKEVTRVLMVETHNEIYDKFKQSTHSIYMFFNTSELREAVPEPVLLSRAELRLLRLKLKVEQHVELYQKYSNNSWRYLSNRLLAPSDSPEWLSFDVTGVVRQWLSRGGEIEGFRLSAHCSCDSRDNTLQVDINGFTTGRRGDLATIHGMNRPFLLLMATPLERAQHLQSSRRKRSLDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 111 - DNA, 2529 bp, synthetic construct
  source 1..2529: mol_type = other DNA
atgcacagacctagacgtcgtggaactcgtccacctccactggcactgctcgctgctctcctcctggctgcacgtggtgctgatgcagaggtgcagctgttggagtctgggggaggcttggtacagcctggggggtccctgagactctcctgtgcagcctctggattcacctttagcagtttttcgatgagctgggtccgccaggctccagggaaggggctggagtgggtctcatctattagtggtagttcgggtaccacatactacgcagactccgtgaagggccggttcaccatctccagagacaattccaagaacacgctgtatctgcaaatgaacagcctgagagccgaagacacggccgtatattactgtgcgaaaccgtttccgtattttgactactggggccagggaaccctggtcaccgtctccagtgcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccgtgcccagcaccaggcggtggcggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaaggaagcggcgaaagcggcggtggcagcggactatccaccagcaagactatcgacatggagctggtgaagcggaagcgcatcgaggccatccgcggccagatcctgtccaagctgcggctcgccagccccccgagccagggggaggtgccgcccggcccgctgcccgaggccgtgctcgccctgtacaacagcacccgcgaccgggtggccggggagagtgcagaaccggagcccgagcctgaggccgactactacgccaaggaggtcacccgcgtgctaatggtggaaacccacaacgaaatctatgacaagttcaagcagagtacacacagcatatatatgttcttcaacacatcagagctccgagaagcggtacctgaacccgtgttgctctcccgggcagagctgcgtctgctgaggctcaagttaaaagtggagcagcacgtggagctgtaccagaaatacagcaacaattcctggcgatacctcagcaaccggctgctggcacccagcgactcgccagagtggttatcttttgatgtcaccggagttgtgcggcagtggttgagccgtggaggggaaattgagggctttcgccttagcgcccactgctcctgtgacagcagggataacacactgcaagtggacatcaacgggttcactaccggccgccgaggtgacctggccaccattcatggcatgaaccggcctttcctgcttctcatggccaccccgctggagagggcccagcatctgcaaagctcccggaggaagcgatccctggacaccaactattgcttcagctccacggagaagaactgctgcgtgcggcagctgtacattgacttccgcaaggacctcggctggaagtggatccacgagcccaagggctaccatgccaacttctgcctcgggccctgcccctacatttggagcctggacacgcagtacagcaaggtcctggccctgtacaaccagcataacccgggcgcctcggcggcgccgtgctgcgtgccgcaggcgctggagccgctgcccatcgtgtactacgtgggccgcaagcccaaggtggagcagctgtccaacatgatcgtgcgctcctgcaagtgcagctga

SEQ ID NO: 112 - protein, 875 aa, synthetic construct
MHRPRRRGTRPPPLALLAALLLAARGADAEVQLLESGGGLVQPGGSLRLSCAASGFTFSSFSMSWVRQAPGKGLEWVSSISGSSGTTYYADSVKGRFTISRDNSKNTLYLQMNSLRAEDTAVYYCAKPFPYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGSGESGGGSGLSTSSTLDMDQFMRKRIEAIRGQILSKLKLTSPPEDYPEPEEVPPEVISIYNSTRDLLQEKASRRAAACERERSDEEYYAKEVYKIDMPPFFPSENAIPPTFYRPYFRIVRFDVSAMEKNASNLVKAEFRVFRLQNPKARVPEQRIELYQILKSKDLTSPTQRYIDSKVVKTRAEGEWLSFDVTDAVHEWLHHKDRNLGFKISLHCPCCTFVPSNNYIIPNKSEELEARFAGIDGTSTYTSGDQKTIKSTRKKNSGKTPHLLLMLLPSYRLESQQTNRRKKRALDAAYCFRNVQDNCCLRPLYIDFKRDLGWKWIHEPKGYNANFCAGACPYLWSSDTQHSRVLSLYNTINPEASASPCCVSQDLEPLTILYYIGKTPKIEQLSNMIVKSCKCS

SEQ ID NO: 113 - DNA, 2628 bp, synthetic construct
  source 1..2628: mol_type = other DNA
atgcacagacctagacgtcgtggaactcgtccacctccactggcactgctcgctgctctcctcctggctgcacgtggtgctgatgcagaggtgcagctgttggagtctgggggaggcttggtacagcctggggggtccctgagactctcctgtgcagcctctggattcacctttagcagtttttcgatgagctgggtccgccaggctccagggaaggggctggagtgggtctcatctattagtggtagttcgggtaccacatactacgcagactccgtgaagggccggttcaccatctccagagacaattccaagaacacgctgtatctgcaaatgaacagcctgagagccgaagacacggccgtatattactgtgcgaaaccgtttccgtattttgactactggggccagggaaccctggtcaccgtctccagtgcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccgtgcccagcaccaggcggtggcggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaaggaagcggcgaaagcggcggtggcagcggactgtctaccagcagcacactcgatatggaccagttcatgcgcaagaggatcgaggcgatccgcgggcagatcctgagcaagctgaagctcaccagtcccccagaagactatcctgagcccgaggaagtccccccggaggtgatttccatctacaacagcaccagggacttgctccaggagaaggcgagccggagggcggccgcctgcgagcgcgagaggagcgacgaagagtactacgccaaggaggtttacaaaatagacatgccgcccttcttcccctccgaaaatgccatcccgcccactttctacagaccctacttcagaattgttcgatttgacgtctcagcaatggagaagaatgcttccaatttggtgaaagcagagttcagagtctttcgtttgcagaacccaaaagccagagtgcctgaacaacggattgagctatatcagattctcaagtccaaagatttaacatctccaacccagcgctacatcgacagcaaagttgtgaaaacaagagcagaaggcgaatggctctccttcgatgtaactgatgctgttcatgaatggcttcaccataaagacaggaacctgggatttaaaataagcttacactgtccctgctgcacttttgtaccatctaataattacatcatcccaaataaaagtgaagaactagaagcaagatttgcaggtattgatggcacctccacatataccagtggtgatcagaaaactataaagtccactaggaaaaaaaacagtgggaagaccccacatctcctgctaatgttattgccctcctacagacttgagtcacaacagaccaaccggcggaagaagcgtgctttggatgcggcctattgctttagaaatgtgcaggataattgctgcctacgtccactttacattgatttcaagagggatctagggtggaaatggatacacgaacccaaagggtacaatgccaacttctgtgctggagcatgcccgtatttatggagttcagacactcagcacagcagggtcctgagcttatataataccataaatccagaagcatctgcttctccttgctgcgtgtcccaagatttagaacctctaaccattctctactacattggcaaaacacccaagattgaacagctttctaatatgattgtaaagtcttgcaaatgcagctaa

SEQ ID NO: 114 - protein, 244 aa, synthetic construct
MHRPRRRGTRPPPLALLAALLLAARGADAEIVLTQSPGTLSLSPGERATLSCRASQSVSSSFLAWYQQKPGQAPRLLIYYASSRATGIPDRFSGSGSGTDFTLTISRLEPEDFAVYYCQQTGRIPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 115 - DNA, 735 bp, synthetic construct
  source 1..735: mol_type = other DNA
atgcacagacctagacgtcgtggaactcgtccacctccactggcactgctcgctgctctcctcctggctgcacgtggtgctgatgcagaaattgtgttgacgcagtctccaggcaccctgtctttgtctccaggggaaagagccaccctctcctgcagggccagtcagagtgttagcagcagctttttagcctggtaccagcagaaacctggccaggctcccaggctcctcatctattatgcatccagcagggccactggcatcccagacaggttcagtggcagtgggtctgggacagacttcactctcaccatcagcagactggagcctgaagattttgcagtgtattactgtcagcagacgggtcgtattccgccgacgttcggccaagggaccaaggtggaaatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 116 - protein, 298 aa, Homo sapiens
MHYCVLSAFLILHLVTVALSLSTCSTLDMDQFMRKRIEAIRGQILSKLKLTSPPEDYPEPEEVPPEVISIYNSTRDLLQEKASRRAAACERERSDEEYYAKEVYKIDMPPFFPSENAIPPTFYRPYFRIVRFDVSAMEKNASNLVKAEFRVFRLQNPKARVPEQRIELYQILKSKDLTSPTQRYIDSKVVKTRAEGEWLSFDVTDAVHEWLHHKDRNLGFKISLHCPCCTFVPSNNYIIPNKSEELEARFAGIDGTSTYTSGDQKTIKSTRKKNSGKTPHLLLMLLPSYRLESQQTNR

SEQ ID NO: 117 - DNA, 894 bp, Homo sapiens
  source 1..894: mol_type = genomic DNA
atgcactactgtgtgctgagcgcttttctgatcctgcatctggtcacggtcgcgctcagcctgtctacctgcagcacactcgatatggaccagttcatgcgcaagaggatcgaggcgatccgcgggcagatcctgagcaagctgaagctcaccagtcccccagaagactatcctgagcccgaggaagtccccccggaggtgatttccatctacaacagcaccagggacttgctccaggagaaggcgagccggagggcggccgcctgcgagcgcgagaggagcgacgaagagtactacgccaaggaggtttacaaaatagacatgccgcccttcttcccctccgaaaatgccatcccgcccactttctacagaccctacttcagaattgttcgatttgacgtctcagcaatggagaagaatgcttccaatttggtgaaagcagagttcagagtctttcgtttgcagaacccaaaagccagagtgcctgaacaacggattgagctatatcagattctcaagtccaaagatttaacatctccaacccagcgctacatcgacagcaaagttgtgaaaacaagagcagaaggcgaatggctctccttcgatgtaactgatgctgttcatgaatggcttcaccataaagacaggaacctgggatttaaaataagcttacactgtccctgctgcacttttgtaccatctaataattacatcatcccaaataaaagtgaagaactagaagcaagatttgcaggtattgatggcacctccacatataccagtggtgatcagaaaactataaagtccactaggaaaaaaaacagtgggaagaccccacatctcctgctaatgttattgccctcctacagacttgagtcacaacagaccaaccgg

SEQ ID NO: 118 - protein, 278 aa, Homo sapiens
LSTCSTLDMDQFMRKRIEAIRGQILSKLKLTSPPEDYPEPEEVPPEVISIYNSTRDLLQEKASRRAAACERERSDEEYYAKEVYKIDMPPFFPSENAIPPTFYRPYFRIVRFDVSAMEKNASNLVKAEFRVFRLQNPKARVPEQRIELYQILKSKDLTSPTQRYIDSKVVKTRAEGEWLSFDVTDAVHEWLHHKDRNLGFKISLHCPCCTFVPSNNYIIPNKSEELEARFAGIDGTSTYTSGDQKTIKSTRKKNSGKTPHLLLMLLPSYRLESQQTNR

SEQ ID NO: 119 - DNA, 894 bp, Homo sapiens; identical to SEQ ID NO: 117
  source 1..894: mol_type = genomic DNA

SEQ ID NO: 120 - protein, 847 aa, synthetic construct
MHRPRRRGTRPPPLALLAALLLAARGADAEVQLAESGGGLVQPGRSMKLSCAASGFTFSNFPMAWVRQAPTKGLEWVATISTSGGSTYYRDSVKGRFTISRDNAKSTLYLQMNSLRSEDTATYYCTRTLYILRVFYFDYWGQGVMVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGKGGGSGGGSGGGSLSTSKTIDMELVKRKRIEAIRGQILSKLRLASPPSQGEVPPGPLPEAVLALYNSTRDRVAGESAEPEPEPEADYYAKEVTRVLMVETHNEIYDKFKQSTHSIYMFFNTSELREAVPEPVLLSRAELRLLRLKLKVEQHVELYQKYSNNSWRYLSNRLLAPSDSPEWLSFDVTGVVRQWLSRGGEIEGFRLSAHCSCDSRDNTLQVDINGFTTGRRGDLATIHGMNRPFLLLMATPLERAQHLQSSRHRRALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 121 - DNA, 2544 bp, synthetic construct
  source 1..2544: mol_type = other DNA
atgcatcgaccacgacgacgaggtactcgaccacctcctctggccctgctggcagccctgctgctggctgccagaggcgccgacgccgaggtgcagctggctgagtctggcggaggcctggtgcagcccggccggtccatgaagctgagctgcgctgcttctggattcaccttctccaactttcctatggcctgggtcagacaggctcccaccaagggcctggaatgggtggctaccatctccacctcaggcgggtccacctactacagagactctgtgaagggcagattcaccatctctcgggataacgccaagtccaccctgtacctgcagatgaactccctgcggtctgaggataccgccacctactactgtaccaggaccctgtacatcctgagagtgttctacttcgactactggggccagggcgtgatggtgaccgtctcctccgccaagacaacccctccttctgtgtatcctctggcccctggctctgccgctcaaacaaattccatggtcaccctgggctgtctggtgaagggctactttcccgagcctgtgacggtgacctggaactccggctccctgagttctggcgtgcacacattccctgctgtgctgcagtccgacctgtataccctgtcttcttcggtcaccgtgccctctagcacctggccttccgaaaccgtgacctgcaacgtggcccatcctgccagttcgaccaaagtggacaagaagatcgtgccccgggactgcggctgcaaaccttgcatctgcacggtgcctgaagtgagtagcgtgttcatcttccctcctaagcccaaggacgtgctgaccatcaccctgacccctaaggtgacatgtgttgtggtggatatcagcaaggacgatcctgaagtgcagttttcctggttcgtggacgacgtggaagtccacaccgctcagacacagcctagagaggaacagttcaactccaccttccggtccgtgtccgagctgcccatcatgcaccaggactggctgaatgggaaagagttcaagtgcagagtgaactccgccgccttccctgccccaatcgaaaagaccatcagcaagaccaagggccgtcccaaggcccctcaggtgtacaccatccctccacctaaggagcagatggccaaggataaggtatccctgacatgcatgatcaccgacttctttcctgaggacatcaccgtggagtggcagtggaacggccagcctgccgagaactacaagaacacccagcctatcatggacaccgatggatcctacttcgtgtactccaagctgaacgtgcaaaaaagcaattgggaggctggcaacacctttacctgttctgtgctgcatgaaggcctgcacaaccaccacaccgagaagtctctctcccacagccctggcaagggcggcggcagcggtggcggatccggcggcggctctctgtccacctcgaagacaatcgacatggaactggtcaagagaaagcggatcgaggctatcagaggacagatcctgtccaagctgagactggccagccccccttctcagggcgaggtgcctccaggccccctgcctgaggccgtactggctctgtacaactccaccagagatagagtggctggcgaatccgcagagcctgagcccgagcctgaggccgactactacgccaaagaggtgacccgggtgctgatggtggagacacacaacgagatctacgacaagttcaagcagtctacccactccatctacatgttcttcaacacttctgagctccgggaagccgtgcctgaaccagtcttgttgtctagagccgagctgcgcctcctgagactgaagcttaaagttgagcagcacgtggaactgtaccagaagtactccaataactcttggcggtacctgtccaaccggctactggctccttctgattctcccgaatggttatccttcgacgtgaccggagtggtgcggcagtggctgtctcggggcggcgagattgagggcttcagactgtccgcccactgctcttgtgactccagagataacaccctgcaagtggacatcaacggcttcaccaccggcagaaggggcgacctggctaccatccacggcatgaatagacctttcctgctgctgatggctacacctctggagcgggcccagcacctgcagtcttctagacacagaagagctctggacacaaactactgcttctcctccacagagaagaactgctgtgtcagacaactctacatcgacttccggaaggacctgggatggaagtggatccatgagcctaagggctaccacgccaacttctgcctgggcccttgtccttacatctggtccctggatacccagtactccaaagtgctggccctgtacaaccagcataaccccggcgcctctgctgctccatgctgcgtgcctcaggccctggaaccactgcctatcgtgtattacgtgggccggaagcctaaggtggagcagctgagcaacatgatcgtgcgaagctgcaagtgctcctga

SEQ ID NO: 122 - protein, 243 aa, synthetic construct
MHRPRRRGTRPPPLALLAALLLAARGADADIQMTQSPASLSASLGETVSIECLASEGISNDLAWYQQKSGKSPQLLIYATSRLQDGVPSRFSGSGSGTRYSLKISGMQPEDEADYFCQQSYKYPWTFGGGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRGEC

SEQ ID NO: 123 - DNA, 732 bp, synthetic construct
  source 1..732: mol_type = other DNA
atgcatcgaccacgacgacgaggtactcgaccacctcctctggccctgctggctgctctgctgttggctgccaggggcgctgatgctgacatccagatgacccagtcccctgcttccctgtccgcctctctcggcgaaaccgtgtctatcgagtgcctggccagcgaaggcatctccaacgacctggcctggtatcaacaaaaatccggcaagtctcctcagctgctgatctacgccacatctcggctgcaggatggcgtcccctccagattctccggctccggaagcggaaccagatacagcctgaagatctctggcatgcagcctgaggacgaagccgactacttctgccagcagtcttacaagtacccttggaccttcggcggcggcactaagctggaactgaagagagccgatgccgctcctacagtgtccatcttccctccttcttctgagcagctgaccagcggaggcgcctccgtggtgtgctttctgaacaacttctaccccaaggatatcaacgtgaagtggaagatcgacggctccgagcggcagaacggcgtgctgaattcttggaccgaccaggactccaaggacagcacctactccatgtcttccaccctgacactgaccaaggacgagtacgagagacacaactcctacacctgcgaggccacccacaagacctctacctctcccatcgtgaaatccttcaaccggggcgagtgttga

SEQ ID NO: 124 - protein, 121 aa, synthetic construct
EVQLAESGGGLVQPGRSMKLSCAASGFTFSNFPMAWVRQAPTKGLEWVATISTSGGSTYYRDSVKGRFTISRDNAKSTLYLQMNSLRSEDTATYYCTRTLYILRVFYFDYWGQGVMVTVSS

SEQ ID NO: 125 - protein, 7 aa, synthetic construct
GFTFSNF

SEQ ID NO: 126 - protein, 6 aa, synthetic construct
STSGGS

SEQ ID NO: 127 - protein, 12 aa, synthetic construct
TLYILRVFYFDY

SEQ ID NO: 128 - protein, 324 aa, Mus musculus
AKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 129 - protein, 107 aa, synthetic construct
DIQMTQSPASLSASLGETVSIECLASEGISNDLAWYQQKSGKSPQLLIYATSRLQDGVPSRFSGSGSGTRYSLKISGMQPEDEADYFCQQSYKYPWTFGGGTKLELK

SEQ ID NO: 130 - protein, 11 aa, synthetic construct
LASEGISNDLA

SEQ ID NO: 131 - protein, 7 aa, synthetic construct
ATSRLQD

SEQ ID NO: 132 - protein, 9 aa, synthetic construct
QQSYKYPWT

SEQ ID NO: 133 - protein, 107 aa, Mus musculus
RADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRGEC

SEQ ID NO: 134 - protein, 843 aa, synthetic construct
MHRPRRRGTRPPPLALLAALLLAARGADAEVQLVESGGGLVQPGNSLKLSCAASGFTFSDSAMAWVRLSPKKGLEWVATITYDGSNTYYRDSVKGRFTISRDKPKSTLYLQMDSLRSEDTATYYCATLLGGYFDYWGQGVMVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGKGGGSGGGSGGGSLSTSKTIDMELVKRKRIEAIRGQILSKLRLASPPSQGEVPPGPLPEAVLALYNSTRDRVAGESAEPEPEPEADYYAKEVTRVLMVETHNEIYDKFKQSTHSIYMFFNTSELREAVPEPVLLSRAELRLLRLKLKVEQHVELYQKYSNNSWRYLSNRLLAPSDSPEWLSFDVTGVVRQWLSRGGEIEGFRLSAHCSCDSRDNTLQVDINGFTTGRRGDLATIHGMNRPFLLLMATPLERAQHLQSSRHRRALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 135 - DNA, 2535 bp, synthetic construct
  source 1..2535: mol_type = other DNA
atgcatcgaccacgacgacgaggtactcgaccccctccactggctctgctcgccgctctgctgctggctgcccggggcgccgacgccgaggtgcagctggtcgaatccggcggaggcctggtgcagcctggcaactccctgaagctgagttgcgccgcctctggcttcaccttttctgactctgccatggcttgggtccggctttcccccaagaagggcctggaatgggtggctaccatcacctacgatggcagcaacacctactaccgggacagcgtgaaaggcagattcactatctccagagataagcctaagtccaccctgtacctgcagatggactctctgagatctgaggacaccgccacctactactgtgccaccctgcttggcggctactttgactactggggccagggagtgatggtgaccgtgtcctccgccaagacaacccctccttctgtttaccctctggctcctggctctgctgcccagaccaactccatggtgaccctgggctgtctggtgaaaggctacttcccggagcctgtgaccgtcacctggaactccggatctctgtcctctggcgtgcacacctttcctgccgtgctgcagtccgatctgtacaccttgtcttcctccgtgaccgtgccttctagtacctggcctagcgagaccgtgacctgcaacgtggcccaccctgccagcagcacaaaggtggacaagaagatcgtgcctagagactgcgggtgcaagccctgcatctgcaccgttcctgaagtgtccagcgtgttcatcttccccccaaagcccaaggatgtcctgacaatcaccctgacccctaaagtcacctgtgtggttgtggacatcagcaaggacgaccctgaggtccaattctcctggttcgtggatgacgtggaagtccataccgcacaaacccagcctcgcgaggaacaattcaactccaccttcagatccgtgtctgagctgcctatcatgcaccaggactggctgaacggcaaggagttcaagtgcagagtgaactctgccgcttttcctgcccctattgagaagacaatcagcaagaccaagggcagacctaaggcccctcaggtgtacaccatcccaccacctaaagagcagatggccaaggacaaggtgtcccttacatgcatgatcaccgacttcttccctgaggatatcacagtggaatggcagtggaatggccagcccgctgagaactacaaaaacacacagcccatcatggataccgatggctcctactttgtgtattctaagctgaacgtgcagaaatccaattgggaggctggcaatacgttcacctgttctgtgctgcatgagggcctgcacaaccaccacaccgagaagtccctgagtcactctcccggcaagggcggcggctccggcggaggctctggcggaggcagcctctctacctctaagaccatcgacatggaactggtgaagagaaagcggatcgaagccatccggggccagatcctgtcaaagctgaggctggcctctcctccttctcagggcgaggtgcccccaggccctctgcccgaggccgtgctggccctgtacaacagcacccgggacagagtggccggcgagtccgcggaaccagagcctgagcctgaagctgactactacgccaaggaggtgaccagagtgctgatggtcgagacacacaacgagatctacgacaagttcaagcagagcacccactccatctacatgttcttcaacacctccgagctgagagaggccgtgcccgagcccgtgctgctgtcccgggccgagctgaggctcctgcggctgaagctgaaggtggaacagcacgtggaactataccagaagtactctaacaactcttggcggtatctgtctaacagactgctggccccatccgacagtcctgagtggctgtccttcgacgtgactggcgtggtgagacagtggctgtctcggggcggagaaatcgagggtttccggctgtccgctcactgttcctgcgatagccgcgataacacactgcaagtggacatcaacgggttcaccactggccggcggggcgacctggctaccatccacggcatgaacagacctttcctgctgctgatggctacacctctggaaagagctcagcatctgcagtccagccggcacagaagagccctggacacaaactactgcttctcctcaaccgagaaaaactgctgtgtgcggcagctgtacatcgacttcagaaaggacctgggctggaaatggatccacgaacctaagggctaccacgccaacttctgcctgggcccttgtccttacatctggtccctggatacccagtactccaaagtgctggctctgtacaaccagcacaatcctggagccagcgctgctccttgctgcgtgcctcaggccctcgaacccctgcctatcgtgtattacgtaggtagaaagcctaaggtggagcagctgtccaacatgatcgtgcggtcttgcaagtgctcttgataa

SEQ ID NO: 136 - protein, 243 aa, synthetic construct
MHRPRRRGTRPPPLALLAALLLAARGADADIQMTQSPASLSASPEEIVTITCQASQDIGNWLAWYQQKPGKSPQLLIYSATSLADGIPSRFSGSRSGTQYSLKISRLQVEETGIYYCLQRYSNPWTFGGGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRGEC

SEQ ID NO: 137 - DNA, 735 bp, synthetic construct
  source 1..735: mol_type = other DNA
atgcatcgaccacgacgacgaggtactcgaccacctcctctggccctgctggctgctctgctgctggctgccagaggcgccgatgccgacatccagatgacccagtcccctgcttctctgtctgcttctcccgaggaaatcgtgaccatcacctgtcaggcctctcaggatatcggcaactggctggcctggtaccagcagaagcctggcaagtcccctcagctgctgatctactctgccacaagcctggctgatggcatccccagccggttctccggctctagatccggcacccagtacagcctgaagatcagcagactgcaagtggaagaaaccggaatctactactgcctgcagagatactccaacccttggacatttggcggtggaaccaagctggaattgaagcgcgccgacgccgcccctaccgtgtccatcttccctccaagctctgagcagctgacctctggcggcgcttccgtggtgtgcttcctgaacaacttctaccccaaagacatcaacgtgaagtggaagatcgacggctctgagcggcagaacggcgtgctgaattcttggaccgaccaagactccaaggactctacttattccatgtcctccaccctgaccctcaccaaggacgagtacgagagacacaactcctacacctgcgaggccacccacaagacctccacatctcctattgtcaaatccttcaaccggggcgagtgctgataa

SEQ ID NO: 138 - protein, 117 aa, synthetic construct
EVQLVESGGGLVQPGNSLKLSCAASGFTFSDSAMAWVRLSPKKGLEWVATITYDGSNTYYRDSVKGRFTISRDKPKSTLYLQMDSLRSEDTATYYCATLLGGYFDYWGQGVMVTVSS

SEQ ID NO: 139 - protein, 8 aa, synthetic construct
GFTFSDSA

SEQ ID NO: 140 - protein, 8 aa, synthetic construct
ITYDGSNT

SEQ ID NO: 141 - protein, 10 aa, synthetic construct
ATLLGGYFDY

SEQ ID NO: 142 - protein, 107 aa, synthetic construct
DIQMTQSPASLSASPEEIVTITCQASQDIGNWLAWYQQKPGKSPQLLIYSATSLADGIPSRFSGSRSGTQYSLKISRLQVEETGIYYCLQRYSNPWTFGGGTKLELK

SEQ ID NO: 143 - protein, 6 aa, synthetic construct
QDIGNW

SEQ ID NO: 144 - skipped

SEQ ID NO: 145 - protein, 9 aa, synthetic construct
LQRYSNPWT

SEQ ID NO: 146 - protein, 844 aa, synthetic construct
MHRPRRRGTRPPPLALLAALLLAARGADAEVQLQQSGPELVKPGASVKISCKTSGYTFTEYTMHWVRQSHGKSLEWIGGINPNNGGSTYNQKFMVKATLTVDKSSSTAYMELRSLTSEDSAVYYCARDYDEAWFAYWGQGTLVTVSAAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGKGGGSGGGSGGGSLSTSKTIDMELVKRKRIEAIRGQILSKLRLASPPSQGEVPPGPLPEAVLALYNSTRDRVAGESAEPEPEPEADYYAKEVTRVLMVETHNEIYDKFKQSTHSIYMFFNTSELREAVPEPVLLSRAELRLLRLKLKVEQHVELYQKYSNNSWRYLSNRLLAPSDSPEWLSFDVTGVVRQWLSRGGEIEGFRLSAHCSCDSRDNTLQVDINGFTTGRRGDLATIHGMNRPFLLLMATPLERAQHLQSSRHRRALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 147 - DNA, 2535 bp, synthetic construct
  source 1..2535: mol_type = other DNA
atgcatcgaccacgacgacgaggtactcgaccccctccacttgcactgctggctgccctgctgctggctgcccggggagccgacgccgaagtgcagctgcagcagtctggccccgaactggtgaagcccggtgcctccgtcaagattagctgtaagacatctggatatacctttaccgagtacaccatgcactgggtgagacagtctcacggcaagtctctggagtggatcggcggcatcaaccctaacaacggcgggtctacctacaaccagaagttcatggtgaaggctaccctgaccgtggacaagagctcttctaccgcgtacatggaactgagatctctgacctccgaggattctgctgtgtactactgcgccagagattacgacgaggcctggttcgcctactggggccagggcaccctggtcaccgtgtccgctgctaagaccaccccccccagcgtgtaccccctggcccccggttctgccgcccagacgaattccatggtcacattgggctgtctggtgaagggctattttcccgagcctgtaaccgtgacctggaacagcggcagcctgtccagcggcgtgcacaccttccctgccgtgctgcagagcgacctgtacaccctgtcctcctccgttacagtgccttcatctacctggccttccgagacagtgacttgcaacgtggctcaccctgcttcatccaccaaggtcgataagaagatcgttcctcgggactgcggctgcaaaccctgcatctgcaccgtgcctgaagtgtcctctgtgttcatcttccctcccaaaccaaaggacgtgctcaccatcaccctgaccccaaaggtgacctgtgtggtcgtggacatctccaaggacgatcctgaggtgcagttcagctggttcgtggacgacgtcgaagtgcatacagctcagacccagcctagagaggaacagttcaactccacctttagatctgtgtctgagctgcctatcatgcaccaggattggctgaatggcaaagagttcaagtgcagagtgaactctgccgctttccctgctcctatcgagaagaccatctccaagaccaagggcagacccaaggcccctcaagtgtatactatccctcctcctaaggagcagatggccaaggacaaggtgtctctgacatgcatgatcaccgacttctttcctgaagatatcaccgtggaatggcagtggaacggacaacctgccgagaactacaagaacacccaacccatcatggacacagacggctcctacttcgtgtacagcaagctgaacgtgcaaaagtctaattgggaagctggcaacaccttcacctgttctgtgctgcacgagggactccacaaccaccataccgagaagagtctgtctcactcccctggaaagggcggcggctccggaggcggctctggcggcggctctctatctacctctaaaacaatcgacatggaactggtgaagcggaagcggatcgaggccatcagagggcagatcctgtcgaagctgagactggcttcccctccatctcagggcgaggtgcctcctggccctctgccagaggccgtcctggctctgtacaactccacacgagacagagtggctggcgagagtgccgaaccagagccagaaccagaggctgactactacgccaaagaggtgaccagagtgctgatggtagaaacccacaacgagatctacgacaagttcaagcagtctacccattccatctacatgttcttcaacacctccgaactgcgggaggccgtgcctgagcctgtgctgctttcccgggctgagctgcggctgctgcggctgaagctgaaagtggaacagcacgtggagctgtaccagaaatactccaacaactcctggcggtacctgagcaacagactgctggctccttccgactcccccgagtggctctccttcgacgtgacgggcgtggtgcggcagtggctgtccagaggcggcgaaatcgagggctttagactgtctgcccactgctcctgcgactcccgcgacaacaccttacaggtggatatcaacggcttcaccactggcagaagaggcgacctggccaccatccacggcatgaaccggcctttcctgctgctgatggccacccctctggagcgggcccaacacctgcagtcctctcggcacagacgcgctctggacaccaactactgcttctcctccacagaaaagaactgctgtgtgaggcagctgtacatcgacttcagaaaggatctgggctggaagtggatccacgagcctaaaggctaccacgccaacttctgcctgggaccttgtccttatatctggtccctggatacccagtactccaaggtgctggccctgtacaaccagcataatcctggcgctagcgctgccccgtgctgcgtgcctcaggccctggaacctctgcctattgtgtactacgtgggcagaaagcctaaagtggaacagctgagcaacatgatcgtacggtcctgcaagtgctcctga

SEQ ID NO: 148 - protein, 247 aa, synthetic construct
MHRPRRRGTRPPPLALLAALLLAARGADADIVLTQSPASLAVSLGQRATISCRASESIEYSGTSLMQWYQQKPGQPPKLLIYVASNVESGVPARFSGSGSGTDFSLNIHPVEEDDIGMYFCQQSRKLPYTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRGEC

SEQ ID NO: 149 - DNA, 744 bp, synthetic construct
  source 1..744: mol_type = other DNA
atgcatcgaccacgacgacgaggtactcgacccccccctctggctctgctggctgccctgctgctggccgccagaggcgccgatgctgatatcgtgctgacccagtctcctgcttctctggccgtcagcctgggccagagagctactatctcctgcagagcctctgagtccatcgagtattctggcacatctctgatgcagtggtaccagcagaagcctggccaacctccaaaattgctgatctacgtggcctccaacgtggaaagcggcgtgcctgcccgcttttctggctccggatctggtacagacttctccctgaacatccatcctgtggaagaggacgacatcggcatgtacttctgccagcaatctagaaagctgccttacaccttcggcggcggcaccaagctggaaatcaagcgggccgacgccgctcctaccgtgtccatctttcctccatctagcgagcagctgacctccggcggcgcttccgtggtgtgcttcctgaacaacttctaccctaaggacatcaacgtcaaatggaagatcgacggctccgaacggcagaacggagtgctgaattcctggaccgaccaggactccaaggattctacctacagcatgtcctccaccctgaccctcaccaaggacgagtacgagagacacaactcctacacctgtgaggccacacacaagaccagcacctctcccattgtgaagtccttcaaccggggcgagtgctga

SEQ ID NO: 150 - protein, 118 aa, synthetic construct
EVQLQQSGPELVKPGASVKISCKTSGYTFTEYTMHWVRQSHGKSLEWIGGINPNNGGSTYNQKFMVKATLTVDKSSSTAYMELRSLTSEDSAVYYCARDYDEAWFAYWGQGTLVTVSA

SEQ ID NO: 151 - protein, 8 aa, synthetic construct
GYTFTEYT

SEQ ID NO: 152 - protein, 8 aa, synthetic construct
INPNNGGS

SEQ ID NO: 153 - protein, 11 aa, synthetic construct
ARDYDEAWFAY

SEQ ID NO: 154 - protein, 111 aa, synthetic construct
DIVLTQSPASLAVSLGQRATISCRASESIEYSGTSLMQWYQQKPGQPPKLLIYVASNVESGVPARFSGSGSGTDFSLNIHPVEEDDIGMYFCQQSRKLPYTFGGGTKLEIK

SEQ ID NO: 155 - protein, 10 aa, synthetic construct
ESIEYSGTSL

SEQ ID NO: 156 - skipped

SEQ ID NO: 157 - protein, 9 aa, synthetic construct
QQSRKLPYT

SEQ ID NO: 158 - DNA, 354 bp, synthetic construct
  source 1..354: mol_type = other DNA
gaggtccagctgcaacagtctggacctgagctggtgaagcctggggcttcagtgaagatatcctgcaagacttctggatacacattcactgaatacaccatgcactgggtgaggcagagccatggaaagagccttgagtggattggaggtataaatcctaacaatggtggttctacctacaaccagaagttcatggtcaaggccacattgactgtagacaagtcctccagcacagcctacatggagctccgcagcctgacatctgaggattctgcagtctattactgtgcaagagattacgacgaggcctggtttgcttactggggccaagggactctggtcactgtctctgca

SEQ ID NO: 159 - DNA, 24 bp, synthetic construct
  source 1..24: mol_type = other DNA
ggatacacattcactgaatacacc

SEQ ID NO: 160 - DNA, 24 bp, synthetic construct
  source 1..24: mol_type = other DNA
ataaatcctaacaatggtggttct

SEQ ID NO: 161 - DNA, 33 bp, synthetic construct
  source 1..33: mol_type = other DNA
gcaagagattacgacgaggcctggtttgcttac

SEQ ID NO: 162 - DNA, 333 bp, synthetic construct
  source 1..333: mol_type = other DNA
gacattgtgctcacccaatctccagcttctttggctgtgtctcttgggcagagagccaccatctcctgcagagccagtgaaagtattgaatattctggcacaagtttaatgcagtggtaccaacagaaaccaggacagccacccaaactcctcatctatgttgcatccaacgtagaatctggggtccctgccaggtttagtggcagtgggtctgggacagacttcagcctcaacatccatcctgtggaggaggatgatattggaatgtatttctgtcagcaaagtaggaagcttccctacacgttcggaggggggaccaagctggaaataaaa

SEQ ID NO: 163 - DNA, 30 bp, synthetic construct
  source 1..30: mol_type = other DNA
gaaagtattgaatattctggcacaagttta

SEQ ID NO: 164 - skipped

SEQ ID NO: 165 - DNA, 27 bp, synthetic construct
  source 1..27: mol_type = other DNA
cagcaaagtaggaagcttccctacacg

SEQ ID NO: 166 - DNA, 351 bp, synthetic construct
  source 1..351: mol_type = other DNA
gaggtgcaacttgtggagtctggcggcggacttgtacagcccggaaattccctgaaactctcatgtgctgcatccggctttaccttttctgattctgctatggcctgggtaagactctcccctaagaaaggtcttgaatgggtggccacaattacatacgacggatctaacacatactatagagacagcgttaaaggaagatttacaattagtcgagataaaccaaaatctaccctctacttgcaaatggactctcttcgctctgaagatactgcaacctactattgtgcaacattgctcggcggctattttgattattggggacagggtgtgatggtgaccgtttcatca

SEQ ID NO: 167 - DNA, 24 bp, synthetic construct
  source 1..24: mol_type = other DNA
ggctttaccttttctgattctgct

SEQ ID NO: 168 - DNA, 24 bp, synthetic construct
  source 1..24: mol_type = other DNA
attacatacgacggatctaacaca

SEQ ID NO: 169 - DNA, 30 bp, synthetic construct
  source 1..30: mol_type = other DNA
gcaacattgctcggcggctattttgattat

SEQ ID NO: 170 - DNA, 321 bp, synthetic construct
  source 1..321: mol_type = other DNA
gacatacaaatgacccaatcccccgcttcacttagcgcatcccccgaagaaatcgtaaccataacatgccaagcctctcaggacattggaaattggttggcctggtatcagcaaaaacccggcaaaagcccacaactccttatttattccgcaacctccctcgccgatggcataccctcaagattctctggctcaagatctggtacacagtattccctgaaaatctcccgtctccaagtagaagaaactggcatttattattgtctccaaagatatagcaatccctggacctttggcggtggcacaaagctcgaattgaaa

SEQ ID NO: 171 - DNA, 18 bp, synthetic construct
  source 1..18: mol_type = other DNA
caggacattggaaattgg

SEQ ID NO: 172 - skipped

SEQ ID NO: 173 - DNA, 27 bp, synthetic construct
  source 1..27: mol_type = other DNA
ctccaaagatatagcaatccctggacc

SEQ ID NO: 174 - protein, 40 aa, synthetic construct
  VARIANT 1..40: note = This sequence may encompass 1-20 GS repeating units
GSGSGSGSGSGSGSGSGSGSGSGSGSGSGSGSGSGSGSGS

(19) United States
(12) Patent Application Publication
(10) Pub. No.: US 2026/0217778 A1
(43) Pub. Date: Jul. 30, 2026

(54) Compositions and Methods for Targeted Delivery of TGFß

(71) Applicant: Regeneron Pharmaceuticals, Inc., Tarrytown, NY (US)

(72) Inventors: Senem AYKUL, Ossining, NY (US); Vincent IDONE, Ridgefield, CT (US); Christopher SCHOENHERR, Piermont, NY (US); Aristides N. ECONOMIDES, Tarrytown, NY (US); Andrew J. MURPHY, Croton-on-Hudson, NY (US); Silvia SMALDONE, Scarsdale, NY (US)

(73) Assignee: Regeneron Pharmaceuticals, Inc., Tarrytown, NY (US)

(21) Appl. No.: 19/147,495

(22) PCT Filed: Jan. 11, 2024
(86) PCT No.: PCT/US2024/011162

§ 371 Date: Jul. 11, 2025

Related U.S. Application Data
(60) Provisional application No. 63/479,679, filed on Jan. 12, 2023.
(60) Provisional application No. 63/526,021, filed on Jul. 11, 2023.

(51) Int. Cl. C07K 14/495; A61K 38/00; A61P 17/02; C07K 16/18; C07K 16/28; C07K 16/30; C12N 15/864

(52) U.S. Cl. CPC C07K 14/495; A61K 38/00; A61P 17/02; C07K 16/18; C07K 16/2851; C07K 16/2896; C07K 16/30; C12N 15/8645; C07K 2319/50; C12N 2830/34

(57) ABSTRACT

The present disclosure provides a polypeptide complex comprising a target-binding polypeptide that binds a molecule on a target cell or a molecule in an extracellular matrix (ECM), and a small latent complex comprising, in particular, a dimeric latency associated polypeptide (LAP), or a fragment or derivative thereof, and a dimeric mature Transforming Growth Factor β (TGFP) family polypeptide, or a fragment or derivative thereof. Further provided are polypeptides (e.g., fusion polypeptides), as well as related polynucleotides, vectors, cells, and pharmaceutical compositions. Methods for treating subjects using, e.g., the polypeptide complexes and/or fusion polypeptides, or pharmaceutical compositions thereof, are also provided.

### CROSS-REFERENCE TO RELATED APPLICATIONS

This application claims priority to U.S. Provisional Application No. 63/479,679, filed Jan. 12, 2023, and U.S. Provisional Application No. 63/526,021, filed Jul. 11, 2023, the disclosures of both of which are herein incorporated by reference in their entireties.

### SEQUENCE LISTING

The instant application contains a Sequence Listing which has been submitted electronically in XML format and is hereby incorporated by reference in its entirety. Said XML copy, created on Jan. 8, 2024, is named 250298_000604_SL.xml and is 204,303 bytes in size.

### FIELD

The present disclosure provides a polypeptide complex comprising a target-binding polypeptide that binds a molecule on a target cell or a molecule in an extracellular matrix (ECM), and a small latent complex (SLC) comprising, in particular, a dimeric latency associated polypeptide (LAP), or a fragment or derivative thereof, and a dimeric mature Transforming Growth Factor β (TGFβ) family polypeptide, or a fragment or derivative thereof. Further provided are polypeptides (e.g., fusion polypeptides), as well as related polynucleotides, vectors, cells, and pharmaceutical compositions. Methods for treating subjects using, e.g., the polypeptide complexes and/or fusion polypeptides, or pharmaceutical compositions thereof, are also provided.

### BACKGROUND

Transforming growth factor beta (TGFβ, also known as TGFb, TGFB, TGFbeta, TGFBeta, and the like) is composed of a signal peptide, a latency-associated peptide (LAP), and a mature TGFβ domain. After furin cleavage at a furin cleavage site at the junction between the LAP and mature domain during secretion, the LAP stays non-covalently associated with the mature domain, thereby rendering the mature domain inactive. A complex of the LAP (e.g., the furin-cleaved LAP) and the mature domain is called a small latent complex (SLC). A SLC secreted in complex with a milieu molecule(s) is called a large latent complex (LLC), which can be expressed on different cell types (e.g., endothelial cells, T cells, macrophages, and microglia), and incorporated in the extracellular matrix (ECM). LLCs can be proteolytically or mechanically activated to release mature TGFβ which is capable of inducing downstream TGFβ signaling. Decreased TGFβ signaling has been identified in various TGFβ dysregulation disorders including inflammatory bowel disease (IBD), Marfan syndrome (MFS), autoimmune diseases, and other diseases associated with TGFβ loss-of-function mutations. Therefore, targeted TGFβ delivery to specific cell types which would be activated only once the TGFβ reaches a desired location is needed to benefit diseases and/or biological processes exacerbated by decreased TGFβ signaling.

### SUMMARY OF THE DISCLOSURE

As specified in the Background section above, there is a great need in the art for development of compositions and methods for the targeted delivery of TGFβ to specific cell types, in particular, in the context of decreased TGFβ signaling such as that which occurs in various TGFβ dysregulation disorders. The present application addresses these and other needs.

In one aspect, provided herein is a polypeptide complex comprising:
a. a target-binding polypeptide that binds a molecule on a target cell or a molecule in an extracellular matrix (ECM); and
b. a small latent complex (SLC) comprising:
   i. a dimeric latency associated polypeptide (LAP), or a fragment or derivative thereof, and
   ii. a dimeric mature Transforming Growth Factor β (TGFβ) family polypeptide, or a fragment or derivative thereof, wherein the mature TGFβ family polypeptide, or the fragment or derivative thereof, is inactive as a result of an interaction with the dimeric LAP, or the fragment or derivative thereof.

In some embodiments, the LAP, or the fragment or derivative thereof, is covalently attached to the target-binding polypeptide.

In some embodiments, the LAP, or the fragment or derivative thereof, is covalently attached to the target-binding polypeptide via a linker.

In some embodiments, the linker comprises the sequence (GGGGS)n (SEQ ID NO: 46), (GGGS)n (SEQ ID NO: 51), or GSGESGGGSG (SEQ ID NO: 96).

In some embodiments, the linker consists of the sequence (GGGGS)n (SEQ ID NO: 46), (GGGS)n (SEQ ID NO: 51), or GSGESGGGSG (SEQ ID NO: 96).

In some embodiments, the linker comprises the sequence GGGSGGGSGGGS (SEQ ID NO: 19).

In some embodiments, the linker consists of the sequence GGGSGGGSGGGS (SEQ ID NO: 19).

In some embodiments, the linker comprises the sequence GSGESGGGSG (SEQ ID NO: 96).

In some embodiments, the linker consists of the sequence GSGESGGGSG (SEQ ID NO: 96).

In some embodiments, the target-binding polypeptide binds both the LAP, or the fragment or derivative thereof, and the molecule on the target cell or the molecule in the ECM.

In some embodiments, the target-binding polypeptide is an antibody or a fragment or derivative thereof.

In some embodiments of any of the above-described polypeptide complexes, the mature TGFβ family polypeptide, or the fragment or derivative thereof, and the LAP, or the fragment or derivative thereof, are associated via a noncovalent interaction.

In some embodiments of any of the above-described polypeptide complexes, the mature TGFβ family polypeptide, or the fragment or derivative thereof, and the LAP, or the fragment or derivative thereof, are separated by a protease cleavage site.

In some embodiments, the protease cleavage site is a furin cleavage site.

In some embodiments, the furin cleavage site comprises the sequence RXXR (SEQ ID NO: 35).

In some embodiments, the furin cleavage site consists of the sequence RXXR (SEQ ID NO: 35).

In some embodiments, the RXXR (SEQ ID NO: 35) is RHRR (SEQ ID NO: 85), RRKR (SEQ ID NO: 86), or RKKR (SEQ ID NO: 87).

In some embodiments of any of the above-described polypeptide complexes, the mature TGFβ family polypeptide, or the fragment or derivative thereof, binds a Transforming Growth Factor β Receptor (TGFβR) upon release of the mature TGFβ family polypeptide, or the fragment or derivative thereof, from the SLC.

In some embodiments of any of the above-described polypeptide complexes, the mature TGFβ family polypeptide, or the fragment or derivative thereof, induces Smad2/3 signaling in the target cell or a cell adjacent to the target cell upon release from the SLC.

In some embodiments of any of the above-described polypeptide complexes, the LAP, or the fragment or derivative thereof, comprises an integrin binding motif.

In some embodiments, the integrin binding motif comprises the sequence RGD.

In some embodiments, the integrin is αvβ6 integrin or αvβ8 integrin.

In some embodiments of any of the above-described polypeptide complexes, the LAP, or the fragment or derivative thereof, does not comprise an integrin binding motif.

In some embodiments of any of the above-described polypeptide complexes, the target-binding polypeptide is an antigen-binding polypeptide or antigen-binding fragment thereof.

In some embodiments, the antigen-binding polypeptide is an antibody or antigen-binding fragment thereof.

In some embodiments, the antibody or antigen-binding fragment thereof comprises a heavy chain variable region.

In some embodiments, the antibody or antigen-binding fragment thereof comprises a light chain variable region.

In some embodiments, the antibody or antigen-binding fragment thereof comprises an immunoglobulin heavy chain constant domain.

In some embodiments, the immunoglobulin heavy chain constant domain is an IgG1 domain.

In some embodiments, the immunoglobulin heavy chain constant domain is an IgG4 domain.

In some embodiments, the antigen-binding polypeptide or the antigen-binding fragment thereof binds to CD63.

In some embodiments, the antigen-binding polypeptide or the antigen-binding fragment thereof binds to extra domain B of fibronectin (EDB-FN).

In some embodiments, the antigen-binding polypeptide or the antigen-binding fragment thereof binds to epithelial cell adhesion molecule (Epcam).

In some embodiments, the antigen-binding polypeptide or the antigen-binding fragment thereof binds to C-type lectin domain family 9 member A (Clec9a).

In some embodiments of any of the above-described polypeptide complexes, the target-binding polypeptide is not internalizing.

In some embodiments of any of the above-described polypeptide complexes, the mature TGFβ family polypeptide is a mature TGFβ polypeptide.

In some embodiments, the mature TGFβ polypeptide is a mature TGFβ1 polypeptide.

In some embodiments, the mature TGFβ1 polypeptide comprises the sequence of SEQ ID NO: 23.

In some embodiments, the mature TGFβ1 polypeptide consists of the sequence of SEQ ID NO: 23.

In some embodiments, the mature TGFβ1 polypeptide comprises the sequence of SEQ ID NO: 90.

In some embodiments, the mature TGFβ1 polypeptide consists of the sequence of SEQ ID NO: 90.

In some embodiments, the mature TGFβ polypeptide is a mature TGFβ2 polypeptide.

In some embodiments, the mature TGFβ2 polypeptide comprises the sequence of SEQ ID NO: 27.

In some embodiments, the mature TGFβ2 polypeptide consists of the sequence of SEQ ID NO: 27.

In some embodiments, the mature TGFβ family polypeptide is a mature Growth Differentiation Factor 8 (GDF8), a mature Growth Differentiation Factor 11 (GDF11) polypeptide, or a mature Bone Morphogenetic Protein 4 (BMP4).

In some embodiments of any of the above-described polypeptide complexes, the LAP comprises the sequence of positions 30-274 of the sequence of SEQ ID NO: 82.

In some embodiments, the LAP consists of the sequence of positions 30-274 of the sequence of SEQ ID NO: 82.

In some embodiments of any of the above-described polypeptide complexes, the LAP comprises the sequence of positions 21-298 of the sequence of SEQ ID NO: 116.

In some embodiments, the LAP consists of the sequence of positions 21-298 of the sequence of SEQ ID NO: 116.

In some embodiments of any of the above-described polypeptide complexes, the LAP, or the fragment or derivative thereof, is heterologous to the mature TGFβ family polypeptide, or the fragment or derivative thereof.

In some embodiments of any of the above-described polypeptide complexes, the LAP, or the fragment or derivative thereof, comprises one or more mutations, wherein the one or more mutations allow for proteolytic activation of the mature TGFβ family polypeptide, or the fragment or derivative thereof.

In some embodiments of any of the above-described polypeptide complexes, the LAP, or the fragment or derivative thereof, comprises one or more mutations, wherein the one or more mutations allow for mechanical activation of the mature TGFβ family polypeptide, or the fragment or derivative thereof.

In some embodiments of any of the above-described polypeptide complexes, the LAP, or the fragment or derivative thereof, comprises one or more mutations, wherein the one or more mutations eliminate one or more protease cleavage sites within the LAP, or the fragment or derivative thereof.

In some embodiments of any of the above-described polypeptide complexes, the LAP, or the fragment or derivative thereof, comprises one or more mutations, wherein the one or more mutations decrease binding of the LAP, or the fragment or derivative thereof, to a latency associated binding protein (LTBP).

In some embodiments, the one or more mutations comprise a C33S mutation, wherein position 33 is in relation to the sequence of SEQ ID NO: 82.

In some embodiments, the one or more mutations comprise a C24S mutation, wherein position 24 is in relation to SEQ ID NO: 116.

In some embodiments of any of the above-described polypeptide complexes, the LAP comprises the sequence of SEQ ID NO: 31.

In some embodiments, the LAP consists of the sequence of SEQ ID NO: 31.

In some embodiments of any of the above-described polypeptide complexes, the LAP comprises the sequence of SEQ ID NO: 94.

In some embodiments, the LAP consists of the sequence of SEQ ID NO: 94.

In some embodiments of any of the above-described polypeptide complexes, the mature TGFβ family polypeptide, or the fragment or derivative thereof, is chemically dissociated from the LAP, or the fragment or derivative thereof, and released in an active form from the SLC.

In some embodiments, the chemical dissociation comprises a protease treatment, a temperature treatment, an acid treatment, or any combination thereof.

In some embodiments of any of the above-described polypeptide complexes, the mature TGFβ family polypeptide, or the fragment or derivative thereof, is mechanically dissociated from the LAP, or the fragment or derivative thereof, and released in an active form from the SLC.

In some embodiments, the mechanical dissociation occurs a result of an interaction between the LAP, or the fragment or derivative thereof, and an integrin polypeptide.

In another aspect, provided herein is a pharmaceutical composition comprising a polypeptide complex described herein.

In some embodiments, a pharmaceutical composition described herein may further comprising a pharmaceutically acceptable carrier or diluent.

In another aspect, provided herein is a fusion polypeptide comprising:
a. a target-binding polypeptide that binds a molecule on a target cell or a molecule in an extracellular matrix (ECM);
b. a latency associated polypeptide (LAP), or a fragment or derivative thereof, and
c. a mature Transforming Growth Factor β (TGFβ) family polypeptide, or a fragment or derivative thereof.

In some embodiments, the mature TGFβ family polypeptide, or the fragment or derivative thereof, is inactive as a result of an interaction with the LAP, or the fragment or derivative thereof.

In some embodiments, the mature TGFβ family polypeptide, or the fragment or derivative thereof, binds a Transforming Growth Factor β Receptor (TGFβR) upon release of the mature TGFβ family polypeptide, or the fragment or derivative thereof, from the LAP, or the fragment or derivative thereof.

In some embodiments of any of the above described fusion polypeptides, the mature TGFβ family polypeptide, or the fragment or derivative thereof, induces Smad2/3 signaling in the target cell or a cell adjacent to the target cell upon release of the mature TGFβ family polypeptide, or the fragment or derivative thereof, from the LAP, or the fragment or derivative thereof.

In some embodiments of any of the above described fusion polypeptides, the fusion polypeptide comprises a linker.

In some embodiments, the linker is located between the target-binding polypeptide and the LAP, or the fragment or derivative thereof.

In some embodiments, the fusion polypeptide comprises, from N-terminus to C-terminus, (i) the target-binding polypeptide, (ii) the linker, (iii) the LAP, or the fragment or derivative thereof, and (iv) the mature TGFβ family polypeptide, or the fragment or derivative thereof.

In some embodiments, the fusion polypeptide comprises, from N-terminus to C-terminus, (i) the mature TGFβ family polypeptide, or the fragment or derivative thereof, (ii) the LAP, or the fragment or derivative thereof, (iii) the linker, and (iv) the target-binding polypeptide.

In some embodiments of any of the above described fusion polypeptides, the linker comprises the sequence (GGGGS)n (SEQ ID NO: 46), (GGGS)n (SEQ ID NO: 51), or GSGESGGGSG (SEQ ID NO: 96).

In some embodiments, the linker consists of the sequence (GGGGS)n (SEQ ID NO: 46), (GGGS)n (SEQ ID NO: 51), or GSGESGGGSG (SEQ ID NO: 96).

In some embodiments, the linker comprises the sequence GGGSGGGSGGGS (SEQ ID NO: 19).

In some embodiments, the linker consists of the sequence GGGSGGGSGGGS (SEQ ID NO: 19).

In some embodiments, the linker comprises the sequence GSGESGGGSG (SEQ ID NO: 96).

In some embodiments, the linker consists of the sequence GSGESGGGSG (SEQ ID NO: 96).

In some embodiments of any of the above-described fusion polypeptides, the mature TGFβ family polypeptide, or the fragment or derivative thereof, and the LAP, or the fragment or derivative thereof, are separated by a protease cleavage site.

In some embodiments, the protease cleavage site is a furin cleavage site.

In some embodiments, the furin cleavage site comprises the sequence RXXR (SEQ ID NO: 35).

In some embodiments, the furin cleavage site consists of the sequence RXXR (SEQ ID NO: 35).

In some embodiments, the RXXR (SEQ ID NO: 35) is RHRR (SEQ ID NO: 85), RRKR (SEQ ID NO: 86), or RKKR (SEQ ID NO: 87).

In some embodiments of any of the above-described fusion polypeptides, the fusion polypeptide further comprises a signal peptide.

In some embodiments, the signal peptide is mROR signal peptide.

In some embodiments, the mROR signal peptide comprises the sequence MHRPRRRGTRPPPLALLAALLLAARGADA (SEQ ID NO: 1).

In some embodiments, the mROR signal peptide consists of the sequence MHRPRRRGTRPPPLALLAALLLAARGADA (SEQ ID NO: 1).

In some embodiments of any of the above-described fusion polypeptides, the LAP, or the fragment or derivative thereof, comprises an integrin binding motif.

In some embodiments, the integrin binding motif comprises the sequence RGD.

In some embodiments, the integrin is αvβ6 integrin or αvβ8 integrin.

In some embodiments of any of the above-described fusion polypeptides, the LAP, or the fragment or derivative thereof, does not comprise an integrin binding motif.

In some embodiments of any of the above-described fusion polypeptides, the target-binding polypeptide is an antigen-binding polypeptide or antigen-binding fragment thereof.

In some embodiments, the antigen-binding polypeptide is an antibody or antigen-binding fragment thereof.

In some embodiments, the antibody or antigen-binding fragment thereof comprises a heavy chain variable region.

In some embodiments, the antibody or antigen-binding fragment thereof comprises a light chain variable region.

In some embodiments, the antibody or antigen-binding fragment thereof comprises an immunoglobulin heavy chain constant domain.

In some embodiments, the immunoglobulin heavy chain constant domain is an IgG1 domain.

In some embodiments, the immunoglobulin heavy chain constant domain is an IgG4 domain.

In some embodiments, the antigen-binding polypeptide or the antigen-binding fragment thereof binds to CD63.

In some embodiments, the antigen binding polypeptide of the antigen-binding fragment thereof binds to extra domain B of fibronectin (EDB-FN).

In some embodiments, the antigen binding polypeptide of the antigen-binding fragment thereof binds to epithelial cell adhesion molecule (Epcam).

In some embodiments, the antigen binding polypeptide of the antigen-binding fragment thereof binds to C-type lectin domain family 9 member A (Clec9a).

In some embodiments of any of the above-described fusion polypeptides, the target-binding polypeptide is not internalizing.

In some embodiments of any of the above-described fusion polypeptides, the mature TGFβ family polypeptide is a mature TGFβ polypeptide.

In some embodiments, the mature TGFβ polypeptide is a mature TGFβ1 polypeptide.

In some embodiments, the mature TGFβ1 polypeptide comprises the sequence of SEQ ID NO: 23.

In some embodiments, the mature TGFβ1 polypeptide consists of the sequence of SEQ ID NO: 23.

In some embodiments, the mature TGFβ1 polypeptide comprises the sequence of SEQ ID NO: 90.

In some embodiments, the mature TGFβ1 polypeptide comprises the sequence of SEQ ID NO: 90.

In some embodiments, the mature TGFβ polypeptide is a mature TGFβ2 polypeptide.

In some embodiments, the mature TGFβ2 polypeptide comprises the sequence of SEQ ID NO: 27.

In some embodiments, the mature TGFβ2 polypeptide consists of the sequence of SEQ ID NO: 27.

In some embodiments of any of the above-described fusion polypeptides, the mature TGFβ family polypeptide is a mature Growth Differentiation Factor 8 (GDF8), a mature Growth Differentiation Factor 11 (GDF11) polypeptide, or a mature Bone Morphogenetic Protein 4 (BMP4).

In some embodiments of any of the above-described fusion polypeptides, the LAP comprises the sequence of positions 30-274 of the sequence of SEQ ID NO: 82.

In some embodiments, the LAP consists of the sequence of positions 30-274 of the sequence of SEQ ID NO: 82.

In some embodiments of any of the above-described fusion polypeptides, the LAP comprises the sequence of positions 21-298 of the sequence of SEQ ID NO: 116.

In some embodiments, the LAP consists of the sequence of positions 21-298 of the sequence of SEQ ID NO: 116.

In some embodiments of any of the above-described fusion polypeptides, the LAP, or the fragment or derivative thereof, is heterologous to the mature TGFβ family polypeptide, or the fragment or derivative thereof.

In some embodiments of any of the above-described fusion polypeptides, the LAP, or the fragment or derivative thereof, comprises one or more mutations, wherein the one or more mutations allow for proteolytic activation of the mature TGFβ family polypeptide, or the fragment or derivative thereof.

In some embodiments of any of the above-described fusion polypeptides, the LAP, or the fragment or derivative thereof, comprises one or more mutations, wherein the one or more mutations allow for mechanical activation of the mature TGFβ family polypeptide, or the fragment or derivative thereof.

In some embodiments of any of the above-described fusion polypeptides, the LAP, or the fragment or derivative thereof, comprises one or more mutations, wherein the one or more mutations eliminate one or more protease cleavage sites within the LAP, or the fragment or derivative thereof.

In some embodiments of any of the above-described fusion polypeptides, the LAP, or the fragment or derivative thereof, comprises one or more mutations, wherein the one or more mutations decrease binding of the LAP, or the fragment or derivative thereof, to a latency associated binding protein (LTBP).

In some embodiments, the one or more mutations comprise a C33S mutation, wherein position 33 is in relation to the sequence of SEQ ID NO: 82.

In some embodiments, the one or more mutations comprise a C24S mutation, wherein position 24 is in relation to SEQ ID NO: 116

In some embodiments, the LAP comprises the sequence of SEQ ID NO: 31.

In some embodiments, the LAP consists of the sequence of SEQ ID NO: 31.

In some embodiments, the LAP comprises the sequence of SEQ ID NO: 94.

In some embodiments, the LAP consists of the sequence of SEQ ID NO: 94.

In another aspect, provided herein is a polynucleotide encoding a fusion polypeptide described herein.

In another aspect, provided herein is a vector comprising a polynucleotide described herein.

In some embodiments, the sequence encoding the fusion polypeptide is operably linked to a promoter, wherein the promoter mediates expression of the fusion polypeptide.

In some embodiments, the vector is a viral vector.

In some embodiments, the viral vector is an adeno-associated virus (AAV) vector.

In another aspect, provided herein is a cell comprising a polypeptide complex described herein, a fusion polypeptide described herein, a polynucleotide described herein, or a vector described herein.

In another aspect, provided herein is a method of making a polypeptide complex described herein comprising incubating a cell comprising the polynucleotide described herein, or a vector described herein, under conditions allowing for production of the polypeptide complex.

In some embodiments, the method further comprises collecting the cell culture medium and isolating the produced polypeptide complex by a process comprising affinity chromatography.

In some embodiments, the affinity chromatography comprises a Protein A or a Protein G column or beads.

In another aspect, provided herein is a method for treating a TGFβ dysregulation disorder in a subject in need thereof, comprising administering to the subject a therapeutically effective amount of a polypeptide complex described herein, a pharmaceutical composition described herein, a polynucleotide described herein, or a vector described herein.

In some embodiments, the polypeptide complex, pharmaceutical composition, polynucleotide, or vector is administered via injection.

In some embodiments, the injection is intravenous, intramuscular, subcutaneous, or intraperitoneal.

In some embodiments of any of the above-described methods for treating a TGFβ dysregulation disorder in a subject in need thereof, the polypeptide complex, pharmaceutical composition, polynucleotide, or vector is administered via hydrodynamic delivery (HDD).

In some embodiments of any of the above-described methods for treating a TGFβ dysregulation disorder in a subject in need thereof, the polypeptide complex, pharmaceutical composition, polynucleotide, or vector is administered to the liver of the subject.

In some embodiments of any of the above-described methods for treating a TGFβ dysregulation disorder in a subject in need thereof, the polypeptide complex, pharmaceutical composition, polynucleotide, or vector is administered in combination with an additional therapeutic agent.

In some embodiments of any of the above-described methods for treating a TGFβ dysregulation disorder in a subject in need thereof, the TGFβ dysregulation disorder is an inflammatory bowel disease (IBD).

In some embodiments of any of the above-described methods for treating a TGFβ dysregulation disorder in a subject in need thereof, the TGFβ dysregulation disorder is Marfan syndrome.

In some embodiments of any of the above-described methods for treating a TGFβ dysregulation disorder in a subject in need thereof, the TGFβ dysregulation disorder is an autoimmune disorder.

In some embodiments of any of the above-described methods for treating a TGFβ dysregulation disorder in a subject in need thereof, the TGFβ dysregulation disorder is a wound healing disorder.

In another aspect, provided herein is a method for promoting wound healing in a subject in need thereof, comprising administering to the subject a therapeutically effective amount of a polypeptide complex described herein, a pharmaceutical composition described herein, a polynucleotide described herein, or a vector described herein.

In some embodiments, the polypeptide complex, pharmaceutical composition, polynucleotide, or vector is administered to the wound of the subject.

In some embodiments, the subject is human.

### BRIEF DESCRIPTION OF THE DRAWINGS

FIG. 1 is a schematic representation of TGFβ1 in complex with the latency associated peptide (LAP).

FIG. 2 demonstrates that a TGFβ latent complex can be proteolytically or mechanically activated.

FIGS. 3A-3B show antibody-TGFβ small latent complex (SLC) fusion construct designs. FIG. 3A shows an antibody-TGFβ SLC fusion construct design. A large latent complex (LLC) in both closed and open conformation is also depicted. FIG. 3B shows the processing and activation profile of anti-CD63-TGFβ1 SLC fusion proteins. The unprocessed anti-CD63-TGFβ1 SLC fusion (left) is an expressed form which can be present, e.g., in both cellular lysate and conditioned media (CM), and cannot be activated by integrin. The processed anti-CD63-TGFβ1 SLC fusion (right) is a target form, which can be purified, present only in the CM, and activated by integrin (e.g., integrin αvβ6). LAP, latency associated peptide; SLC, small latent complex (LAP+mature domain).

FIGS. 4A-4C show a construct design of an anti-hCD63-TGFβ construct and amino acid sequences. FIG. 4A shows an amino acid sequence corresponding to an anti-hCD63-TGFβ1.C33S Chain 1 comprising mROR SP (signal peptide)+VH (heavy chain variable domain) anti-hCD63+hIgG4+linker+TGFβ1 [LAP.C33S+mature peptide]. FIG. 4B shows an amino acid sequence corresponding to an anti-hCD63-TGFβ2.C33S Chain 1 comprising mROR SP+VH anti-hCD63+hIgG4+linker+TGFβ2 [LAP.C33S+mature peptide]. FIG. 4C shows an amino acid sequence corresponding to an anti-hCD63-TGFβ.C33S Chain 2 comprising mROR SP+VK (K light-chain variable domain) anti-hCD63+hKappa).

FIG. 5 shows secretion of an anti-hCD63-TGFβ construct in the conditioned media of ExpiCHO cells.

FIG. 6 shows expression of anti-hCD63-TGFβ1 in FreedomCHO cells.

FIG. 7 shows mature TGFβ1 induced Smad2/3 signaling in Hek293-CAGA reporter cells.

FIG. 8 shows both heat and acid activation of anti-hCD63-TGFβ1-expressing ExpiCHO conditioned media (CM) induced Smad2/3 signaling.

FIG. 9 shows anti-hCD63-TGFβ1 SLC was activated in CD63.Y235A (non-internalizing mutant) expressing cells co-cultured with integrin αvβ6 expressing cells.

FIG. 10 shows an amino acid sequence corresponding to an anti-hCD63-TGFβ1.C33S uber stealth Chain 1 comprising mROR SP+VH anti-hCD63+hIgG4+linker+TGFβ1 [LAP.C33S+mature peptide].

FIG. 11 shows an amino acid sequence corresponding to an anti-hCD63-TGFβ1.C33S uber stealth Chain 2 comprising mROR SP+VK anti-hCD63+hKappa.

FIG. 12 demonstrates anti-hCD63-TGFβ1 secreted from FreedomCHO cells is completely processed.

FIG. 13 shows a size-exclusion chromatography (SEC) profile of purified anti-hCD63-TGFβ1 fusion proteins (top panel) and accompanying SDS-PAGE (sodium dodecyl sulfate polyacrylamide gel electrophoresis) analysis of eluates (bottom panel).

FIG. 14 shows that anti-hCD63-TGFβ1 was bound to hCD63.Y235A expressing cells but not hCD63 knockout (KO) Hek293 cells.

FIG. 15 shows heat activation data of purified anti-CD63-TGFβ1 in NBL7 cells (American Mink lung epithelial cells) carrying the Smad2/3 reporter vector.

FIG. 16 illustrates mechanical activation of anti-CD63-TGFβ1 SLC by integrin αvβ6 induces Smad2/3 signaling.

FIG. 17 shows a schematic representation of a REGN14660-TGFβ1 and REGN14660-TGFβ2 fusions described herein. The REGN14660-TGFβ1 fusion contains a tripeptide Arg-Gly-Asp (Arginine, Glycine, and Aspartate) ‘RGD’ binding domain in the latency associated polypeptide (LAP), which allows for integrin-mediated activation of TGFβ1.

FIG. 18 shows REGN14660-TGFβ1 purification by high-performance liquid chromatography (HPLC).

FIG. 19 shows REGN14660-TGFβ1 western blot analysis.

FIG. 20 shows REGN14660-TGFβ2 purification by HPLC.

FIG. 21 shows REGN14660-TGFβ2 western blot analysis.

FIG. 22 shows a Smad2/3 reporter assay showing latency of the REGN14660-TGFβ1 fusion (HA=heat activation). The Smad2/3 reporter assay illustrates that the REGN14660-TGFβ1 retains TGFβ in a latent form (boxes). TGFβ is released from LAP by heat activation (HA). Circles indicate “free” active TGFβ present in the antibody preparation.

FIG. 23 shows a Smad2/3 reporter assay showing latency of the REGN14660-TGFβ2 fusion (HA=heat activation). The Smad2/3 reporter assay illustrates that the REGN14660-TGFβ2 retains TGFβ in a latent form (boxes). TGFβ is released from LAP by heat activation (HA). Circles indicate “free” active TGFβ present in the antibody preparation.

FIG. 24 illustrates that REGN14660-TGFβ1 binds to fibronectin extra domain B (EDB-FN; extracellular matrix FN) with higher affinity (black line with closed circle) as compared to plasma (soluble) FN (black line with open square). TGFβ1 could be activated from the SLC by αvβ6 integrin expressed by CHO (black line with closed circle) cells but not from the parental cell line Chinese hamster ovary cells CHO (black line with open triangle).

FIG. 25 shows images of an aorta from wild-type (WT) and Fibrillin 1 knockout (Fbn1KO) mice. The top panel shows an aneurysm in the ascending aorta of an Fbn1KO mouse. The bottom panel shows immunostaining of WT and Fbn1KO aortic sections using the REGN14660 antibody and shows extra domain B of fibronectin (EDB-FN) staining in the diseased area (ascending) of the Fbn1KO aorta.

FIGS. 26A-26B demonstrate EDB-FN detection can be associated with the presence of abnormal microvessels (positive for CD31) formed within the media layer of the Fbn1KO aorta (FIG. 26A). EDB-FN was not detected in wild-type (WT) P15 aorta (FIG. 26B).

FIG. 27 shows a survival curve plot for Fibrillin 1 knockout (Fbn1KO) mice (MAID 9419).

FIG. 28 depicts a schematic representation of fibrillin microfibril functions. TGFβ, Transforming Growth Factor 3; BMP, Bone Morphogenetic Protein; LTBP, latency associated binding protein; Magp1/2, Microfibril-associated glycoproteins 1 and 2; ECM, extracellular matrix.

FIG. 29 depicts a model for aortic aneurysm in Marfan Syndrome (MFS) associated with reduced TGFβ signaling. Fewer fibrillin microfibrils in MFS result in reduced incorporation of the large latent complex (LLC) in the extracellular matrix (ECM), thereby leading to decreased TGFβ signaling and ultimately to aneurysm.

FIG. 30 illustrates use of fibronectin (FN) as a docking platform to deliver antibody-latent TGFβ fusions for restoration of local TGFβ in MFS. Fibronectin is the matrix template for deposition of fibrillin-1 microfibers. Fibronectin and fibrillin coexist in all the tissues in which they are co-expressed.

FIG. 31 illustrates that plasma and cellular fibronectin (FN) differ in three domains introduced by alternative splicing. Fibronectin is encoded by a single gene but different forms of fibronectin arise through alternative splicing. Plasma FN is produced and secreted by hepatocytes in a soluble dimeric form. Cellular FN is expressed by several mesenchymal cells as dimeric or cross-linked multimeric forms and it is deposited as fibrils in the ECM, which is required for fibrillin-1 and type I collagen (Col Type I) deposition in the ECM. Fibronectins are involved in, e.g., cell adhesion, cell motility, maintenance of cell shape, development, and wound healing. FN KO mice exhibit embryonic lethality at approximately day 8.5.

FIG. 32 shows fibronectin isoforms (cellular FN) containing the EDB domain are expressed in growing and remodeling tissues. EDB is a small domain of 91 amino acids (SEQ ID NO: 84) that is part of the fibronectin isoforms created by alternative splicing. The sequence of EDB is identical in mouse and humans. EDB-containing isoforms are expressed during embryonic and postnatal development and in a variety of solid tumors (and can be a marker of newly forming vessels) but are virtually undetectable in normal adult tissues, except for in the endometrium during the proliferative phase and some vessels in the ovaries.

FIG. 33 shows an example of a single dose pharmacokinetics (PK) and tissue distribution study design.

FIG. 34 depicts use of an enzyme linked immunosorbent assay (ELISA) to determine whether antibody-TGFβ SLC fusions disclosed herein stayed latent in circulation.

FIG. 35 demonstrates antibody-TGFβ1 SLC fusions disclosed herein stayed latent in circulation.

FIG. 36 illustrates Fc-fusion levels of antibody-TGFβ1 SLCs disclosed herein in circulation.

FIG. 37 shows expression of C-type lectin domain family 9 member A (Clec9a) and epithelial cell adhesion molecule (Epcam) in the ileum, colon, and heart (ArrayStudio).

FIG. 38 depicts use of a tissue ELISA to quantify the biodistribution of antibody-TGFβ SLC fusions disclosed herein.

FIG. 39 shows the delivered amount of antibody-TGFβ1 SLC fusions in the ileum and colon 2 hours (hrs) post-injection (top) and 18 hrs post-injection (bottom).

FIG. 40 demonstrates that the anti-mEpcam-TGFβ1 SLC strongly induced Smad2/3 phosphorylation in the ileum.

FIG. 41 demonstrates that the anti-mClec9a-TGFβ1 SLC induced Smad2/3 phosphorylation in the colon.

FIG. 42 demonstrates that the antibody-TGFβ1 SLC fusions did not induce Smad2/3 phosphorylation in the heart.

FIG. 43 demonstrates that the anti-mEpcam-TGFβ1 SLC fusion was delivered to the colon.

FIG. 44 demonstrates that the anti-mEpcam-TGFβ1 SLC fusion was delivered to the ileum.

FIG. 45 demonstrates that anti-mEpcam-TGFβ1 SLC fusion induced phosphorylated (P)-Smad2/3 in the ileum.

FIG. 46 shows an amino acid sequence corresponding to an anti-mEpcam-TGFβ1.C33S, Chain 1 comprising mROR SP+VH anti-mEpcam+mIgG1+linker+TGFβ1 [LAP.C33S+mature peptide].

FIG. 47 shows an amino acid sequence corresponding to an anti-mEpcam-TGFβ1.C33S, Chain 2 comprising mROR SP+VK anti-mEpcam+mKappa.

FIG. 48 shows an amino acid sequence corresponding to an anti-mClec9a-TGFβ1.C33S, Chain 1 comprising mROR SP+VH anti-mClec9a+mIgG1+linker+TGFβ1 [LAP.C33S+mature peptide].

FIG. 49 shows an amino acid sequence corresponding to an anti-mClec9a-TGFβ1.C33S, Chain 2 comprising mROR SP+VK anti-mClec9a+mKappa.

FIG. 50 shows an amino acid sequence corresponding to an mIgG1 isotype control antibody-TGFβ1.C33S, Chain 1 comprising mROR SP+VH mIgG1 isotype control antibody+mIgG1+linker+TGFβ1 [LAP.C33S+mature peptide].

FIG. 51 shows an amino acid sequence corresponding to an mIgG1 isotype control antibody-TGFβ1.C33S, Chain 2 comprising mROR SP+VK mIgG1 isotype control antibody+mKappa.

### DETAILED DESCRIPTION

The present application provides, among other things, compositions and methods related to a polypeptide complex comprising a target-binding polypeptide that binds a molecule on a target cell or a molecule in an extracellular matrix (ECM) and a small latent complex (SLC). The SLC, in particular, comprises a dimeric latency associated polypeptide (LAP), or a fragment or derivative thereof, and a dimeric mature Transforming Growth Factor β (TGFβ) family polypeptide, or a fragment or derivative thereof. The mature TGFβ family polypeptide, or the fragment or derivative thereof, can be inactive as a result of an interaction with the dimeric LAP. Upon activation, i.e., release from the SLC, the mature TGFβ family polypeptide, or the fragment or derivative thereof, may induce TGFβ signaling such as, but not limited to, Smad2/3 signaling. Further provided are fusion polypeptides comprising a target-binding polypeptide that binds a molecule on a target cell or a molecule in an extracellular matrix (ECM) a latency associated polypeptide (LAP) or a fragment or derivative thereof, and a mature Transforming Growth Factor β (TGFβ) family polypeptide or a fragment or derivative thereof. Also included are related polynucleotides, e.g., polynucleotides encoding the fusion polypeptide described herein, vectors, cells and pharmaceutical compositions. A method for delivering a mature TGFβ family polypeptide, or the fragment or derivative thereof to a target cell within a subject in need thereof is also disclosed. Specifically, the method comprises administering the polypeptide complex(es), pharmaceutical composition(s), polynucleotide(s) and/or vector(s) described herein such that, for example, the target-binding polypeptide within the polypeptide complex binds a molecule on a target cell. In certain aspects, the present disclosure also provides methods for treating a TGFβ dysregulation disorder (e.g., inflammatory bowel disease (IBD), Marfan syndrome, an autoimmune disease, and/or a wound healing disorder) in a subject in need thereof. Specifically, the method comprises administering to the subject a therapeutically effective amount of the polypeptide complex(es), the pharmaceutical composition(s), the polynucleotide(s), or the vector(s) described herein. A method for promoting wound healing in a subject involving administering to a subject in need thereof (e.g., a subject having a wound) a therapeutically effective amount of the polypeptide complex(es), the pharmaceutical composition(s), the polynucleotide(s), or the vector(s) described herein is also included.

### Definitions

Unless defined otherwise, all technical and scientific terms used herein have the same meaning as commonly understood by one of ordinary skill in the art to which this disclosure belongs.

Singular forms “a”, “an”, and “the” include plural references unless the context clearly dictates otherwise. Thus, for example, a reference to “a method” includes one or more methods, and/or steps of the type described herein and/or which will become apparent to those persons skilled in the art upon reading this disclosure.

The term “about” or “approximately” includes being within a statistically meaningful range of a value. Such a range can be within an order of magnitude, preferably within 50%, more preferably within 20%, still more preferably within 10%, and even more preferably within 5% of a given value or range. The allowable variation encompassed by the term “about” or “approximately” depends on the particular system under study, and can be readily appreciated by one of ordinary skill in the art.

The term “antigen” refers to any agent (e.g., protein, peptide, polysaccharide, glycoprotein, glycolipid, nucleic acid, portions thereof, or combinations thereof) that, when introduced into a host, animal or human, having an immune system (directly or upon expression as in, e.g., DNA vaccines), is recognized by the immune system of the host and is capable of eliciting an immune response.

The term “antigen-binding polypeptide” refers to an antigen-specific binding element that may be any ligand or receptor fragment that binds to the antigen of interest or a polypeptide or fragment thereof. In some embodiments, the ligand and/or receptor fragment may be naturally derived. In some embodiments, the ligand and/or receptor fragment may be synthetic. Non-limiting examples of antigen-binding polypeptides include, for example, antibodies; polypeptides derived from antibodies, e.g., Fab, Fab′, F(ab′)2, single chain variable fragments (scFv), and Fv fragments; polypeptides derived from T-cell receptors (TCRs), e.g., TCR variable domains; secreted factors (e.g., growth factors, cytokines) which may be artificially fused to signaling domains; and any ligand and/or receptor fragment that binds to an antigen of interest.

The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds an antigen to form a complex.

Terms “antibody”, “antibodies”, “immunoglobulin”, and the like, refer to immunoglobulin molecules and immunologically active portions of immunoglobulin molecules, i.e., molecules that contain an antigen binding site that specifically binds an antigen, whether natural or partly or wholly synthetically produced. The terms include monoclonal antibodies, multispecific antibodies (e.g., bispecific antibodies), human antibodies, humanized antibodies, chimeric antibodies, single-chain Fvs (scFv), single chain antibodies, Fab fragments, F(ab′) fragments, disulfide-linked Fvs (sdFv), intrabodies, minibodies, diabodies and anti-idiotypic (anti-Id) antibodies (including, e.g., anti-Id antibodies to antigen specific TCR), and epitope-binding fragments of any of the above. The terms “antibody” and “antibodies” also refer to covalent diabodies such as those disclosed in U.S. Pat. Appl. Pub. 2007/0004909, incorporated herein by reference in its entirety, and Ig-DARTS such as those disclosed in U.S. Pat. Appl. Pub. 2009/0060910, incorporated herein by reference in its entirety. Antibodies useful in the present disclosure include immunoglobulin molecules and immunologically active fragments of immunoglobulin molecules, i.e., molecules that contain an antigen binding site. Immunoglobulin molecules can be of any type (e.g., IgG, IgE, IgM, IgD, IgA and IgY), class (e.g., IgG1, IgG2, IgG3, IgG4, IgA1 and IgA2) or subclass.

The term “human antibody”, as used herein, is intended to include antibodies having variable and constant regions derived from human germline immunoglobulin sequences. The human monoclonal antibodies (mAbs) of the disclosure may include amino acid residues not encoded by human germline immunoglobulin sequences (e.g., mutations introduced by random or site-specific mutagenesis in vitro or by somatic mutation in vivo), for example in the complementarity determining regions (CDRs) and in particular CDR3. However, the term “human antibody”, as used herein, is not intended to include mAbs in which CDR sequences derived from the germline of another mammalian species (e.g., mouse), have been grafted onto human framework (FR) sequences. The term includes antibodies recombinantly produced in a non-human mammal, or in cells of a non-human mammal. The term is not intended to include antibodies isolated from or generated in a human subject.

The term “epitope” refers to an antigenic determinant that interacts with a specific antigen binding site in the variable region of an antibody molecule known as a paratope. A single antigen may have more than one epitope. Thus, different antibodies may bind to different areas on an antigen and may have different biological effects. The term “epitope” also refers to a site on an antigen to which B and/or T cells respond. It also refers to a region of an antigen that is bound by an antibody. Epitopes may be defined as structural or functional. Functional epitopes are generally a subset of the structural epitopes and have those residues that directly contribute to the affinity of the interaction. Epitopes may also be conformational, that is, composed of nonlinear amino acids. In certain embodiments, epitopes may include determinants that are chemically active surface groupings of molecules such as amino acids, sugar side chains, phosphoryl groups, or sulfonyl groups, and, in certain embodiments, may have specific three-dimensional structural characteristics, and/or specific charge characteristics.

The term “host cell” refers to any cell that comprises a heterologous nucleic acid. By way of a non-limiting example, the heterologous nucleic acid may be a vector. A host cell, for example, without limitation, may be a cell from any organism that is used, manipulated, modified, selected, transformed, or grown, to produce a substance by the cell, e.g., the expression by the cell of, an RNA or DNA sequence, a gene, a protein, or an enzyme

By “decreased” or “reduced” or “lowered” or “lessened” or “abated” is intended any decrease in the level or activity of the gene/protein (e.g., encoded at the locus of interest). For example, a decrease in activity can comprise a decrease in the overall level or activity of a given protein including, for example, a decreased level or activity of 0.5%, 1%, 5%, 10%, 20%, 30%, 40%, 50%, 60%, 70%, 80%, 90%, 100%, 120% or greater when compared to an appropriate control.

By “increased” is intended any increase in the level or activity of the gene/protein (e.g., encoded at the locus of interest). For example, an increase in activity can comprise an increase in the overall level or activity of a given protein including, for example, an increased level of activity of 0.5%1, %, 5%10%, 20%, 30%, 40%, 50%, 60%, 70%, 80%, 90%, 100%, 120% or greater when compared to an appropriate control.

The term “specifically binds”, “binds in a specific manner”, “antigen-specific”, or the like, indicates that the molecules involved in the specific binding are able to form a complex with each other that is relatively stable under physiological conditions, and are unable to form stable complexes non-specifically with other molecules outside the specified binding pair. Specific binding can be characterized by an equilibrium dissociation constant (K_{D}) in the low micromolar to picomolar range (i.e., a smaller K_{D} denotes a tighter binding). High specificity may be in the low nanomolar range, with very high specificity being in the picomolar range. Methods for determining whether two molecules specifically bind to one another are well known in the art and include, for example, equilibrium dialysis, surface plasmon resonance, and the like.

The terms “protein” and “polypeptide”, used interchangeably herein, encompass all kinds of naturally occurring and synthetic proteins, including protein fragments of all lengths, fusion proteins and modified proteins, including without limitation, glycoproteins, as well as all other types of modified proteins (e.g., proteins resulting from phosphorylation, acetylation, myristoylation, palmitoylation, glycosylation, oxidation, formylation, amidation, polyglutamylation, ADP-ribosylation, PEGylation, biotinylation, etc.). Small polypeptides of less than 100 amino acids, preferably less than 50 amino acids, may be referred to as “peptides”.

Proteins are said to have an “N-terminus” and a “C-terminus.” The term “N-terminus” relates to the start of an amino acid chain of a protein, terminated by an amino acid with a free amine group (—NH₂). The term “C-terminus” relates to the end of an amino acid chain of a protein, terminated by a free carboxyl group (—COOH).

The terms “nucleic acid”, “polynucleotide”, and “nucleotide” used interchangeably herein, include polymeric forms of nucleotides of any length, including ribonucleotides (RNA), deoxyribonucleotides (DNA), or analogs or modified versions thereof. They include single-, double-, and multi-stranded DNA or RNA, genomic DNA, cDNA, DNA-RNA hybrids, and polymers comprising purine bases, pyrimidine bases, or other natural, chemically modified, biochemically modified, non-natural, or derivatized nucleotide bases. A single-stranded nucleic acid can be the sense strand or the antisense strand.

Nucleic acids are said to have a “5′ end” and a “3′ end” because mononucleotides are reacted to make oligonucleotides in a manner such that the 5′ phosphate of one mononucleotide pentose ring is attached to the 3′ oxygen of its neighbor in one direction via a phosphodiester linkage. An end of an oligonucleotide is referred to as the “5′ end” if its 5′ phosphate is not linked to the 3 oxygen of a mononucleotide pentose ring. An end of an oligonucleotide is referred to as the “3′ end” if its 3′ oxygen is not linked to a 5′ phosphate of another mononucleotide pentose ring. A nucleic acid sequence, even if internal to a larger oligonucleotide, also may be said to have 5′ and 3′ ends. In either a linear or circular DNA molecule, discrete elements are referred to as being “upstream” or 5′ of the “downstream” or 3′ elements.

The term “fragment” when referring to a protein means a protein that is shorter or has fewer amino acids than the full-length protein. A fragment can be, for example, an N-terminal fragment (i.e., removal of a portion of the C-terminal end of the protein), a C-terminal fragment (i.e., removal of a portion of the N-terminal end of the protein), or an internal fragment. The term “fragment” when referring to a nucleic acid means a nucleic acid that is shorter or has fewer nucleotides than the full-length nucleic acid. A fragment can be, for example, a 5′ fragment (i.e., removal of a portion of the 3′ end of the nucleic acid), a 3′ fragment (i.e., removal of a portion of the 5′ end of the protein), or an internal fragment.

The terms “derivative” and “variant” are used herein interchangeably to refer to an entity that has significant structural identity with a reference entity but differs structurally from the reference entity in the presence or level of one or more chemical moieties as compared with the reference entity. In many embodiments, a derivative also differs functionally from its reference entity. In general, whether a particular entity is properly considered to be a “derivative” of a reference entity is based on its degree of structural identity with the reference entity. As will be appreciated by those skilled in the art, any biological or chemical reference entity has certain characteristic structural elements. A derivative, by definition, is a distinct entity that shares one or more such characteristic structural elements. To give but a few examples, a small molecule may have a characteristic core structural element (e.g., a macrocycle core) and/or one or more characteristic pendent moieties so that a derivative of the small molecule is one that shares the core structural element and the characteristic pendent moieties but differs in other pendent moieties and/or in types of bonds present (single vs double, E vs Z, etc.) within the core. A derivative nucleic acid may have a characteristic sequence element comprised of a plurality of nucleotide residues having designated positions relative to one another in linear or three-dimensional space. In some embodiments, the nucleic acid sequence of a derivative may be 50%, 60%, 70%, 80%, 81%, 82%, 83%, 84%, 85%, 86%, 87%, 88%, 89%, 90%, 91%, 92%, 93%, 94%, 95%, 96%, 97%, 98%, 99%, 99.5%, or more identical over the full length of the reference sequence or a fragment thereof. A derivative peptide or polypeptide may have a characteristic sequence element comprised of a plurality of amino acids having designated positions relative to one another in linear or three-dimensional space and/or contributing to a particular biological function. Derivative peptides and polypeptides include peptides and polypeptides that differ in amino acid sequence from the reference peptide or polypeptide by the insertion, deletion, and/or substitution of one or more amino acids, but retain at least one biological activity of such reference peptide or polypeptide (e.g., the ability to mediate cell infection by a virus, the ability to mediate membrane fusion, the ability to be bound by a specific antibody or to promote an immune response, etc.). In some non-limiting embodiments, a derivative peptide or polypeptide shows the sequence identity over the full length with the reference peptide or polypeptide (or a fragment thereof) that is at least 50%, 60%, 70%, 80%, 81%, 82%, 83%, 84%, 85%, 86%, 87%, 88%, 89%, 90%, 91%, 92%, 93%, 94%, 95%, 96%, 97%, 98%, 99%, 99.5%, or more. Alternatively, or in addition, a derivative peptide or polypeptide may differ from a reference peptide or polypeptide as a result of one or more and/or one or more differences in chemical moieties attached to the polypeptide backbone (e.g., in glycosylation, phosphorylation, acetylation, myristoylation, palmitoylation, oxidation, formylation, amidation, polyglutamylation, ADP-ribosylation, pegylation, biotinylation, etc.). In some embodiments, a derivative peptide or polypeptide lacks one or more of the biological activities of the reference polypeptide or has a reduced or increased level of one or more biological activities as compared with the reference polypeptide. Derivatives of a particular peptide or polypeptide may be found in nature or may be synthetically or recombinantly produced. As used herein, the term “derivative” or “variant” also encompassed various fusion proteins and conjugates, including fusions or conjugates with detection tags (e.g., HA tag, histidine tag, biotin, fusions with fluorescent or luminescent domains, etc.), dimerization/multimerization sequences, Fc, signaling sequences, etc. [00229]“Sequence identity” or “identity” in the context of two polynucleotides or polypeptide sequences makes reference to the residues in the two sequences that are the same when aligned for maximum correspondence over a specified comparison window. When percentage of sequence identity is used in reference to proteins, residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity.” Means for making this adjustment are well known. Typically, this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE. [00230]“Percentage of sequence identity” includes the value determined by comparing two optimally aligned sequences (greatest number of perfectly matched residues) over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity. Unless otherwise specified (e.g., the shorter sequence includes a linked heterologous sequence), the comparison window is the full length of the shorter of the two sequences being compared.

The term “latency associated peptide” or “LAP” can refer to a peptide pro-domain which prohibits binding of TGFβ to a Transforming Growth Factor β Receptor (TGFβR).

The term “integrin binding motif” or “integrin binding site” when used in connection with a latency associated peptide (LAP) disclosed herein can refer to a region on LAP comprising a site capable of being recognized by integrin.

The term “small latent complex” or “SLC” can refer to a complex formed by a TGFβ family polypeptide, or a fragment or a derivative thereof, and a latency associated peptide (LAP), or a fragment or derivative thereof. In some embodiments, TGFβ may be non-covalently associated with LAP. The SLC may be linked to an additional protein, e.g., a target-binding polypeptide.

The term “inactive” or “inactive form” or “latent” in the context of TGFβ can refer to a TGFβ family polypeptide, or a fragment or derivative thereof, which is incapable of binding to a Transforming Growth Factor β Receptor (TGFβR) and/or initiating TGFβ signaling (e.g., Smad2/3 signaling and/or ERK signaling, or the like). As non-limiting example, a TGFβ family polypeptide, or a fragment or derivative thereof, may be inactive as a result of an interaction with LAP, or a fragment or derivative thereof.

The term “active” or “active form” in the context of TGFβ can refer to a form of a TGFβ family polypeptide, or a fragment or derivative thereof, which is capable of binding to a Transforming Growth Factor β Receptor (TGFβR) and/or inducing TGFβ signaling (e.g., Smad2/3 signaling and/or ERK signaling, or the like), e.g., upon release from the small latent complex (SLC).

The term “dimeric” refers to any compound or molecule comprised of two subunits. A dimeric compound or molecule, e.g., a protein dimer, may be considered a homodimer (e.g., formed by two identical proteins) or a heterodimer (e.g., formed by two different proteins) depending on the nature of the subunits forming the dimeric compound or molecule. In some embodiments, the dimer subunits may be attached via, e.g., a disulfide bond. In some embodiments, the dimer subunits may be non-covalently bound together.

The term “mature TGFβ family polypeptide” refers to any TGFβ family polypeptide, or a fragment or derivative thereof, that has undergone dimerization, i.e., a process whereby, in the context of TGFβ, two TGFβ subunits are joined to form a single dimeric molecule. In some embodiments, the mature TGFβ polypeptide, or the fragment or derivative thereof, may be inactive as a result of, for example, an interaction with a latency associated peptide (LAP), or a fragment or a derivative thereof, e.g., a dimeric LAP. In some embodiments, the dimeric mature TGFβ family polypeptide and the dimeric LAP may be in complex (i.e., associated) to form a small latent complex (SLC). In some embodiments, the mature TGFβ family polypeptide and the dimeric LAP may be associated via a non-covalent interaction. In some embodiments, the mature TGFβ family polypeptide and the dimeric LAP may be covalently linked and may be separated, for example, by a protease cleavage site such as, but not limited to, a furin cleavage site. Other non-limiting examples of protease cleavage sites include PC1/3, PC2, PC4, PC5/6, PACE4, PC7, SKI-1/SIP, and PCSK9 cleavage sites. In some embodiments, after protease cleavage, the LAP may remain non-covalently associated with the mature TGFβ family polypeptide, or the fragment or derivative thereof. In some embodiments, the mature TGFβ family polypeptide, or the fragment or derivative thereof, may be released from the SLC. In some embodiments, the mature TGFβ family polypeptide, or the fragment or derivative thereof, may induce Smad2/3 signaling in a target cell or in a cell adjacent to the target cell upon release from the SLC. Non-limiting examples of a mature TGFβ family polypeptide include a mature Growth Differentiation Factor 8 (GDF8), a mature Growth Differentiation Factor 11 (GDF11), and a mature Bone Morphogenetic Protein 4 (BMP4) polypeptide. Members of the TGFβ family include, e.g., nodal, activins, inhibins, bone morphogenetic proteins (BMPs) and growth differentiation factors (GDFs), TGF-β1, TGF-β2 and TGF-β3. In some embodiments, the mature TGFβ family polypeptide may include, for example, TGFβ1, TGFβ2, TGFβ3, BMP4, and/or GDF11. In some embodiments, the mature TGFβ family polypeptide may comprise a latent BMP/TGF-β family ligand described herein. In some embodiments, the mature TGFβ family polypeptide may be a mature TGFβ polypeptide. In some embodiments, the mature TGFβ polypeptide may be a mature TGFβ1 polypeptide. In some embodiments, the mature TGFβ polypeptide may be a mature TGFβ2 polypeptide. In some embodiments, the mature TGFβ polypeptide may be a mature Growth Differentiation Factor 8 (GDF8) or a mature Growth Differentiation Factor 11. In some embodiments, the mature TGFβ polypeptide may be a mature Bone Morphogenetic Protein 4 (BMP4).

The term “operably linked” or the like refers to a juxtaposition wherein the components described are in a relationship permitting them to function in their intended manner. For example, a control sequence “operably linked” to a coding sequence is ligated in such a way that expression of the coding sequence is achieved under conditions compatible with the control sequences. “Operably linked” sequences include both expression control sequences that are contiguous with a gene of interest and expression control sequences that act in trans or at a distance to control a gene of interest (or sequence of interest). The term “expression control sequence” includes polynucleotide sequences, which are necessary to affect the expression and processing of coding sequences to which they are ligated. “Expression control sequences” include appropriate transcription initiation, termination, promoter and enhancer sequences; efficient RNA processing signals such as splicing and polyadenylation signals; sequences that stabilize cytoplasmic mRNA; sequences that enhance translation efficiency (i.e., Kozak consensus sequence); sequences that enhance polypeptide stability; and when desired, sequences that enhance polypeptide secretion. The nature of such control sequences differs depending upon the host organism. For example, in prokaryotes, such control sequences generally include promoter, ribosomal binding site and transcription termination sequence, while in eukaryotes typically such control sequences include promoters and transcription termination sequence. The term “control sequences” is intended to include components whose presence is essential for expression and processing and can also include additional components whose presence is advantageous, for example, leader sequences and fusion partner sequences.

The terms “vector”, “expression vector”, and “cloning vector” refer to any vehicle by which a nucleotide sequence, e.g., an RNA sequence or a DNA sequence, encoding for example, a foreign gene, may be introduced into a cell (e.g., a host cell) in order to genetically modify the cell and promote expression (e.g., transcription and translation) of said introduced nucleotide sequence. Non-limiting examples of vectors include synthesized RNA and DNA molecules plasmids, viruses, phages, and the like. In some embodiments, the vector may be a viral vector including, without limitation, a baculoviral vector, a herpes virus vector, a lentiviral vector, a retroviral vector, a vaccinia virus vector, an adeno-associated virus (AAV) vector, an adenoviral vector, and an alphaviral vector.

The term “isolated” refers to a homogenous population of molecules (such as polynucleotides or polypeptides) which have been substantially separated and/or purified away from other components of the system the molecules are produced in, such as a recombinant cell, as well as a protein that has been subjected to at least one purification or isolation step. “Isolated” refers to a molecule that is substantially free of other cellular material and/or chemicals and encompasses molecules that are isolated to a higher purity, such as to 80%, 81%, 82%, 83%, 84%, 85%, 86%, 87%, 88%, 89%, 90%, 91%, 92%, 93%, 94%, 95%, 96%, 97%, 98%, 99% or 100% purity.

The terms “treat” or “treatment” of a state, disorder, disease, or condition include: (1) preventing, delaying, or reducing the incidence and/or likelihood of the appearance of at least one clinical or sub-clinical symptom of the state, disorder, disease, or condition developing in a subject that may be afflicted with or predisposed to the state, disorder, disease, or condition, but does not yet experience or display clinical or subclinical symptoms of the state, disorder, disease, or condition; or (2) inhibiting the state, disorder, disease, or condition, i.e., arresting, reducing or delaying the development of the disease or a relapse thereof or at least one clinical or sub-clinical symptom thereof; or (3) relieving the state, disorder, disease, or condition, i.e., causing regression of the state, disorder, disease, or condition or at least one of the clinical or sub-clinical symptoms of the state, disorder, disease, or condition. The benefit to a subject to be treated is either statistically significant or at least perceptible to the patient or to the physician.

The term “TGFβ dysregulation disorder” refers to a state, disorder, disease, or condition associated with dysregulation of TGFβ, including, e.g., low TGFβ expression and/or expression of variant forms of TGFβ. In some embodiments, the TGFβ dysregulation disorder can be treated by targeted delivery of TGFβ. Non-limiting examples of TGFβ dysregulation disorders include Type 1 diabetes mellitus (T1D), inflammatory bowel disease (IBD), colitis, Marfan syndrome (MFS), aortic dilation and rupture (aortic aneurysm), an autoimmune disorder, an arthritis (e.g., rheumatoid arthritis or osteoarthritis), lupus (e.g., systemic lupus), and a wound healing disorder.

An “individual” or “subject” or “animal” refers to humans, veterinary animals (e.g., cats, dogs, cows, horses, sheep, pigs, etc.) and experimental animal models of diseases (e.g., mice, rats). In a preferred embodiment, the subject is a human.

The term “effective” applied to dose or amount refers to that quantity of a compound or pharmaceutical composition that is sufficient to result in a desired activity upon administration to a subject in need thereof. Note that when a combination of active ingredients is administered, the effective amount of the combination may or may not include amounts of each ingredient that would have been effective if administered individually. The exact amount required will vary from subject to subject, depending on the species, age, and general condition of the subject, the severity of the condition being treated, the particular drug or drugs employed, the mode of administration, and the like.

The phrase “pharmaceutically acceptable”, as used in connection with compositions described herein, refers to molecular entities and other ingredients of such compositions that are physiologically tolerable and do not typically produce untoward reactions when administered to a mammal (e.g., a human). Preferably, the term “pharmaceutically acceptable” means approved by a regulatory agency of the Federal or a state government or listed in the U.S. Pharmacopeia or other generally recognized pharmacopeia for use in mammals, and more particularly in humans.

The term “administration” and the like refers to and includes the administration of a composition to a subject or system (e.g., to a cell, organ, tissue, organism, or relevant component or set of components thereof). The skilled artisan will appreciate that route of administration may vary depending, for example, on the subject or system to which the composition is being administered, the nature of the composition, the purpose of the administration, etc. For example, in certain embodiments, administration to an animal subject (e.g., to a human or a rodent) may be bronchial (including by bronchial instillation), buccal, enteral, interdermal, intra-arterial, intradermal, intragastric, intramedullary, intramuscular, intranasal, intraperitoneal, intrathecal, intravenous, intraventricular, mucosal, nasal, oral, rectal, subcutaneous, sublingual, topical, tracheal (including by intratracheal instillation), transdermal, vaginal and/or vitreal. In some embodiments, administration may involve intermittent dosing. In some embodiments, administration may involve continuous dosing (e.g., perfusion) for at least a selected period of time.

In accordance with the disclosure herein, there may be employed conventional molecular biology, microbiology, and recombinant DNA techniques within the skill of the art. Such techniques are explained fully in the literature. See, e.g., Sambrook, Fritsch & Maniatis, Molecular Cloning: A Laboratory Manual, Second Edition. Cold Spring Harbor, NY: Cold Spring Harbor Laboratory Press, 1989 (herein “Sambrook et al., 1989”); DNA Cloning: A Practical Approach, Volumes I and II (D. N. Glover ed. 1985); Oligonucleotide Synthesis (M. J. Gait ed. 1984); Nucleic Acid Hybridization [B. D. Hames & S. J. Higgins eds. (1985)]; Transcription And Translation [B. D. Hames & S. J. Higgins, eds. (1984)]; Animal Cell Culture [R. I. Freshney, ed. (1986)]; Immobilized Cells And Enzymes [IRL Press, (1986)]; B. Perbal, A Practical Guide To Molecular Cloning (1984); Ausubel, F. M. et al. (eds.). Current Protocols in Molecular Biology. John Wiley & Sons, Inc., 1994. These techniques include site directed mutagenesis as described in Kunkel, Proc. Natl. Acad. Sci. USA 82: 488-492 (1985), U.S. Pat. No. 5,071,743, Fukuoka et al., Biochem. Biophys. Res. Commun. 263: 357-360 (1999); Kim and Maas, BioTech. 28: 196-198 (2000); Parikh and Guengerich, BioTech. 24: 4 28-431 (1998); Ray and Nickoloff, BioTech. 13: 342-346 (1992); Wang et al., BioTech. 19: 556-559 (1995); Wang and Malcolm, BioTech. 26: 680-682 (1999); Xu and Gong, BioTech. 26: 639-641 (1999), U.S. Pat. Nos. 5,789,166 and 5,932,419, Hogrefe, Strategies 14. 3: 74-75 (2001), U.S. Pat. Nos. 5,702,931, 5,780,270, and 6,242,222, Angag and Schutz, Biotech. 30: 486-488 (2001), Wang and Wilkinson, Biotech. 29: 976-978 (2000), Kang et al., Biotech. 20: 44-46 (1996), Ogel and McPherson, Protein Engineer. 5: 467-468 (1992), Kirsch and Joly, Nucl. Acids. Res. 26: 1848-1850 (1998), Rhem and Hancock, J. Bacteriol. 178: 3346-3349 (1996), Boles and Miogsa, Curr. Genet. 28: 197-198 (1995), Barrenttino et al., Nuc. Acids. Res. 22: 541-542 (1993), Tessier and Thomas, Meths. Molec. Biol. 57: 229-237, and Pons et al., Meth. Molec. Biol. 67: 209-218.

### Polypeptide Complexes

In certain aspects, the present disclosure provides a polypeptide complex comprising:
a) a target-binding polypeptide that binds a molecule on a target cell or a molecule in an extracellular matrix (ECM); and
b) a small latent complex (SLC) comprising:
   (i) a dimeric latency associated polypeptide (LAP), or a fragment or derivative thereof, and
   (ii) a dimeric mature Transforming Growth Factor β (TGFβ) family polypeptide, or a fragment or derivative thereof,
      wherein the mature TGFβ family polypeptide, or the fragment or derivative thereof, is inactive as a result of an interaction with the dimeric LAP.

In some embodiments, the LAP, or the fragment or derivative thereof, is covalently attached to the target-binding polypeptide. In some embodiments, the LAP, or the fragment or derivative thereof, is covalently attached to the target-binding polypeptide via a linker. In some embodiments the LAP, or the fragment or derivative thereof, is noncovalently bound to the target-binding polypeptide. In some embodiments, the target-binding polypeptide binds both the LAP and the molecule on the target cell or the molecule in the ECM. In some embodiments, the target may comprise, for example, without limitation a molecule on a target cell or a molecule in an extracellular matrix (ECM).

In some embodiments, the mature TGFβ family polypeptide, or the fragment or derivative thereof, and the dimeric LAP are associated via a noncovalent interaction.

In some embodiments, the mature TGFβ family polypeptide, or the fragment or derivative thereof, and the LAP are covalently linked and are separated by a protease cleavage site Non-limiting examples of protease cleavage sites include furin, PC1/3, PC2, PC4, PC5/6, PACE4, PC7, SKI-1/S1P, and PCSK9 cleavage sites. In some embodiments, the mature TGFβ family polypeptide, or the fragment or derivative thereof, binds a Transforming Growth Factor R Receptor (TGFβR) upon release of the mature TGFβ family polypeptide, or the fragment or derivative thereof, from the SLC.

In some embodiments, the mature TGFβ family polypeptide, or the fragment or derivative thereof, induces Smad2/3 signaling in the target cell or a cell adjacent to the target cell upon activation of the mature TGFβ family polypeptide. In some embodiments, the mature TGFβ family polypeptide, or the fragment or derivative thereof, induces Smad2/3 signaling in the target cell or a cell adjacent to the target cell upon release of the mature TGFβ family polypeptide from the SLC.

### Small Latent Complex (SLC)

In certain aspects, the polypeptide complex described herein may comprise a small latent complex (SLC) comprising a dimeric latency associated peptide (LAP), or a fragment or derivative thereof, and a dimeric mature Transforming Growth Factor 1 (TGFβ) family polypeptide, or a fragment or derivative thereof. In one aspect, the mature TGFβ family polypeptide, or the fragment or derivative thereof, may be inactive as a result of an interaction with the dimeric LAP.

Without wishing to be bound by theory, a small latent complex (SLC) may comprise a complex of an LAP domain and a mature TGFβ domain. During secretion, furin-like pro-protein convertases may cleave TGFβ at a protease cleavage site located, e.g., at the junction between the mature domain and the LAP. After furin cleavage, the LAP may remain non-covalently associated with the mature TGFβ domain thereby rendering the mature TGFβ domain inactive by blocking the binding of the mature TGFβ domain to the TGFβ signaling receptors. Thus, if the LAP remains associated with the mature TGFβ domain, the activity of the mature domain may be blocked. The SLC can be secreted alone as a soluble molecule but may be secreted while tethered to, for example, a cell milieu molecule(s) which may be surface bound. In some embodiments, the milieu molecules can be covalently linked to the SLC. Non-limiting examples of milieu molecules include latency associated binding protein (LTBP), glycoprotein-A repetition predominant protein (GARP), leucine rich repeat containing protein 32 (LRRC32), and leucine rich repeat containing protein 33 (LRRC33/NRROS). An SLC secreted in complex with, e.g., a milieu molecule(s), may be called a large latent complex (LLC). Milieu molecules may each bind to the same epitope on LAP such as via disulfide bonds (e.g., C33 in the LAP domain disclosed herein).

The TGFβ protein family is encoded by 33 genes. Members of the TGF-β family, including include activins, anti-Müllerian hormone (AMH), bone morphogenetic proteins (BMPs), inhibins, Nodal, as well as growth and differentiation factors (GDFs), may participate in, e.g., the specification of the anterior/posterior and dorsal/ventral axes, ectoderm, mesoderm, and endoderm, as well as left-right asymmetry and various features of individual organs. TGFβ protein family members TGFβ1, TGFβ2 and TGFβ3 play a key role in immune responses, wound healing, development, and tumor-cell growth and inhibition. In particular, TGFβ1, TGFβ2, and TGFβ3 may participate in cellular processes such as, but not limited to, extracellular matrix (ECM) remodeling, cell migration, invasion, epithelial-mesenchymal transition (EMT), and growth inhibition and/or immune-suppression.

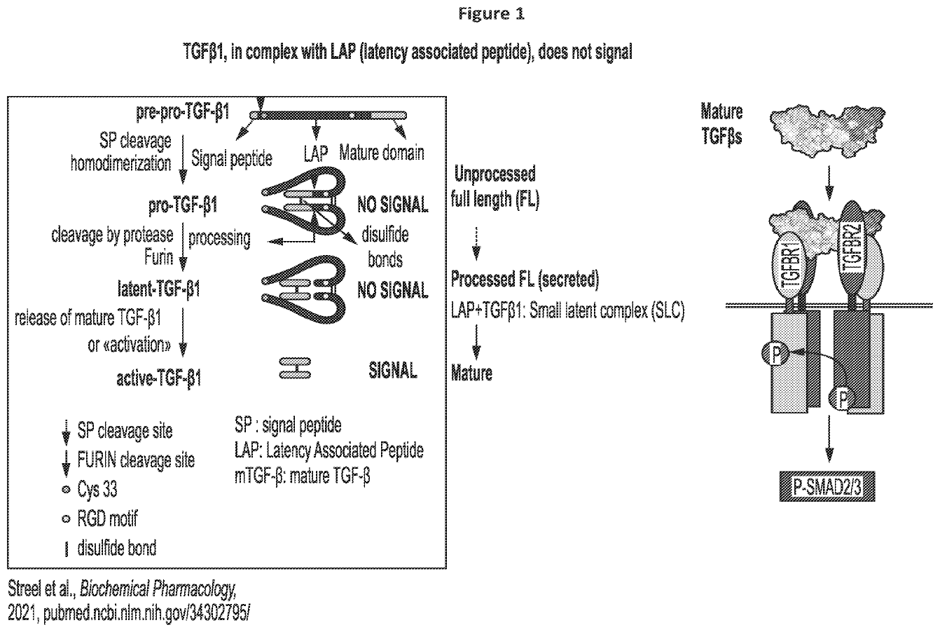

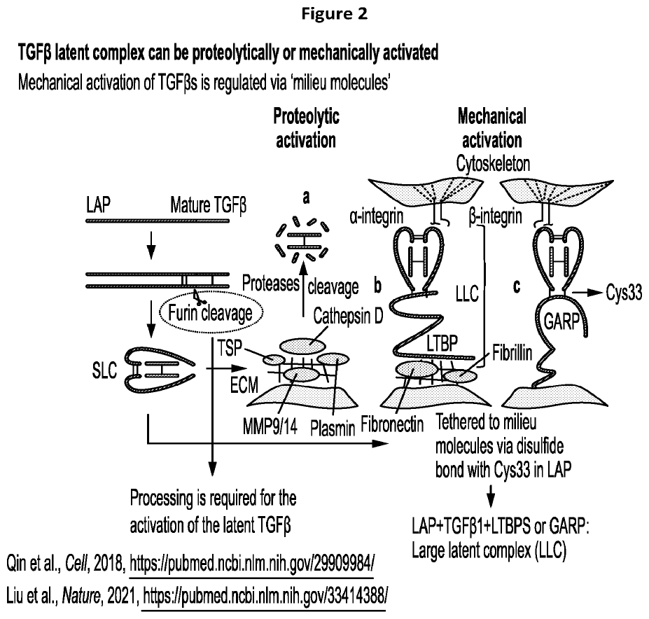

By way of a non-liming example, transforming growth factor beta 1 (TGFβ1) can be a secreted disulfide-bonded homodimeric protein. Full-length TGFβ1 may comprise a signal peptide (amino acids 1-29), a latency-associated peptide (LAP or pro-domain; amino acids 30-274), and a mature domain (amino acids 279-390). At the junction between the LAP and mature domain, there may be an ‘RXXR’(SEQ ID NO: 35) furin processing site, and ‘X’ can be any amino acid. Non limiting examples of ‘RXXR’ (SEQ ID NO: 35) furin processing sites include, e.g., RHRR (SEQ ID NO: 85), RRKR (SEQ ID NO: 86), and RKKR (SEQ ID NO: 87). During secretion, furin-like pro-protein convertases may cleave TGFβ1 at the junction between the mature domain and the LAP. After furin cleavage, the LAP may remain non-covalently associated with the mature domain thereby rendering the mature domain inactive by blocking the binding of the mature domain to a signaling receptor(s). Thus, if the LAP remains associated with the mature domain, the activity of the mature domain may be blocked. The LAP can be removed from processed TGFβ complexes by, e.g., proteolytic and mechanical methods. LAP removal thus may release an active mature TGFβ which may be capable of inducing downstream signaling, e.g., Smad2/3 signaling (see, e.g., FIG. 1 and FIG. 2).

In some embodiments, upon release of the mature TGFβ family polypeptide, or the fragment or derivative thereof disclosed herein, from a SLC disclosed herein, the mature TGFβ family polypeptide, or the fragment or derivative thereof, may bind a Transforming Growth Factor R Receptor (TGFβR). Without wishing to be bound by theory, seven TGFβ superfamily type I receptors and five type II receptors have been identified in mammals. The type I receptor family includes activin-like kinases (ALKs) 1 through 7. The type II receptors include TGFβRII, activin RIIA, activin RIIB, BMPRII, and AMHRII. The type I and type II receptors are structurally related transmembrane glycoproteins comprising an extracellular N-terminal ligand-binding domain with greater than ten cysteine residues which may regulate the dimeric structure, a transmembrane region, and a C-terminal serine/threonine kinase domain. The type I receptors have a highly conserved region GS domain that is rich in glycine and serine residues in the juxta-membrane domain proximal to the N-terminus of the kinase domain. In some embodiments, TGFβ signaling (e.g., Smad2/3 signaling) may be initiated by the binding of the mature TGFβ family polypeptide to a TGFβ receptor type I (TGFβRI) and/or a TGFβ receptor type II (TGFβRII) receptor(s), for example, on the membrane of a cell.

In some embodiments, upon release from the SLC disclosed herein, the mature TGFβ family polypeptide, or the fragment or derivative thereof disclosed herein, may induce TGFβ signaling, e.g., Smad2/3 signaling and/or ERK signaling, or the like, in a cell, e.g., a target cell or a cell adjacent to the target cell.

In some embodiments, the mature TGFβ family polypeptide, or the fragment or derivative thereof, may be chemically dissociated from the LAP, or the fragment or derivative thereof, and released in an active form from the SLC. Non-limiting examples of chemical dissociation comprise a protease treatment, a temperature treatment, an acid treatment, or any combination thereof.

In some embodiments, the mature TGFβ family polypeptide, or the fragment or derivative thereof, may be mechanically dissociated from the LAP, or the fragment or derivative thereof, and released in an active form from the SLC. By way of a non-limiting example, the mechanical dissociation may occur as a result of an interaction between the LAP, or the fragment or derivative thereof, and an integrin polypeptide such as via an integrin binding motif in the LAP. Non-limiting examples of integrins include αvβ6 integrin, αvβ8 integrin, and αvβ1 integrin.

In some embodiments, the small latent complex (SLC) comprises the amino acid sequence of SEQ ID NO: 21, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 21. In certain embodiments, the nucleotide sequence that encodes the small latent complex (SLC) comprises the nucleotide sequence that encodes the amino acid sequence of SEQ ID NO: 21, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 21. In certain embodiments, the nucleotide sequence that encodes the small latent complex (SLC) comprises the nucleotide sequence of SEQ ID NO: 22, or a nucleotide sequence having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 22. In certain embodiments, the small latent complex (SLC) comprises the amino acid sequence of SEQ ID NO: 21. In certain embodiments, the nucleotide sequence that encodes the small latent complex (SLC) comprises the nucleotide sequence of SEQ ID NO: 22.

In some embodiments, the small latent complex (SLC) comprises the amino acid sequence of SEQ ID NO: 25, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 25. In certain embodiments, the nucleotide sequence that encodes the small latent complex (SLC) comprises the nucleotide sequence that encodes the amino acid sequence of SEQ ID NO: 25, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 25. In certain embodiments, the nucleotide sequence that encodes the small latent complex (SLC) comprises the nucleotide sequence of SEQ ID NO: 26, or a nucleotide sequence having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 26. In certain embodiments, the small latent complex (SLC) comprises the amino acid sequence of SEQ ID NO: 25. In certain embodiments, the nucleotide sequence that encodes the small latent complex (SLC) comprises the nucleotide sequence of SEQ ID NO: 26.

In some embodiments, the small latent complex (SLC) comprises the amino acid sequence of SEQ ID NO: 92, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 92. In certain embodiments, the nucleotide sequence that encodes the small latent complex (SLC) comprises the nucleotide sequence that encodes the amino acid sequence of SEQ ID NO: 92, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 92. In certain embodiments, the nucleotide sequence that encodes the small latent complex (SLC) comprises the nucleotide sequence of SEQ ID NO: 93, or a nucleotide sequence having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 93. In certain embodiments, the small latent complex (SLC) comprises the amino acid sequence of SEQ ID NO: 92. In certain embodiments, the nucleotide sequence that encodes the small latent complex (SLC) comprises the nucleotide sequence of SEQ ID NO: 93.

### Latency-Associated Peptide (LAP)

In some embodiments, the polypeptide complex described herein may comprise a small latent complex (SLC) comprising a dimeric latency associated polypeptide (LAP), or a fragment or derivative thereof.

In some embodiments, the LAP, or the fragment or derivative thereof, may be attached to a target-binding polypeptide disclosed herein. In some embodiments, the LAP, or the fragment or derivative thereof, may be covalently attached to the target-binding polypeptide. In some embodiments, the LAP, or the fragment or derivative thereof, may be covalently attached to the target-binding polypeptide via a linker. In some embodiments, the linker may be located between the target-binding polypeptide and the LAP, or the fragment or derivative thereof.

In some embodiments, the linker may be between 1-10 amino acids long. In some embodiments, the linker may be between 1-20 amino acids long. As a non-limiting example, the linker may be 1, 2, 3, 4, 5, 6, 7, 8, 9, 10, 11, 12, 13, 14, 15, 16, 17, 18, 19, or 20 amino acids long. In some embodiments, the linker may be between 1-30 amino acids long. In some embodiments, the linker may be 21, 22, 23, 24, 25, 26, 27, 28, 29, or 30 amino acids long. In some embodiments, the linkers may flexible linkers. In some embodiments, the linkers may rigid linkers. In some embodiments, linkers may be from 1 amino acid to 20 amino acids long, from 2 amino acids to 15 amino acids long, from 3 amino acids to 12 amino acids long, including 4 amino acids to 10 amino acids long, 5 amino acids to 9 amino acids long, 6 amino acids to 8 amino acids long, or 7 amino acids to 8 amino acids long, and may be 1, 2, 3, 4, 5, 6, or 7 amino acids long. In some embodiments, the linker is 12 amino acids long. In some embodiments, the linker may be optimized such that the linker does not impose any constraints on the conformation and/or interactions of the linked partners.

In some embodiments, the linkers are flexible linkers. Suitable linkers can be readily selected and can be of any of a suitable of different lengths, such as from 1 amino acid (e.g., Gly) to 20 amino acids, from 2 amino acids to 15 amino acids, from 3 amino acids to 12 amino acids, including 4 amino acids to 10 amino acids, 5 amino acids to 9 amino acids, 6 amino acids to 8 amino acids, or 7 amino acids to 8 amino acids, and may be 1, 2, 3, 4, 5, 6, or 7 amino acids. Example flexible linkers include glycine polymers (G)n, glycine-serine polymers (GS)n, where n is an integer of at least one (e.g., from 1-20) (SEQ ID NO: 174), glycine-alanine polymers, alanine-serine polymers, and other flexible linkers known in the art.

In some embodiments, the linker is a cleavable linker. In some embodiments, the linker is a non-cleavable linker.

Non-limiting examples of linkers that may be used include any of SEQ ID NOs: 19, 46-79, and 96. In some embodiments, the linker comprises the amino acid sequence set forth in any of SEQ ID NOs: 19, 46-79, or 96, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NOs: 19, 46-79, or 96.

In some embodiments, the linker may comprise the sequence (GGGGS)n (SEQ ID NO: 46), wherein n=1-10, or n is 1, 2, 3, 4, 5, 6, 7, 8, 9, or 10. In some embodiments, the linker may consist of the sequence (GGGGS)n (SEQ ID NO: 46). As a non-limiting example, the linker may comprise the sequence GGGGS (SEQ ID NO: 47); GGGGSGGGGS ((G₄S)₂; SEQ ID NO: 48); GGGGSGGGGSGGGGS ((G₄S)₃; SEQ ID NO: 49); or GGGGSGGGGSGGGGSGGGGS ((G₄S)₄; SEQ ID NO: 50).

In some embodiments, the linker may comprise the sequence (GGGS)n (SEQ ID NO: 51), wherein n=1-10, or n is 1, 2, 3, 4, 5, 6, 7, 8, 9, or 10. In some embodiments, the linker may consist of the sequence (GGGS)n (SEQ ID NO: 51). In some embodiments, the linker may comprise the sequence GGGSGGGSGGGS (G₃S)₃ SEQ ID NO: 19). In some embodiments, the linker may consist of the sequence GGGSGGGSGGGS (G₃S)₃ SEQ ID NO: 19).

In some embodiments, the linker comprises the amino acid sequence of SEQ ID NO: 19, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 19. In certain embodiments, the nucleotide sequence that encodes the linker comprises the nucleotide sequence that encodes the amino acid sequence of SEQ ID NO: 19, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 19. In certain embodiments, the nucleotide sequence that encodes the linker comprises the nucleotide sequence of SEQ ID NO: 20, or a nucleotide sequence having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 20. In certain embodiments, the linker comprises the amino acid sequence of SEQ ID NO: 19. In certain embodiments, the nucleotide sequence that encodes the linker comprises the nucleotide sequence of SEQ ID NO: 20.

In some embodiments, the linker may comprise the sequence GSGESGGGSG (SEQ ID NO: 96). In some embodiments, the linker may consist of the sequence GSGESGGGSG (SEQ ID NO: 96).

In some embodiments, the linker comprises the amino acid sequence of SEQ ID NO: 96, or a variant thereof having at least about 50%, at least about 60%, at least about 70%, at least about 80%, at least about 90%, sequence identity with SEQ ID NO: 96. In certain embodiments, the nucleotide sequence that encodes the linker comprises the nucleotide sequence that encodes the amino acid sequence of SEQ ID NO: 96, or a variant thereof having at least about 50%, at least about 60%, at least about 70%, at least about 80%, at least about 90%, sequence identity with SEQ ID NO: 96. In certain embodiments, the nucleotide sequence that encodes the linker comprises the nucleotide sequence of SEQ ID NO: 97, or a nucleotide sequence having at least about 50%, at least about 60%, at least about 70%, at least about 80%, at least about 90%, sequence identity with SEQ ID NO: 97. In certain embodiments, the linker comprises the amino acid sequence of SEQ ID NO: 96. In certain embodiments, the nucleotide sequence that encodes the linker comprises the nucleotide sequence of SEQ ID NO: 97.

In some embodiments the LAP, or the fragment or derivative thereof, may be noncovalently bound to the target-binding polypeptide. In some embodiments, the target-binding polypeptide binds both the LAP and the molecule on the target cell or the molecule in the ECM. In some embodiments, the target-binding polypeptide may comprise an antigen-binding polypeptide. In some embodiments, the antigen-binding polypeptide may comprise an antibody or a fragment or derivative thereof such as an antigen-binding fragment thereof.

In some embodiments, the dimeric LAP of the present disclosure may be associated, for example, via a noncovalent interaction, with a mature TGFβ family polypeptide, or a fragment or derivative thereof, disclosed herein. In some embodiments, the LAP and the mature TGFβ family polypeptide, or the fragment or derivative thereof, may be covalently linked. In some embodiments, the LAP and the mature TGFβ family polypeptide, or the fragment or derivative thereof, may be covalently linked and may be separated by, e.g., a protease cleavage site.

In some embodiments, when the LAP or the fragment or derivative thereof may be attached (e.g., covalently attached) or bound (e.g., non-covalently bound) to the target-binding polypeptide, the target-binding polypeptide may bind both the LAP and the target.

In some embodiments, the protease cleavage site that can separate the LAP and the mature TGFβ family polypeptide may comprise a furin cleavage site. In some embodiments, the furin cleavage site may be located at the junction between the LAP and the mature TGFβ family polypeptide. In some embodiments, the furin cleavage site may comprise the sequence RXXR (SEQ ID NO: 35). In some embodiments, the protease cleavage site is a furin cleavage site. In some embodiments, the furin cleavage site comprises the sequence RXXR (SEQ ID NO: 35). In some embodiments, the furin cleavage site comprises the sequence RHRR (SEQ ID NO: 85). In some embodiments, the furin cleavage site comprises the sequence RRKR (SEQ ID NO: 86). In some embodiments, the furin cleavage site comprises the sequence RKKR (SEQ ID NO: 87).

Without wishing to be bound by theory, during secretion in the endoplasmic reticulum (ER)/Golgi, furin may cleave the mature TGFβ family polypeptide from the LAP, leaving the LAP non-covalently associated with the mature TGFβ family polypeptide. If the LAP remains associated with the mature TGFβ family polypeptide, the activity of the mature TGFβ family polypeptide may be blocked, thereby rending the mature TGFβ family polypeptide inactive. The LAP may be removed from mature TGFβ family polypeptide, e.g., via proteolytic and/or mechanical methods. LAP removal may release an active mature TGFβ, e.g., to induce downstream Smad2/3 signaling. In proteolytic activation, proteases may either degrade or lead to a conformational change of the LAP domain to release mature TGFβ. In mechanical activation, integrins (e.g., αvβ6 integrin, αvβ8 integrin, and/or αvβ1 integrin) may bind to the tripeptide Arg-Gly-Asp (Arginine, Glycine, and Aspartate) ‘RGD’ binding motif at the C-terminus of the LAP. Integrin binding at the C-terminus of the LAP and, e.g., a cell surface-bound milieu molecule association (through covalent bonding) at the N-terminus of the LAP may create a directional pulling force that may open the LAP and releases free mature TGFβ. Mature TGFβ may bind, e.g., TGFβR1 and TGFβR2 on the cell surface and induce signaling such as but not limited to Smad2/3 signaling. An active form of mature TGFβ may induce Smad2/3 signaling. In some embodiments, a mature TGFβ described herein may be internalized within the cell. In some embodiments, when the mature TGFβ is internalized within the cell, the mature TGFβ may induce TGFβ signaling (e.g., Smad2/3 signaling and/or ERK signaling, or the like) such as within an endosome.

In some embodiments, as a result of interaction (e.g., association) with the LAP, or the fragment or derivative thereof, the mature TGFβ family polypeptide, or the fragment or derivative thereof, disclosed herein may be inactive.

In some embodiments, activation of the mature TGFβ family polypeptide, or the fragment or derivative thereof may involve release of the mature TGFβ family polypeptide from the ECM, e.g., release of the LLC from the ECM, followed by further proteolysis of LAP by any of various proteases to release active TGFβ. Non-limiting examples of proteases which may be involved in proteolysis of LAP include matrix metalloproteinase 2 (MMP2), matrix metalloproteinase 9 (MMP9), thrombospondin1 (TSP-1) plasmin (PLN), and plasma kallikrein (PLK).

In some embodiments, upon activation, the mature TGFβ family polypeptide, or the fragment or derivative thereof, disclosed herein may bind a TGFβR disclosed herein. In some embodiments, upon activation, the mature TGFβ family polypeptide, or the fragment or derivative thereof disclosed herein, may induce Smad2/3 signaling in a cell (e.g., a target cell or a cell adjacent to the target cell). Upon activation, the mature TGFβ family polypeptide may be considered as an active form.

In some embodiments, the LAP, or the fragment or derivative thereof, may interact with a latency associated binding protein (LTBP), a glycoprotein-A repetition predominant protein (GARP), leucine rich repeat containing protein 32 (LRRC32), or leucine rich repeat containing protein 33 (LRRC33/NRROS).

In some embodiments, the LAP, or the fragment or derivative thereof, comprises an integrin binding motif. In some embodiments, the integrin-binding motif may comprise an RGD sequence (i.e., a sequence composed of an Arginine residue, a Glycine residue, and an Aspartate residue). In some embodiments, the integrin binding motif in the LAP may be a region that is composed of 1 to 30 amino acid residues including an RGD sequence. In some embodiments, the integrin binding motif in the LAP may be a region that is composed of 1 to 30 amino acid residues excluding an RGD sequence. In some embodiments, the integrin binding motif comprises the sequence RGD. In some embodiments, the integrin is αvβ6 integrin. In some embodiments, the integrin is αvβ8 integrin.

In some embodiments, the integrin binding motif in the LAP may be a region that may be between 1-10 amino acids long. In some embodiments, the integrin binding motif may be between 1-20 amino acids long. As a non-limiting example, the integrin binding motif may be 1, 2, 3, 4, 5, 6, 7, 8, 9, 10, 11, 12, 13, 14, 15, 16, 17, 18, 19, or 20 amino acids long. In some embodiments, the integrin binding motif may be between 1-30 amino acids long. In some embodiments, the integrin binding motif may be 21, 22, 23, 24, 25, 26, 27, 28, 29, or 30 amino acids long. In some embodiments, integrin binding motifs may be from 1 amino acid to 20 amino acids long, from 2 amino acids to 15 amino acids long, from 3 amino acids to 12 amino acids long, including 4 amino acids to 10 amino acids long, 5 amino acids to 9 amino acids long, 6 amino acids to 8 amino acids long, or 7 amino acids to 8 amino acids long, and may be 1, 2, 3, 4, 5, 6, or 7 amino acids long. In some embodiments, the integrin binding motif sequence may comprise an RGD sequence. In some embodiments, an RGD sequence may not be include an integrin binding motif.

In some embodiments, the integrin binding motif comprises an RGD sequence. In some embodiments, the integrin binding motif consists of an RGD sequence. Without wishing to be bound by theory, in mechanical activation, integrins may bind to integrin binding motif at the C-terminus of the LAP. Non-limiting examples of integrins comprise αvβ6 integrin, αvβ8 integrin, and αvβ1 integrin. In some embodiments, the integrin is an αvβ6 integrin. In some embodiments, the integrin is an αvβ8 integrin. Integrin binding at the C-terminus of the LAP and cell surface-bound milieu molecule association (e.g., through covalent bonding) at the N-terminus of the LAP creates a directional pulling force that opens LAP and releases free mature TGFβ. Mature TGFβ may bind TGFβR1 and TGFβR2 on the cell surface and induce Smad2/3 signaling.

In some embodiments, a mature TGFβ described herein may bind a TGFβ receptor, e.g., TGFβR1 and/or TGFβR2, at the surface of a cell described herein. In some embodiments, the ligand and the receptor can remain at the cell surface and the TGFβ ligand-receptor complex is not internalized. In some embodiments, when the TGFβ ligand-receptor complex is not internalized, TGFβ signaling (e.g., Smad2/3 signaling and/or ERK signaling, or the like) can be induced within the cell.

In some embodiments, a mature TGFβ described herein may bind a TGFβ receptor, e.g., TGFβR1 and/or TGFβR2, on the cell surface, thereby triggering internalization of the ligand and receptor. The TGFβ ligand-receptor complex may enter the endocytic system such as via clathrin-mediated endocytosis (CME). In some embodiments, endocytosis of the ligand-receptor complex, e.g., in early endosomes, may modulate (e.g., enhance) TGFβ signaling (e.g., Smad2/3 signaling and/or ERK signaling, or the like) described herein.

In some embodiments, a mature TGFβ disclosed herein may be internalized within a cell described herein (e.g., a target cell or a cell adjacent to the target cell). In some embodiments, when the mature TGFβ is internalized within the cell, the mature TGFβ may induce TGFβ signaling such as within an endosome.

In some embodiments, the integrin binding motif comprises an RGD sequence comprising one or more mutations. In some embodiments, the integrin binding motif consists of an RGD sequence comprising one or mutations. In some embodiments, the RGD sequence comprising one or more mutations comprises the sequence RGE. In some embodiments, when the RGD sequences comprises a mutation(s), the mutant RGD sequence may inhibit or block mechanical activation of the LAP. In some embodiments, when the mutant RGD sequence inhibits or blocks mechanical activation of the LAP, activation of the LAP may be limited to, e.g., activation via chemical activation and/or proteolytic activation described herein.

In some embodiments, the LAP, or the fragment or derivative thereof, does not comprise an integrin binding motif.

In some embodiments, the integrin binding motif comprises the amino acid sequence of SEQ ID NO: 33, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 33. In certain embodiments, the nucleotide sequence that encodes the integrin binding motif comprises the nucleotide sequence that encodes the amino acid sequence of SEQ ID NO: 33, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 33. In certain embodiments, the nucleotide sequence that encodes the integrin binding motif comprises the nucleotide sequence of SEQ ID NO: 34, or a nucleotide sequence having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 34. In certain embodiments, the integrin binding motif comprises the amino acid sequence of SEQ ID NO: 33. In certain embodiments, the nucleotide sequence that encodes the integrin binding motif comprises the nucleotide sequence of SEQ ID NO: 34.

In some embodiments, the LAP comprises the amino acid sequence of SEQ ID NO: 29, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 29. In certain embodiments, the nucleotide sequence that encodes the LAP comprises the nucleotide sequence that encodes the amino acid sequence of SEQ ID NO: 29, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 29. In certain embodiments, the nucleotide sequence that encodes the LAP comprises the nucleotide sequence of SEQ ID NO: 30, or a nucleotide sequence having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 30. In certain embodiments, the LAP comprises the amino acid sequence of SEQ ID NO: 29. In certain embodiments, the nucleotide sequence that encodes the LAP comprises the nucleotide sequence of SEQ ID NO: 30.

In some embodiments, the LAP, or the fragment or derivative thereof comprises an amino acid sequence that has at least 80% sequence identity to SEQ ID NO: 29. In some embodiments, the LAP, or the fragment or derivative thereof comprises the amino acid sequence of SEQ ID NO: 29. In some embodiments, the LAP consists of the amino acid sequence of SEQ ID NO: 29.

In some embodiments, the LAP, or the fragment or derivative thereof, comprises the sequence of positions 30-274 of the sequence of SEQ ID NO: 82.

In some embodiments, the LAP comprises the amino acid sequence of positions 30-274 of the sequence of SEQ ID NO: 82, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with the amino acid sequence of positions 30-274 of the sequence of SEQ ID NO: 82. In certain embodiments, the nucleotide sequence that encodes the LAP comprises the nucleotide sequence that encodes the amino acid sequence of positions 30-274 of the sequence of SEQ ID NO: 82, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with the amino acid sequence of positions 30-274 of the sequence of SEQ ID NO: 82. In certain embodiments, the nucleotide sequence that encodes the LAP comprises the nucleotide sequence of SEQ ID NO: 83, or a nucleotide sequence having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 83. In certain embodiments, the LAP comprises the amino acid sequence of positions 30-274 of the sequence of SEQ ID NO: 82. In certain embodiments, the nucleotide sequence that encodes the LAP comprises the nucleotide sequence of SEQ ID NO: 83.

In some embodiments, the LAP, or the fragment or derivative thereof comprises an amino acid sequence that has at least 80% sequence identity to the amino acid sequence of positions 30-274 of the sequence of SEQ ID NO: 82. In some embodiments, the LAP, or the fragment or derivative thereof comprises the amino acid sequence of positions 30-274 of the sequence of SEQ ID NO: 82. In some embodiments, the LAP consists of the amino acid sequence of positions 30-274 of the sequence of SEQ ID NO: 82.

In some embodiments, the LAP comprises the amino acid sequence of SEQ ID NO: 118, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 118. In certain embodiments, the nucleotide sequence that encodes the LAP comprises the nucleotide sequence that encodes the amino acid sequence of SEQ ID NO: 118, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 118. In certain embodiments, the nucleotide sequence that encodes the LAP comprises the nucleotide sequence of SEQ ID NO: 119, or a nucleotide sequence having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 119. In certain embodiments, the LAP comprises the amino acid sequence of SEQ ID NO: 118. In certain embodiments, the nucleotide sequence that encodes the LAP comprises the nucleotide sequence of SEQ ID NO: 119.

In some embodiments, the LAP, or the fragment or derivative thereof comprises an amino acid sequence that has at least 80% sequence identity to SEQ ID NO: 118. In some embodiments, the LAP, or the fragment or derivative thereof comprises the amino acid sequence of SEQ ID NO: 118. In some embodiments, the LAP consists of the amino acid sequence of SEQ ID NO: 118.

In some embodiments, the LAP, or the fragment or derivative thereof comprises the amino acid sequence of positions 21-298 of the amino acid sequence of SEQ ID NO: 116.

In some embodiments, the LAP comprises the amino acid sequence of positions 21-298 of the amino acid sequence of SEQ ID NO: 116, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with the amino acid sequence of positions 21-298 of the amino acid sequence of SEQ ID NO: 116. In certain embodiments, the nucleotide sequence that encodes the LAP comprises the nucleotide sequence that encodes the amino acid sequence of positions 21-298 of the amino acid sequence of SEQ ID NO: 116, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with the amino acid sequence of positions 21-298 of the amino acid sequence of SEQ ID NO: 116. In certain embodiments, the nucleotide sequence that encodes the LAP comprises the nucleotide sequence of SEQ ID NO: 117, or a nucleotide sequence having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 117. In certain embodiments, the LAP comprises the amino acid sequence of positions 21-298 of the amino acid sequence of SEQ ID NO: 116. In certain embodiments, the nucleotide sequence that encodes the LAP comprises the nucleotide sequence of SEQ ID NO: 117.

In some embodiments, the LAP, or the fragment or derivative thereof comprises an amino acid sequence that has at least 80% sequence identity to the amino acid sequence of positions 21-298 of the amino acid sequence of SEQ ID NO: 116. In some embodiments, the LAP, or the fragment or derivative thereof comprises the amino acid sequence of positions 21-298 of the amino acid sequence of SEQ ID NO: 116. In some embodiments, the LAP consists of the amino acid sequence of positions 21-298 of the amino acid sequence of SEQ ID NO: 116.

In some embodiments, the LAP of the present disclosure may include any amino acid sequence having an identity of at least about 60% or more, about 70% or more, 71% or more, 72% or more, 73% or more, 74% or more, 75% or more, 76% or more, 77% or more, 78% or more, 79% or more, 80% or more, 81% or more, 82% or more, 83% or more, 84% or more, 85% or more, 86% or more, 87% or more, 88% or more, 89% or more, 90% or more, 91% or more, 92% or more, 93% or more, 94% or more, 95% or more, 96% or more, 97% or more, 98% or more, 99% or more, 99.1% or more, 99.2% or more, 99.3% or more, 99.4% or more, 99.5% or more, 99.6% or more, 99.7% or more, 99.8% or more, or 99.9% or more to its natural amino acid sequence or parental sequence, and having the activity of normally-occurring (i.e., natural) sequence or parental sequence.

In some embodiments, the LAP, or the fragment or derivative thereof, may be heterologous to the mature TGFβ family polypeptide, or the fragment or derivative thereof disclosed herein.

In some embodiments, the LAP, or the fragment or derivative thereof, comprises amino acid mutation(s) in one or more positions. Non-limiting examples of amino acid mutations comprise amino acid substitutions and/or insertions and/or deletions. In some embodiments, the LAP, or the fragment or derivative thereof, comprises amino acid substitution(s) in one or more positions. As a non-limiting example, the LAP, or the fragment or derivative thereof, comprises may comprise amino acid substitution(s) in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 or more positions.

In some embodiments, the LAP, or the fragment or derivative thereof, comprises may comprise one or more amino acid substitutions and/or insertions and/or deletions. Amino acid substitution means that an amino acid residue is substituted for a replacement amino acid residue at the same position. Inserted amino acid residues may be inserted at any position and may be inserted such that some or all of the inserted amino acid residues are immediately adjacent one another or may be inserted such that none of the inserted amino acid residues is immediately adjacent another inserted amino acid residue. One or more amino acids may be substituted and/or inserted and/or deleted from the sequence of SEQ ID NO: 29. One or more amino acids may be substituted and/or inserted and/or deleted from the sequence of SEQ ID NO: 82. One or more amino acids may be substituted and/or inserted and/or deleted from the sequence of SEQ ID NO: 118. One or more amino acids may be substituted and/or inserted and/or deleted from the sequence of SEQ ID NO: 116.

In some embodiments, the LAP, or the fragment or derivative thereof may comprise one or more amino acid substitutions and/or insertions and/or deletions at one or more locations in the amino acid sequence, e.g., as compared to an amino acid sequence of a reference LAP. For example, the LAP may include a substitution(s) of one or more amino acids in the amino acid sequence of a parent LAP with a similar or homologous amino acid(s) or a dissimilar amino acid(s).

In certain embodiments, amino acid mutations (e.g., substitutions) to a protein or portion thereof are those which: (1) reduce susceptibility to proteolysis, (2) reduce susceptibility to oxidation, (3) alter binding affinity for forming protein complexes, or (4) confer or modify other physicochemical or functional properties. For example, single or multiple amino acid substitutions (e.g., conservative amino acid substitutions) may be made in the normally occurring (i.e., natural) sequence or parental sequence. In some embodiments, the amino acid sequence of the LAP disclosed herein may be mutated, for example, to make the LAP specific to proteolytic activation. In some embodiments, the amino acid sequence of the LAP disclosed herein may be mutated, for example, to allow for mechanical activation of the LAP. In some embodiments, the amino acid sequence of the LAP disclosed herein may be mutated, for example, to eliminate one or more protease cleavage sites within the LAP, or the fragment or derivative thereof. In some embodiments, the amino acid sequence of the LAP disclosed herein may be mutated, for example, to decrease binding of the LAP to, for example, a milieu molecules such as but not limited to a latency associated binding protein (LTBP).

In some embodiments, the LAP, or the fragment or derivative thereof, comprises one or more mutations which make the LAP specific to proteolytic activation. In some embodiments, the LAP, or the fragment or derivative thereof, comprises one or more mutations that allow for proteolytic activation of the LAP. In some embodiments, the LAP, or the fragment or derivative thereof, comprises one or more mutations which allow for mechanical activation of the LAP. In some embodiments, the LAP, or the fragment or derivative thereof, comprises one or more mutations which facilitate mechanical activation of the LAP. In some embodiments, the LAP, or the fragment or derivative thereof, comprises one or more mutations which inhibit or block mechanical activation of the LAP. In some embodiments, the LAP, or the fragment or derivative thereof, comprises one or more mutations which eliminate one or more protease cleavage sites within the LAP, or the fragment or derivative thereof. In some embodiments, the LAP, or the fragment or derivative thereof, comprises one or more mutations that decrease binding of the LAP to a latency associated binding protein (LTBP).

In various embodiments, the LAP, or the fragment or derivative thereof described herein, may comprise one more mutations described herein. In some embodiments, the one or more mutations may allow for proteolytic activation of a mature TGFβ family polypeptide, or a fragment or derivative thereof, described herein. In some embodiments, the one or more mutations may allow for mechanical activation of a mature TGFβ family polypeptide, or a fragment or derivative thereof, described herein. In some embodiments, the LAP, or the fragment or derivative thereof, comprises one or more mutations which facilitate mechanical activation of the LAP. In some embodiments, the LAP, or the fragment or derivative thereof, comprises one or more mutations which inhibit or block mechanical activation of the LAP. In some embodiments, the LAP, or the fragment or derivative thereof described herein may be mutated, for example, to produce an autoactive form of TGF-β, i.e., a TGFβ that does not require activation (such as by way of proteolytic or mechanical activation) to induce downstream signaling (e.g., Smad2/3 signaling and/or ERK signaling, or the like), described herein.

In some embodiments, the LAP or the fragment or derivative thereof may comprise one or more mutations which may, for example, eliminate one or more protease cleavage sites within the LAP, or the fragment or derivative thereof. In some embodiments, the one or more mutations may, for example, decrease binding of the LAP, or the fragment or derivative thereof, to a latency associated binding protein (LTBP).

In some embodiments, the LAP or the fragment or derivative thereof may comprise one or more mutations which may, for example, introduce one or more protease cleavage sites into the LAP, or the fragment or derivative thereof such as via insertion of a protease cleavage site described herein. In some embodiments, the insertion may comprise a PLGL insertion such as that which may be useful for MMP2 cleavage. In some embodiments, the insertion may comprise a PLGI insertion such as that which may be use for MMP3, MMP7 and/or MMP8 cleavage

In some embodiments, the LAP disclosed herein may include conservative modifications and/or substitutions. A conservative amino acid modifications and/or substitution should not substantially change the structural characteristics of the parent sequence. For example, amino acids belonging to one of the following groups represent conservative mutations: Group I: Ala, Pro, Gly, Gln, Asn, Ser, Thr; Group II: Cys, Ser, Tyr, Thr; Group III: Val, Ile, Leu, Met, Ala, Phe; Group IV: Lys, Arg, His; Group V: Phe, Tyr, Trp, His; and Group VI: Asp, Glu.

In some embodiments, the LAP, or the fragment or derivative thereof may comprise an amino acid substitution at a position corresponding, for example, to amino acid residue C33 of the LAP. In some embodiments, the LAP, or the fragment or derivative thereof, may comprise a C33S mutation (cysteine-to-serine mutation at position 33), and position 33 is in relation to the sequence of SEQ ID NO: 82. In some embodiments, the LAP, or the fragment or derivative thereof, may comprise a C33S mutation (cysteine-to-serine mutation at position 33), and position 33 is in relation to the sequence of SEQ ID NO: 116. In some embodiments, a LAP comprising a C33S mutation may comprise the sequence of SEQ ID NO: 31. In some embodiments, a LAP comprising a C33S mutation may comprise the sequence of SEQ ID NO: 80. In some embodiments, a LAP comprising a C33S mutation may comprise the sequence of SEQ ID NO: 88.

In some embodiments, the LAP, or the fragment or derivative thereof, may comprise an amino acid substitution at a position corresponding, for example, to amino acid residue C24 of the LAP. In some embodiments, the LAP, or the fragment or derivative thereof, may comprise a C24S mutation (cysteine-to-serine mutation at position 24), and position 24 is in relation to the sequence of SEQ ID NO: 116. In some embodiments, a LAP comprising a C24S mutation may comprise the sequence of SEQ ID NO: 94.

Since the LAP may be covalently linked to cell surface-bound milieu molecules at C33 or C24, the C33S mutation or the C24S mutation may prevent disulfide bonding of the LAP to a milieu molecule(s) during secretion, which prevents incorporation of milieu molecules into TGFβ SLC. There are three classes of milieu molecules expressed on different cell types: (i) LTBP (latency-associated binding protein); (ii) GARP (LRRC32, glycoprotein-A repetition predominant protein); and (iii) NRROS (LRRC33, leucine rich repeat containing protein 33). TGFβ in complex with LTBP1, LTBP3 and LTBP4 is stored in the extracellular matrix (ECM). TGFβ in complex with GARP is stored on the surface of endothelium and activated regulatory T cells. TGFβ in complex with LRRC33 is stored on macrophages and microglia.

In some embodiments, the one or more mutations of the LAP disclosed herein may comprise a C33S mutation, and position 33 is in relation to the sequence of SEQ ID NO: 82. In some embodiments, the LAP comprising a C33S mutation comprises the sequence of SEQ ID NO: 31. In some embodiments, the LAP comprising a C33S mutation comprises the sequence of SEQ ID NO: 80. In some embodiments, the LAP comprising a C33S mutation comprises the sequence of SEQ ID NO: 88.

In some embodiments, the one or more mutations of the LAP disclosed herein may comprise a C24S mutation, and position 24 is in relation to the sequence of SEQ ID NO: 116. In some embodiments, the LAP comprising a C24S mutation comprises the sequence of SEQ ID NO: 94.

In some embodiments, the LAP comprising the one or more mutations disclosed herein comprises the amino acid sequence of SEQ ID NO: 31, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 31. In certain embodiments, the nucleotide sequence that encodes the LAP comprising the one or more mutations comprises the nucleotide sequence that encodes the amino acid sequence of SEQ ID NO: 31, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 31. In certain embodiments, the nucleotide sequence that encodes the LAP comprising the one or more mutations comprises the nucleotide sequence of SEQ ID NO: 32, or a nucleotide sequence having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 32. In certain embodiments, the LAP comprising the one or more mutations comprises the amino acid sequence of SEQ ID NO: 31. In certain embodiments, the nucleotide sequence that encodes the LAP comprises the nucleotide sequence of SEQ ID NO: 32.

In some embodiments, the LAP, or the fragment or derivative thereof comprises an amino acid sequence that has at least 80% sequence identity to SEQ ID NO: 31. In some embodiments, the LAP, or the fragment or derivative thereof comprises the amino acid sequence of SEQ ID NO: 31. In some embodiments, the LAP consists of the amino acid sequence of SEQ ID NO: 31.

In some embodiments, the LAP comprising the one or more mutations disclosed herein comprises the amino acid sequence of SEQ ID NO: 80, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 80. In certain embodiments, the nucleotide sequence that encodes the LAP comprising the one or more mutations comprises the nucleotide sequence that encodes the amino acid sequence of SEQ ID NO: 80, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 80. In certain embodiments, the nucleotide sequence that encodes the LAP comprising the one or more mutations comprises the nucleotide sequence of SEQ ID NO: 81, or a nucleotide sequence having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 81. In certain embodiments, the LAP comprising the one or more mutations comprises the amino acid sequence of SEQ ID NO: 80. In certain embodiments, the nucleotide sequence that encodes the LAP comprises the nucleotide sequence of SEQ ID NO: 81.

In some embodiments, the LAP, or the fragment or derivative thereof comprises an amino acid sequence that has at least 80% sequence identity to SEQ ID NO: 80. In some embodiments, the LAP, or the fragment or derivative thereof comprises the amino acid sequence of SEQ ID NO: 80. In some embodiments, the LAP consists of the amino acid sequence of SEQ ID NO: 80.

In some embodiments, the LAP comprising the one or more mutations disclosed herein comprises the amino acid sequence of SEQ ID NO: 88, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 88. In certain embodiments, the nucleotide sequence that encodes the LAP comprising the one or more mutations comprises the nucleotide sequence that encodes the amino acid sequence of SEQ ID NO: 88, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 88. In certain embodiments, the nucleotide sequence that encodes the LAP comprising the one or more mutations comprises the nucleotide sequence of SEQ ID NO: 89, or a nucleotide sequence having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 89. In certain embodiments, the LAP comprising the one or more mutations comprises the amino acid sequence of SEQ ID NO: 88. In certain embodiments, the nucleotide sequence that encodes the LAP comprises the nucleotide sequence of SEQ ID NO: 89.

In some embodiments, the LAP, or the fragment or derivative thereof comprises an amino acid sequence that has at least 80% sequence identity to SEQ ID NO: 88. In some embodiments, the LAP, or the fragment or derivative thereof comprises the amino acid sequence of SEQ ID NO: 88. In some embodiments, the LAP consists of the amino acid sequence of SEQ ID NO: 88.

In some embodiments, the LAP comprising the one or more mutations disclosed herein comprises the amino acid sequence of SEQ ID NO: 94, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 94. In certain embodiments, the nucleotide sequence that encodes the LAP comprising the one or more mutations comprises the nucleotide sequence that encodes the amino acid sequence of SEQ ID NO: 94, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 94. In certain embodiments, the nucleotide sequence that encodes the LAP comprising the one or more mutations comprises the nucleotide sequence of SEQ ID NO: 95, or a nucleotide sequence having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 95. In certain embodiments, the LAP comprising the one or more mutations comprises the amino acid sequence of SEQ ID NO: 94. In certain embodiments, the nucleotide sequence that encodes the LAP comprises the nucleotide sequence of SEQ ID NO: 95.

In some embodiments, the LAP, or the fragment or derivative thereof comprises an amino acid sequence that has at least 80% sequence identity to SEQ ID NO: 94. In some embodiments, the LAP, or the fragment or derivative thereof comprises the amino acid sequence of SEQ ID NO: 94. In some embodiments, the LAP consists of the amino acid sequence of SEQ ID NO: 94.

In some embodiments, the one or more mutations of the LAP disclosed herein may comprise a C33S mutation, a C223S mutation, and/or a C225S mutation, and positions 33, 223 and/or 225 are in relation to the sequence of SEQ ID NO: 82. In some embodiments, the LAP comprising a C33S, C223S and a C225S mutation comprises the sequence of SEQ ID NO: 80.

In some embodiments, the LAP comprising the one or more mutations disclosed herein comprises the amino acid sequence of SEQ ID NO: 80, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 80. In certain embodiments, the nucleotide sequence that encodes the LAP comprising the one or more mutations comprises the nucleotide sequence that encodes the amino acid sequence of SEQ ID NO: 80, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 80. In certain embodiments, the nucleotide sequence that encodes the LAP comprising the one or more mutations comprises the nucleotide sequence of SEQ ID NO:81, or a nucleotide sequence having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 81. In certain embodiments, the LAP comprising the one or more mutations comprises the amino acid sequence of SEQ ID NO: 80. In certain embodiments, the nucleotide sequence that encodes the LAP comprises the nucleotide sequence of SEQ ID NO: 81.

In some embodiments, the LAP, or the fragment or derivative thereof comprises an amino acid sequence that has at least 80% sequence identity to SEQ ID NO: 80. In some embodiments, the LAP, or the fragment or derivative thereof comprises the amino acid sequence of SEQ ID NO: 80. In some embodiments, the LAP consists of the amino acid sequence of SEQ ID NO: 80.

### Transforming Growth Factor β (TGFβ) Family Polypeptides

In some embodiments, the polypeptide complex described herein may comprise a Transforming Growth Factor β (TGFβ) family polypeptide, or a fragment or derivative thereof. In some embodiments, the polypeptide complex may comprise a dimeric and/or mature Transforming Growth Factor β (TGFβ) family polypeptide, or a fragment or derivative thereof. In some embodiments, the TGFβ family polypeptide (e.g., a mature dimeric TGFβ family polypeptide), or the fragment or derivative thereof, may be inactive as a result of an interaction with a LAP described herein. In some embodiments, the LAP is a dimeric LAP.

Non-limiting examples of members of the TGFβ family include activins, anti-Müllerian hormone (AMH), bone morphogenetic proteins (BMPs), inhibins, Nodal, growth and differentiation factors (GDFs), and TGFβ isoforms (e.g., TGFβ1, TGFβ2, and TGFβ3). The TGFβ isoforms (e.g., TGFβ1, TGFβ2, and TGFβ3) have each have been identified in mammals and can share 70-82% homology at the amino acid level. The TGFβ isoforms may participate in various cellular processes such as, but not limited to, extracellular matrix (ECM) remodeling, cell migration, invasion, epithelial-mesenchymal transition (EMT), growth inhibition, and/or immune-suppression.

From a mechanistic standpoint, TGFβ, as with other members of the TGFβ family, can be synthesized as a precursor protein, which may form a homodimer that interacts with a latency-associated peptide (LAP) to form a small latent complex (SLC). Milieu molecule(s), e.g., a latent TGF-beta-binding protein (LTBP) may bind to the LAP to form a larger complex called a large latent complex (LLC). The TGFβ gene encodes a preproprotein sequence consisting of, e.g., a signal peptide, a propeptide that ends with a protease cleavage site, and the mature TGFβ sequence. Furin may hydrolyze the protease cleavage site, thereby producing separate TGFβ- and propeptide-derived homodimers. The two homodimers remain noncovalently associated and may be secreted. This latent complex can maintain TGFβ in an inactive form that is incapable of binding to its receptors.

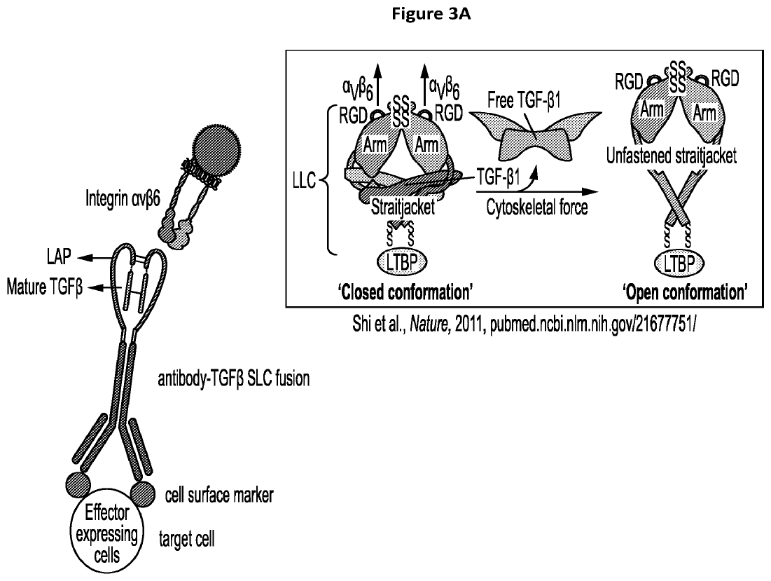

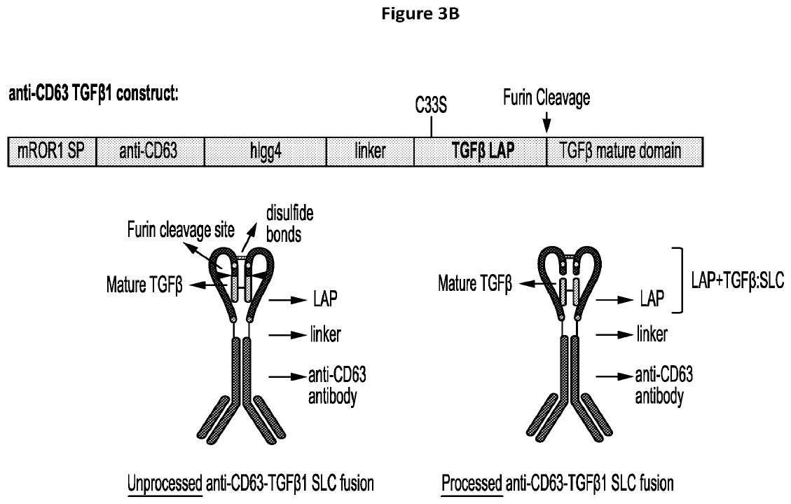

LLCs can be activated proteolytically or mechanically. In proteolytic activation, proteases may either degrade or lead to a conformational change of the LAP domain to release mature TGFβ. Non-limiting examples of proteases which may be involved in proteolysis of LAP include matrix metalloproteinase 2 (MMP2), matrix metalloproteinase 9 (MMP9), thrombospondin1 (TSP-1) plasmin (PLN), and plasma kallikrein (PLK). In mechanical activation, integrins (αvβ6 integrin, αvβ8 integrin, αvβ1 integrin) bind to the ‘RGD’ binding motif at the C-terminus of LAP. Integrin binding at the C-terminus of LAP and cell surface-bound milieu molecule association (through covalent bonding) at the N-terminus of LAP creates a directional pulling force that opens LAP thereby releasing free mature TGFβ (see, e.g., FIG. 3A, box). Mature TGFβ may then bind to the extracellular domain(s) of, e.g., TGFβR1 (TGFβ type I receptor) and/or TGFβR2 (TGFβ type II receptor) such as on the cell surface. Binding TGFβR1 and/or TGFβR2 by mature TGFβ may bring the receptors in close proximity to one another, thereby placing the intracellular serine/threonine kinase domains of the TGFβ receptors in a conformation that can facilitate phosphorylation and/or activation of the receptor(s). In some instances, however, the TGFβRs may already be in close proximity to one another in the absence of the ligand (i.e., a mature TGFβ). In some embodiments, upon binding of the ligand, constitutively active TGFβR2 may phosphorylate TGFβR1 which upon such activation may, in turn, phosphorylate intracellular Smad2/3. By way of a non-limiting example, binding of active mature TGFβ may induce signaling via a Smad-dependent canonical signaling pathway. In some embodiments, active mature TGFβ is capable of inducing Smad2/3 signaling (see, e.g., FIG. 1). In non-canonical pathway(s), an activated TGFβ receptor complex, e.g., comprising mature active TGFβ in complex with TGFβR1 and/or TGFβR2, may transmit a signal via other factors such as, but not limited to, nuclear factor-kappa B (NF-kappa B), TRAF6, extracellular signal-regulated kinase (ERK)TGFβ-activated kinase 1 (TAK1, also known as MAP3K7), tumor necrosis factor (TNF) receptor-associated factor 4 (TRAF4), RHO, phosphoinositide 3-kinase (PI3K), p38 mitogen-activated protein kinase (p38 MAPK AKT (also known as protein kinase B), and/or JUN N-terminal kinase (INK).

In some embodiments, the mature TGFβ family polypeptide, or the fragment or derivative thereof disclosed herein, and the dimeric LAP disclosed herein may be associated via a noncovalent interaction. In some embodiments, the mature TGFβ family polypeptide, or the fragment or derivative thereof, may be inactive as a result of the interaction of the mature TGFβ family polypeptide, or the fragment or derivative thereof, with the LAP, or the fragment or derivative thereof.

In some embodiments, a TGFβ family polypeptide, or a fragment or derivative thereof described herein, may be an autoactive TGFβ family polypeptide, i.e., a TGFβ family polypeptide that does not require activation (such as by way of proteolytic or mechanical activation) to induce TGFβ signaling (e.g., Smad2/3 signaling and/or ERK signaling, or the like). In some embodiments, the TGFβ family polypeptide, or the fragment or derivative thereof, may comprise one or more mutations to produce an autoactive form of TGFβ. The one or more mutations which produce the autoactive form of TGFβ may comprise any of various mutations described herein.

In some embodiments, the LAP, or the fragment or derivative thereof, is heterologous to the mature TGFβ family polypeptide, or the fragment or derivative thereof.

In some embodiments, the mature TGFβ family polypeptide, or the fragment or derivative thereof, and the LAP may be covalently linked and may be separated by a protease cleavage site. In some embodiments, the protease cleavage site may comprise, for example, without limitation, a furin cleavage site comprising the sequence RXXR (SEQ ID NO: 35). In some embodiments, the protease cleavage site may consist of the sequence RXXR (SEQ ID NO: 35). In some embodiments, the protease cleavage site is RHRR (SEQ ID NO: 85), RRKR (SEQ ID NO: 86), or RKKR (SEQ ID NO: 87).

In some embodiments, a mature TGFβ family polypeptide, or the fragment or derivative thereof disclosed herein, may bind a Transforming Growth Factor β Receptor (TGFβR) upon release of the mature TGFβ family polypeptide, or the fragment or derivative thereof, from the SLC disclosed herein. The Transforming Growth Factor β Receptor (TGFβR) may comprise any of various TGFβRs disclosed herein.

In some embodiments, the mature TGFβ family polypeptide, or the fragment or derivative thereof, induces signaling via a Smad-dependent canonical signaling pathway in a target cell or a cell adjacent to the target cell upon release from the SLC disclosed herein. In some embodiments, the mature TGFβ family polypeptide, or the fragment or derivative thereof, induces Smad2/3 signaling in a target cell or a cell adjacent to the target cell upon release from the SLC disclosed herein. In some embodiments, the mature TGFβ family polypeptide, or the fragment or derivative thereof, induces signaling via a non-canonical signaling pathway disclosed herein in a target cell or a cell adjacent to the target cell upon release from the SLC disclosed herein.

In some embodiments, the mature TGFβ family polypeptide, or the fragment or derivative thereof, induces signaling via a Smad-dependent canonical signaling pathway in a target cell or a cell adjacent to the target cell upon activation of the mature TGFβ family polypeptide. In some embodiments, the mature TGFβ family polypeptide, or the fragment or derivative thereof, induces Smad2/3 signaling in a target cell or a cell adjacent to the target cell upon activation of the mature TGFβ family polypeptide. In some embodiments, the mature TGFβ family polypeptide, or the fragment or derivative thereof, induces signaling via a non-canonical signaling pathway disclosed herein in a target cell or a cell adjacent to the target cell upon activation of the mature TGFβ family polypeptide. Activation of mature TGFβ family polypeptide may occur by way of any mechanism disclosed herein. The activation may occur, for example, via mechanisms involving proteolytic activation and/or mechanical activation. In some embodiments, the activation may occur by chemical activation. In some embodiments, the activation may occur by acid, e.g., a low pH such as pH 3.0 which can be achieved with HCl, and/or heat activation.

In some embodiments, the mature TGFβ family polypeptide, or the fragment or derivative thereof, may be chemically dissociated from the LAP, or the fragment or derivative thereof, and released in an active form from the SLC. In some embodiments, the chemical dissociation comprises, for example, without limitation, a protease treatment, a temperature treatment, an acid treatment, or any combination thereof.

In some embodiments, the mature TGFβ family polypeptide, or the fragment or derivative thereof, is mechanically dissociated from the LAP, or the fragment or derivative thereof, and released in an active form from the SLC. By way of a non-limiting example, the mechanical release may occur as a result of an interaction between the LAP, or the fragment or derivative thereof, and an integrin polypeptide such as via an integrin binding motif in the LAP. Non-limiting examples of integrins include αvβ6 integrin, αvβ8 integrin, and αvβ1 integrin.

In some embodiments, the mature TGFβ family polypeptide can be a mature TGFβ polypeptide.

In some embodiments, the mature TGFβ polypeptide can be a mature TGFβ1 polypeptide.

In some embodiments, the mature TGFβ1 polypeptide comprises the amino acid sequence of SEQ ID NO: 23, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 23. In certain embodiments, the nucleotide sequence that encodes the mature TGFβ1 polypeptide comprises the nucleotide sequence that encodes the amino acid sequence of SEQ ID NO: 23, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 23. In certain embodiments, the nucleotide sequence that encodes the mature TGFβ1 polypeptide comprises the nucleotide sequence of SEQ ID NO: 24, or a nucleotide sequence having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 24. In certain embodiments, the mature TGFβ1 polypeptide comprises the amino acid sequence of SEQ ID NO: 23. In certain embodiments, the nucleotide sequence that encodes the mature TGFβ1 polypeptide comprises the nucleotide sequence of SEQ ID NO: 24.

In some embodiments, the mature TGFβ1 polypeptide comprises the sequence of SEQ ID NO: 23. In some embodiments, the mature TGFβ1 polypeptide consists of the sequence of SEQ ID NO: 23.

In some embodiments, the mature TGFβ1 polypeptide comprises the amino acid sequence of SEQ ID NO: 90, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 90. In certain embodiments, the nucleotide sequence that encodes the mature TGFβ1 polypeptide comprises the nucleotide sequence that encodes the amino acid sequence of SEQ ID NO: 90, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 90. In certain embodiments, the nucleotide sequence that encodes the mature TGFβ1 polypeptide comprises the nucleotide sequence of SEQ ID NO: 91, or a nucleotide sequence having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 91. In certain embodiments, the mature TGFβ1 polypeptide comprises the amino acid sequence of SEQ ID NO: 90. In certain embodiments, the nucleotide sequence that encodes the mature TGFβ1 polypeptide comprises the nucleotide sequence of SEQ ID NO: 91.

In some embodiments, the mature TGFβ1 polypeptide comprises the sequence of SEQ ID NO: 90. In some embodiments, the mature TGFβ1 polypeptide consists of the sequence of SEQ ID NO: 90.

In some embodiments, the mature TGFβ polypeptide can be a mature TGFβ2 polypeptide.

In some embodiments, the mature TGFβ2 polypeptide comprises the amino acid sequence of SEQ ID NO: 27, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 27. In certain embodiments, the nucleotide sequence that encodes the mature TGFβ2 polypeptide comprises the nucleotide sequence that encodes the amino acid sequence of SEQ ID NO: 27, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 27. In certain embodiments, the nucleotide sequence that encodes the mature TGFβ2 polypeptide comprises the nucleotide sequence of SEQ ID NO: 28, or a nucleotide sequence having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 28. In certain embodiments, the mature TGFβ2 polypeptide comprises the amino acid sequence of SEQ ID NO: 27. In certain embodiments, the nucleotide sequence that encodes the mature TGFβ2 polypeptide comprises the nucleotide sequence of SEQ ID NO: 28.

In some embodiments, the mature TGFβ2 polypeptide comprises the sequence SEQ ID NO: 27. In some embodiments, the mature TGFβ2 polypeptide consists of the sequence SEQ ID NO: 27.

Other non-limiting examples mature TGFβ family polypeptides that may be useful in the practice of the present disclosure include a mature Growth Differentiation Factor 8 (GDF8), a mature Growth Differentiation Factor 11 (GDF11) polypeptide, and a mature Bone Morphogenetic Protein 4 (BMP4).

In some embodiments, the mature TGFβ family polypeptide may be a mature Growth Differentiation Factor 8 (GDF8) polypeptide. In some embodiments, the mature TGFβ family polypeptide may be a mature Growth Differentiation Factor 11 (GDF11) polypeptide. In some embodiments, the mature TGFβ family polypeptide may be a mature Bone Morphogenetic Protein 4 (BMP4).

### Target-Binding Polypeptides

In some embodiments, the polypeptide complex described herein may comprise a target-binding polypeptide. In some embodiments, the target-binding polypeptide may bind a molecule on a target cell. In some embodiments, the target-binding polypeptide that binds a molecule in an extracellular matrix (ECM). In some embodiments, the target-binding peptide is not internalizing. In some embodiments, the target-binding peptide is capable of internalization.

In some embodiments, the polypeptide complex disclosed herein may comprise a small latent complex (SLC) disclosed herein comprising a dimeric LAP disclosed herein, or a fragment or derivative thereof, which may be attached to the target-binding polypeptide. In some embodiments, the LAP, or the fragment or derivative thereof, may be covalently attached to the target-binding polypeptide. In some embodiments, the LAP, or the fragment or derivative thereof, may be covalently attached to the target-binding polypeptide via a linker disclosed herein. In some embodiments, the linker may be located between the target-binding polypeptide and the LAP, or the fragment or derivative thereof disclosed herein. In some embodiments the LAP, or the fragment or derivative thereof, may be noncovalently bound to the target-binding polypeptide. In some embodiments, the target-binding polypeptide binds both the LAP and the target(s) of the target-binding polypeptide, e.g., a molecule(s) on a target cell and/or a molecule in an extracellular matrix (ECM).

In some embodiments, the target-binding polypeptide is an antigen-binding polypeptide or antigen-binding fragment thereof.

In some embodiments, the antigen-binding polypeptide binds to at least one antigen on a target cell. In some embodiments, the antigen-binding moiety binds to two or more antigens on a target cell. In some embodiments, the two or more antigens can be associated with the same target cell. In some embodiments, the two or more antigens can be associated with different target cells. Non-limiting examples of a target cells include fibroblasts, chondroblasts, osteoblasts, myofibroblasts, plasma cells, adipocytes, and a leukocytes. In some embodiments, the target cell may comprise an immune cell for example, without limitation, a T-cell (e.g., an activated regulatory T cell, CD40+ T cells, CD90+ T cells), a natural killer (NK) cell, a macrophage, or a mast cell. In some embodiments, the target cell may be a microglia cell. In some embodiments, the target cell may be an endothelial cell. In some embodiments, the target cell may be an epithelial cell (e.g., an intestinal epithelial cell). In some embodiments, the target cell may be a microglia cell. In some embodiments, the target cell may be a dendritic cell. In some embodiments, the target cell may be a beta cell (e.g., a pancreatic beta cell).

In some embodiments, an antigen on a T cell (e.g., CD4 or CD90) may be targeted by an antigen-binding polypeptide described herein, and a polypeptide complex comprising such antigen-binding polypeptide, or a pharmaceutical composition thereof described herein, may, e.g., promote regulatory T cell (Treg) differentiation and/or maintain immune tolerance such as, e.g., in inflammatory bowel disease (IBD).

In some embodiments, an antigen on an epithelial cell such as an intestinal epithelial cell (e.g., mEpcam or mOlfm4) may be targeted by an antigen-binding polypeptide described herein, and a polypeptide complex comprising such antigen-binding polypeptide, or a pharmaceutical composition thereof described herein, may, e.g., enhance epithelial barrier integrity such as, e.g., in IBD.

In some embodiments, an antigen on a dendritic cell (e.g., mClec9a) may be targeted by an antigen-binding polypeptide described herein, and a polypeptide complex comprising such antigen-binding polypeptide, or a pharmaceutical composition thereof described herein, may, e.g., decrease antigen presentation and/or inhibit goblet cell differentiation such as, e.g., in IBD.

In some embodiments, an antigen on a pancreatic beta cell (e.g., HLA-A2:INS) may be targeted by an antigen-binding polypeptide described herein, and a polypeptide complex comprising such antigen-binding polypeptide, or a pharmaceutical composition thereof described herein, may, e.g., suppress autoreactive immune cells such as, e.g., in Type 1 diabetes mellitus (T1D).

In some embodiments, the antigen-binding polypeptide binds to at least one antigen of the extracellular matrix (ECM). In some embodiments, the antigen-binding moiety binds to two or more antigens of the ECM. In some embodiments, the two or more antigens are associated with the same ECM. In some embodiments, the two or more antigens are associated with different ECM. Examples of antigens associated with ECM may be associated with various ECM molecules such as, but not limited to, collagen (e.g., type X collagen, also termed collagen X), fibrillar collagens, fibronectin, elastin, and/or laminins. In some embodiments, the antigen-binding polypeptide targets collagen. In some embodiments, the antigen-binding polypeptide targets fibronectin.

In some embodiments, an antigen of the ECM (e.g., fibronectin) may be targeted by an antigen-binding polypeptide described herein, and a polypeptide complex comprising such antigen-binding polypeptide, or a pharmaceutical composition thereof described herein, may, e.g., repopulate the newly developing areas of the matrix such as, e.g., in aortic aneurysm in Marfan syndrome (MFS).

In some embodiments, an antigen of the ECM (e.g., type X collagen) may be targeted by an antigen-binding polypeptide described herein, and a polypeptide complex comprising such antigen-binding polypeptide, or a pharmaceutical composition thereof described herein, may, e.g., reduce inflammation in joints such as, e.g., in rheumatoid arthritis (RA).

In some embodiments, the antigen-binding polypeptide binds to at least one antigen associated with a TGFβ dysregulation disorder. In some embodiments, the antigen-binding polypeptide binds to two or more antigens associated with a TGFβ dysregulation disorder. In some embodiments, the two or more antigens associated with a TGFβ dysregulation disorder are associated with the same TGFβ dysregulation disorder. In some embodiments, the two or more antigens associated with a TGFβ dysregulation disorder are associated with different antigens associated with a TGFβ dysregulation disorder. Non-limiting examples of TGFβ dysregulation disorders include Type 1 diabetes mellitus, inflammatory bowel disease (IBD), Marfan syndrome (MFS), aortic aneurysm in MFS, an autoimmune disorder, an arthritis (e.g., rheumatoid arthritis (RA)), lupus (e.g., systemic lupus), and a wound healing disorder.

Examples of antigens that may be targeted by the antigen-binding polypeptide or antigen-binding fragment thereof include, but are not limited to, PD-1, EDB-FN, CD63, CD4, CD90, Epcam, Madcam, a4b7, CCR9, Clec9a, Xcr1, and Olfm4. In some embodiments, the antigen targeted by the antigen-binding polypeptide or antigen-binding fragment thereof may comprise HLA-A2:INS. In some embodiments, the antigen targeted by the antigen-binding polypeptide or antigen-binding fragment thereof may comprise fibronectin. In some embodiments, the antigen targeted by the antigen-binding polypeptide or antigen-binding fragment thereof may comprise collagen-10.

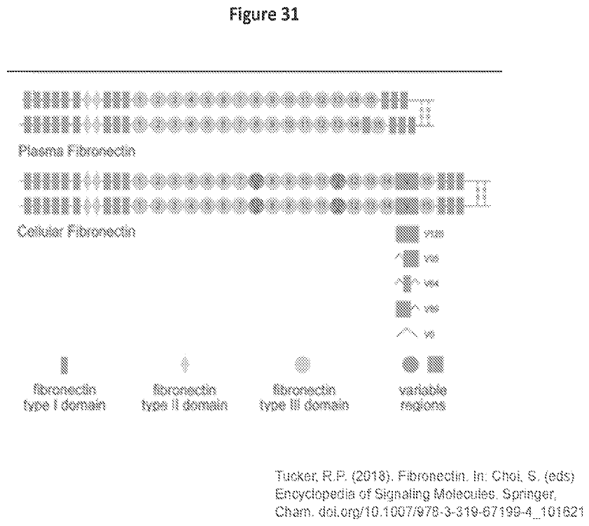

In some embodiments, the antigen-binding polypeptide or antigen-binding fragment thereof can comprise a fibronectin (FN), or a fragment or derivative thereof. Fibronectins (FNs) are multifunctional, high molecular weight glycoprotein components of both bodily fluids (e.g., plasma) and the extracellular matrix (ECM). FNs participate in various biological processes such as, without limitation, cell migration, cell adhesion, thrombosis and haemostasias, and wound healing, as well as in the establishment and maintenance of normal cellular morphology (i.e., cell shape), development, and oncogenic transformation. Structural diversity in plasma and cellular fibronectins (FNs) arises from alternative splicing of three domains of the primary FN transcript which can generate at least 20 different isoforms that may be differentially expressed in, e.g., tumor and normal tissue (see, e.g., FIG. 31). Plasma FN is produced and secreted by hepatocytes as soluble dimeric form. Cellular FN is expressed by several mesenchymal cells as dimeric or cross-linked multimeric forms and is deposited as fibrils in the ECM. Cellular FN is required for fibrillin-1 and Col Type I deposition in the ECM. FN can interact with many other ECM proteins as well as small molecules, growth factors, glycosaminoglycans (GAGs), cell surface receptors and other FN molecules.

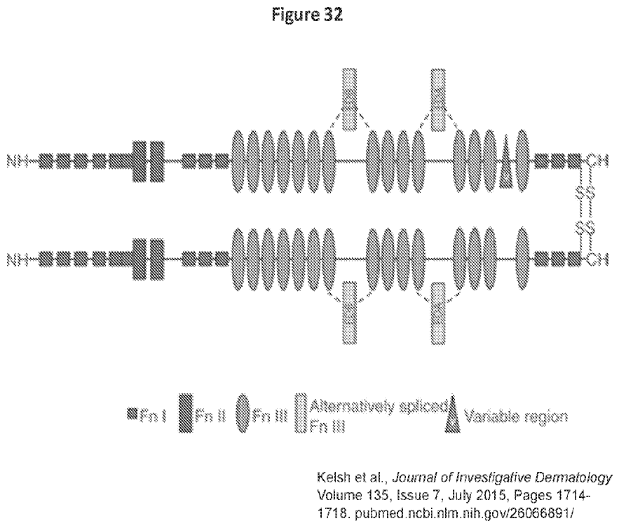

FN isoforms (e.g., cellular FN) containing the EDB domain are expressed in growing and remodeling tissues (see, e.g., FIG. 32). EDB is a small domain of 91 amino acids (see, e.g., SEQ ID NO: 84) that is part of the fibronectin isoforms created by alternative splicing. The sequence of EDB is identical in mouse and humans. EDB-containing isoforms are expressed during embryonic and postnatal development and in a variety of solid tumors (and can be a marker of newly forming vessels) but are virtually undetectable in normal adult tissues, except for in the endometrium during the proliferative phase and some vessels in the ovaries. EDB-containing FN isoforms have been shown, in particular, to play roles in, e.g., protein stability, proliferation, vascularization, inflammation, opsonization (phagocytosis), and cell attachment. Furthermore, EDB-FN can increase the proteolytic sensitivity of FN, suggesting that EDB may increase the rate of ECM turnover. Presence of EDB can also upregulate expression of vascular endothelial growth factor (VEGF) and can be associated with enhanced angiogenesis and endothelial proliferation.

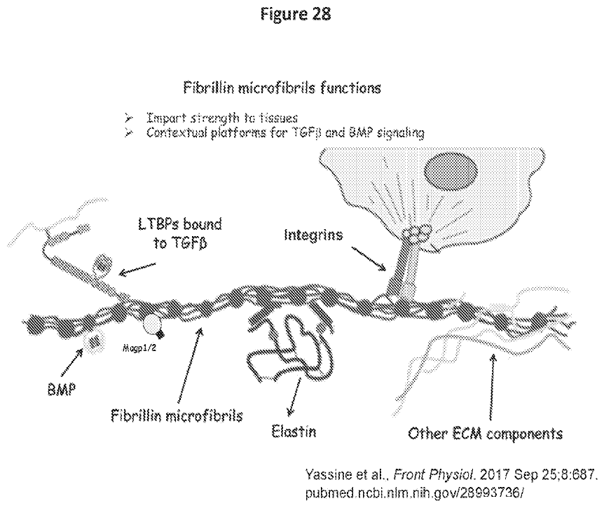

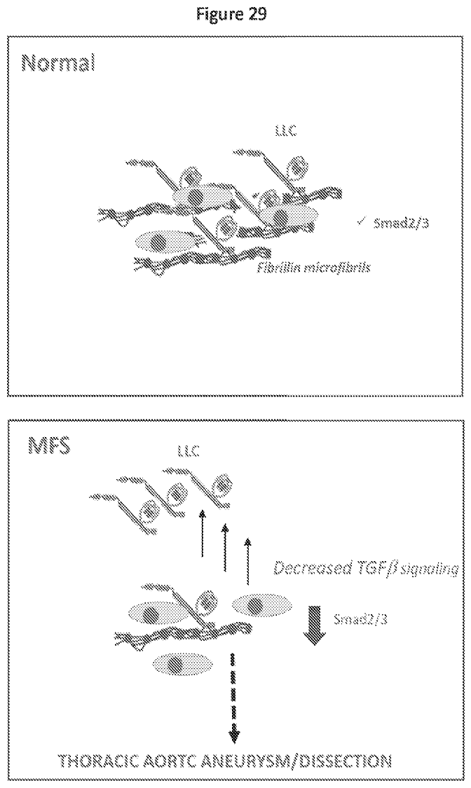

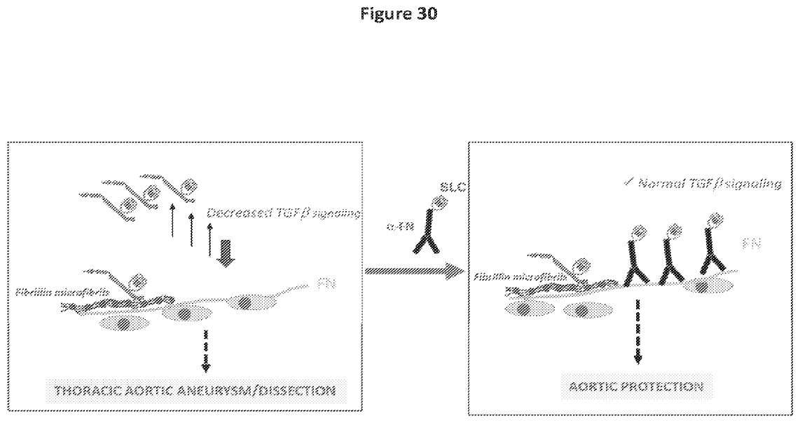

Microfibrils within the ECM are composed of fibrillin polymers which can be associated with elastin, other glycoproteins, as well as growth factors, and FN is essential for microfibril formation. Fibrillin microfibrils impart strength to tissue (see, e.g., FIG. 28) and dysregulation of microfibril assembly can be implicated in disease states. Mutations in the fibrillin glycoproteins, encoded by FBN1, FBN2, and FBN3, can be associated with various connective tissue disorders, the most notable being FBN1 causing Marfan syndrome (MFS) (see, e.g., FIGS. 28-30). FBN1 gene mutations that can, e.g., cause MFS can alter the structure or stability of fibrillin-1, reduce the amount of fibrillin-1 produced by the cell, and/or impair the transport of fibrillin-1 out of the cell. Such mutations can lead to a severe reduction in the amount of fibrillin-1 available to form microfibrils. Aortic root dilatation/dissection is one of the cardinal features of MFS (see, e.g., FIGS. 29-30).

In some embodiments, the antigen-binding polypeptide or antigen-binding fragment thereof can target an antigen which can be associated with MFS.

In some embodiments, the antigen is associated with an autoimmune disease or disorder. An antigen associated with an autoimmune disease or disorder may be derived, for example, from cell receptors and/or cells which produce “self”-directed antibodies. In some embodiments, the antigen is associated with an autoimmune disease or disorder such as, e.g., autoimmune gastritis, vasculitis, Wegener's granulomatosis, Hashimoto's thyroiditis, psoriasis Graves' disease, Guillain-Barre syndrome, chronic inflammatory demyelinating polyneuropathy Crohn's disease, ulcerative colitis, Rheumatoid arthritis (RA), multiple sclerosis (MS), Sjögren's syndrome, sarcoidosis, Systemic lupus erythematosus, Type 1 diabetes mellitus, insulin dependent diabetes mellitus (IDDM), autoimmune thyroiditis, reactive arthritis, Myasthenia gravis, ankylosing spondylitis, scleroderma, polymyositis, or dermatomyositis.

Non-limiting examples of autoimmune antigens include platelet antigen, islet cell antigen, myelin protein antigen, Rheumatoid factor, anticitrullinated protein, glucose-6-phosphate isomerase, receptors such as lipocortin 1, neutrophil nuclear proteins such as lactoferrin and 25-35 kD nuclear protein, Sm antigens, e.g., in snRNPs, granular proteins such as bactericidal permeability increasing protein (BPI), elastase, fibrin, vimentin, filaggrin, fibrinogen, collagen I and II peptides, plasminogen, alpha-enolase, translation initiation factor 4G1, perinuclear factor, keratin, Sa (cytoskeletal protein vimentin), citrullinated proteins and peptides such as CCP-1, CCP-2 (cyclical citrullinated peptides), circulating serum proteins such as RFs (IgG, IgM), components of articular cartilage such as collagen II, IX, and XI, nuclear components such as RA33/hnRNP A2, ferritin, stress proteins such as HSP-65, -70, -90, BiP, inflammatory/immune factors such as B7-H1, IL-1 alpha, and IL-8, enzymes such as alpha-enolase, calpastatin, dipeptidyl peptidase, eukaryotic translation elongation factor 1 alpha 1 aldolase-A, osteopontin, cathepsin G, myeloperoxidase, proteinase 3 antigen, rheumatoid factor, histones, nucleic acids such as, RNA, dsDNA, ssDNA, and ribonuclear particles, ribosomal P proteins, myelin protein, cardiolipin, vimentin, Sm antigens (including, e.g., SmD's and SmB′/B), U1RNP, A2/B1 hnRNP, Ro (SSA), and La (SSB) antigens.

In some embodiments, the antigen is an endogenous molecule of a subject. In some embodiments, the antigen is targeted by an immune response in an autoimmune disease disclosed herein.

In some embodiments, the antigen is associated with a disease related to a TGFβ loss-of-function mutation(s). A non-limiting example of a diseased related to a TGFβ loss-of-function mutation(s) is aortic aneurysm.

In some embodiments, the target-binding polypeptide may comprise an antibody or a fragment or derivative thereof. In some embodiments, the antigen-binding polypeptide may comprise an antibody or antigen-binding fragment thereof.

In some embodiments, the antigen-binding polypeptide can be an antibody of an antigen-binding fragment thereof.

Without wishing to be bound by theory, an antibody disclosed herein may comprise, e.g., immunoglobulin molecules comprised of four polypeptide chains, two immunoglobulin heavy (H) chains (HCs) and two immunoglobulin light chains (LCs) interconnected by disulfide bonds (i.e., “full antibody molecules”), as well as multimers thereof (e.g., IgM) or antigen-binding fragments thereof. Each heavy chain may be comprised of a heavy chain variable region (“HCVR” or “VH”) and a heavy chain constant region (comprised of domains CH1, CH2 and CH3). Each light chain may be comprised of a light chain variable region (“LCVR” or “VL”) and a light chain constant region (CL). The VH and VL regions can be further subdivided into regions of hypervariability, termed complementarity determining regions (CDRs), interspersed with regions that are more conserved, termed framework regions (FR). Each VH and VL may be composed of three CDRs and four FRs, which may be arranged from amino-terminus to carboxy-terminus in the following order: FR1, CDR1, FR2, CDR2, FR3, CDR3, FR4. In certain embodiments of the disclosure, the FRs of the antibody (or antigen-binding fragment thereof) may be identical to the human germline sequences or may be naturally or artificially modified. An amino acid consensus sequence may be defined based on a side-by-side analysis of two or more CDRs. In some embodiments, the antibodies disclosed herein may comprise one or more amino acid substitutions, insertions and/or deletions, for example, in the framework and/or CDR regions of the heavy and light chain variable domains as compared to the corresponding germline sequences.

Antibodies that may be useful in the practice of the disclosure can be full-length (for example, an IgG1 or IgG4 antibody) or may comprise only an antigen-binding portion (for example, a Fab, F(ab′)2 or scFv fragment), and may be modified to affect functionality, e.g., to increase persistence in the host or to eliminate residual effector functions. In certain embodiments, the antibodies may be bispecific.

The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds an antigen to form a complex. An antibody fragment may include a Fab fragment, a F(ab′)2 fragment, a Fv fragment, a dAb fragment, a fragment containing a CDR, or an isolated CDR. In certain embodiments, the term “antigen-binding fragment” refers to a polypeptide fragment of a multi-specific antigen-binding molecule. Antigen-binding fragments of an antibody may be derived, e.g., from full antibody molecules using any suitable standard techniques such as proteolytic digestion or recombinant genetic engineering techniques involving the manipulation and expression of DNA encoding antibody variable and (optionally) constant domains. Such DNA is known and/or is readily available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

Non-limiting examples of antigen-binding fragments include: (i) Fab fragments; (ii) F(ab′)2 fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR) such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g., monovalent nanobodies, bivalent nanobodies, etc.), small modular immunopharmaceuticals (SMIPs), and shark variable IgNAR domains, are also encompassed within the expression “antigen-binding fragment”, as used herein.

An antigen-binding fragment of an antibody will typically comprise at least one variable domain. The variable domain may be of any size or amino acid composition and will generally comprise at least one CDR, which is adjacent to or in frame with one or more framework sequences. In antigen-binding fragments having a VH domain associated with a VL domain, the VH and VL domains may be situated relative to one another in any suitable arrangement. For example, the variable region may be dimeric and contain VH-VH, VH-VL or VL-VL dimers. Alternatively, the antigen-binding fragment of an antibody may contain a monomeric VH or VL domain.

In certain embodiments, an antigen-binding fragment of an antibody may contain at least one variable domain covalently linked to at least one constant domain. Non-limiting, exemplary configurations of variable and constant domains that may be found within an antigen-binding fragment of an antibody of the present disclosure include: (i) VH-CH1; (ii) VH-CH2; (iii) VH-CH3; (iv) VH-CH1-CH2; (V) VH-CH1-CH2-CH3; (vi) VH-CH2-CH3; (vii) VH-CL; (viii) VL-CH1; (ix) VL-CH2; (x) VL-CH3; (xi) VL-CH1-CH2; (xii) VL-CH1-CH2-CH3; (xiii) VL-CH2-CH3; and (xiv) VL-CL. In any configuration of variable and constant domains, including any of the exemplary configurations listed above, the variable and constant domains may be either directly linked to one another or may be linked by a full or partial hinge or linker region. A hinge region may consist of at least 2 (e.g., 5, 10, 15, 20, 40, 60 or more) amino acids, which result in a flexible or semi-flexible linkage between adjacent variable and/or constant domains in a single polypeptide molecule. Moreover, an antigen-binding fragment of an antibody of the present disclosure may comprise a homodimer or heterodimer (or other multimer) of any of the variable and constant domain configurations listed above in non-covalent association with one another and/or with one or more monomeric VH or VL domain (e.g., by disulfide bond(s)).

As with full antibody molecules, antigen-binding fragments may be mono-specific or multi-specific (e.g., bi-specific). A multi-specific antigen-binding fragment of an antibody will typically comprise at least two different variable domains, wherein each variable domain is capable of specifically binding to a separate antigen or to a different epitope on the same antigen. Any multi-specific antibody format, including the exemplary bi-specific antibody formats disclosed herein, may be adapted for use in the context of an antigen-binding fragment of an antibody of the present disclosure using routine techniques available in the art.

In some embodiments, the antibody or antigen-binding fragment thereof disclosed herein comprises a heavy chain variable region (HCVR) In some embodiments, the antibody or antigen-binding fragment thereof comprises a light chain variable region (LCVR). In some embodiments, the antibody or antigen-binding fragment thereof comprises an immunoglobulin heavy chain constant domain (CH) such as, but not limited to, an IgG1 domain or an IgG4 domain. In some embodiments, the immunoglobulin heavy chain constant domain (CH) can be an IgG1 domain. In some embodiments, the immunoglobulin heavy chain constant domain (CH) can be an IgG4 domain. In some embodiments, the antibody or antigen-binding fragment thereof comprises an immunoglobulin light chain constant domain (CL).

In some embodiments, an antibody or antigen-binding fragment thereof of the present disclosure may comprise a heavy chain constant region comprising one or more amino acid alterations in a hinge region. In some embodiments, the amino acid alteration(s) in the hinge region may reduce binding to an Fcγ receptor. Examples of such modifications are disclosed in US 2018/0282411, the content of which is incorporated herein by reference in its entirety for all purposes. In some embodiments, an antigen-binding polypeptide comprises a modification within amino acid positions 233-236 (by EU numbering) by replacing naturally occurring residues with glycine(s) and/or deletion(s). In some embodiments, each of amino acid positions 233-236 by EU number is occupied by G or is unoccupied, for example, GGG- (233-236), GG-- (233-236), G--- (233-236), or ---- (233-236), with “-” representing an unoccupied position. In some embodiments, the heavy chain constant region comprising the modification(s) is of a human IgG1 isotype. In some embodiments, the heavy chain constant region comprising the modification(s) is of a human IgG4 isotype. In some embodiments, the heavy chain constant region comprising the modification(s) is a hybrid in which domains are of different isotypes, e.g., a hybrid of IgG1 and IgG4 isotypes in which one or more domains (e.g., CH1, CH2, or CH3 domain) and/or a hinge region is of one isotype while the remaining domains are of a different isotype.

In some embodiments, the antibody or antigen-binding fragment thereof disclosed herein comprise herein comprises an anti-CD63 antibody, or a fragment or derivative thereof.

In some embodiments, the anti-CD63 antibody HCVR comprises the amino acid sequence of SEQ ID NO: 3, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 3. In certain embodiments, the nucleotide sequence that encodes the anti-CD63 antibody HCVR comprises the nucleotide sequence that encodes the amino acid sequence of SEQ ID NO: 3, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 3. In certain embodiments, the nucleotide sequence that encodes the anti-CD63 antibody HCVR comprises the nucleotide sequence of SEQ ID NO: 4, or a nucleotide sequence having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 4. In certain embodiments, the anti-CD63 antibody HCVR comprises the amino acid sequence of SEQ ID NO: 3. In certain embodiments, the nucleotide sequence that encodes the anti-CD63 antibody HCVR comprises the nucleotide sequence of SEQ ID NO: 4.

In some embodiments, the anti-CD63 antibody LCVR comprises the amino acid sequence of SEQ ID NO: 8, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 8. In certain embodiments, the nucleotide sequence that encodes the anti-CD63 antibody LCVR comprises the nucleotide sequence that encodes the amino acid sequence of SEQ ID NO: 8, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 8. In certain embodiments, the nucleotide sequence that encodes the anti-CD63 antibody LCVR comprises the nucleotide sequence of SEQ ID NO: 9, or a nucleotide sequence having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 9. In certain embodiments, the anti-CD63 antibody LCVR comprises the amino acid sequence of SEQ ID NO: 8. In certain embodiments, the nucleotide sequence that encodes the anti-CD63 antibody LCVR comprises the nucleotide sequence of SEQ ID NO: 9.

In some embodiments, the anti-CD63 antibody CH IgG4 domain comprises the amino acid sequence of SEQ ID NO: 13, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 13. In certain embodiments, the nucleotide sequence that encodes the anti-CD63 antibody CH IgG4 domain comprises the nucleotide sequence that encodes the amino acid sequence of SEQ ID NO: 13, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 13. In certain embodiments, the nucleotide sequence that encodes the anti-CD63 antibody CH IgG4 domain comprises the nucleotide sequence of SEQ ID NO: 14, or a nucleotide sequence having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 14. In certain embodiments, the anti-CD63 antibody CH IgG4 domain comprises the amino acid sequence of SEQ ID NO: 13. In certain embodiments, the nucleotide sequence that encodes the anti-CD63 antibody CH IgG4 domain comprises the nucleotide sequence of SEQ ID NO: 14.

In some embodiments, the anti-CD63 antibody CH IgG4 domain comprises the amino acid sequence of SEQ ID NO: 15, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 15. In certain embodiments, the nucleotide sequence that encodes the anti-CD63 antibody CH IgG4 domain comprises the nucleotide sequence that encodes the amino acid sequence of SEQ ID NO: 15, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 15. In certain embodiments, the nucleotide sequence that encodes the anti-CD63 antibody CH IgG4 domain comprises the nucleotide sequence of SEQ ID NO: 16, or a nucleotide sequence having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 16. In certain embodiments, the anti-CD63 antibody CH IgG4 domain comprises the amino acid sequence of SEQ ID NO: 15. In certain embodiments, the nucleotide sequence that encodes the anti-CD63 antibody CH IgG4 domain comprises the nucleotide sequence of SEQ ID NO: 16.

In some embodiments, the anti-CD63 antibody CH IgG4 domain comprises the amino acid sequence of SEQ ID NO: 98, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 98. In certain embodiments, the nucleotide sequence that encodes the anti-CD63 antibody CH IgG4 domain comprises the nucleotide sequence that encodes the amino acid sequence of SEQ ID NO: 98, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 98. In certain embodiments, the nucleotide sequence that encodes the anti-CD63 antibody CH IgG4 domain comprises the nucleotide sequence of SEQ ID NO: 99, or a nucleotide sequence having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 99. In certain embodiments, the anti-CD63 antibody CH IgG4 domain comprises the amino acid sequence of SEQ ID NO: 98. In certain embodiments, the nucleotide sequence that encodes the anti-CD63 antibody CH IgG4 domain comprises the nucleotide sequence of SEQ ID NO: 99.

In some embodiments, the anti-CD63 antibody CL comprises the amino acid sequence of SEQ ID NO: 17, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 17. In certain embodiments, the nucleotide sequence that encodes the anti-CD63 CL comprises the nucleotide sequence that encodes the amino acid sequence of SEQ ID NO: 17, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 17. In certain embodiments, the nucleotide sequence that encodes the anti-CD63 antibody CL comprises the nucleotide sequence of SEQ ID NO: 18, or a nucleotide sequence having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 18. In certain embodiments, the anti-CD63 CL comprises the amino acid sequence of SEQ ID NO: 17. In certain embodiments, the nucleotide sequence that encodes the anti-CD63 antibody CL comprises the nucleotide sequence of SEQ ID NO: 18.

In some embodiments, the anti-CD63 antibody HCDR1 comprises the amino acid sequence of SEQ ID NO: 5, or a variant thereof having at least about 90%, at least about 91%, at least about 92%, at least about 93%, at least about 94%, at least about 95%, at least about 96%, at least about 97%, at least about 98% or at least about 99%, sequence identity with SEQ ID NO: 5.

In some embodiments, the anti-CD63 antibody HCDR2 comprises the amino acid sequence of SEQ ID NO: 6, or a variant thereof having at least about 90%, at least about 91%, at least about 92%, at least about 93%, at least about 94%, at least about 95%, at least about 96%, at least about 97%, at least about 98% or at least about 99%, sequence identity with SEQ ID NO: 6.

In some embodiments, the anti-CD63 antibody HCDR3 comprises the amino acid sequence of SEQ ID NO: 7, or a variant thereof having at least about 90%, at least about 91%, at least about 92%, at least about 93%, at least about 94%, at least about 95%, at least about 96%, at least about 97%, at least about 98% or at least about 99%, sequence identity with SEQ ID NO: 7.

In some embodiments, the anti-CD63 antibody LCDR1 comprises the amino acid sequence of SEQ ID NO: 10, or a variant thereof having at least about 90%, at least about 91%, at least about 92%, at least about 93%, at least about 94%, at least about 95%, at least about 96%, at least about 97%, at least about 98% or at least about 99%, sequence identity with SEQ ID NO: 10.

In some embodiments, the anti-CD63 antibody LCDR2 comprises the amino acid sequence of SEQ ID NO: 11, or a variant thereof having at least about 90%, at least about 91%, at least about 92%, at least about 93%, at least about 94%, at least about 95%, at least about 96%, at least about 97%, at least about 98% or at least about 99%, sequence identity with SEQ ID NO: 11.

In some embodiments, the anti-CD63 antibody LCDR3 comprises the amino acid sequence of SEQ ID NO: 12, or a variant thereof having at least about 90%, at least about 91%, at least about 92%, at least about 93%, at least about 94%, at least about 95%, at least about 96%, at least about 97%, at least about 98% or at least about 99%, sequence identity with SEQ ID NO: 12.

In some embodiments, the antibody or antigen-binding fragment thereof disclosed herein comprise herein comprises an anti-EDB-FN antibody, or a fragment or derivative thereof.

In some embodiments, the anti-EDB-FN antibody HCVR comprises the amino acid sequence of SEQ ID NO: 100, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 100. In certain embodiments, the nucleotide sequence that encodes the anti-EDB-FN antibody HCVR comprises the nucleotide sequence that encodes the amino acid sequence of SEQ ID NO: 100, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 3. In certain embodiments, the nucleotide sequence that encodes the anti-EDB-FN antibody HCVR comprises the nucleotide sequence of SEQ ID NO: 101, or a nucleotide sequence having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 101. In certain embodiments, the anti-EDB-FN antibody HCVR comprises the amino acid sequence of SEQ ID NO: 100. In certain embodiments, the nucleotide sequence that encodes the anti-EDB-FN antibody HCVR comprises the nucleotide sequence of SEQ ID NO: 101.

In some embodiments, the anti-EDB-FN antibody LCVR comprises the amino acid sequence of SEQ ID NO: 105, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 105. In certain embodiments, the nucleotide sequence that encodes the anti-EDB-FN antibody LCVR comprises the nucleotide sequence that encodes the amino acid sequence of SEQ ID NO: 105, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 105. In certain embodiments, the nucleotide sequence that encodes the anti-EDB-FN antibody LCVR comprises the nucleotide sequence of SEQ ID NO: 106, or a nucleotide sequence having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 106. In certain embodiments, the anti-EDB-FN antibody LCVR comprises the amino acid sequence of SEQ ID NO: 105. In certain embodiments, the nucleotide sequence that encodes the anti-EDB-FN antibody LCVR comprises the nucleotide sequence of SEQ ID NO: 106.

In some embodiments, the anti-EDB-FN antibody CH IgG4 domain comprises the amino acid sequence of SEQ ID NO: 13, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 13. In certain embodiments, the nucleotide sequence that encodes the anti-EDB-FN antibody CH IgG4 domain comprises the nucleotide sequence that encodes the amino acid sequence of SEQ ID NO: 13, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 13. In certain embodiments, the nucleotide sequence that encodes the anti-EDB-FN antibody CH IgG4 domain comprises the nucleotide sequence of SEQ ID NO: 14, or a nucleotide sequence having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 14. In certain embodiments, the anti-EDB-FN antibody CH IgG4 domain comprises the amino acid sequence of SEQ ID NO: 13. In certain embodiments, the nucleotide sequence that encodes the anti-EDB-FN antibody CH IgG4 domain comprises the nucleotide sequence of SEQ ID NO: 14.

In some embodiments, the anti-EDB-FN antibody CH IgG4 domain comprises the amino acid sequence of SEQ ID NO: 15, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 15. In certain embodiments, the nucleotide sequence that encodes the anti-EDB-FN antibody CH IgG4 domain comprises the nucleotide sequence that encodes the amino acid sequence of SEQ ID NO: 15, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 15. In certain embodiments, the nucleotide sequence that encodes the anti-EDB-FN antibody CH IgG4 domain comprises the nucleotide sequence of SEQ ID NO: 16, or a nucleotide sequence having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 16. In certain embodiments, the anti-EDB-FN antibody CH IgG4 domain comprises the amino acid sequence of SEQ ID NO: 15. In certain embodiments, the nucleotide sequence that encodes the anti-EDB-FN antibody CH IgG4 domain comprises the nucleotide sequence of SEQ ID NO: 16.

In some embodiments, the anti-EDB-FN antibody CH IgG4 domain comprises the amino acid sequence of SEQ ID NO: 98, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 98. In certain embodiments, the nucleotide sequence that encodes the anti-EDB-FN antibody CH IgG4 domain comprises the nucleotide sequence that encodes the amino acid sequence of SEQ ID NO: 98, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 98. In certain embodiments, the nucleotide sequence that encodes the anti-EDB-FN antibody CH IgG4 domain comprises the nucleotide sequence of SEQ ID NO: 99, or a nucleotide sequence having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 99. In certain embodiments, the anti-EDB-FN antibody CH IgG4 domain comprises the amino acid sequence of SEQ ID NO: 98. In certain embodiments, the nucleotide sequence that encodes the anti-EDB-FN antibody CH IgG4 domain comprises the nucleotide sequence of SEQ ID NO: 99.

In some embodiments, the anti-EDB-FN antibody CL comprises the amino acid sequence of SEQ ID NO: 17, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 17. In certain embodiments, the nucleotide sequence that encodes the anti-EDB-FN CL comprises the nucleotide sequence that encodes the amino acid sequence of SEQ ID NO: 17, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 17. In certain embodiments, the nucleotide sequence that encodes the anti-EDB-FN antibody CL comprises the nucleotide sequence of SEQ ID NO: 18, or a nucleotide sequence having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 18. In certain embodiments, the anti-EDB-FN CL comprises the amino acid sequence of SEQ ID NO: 17. In certain embodiments, the nucleotide sequence that encodes the anti-EDB-FN antibody CL comprises the nucleotide sequence of SEQ ID NO: 18.

In some embodiments, the anti-EDB-FN antibody HCDR1 comprises the amino acid sequence of SEQ ID NO: 102, or a variant thereof having at least about 90%, at least about 91%, at least about 92%, at least about 93%, at least about 94%, at least about 95%, at least about 96%, at least about 97%, at least about 98% or at least about 99%, sequence identity with SEQ ID NO: 102.

In some embodiments, the anti-EDB-FN antibody HCDR2 comprises the amino acid sequence of SEQ ID NO: 103, or a variant thereof having at least about 90%, at least about 91%, at least about 92%, at least about 93%, at least about 94%, at least about 95%, at least about 96%, at least about 97%, at least about 98% or at least about 99%, sequence identity with SEQ ID NO: 103.

In some embodiments, the anti-EDB-FN antibody HCDR3 comprises the amino acid sequence of SEQ ID NO: 104, or a variant thereof having at least about 90%, at least about 91%, at least about 92%, at least about 93%, at least about 94%, at least about 95%, at least about 96%, at least about 97%, at least about 98% or at least about 99%, sequence identity with SEQ ID NO: 104.

In some embodiments, the anti-EDB-FN antibody LCDR1 comprises the amino acid sequence of SEQ ID NO: 107, or a variant thereof having at least about 90%, at least about 91%, at least about 92%, at least about 93%, at least about 94%, at least about 95%, at least about 96%, at least about 97%, at least about 98% or at least about 99%, sequence identity with SEQ ID NO: 107.

In some embodiments, the anti-EDB-FN antibody LCDR2 comprises the amino acid sequence of SEQ ID NO: 108, or a variant thereof having at least about 90%, at least about 91%, at least about 92%, at least about 93%, at least about 94%, at least about 95%, at least about 96%, at least about 97%, at least about 98% or at least about 99%, sequence identity with SEQ ID NO: 108.

In some embodiments, the anti-EDB-FN antibody LCDR3 comprises the amino acid sequence of SEQ ID NO: 109, or a variant thereof having at least about 90%, at least about 91%, at least about 92%, at least about 93%, at least about 94%, at least about 95%, at least about 96%, at least about 97%, at least about 98% or at least about 99%, sequence identity with SEQ ID NO: 109.

In some embodiments, the antibody or antigen-binding fragment thereof disclosed herein comprises an anti-Epcam antibody, or a fragment or derivative thereof.

In some embodiments, the anti-Epcam antibody HCVR comprises the amino acid sequence of SEQ ID NO: 124, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 124. In certain embodiments, the nucleotide sequence that encodes the anti-Epcam antibody HCVR comprises the nucleotide sequence that encodes the amino acid sequence of SEQ ID NO: 124, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 124. In certain embodiments, the anti-Epcam antibody HCVR comprises the amino acid sequence of SEQ ID NO: 124. In certain embodiments, the nucleotide sequence that encodes the anti-Epcam antibody HCVR comprises the nucleotide sequence that encodes the amino acid sequence of SEQ ID NO: 124.

In some embodiments, the anti-Epcam antibody LCVR comprises the amino acid sequence of SEQ ID NO: 129, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 129. In certain embodiments, the nucleotide sequence that encodes the anti-Epcam antibody LCVR comprises the nucleotide sequence that encodes the amino acid sequence of SEQ ID NO: 129, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 129. In certain embodiments, the anti-Epcam antibody LCVR comprises the amino acid sequence of SEQ ID NO: 129. In certain embodiments, the nucleotide sequence that encodes the anti-Epcam antibody LCVR comprises the nucleotide sequence that encodes the amino acid sequence of SEQ ID NO: 129.

In some embodiments, the anti-Epcam antibody CH IgG1 domain comprises the amino acid sequence of SEQ ID NO: 128, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 128. In certain embodiments, the nucleotide sequence that encodes the anti-Epcam antibody CH IgG1 domain comprises the nucleotide sequence that encodes the amino acid sequence of SEQ ID NO: 128, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 128. In certain embodiments, the anti-Epcam antibody CH IgG1 domain comprises the amino acid sequence of SEQ ID NO: 128. In certain embodiments, the nucleotide sequence that encodes the anti-Epcam antibody CH IgG1 domain comprises the nucleotide sequence that encodes the amino acid sequence of SEQ ID NO: 128.

In some embodiments, the anti-Epcam antibody CL comprises the amino acid sequence of SEQ ID NO: 133, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 133. In certain embodiments, the nucleotide sequence that encodes the anti-Epcam CL comprises the nucleotide sequence that encodes the amino acid sequence of SEQ ID NO: 133, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 133. In certain embodiments, the anti-Epcam CL comprises the amino acid sequence of SEQ ID NO: 133. In certain embodiments, the nucleotide sequence that encodes the anti-Epcam antibody CL comprises the nucleotide sequence of SEQ ID NO: 133.

In some embodiments, the anti-Epcam antibody HCDR1 comprises the amino acid sequence of SEQ ID NO: 125, or a variant thereof having at least about 90%, at least about 91%, at least about 92%, at least about 93%, at least about 94%, at least about 95%, at least about 96%, at least about 97%, at least about 98% or at least about 99%, sequence identity with SEQ ID NO: 125.

In some embodiments, the anti-Epcam antibody HCDR2 comprises the amino acid sequence of SEQ ID NO: 126, or a variant thereof having at least about 90%, at least about 91%, at least about 92%, at least about 93%, at least about 94%, at least about 95%, at least about 96%, at least about 97%, at least about 98% or at least about 99%, sequence identity with SEQ ID NO: 126.

In some embodiments, the anti-Epcam antibody HCDR3 comprises the amino acid sequence of SEQ ID NO: 127, or a variant thereof having at least about 90%, at least about 91%, at least about 92%, at least about 93%, at least about 94%, at least about 95%, at least about 96%, at least about 97%, at least about 98% or at least about 99%, sequence identity with SEQ ID NO: 127.

In some embodiments, the anti-Epcam antibody LCDR1 comprises the amino acid sequence of SEQ ID NO: 130, or a variant thereof having at least about 90%, at least about 91%, at least about 92%, at least about 93%, at least about 94%, at least about 95%, at least about 96%, at least about 97%, at least about 98% or at least about 99%, sequence identity with SEQ ID NO: 130.

In some embodiments, the anti-Epcam antibody LCDR2 comprises the amino acid sequence of SEQ ID NO: 131, or a variant thereof having at least about 90%, at least about 91%, at least about 92%, at least about 93%, at least about 94%, at least about 95%, at least about 96%, at least about 97%, at least about 98% or at least about 99%, sequence identity with SEQ ID NO: 131.

In some embodiments, the anti-Epcam antibody LCDR3 comprises the amino acid sequence of SEQ ID NO: 132, or a variant thereof having at least about 90%, at least about 91%, at least about 92%, at least about 93%, at least about 94%, at least about 95%, at least about 96%, at least about 97%, at least about 98% or at least about 99%, sequence identity with SEQ ID NO: 132.

In some embodiments, the antibody or antigen-binding fragment thereof disclosed herein comprises an anti-mClec9a antibody, or a fragment or derivative thereof.

In some embodiments, the anti-mClec9a antibody HCVR comprises the amino acid sequence of SEQ ID NO: 138, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 138. In certain embodiments, the nucleotide sequence that encodes the anti-mClec9a antibody HCVR comprises the nucleotide sequence that encodes the amino acid sequence of SEQ ID NO: 138, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 138. In certain embodiments, the anti-mClec9a antibody HCVR comprises the amino acid sequence of SEQ ID NO: 138. In certain embodiments, the nucleotide sequence that encodes the anti-mClec9a antibody HCVR comprises the nucleotide sequence that encodes the amino acid sequence of SEQ ID NO: 138.

In some embodiments, the anti-mClec9a antibody LCVR comprises the amino acid sequence of SEQ ID NO: 142, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 142. In certain embodiments, the nucleotide sequence that encodes the anti-mClec9a antibody LCVR comprises the nucleotide sequence that encodes the amino acid sequence of SEQ ID NO: 142, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 142. In certain embodiments, the anti-mClec9a antibody LCVR comprises the amino acid sequence of SEQ ID NO: 142. In certain embodiments, the nucleotide sequence that encodes the anti-mClec9a antibody LCVR comprises the nucleotide sequence that encodes the amino acid sequence of SEQ ID NO: 142.

In some embodiments, the anti-mClec9a antibody CH IgG1 domain comprises the amino acid sequence of SEQ ID NO: 128, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 128. In certain embodiments, the nucleotide sequence that encodes the anti-mClec9a antibody CH IgG1 domain comprises the nucleotide sequence that encodes the amino acid sequence of SEQ ID NO: 128, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 128. In certain embodiments, the anti-mClec9a antibody CH IgG1 domain comprises the amino acid sequence of SEQ ID NO: 128. In certain embodiments, the nucleotide sequence that encodes the anti-mClec9a antibody CH IgG1 domain comprises the nucleotide sequence that encodes the amino acid sequence of SEQ ID NO: 128.

In some embodiments, the anti-mClec9a antibody CL comprises the amino acid sequence of SEQ ID NO: 133, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 133. In certain embodiments, the nucleotide sequence that encodes the anti-mClec9a CL comprises the nucleotide sequence that encodes the amino acid sequence of SEQ ID NO: 133, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 133. In certain embodiments, the anti-mClec9a CL comprises the amino acid sequence of SEQ ID NO: 133. In certain embodiments, the nucleotide sequence that encodes the anti-mClec9a antibody CL comprises the nucleotide sequence of SEQ ID NO: 133.

In some embodiments, the anti-mClec9a antibody HCDR1 comprises the amino acid sequence of SEQ ID NO: 139, or a variant thereof having at least about 90%, at least about 91%, at least about 92%, at least about 93%, at least about 94%, at least about 95%, at least about 96%, at least about 97%, at least about 98% or at least about 99%, sequence identity with SEQ ID NO: 139.

In some embodiments, the anti-mClec9a antibody HCDR2 comprises the amino acid sequence of SEQ ID NO: 140, or a variant thereof having at least about 90%, at least about 91%, at least about 92%, at least about 93%, at least about 94%, at least about 95%, at least about 96%, at least about 97%, at least about 98% or at least about 99%, sequence identity with SEQ ID NO: 140.

In some embodiments, the anti-mClec9a antibody HCDR3 comprises the amino acid sequence of SEQ ID NO: 141, or a variant thereof having at least about 90%, at least about 91%, at least about 92%, at least about 93%, at least about 94%, at least about 95%, at least about 96%, at least about 97%, at least about 98% or at least about 99%, sequence identity with SEQ ID NO: 141.

In some embodiments, the anti-mClec9a antibody LCDR1 comprises the amino acid sequence of SEQ ID NO: 143, or a variant thereof having at least about 90%, at least about 91%, at least about 92%, at least about 93%, at least about 94%, at least about 95%, at least about 96%, at least about 97%, at least about 98% or at least about 99%, sequence identity with SEQ ID NO: 143.

In some embodiments, the anti-mClec9a antibody LCDR2 comprises the amino acid sequence of SEQ ID NO: 144, or a variant thereof having at least about 90%, at least about 91%, at least about 92%, at least about 93%, at least about 94%, at least about 95%, at least about 96%, at least about 97%, at least about 98% or at least about 99%, sequence identity with SEQ ID NO: 144.

In some embodiments, the anti-mClec9a antibody LCDR3 comprises the amino acid sequence of SEQ ID NO: 145, or a variant thereof having at least about 90%, at least about 91%, at least about 92%, at least about 93%, at least about 94%, at least about 95%, at least about 96%, at least about 97%, at least about 98% or at least about 99%, sequence identity with SEQ ID NO: 145.

### Fusion Polypeptides

In certain aspects, the present disclosure provides a fusion polypeptide comprising:
a) a target-binding polypeptide that binds a molecule on a target cell or a molecule in an extracellular matrix (ECM);
b) a latency associated polypeptide (LAP), or a fragment or derivative thereof, and
c) a mature Transforming Growth Factor β (TGFβ) family polypeptide, or a fragment or derivative thereof.

In some embodiments, the mature TGFβ family polypeptide, or the fragment or derivative thereof, may be inactive as a result of its interaction with the LAP, or the fragment or derivative thereof.

In some embodiments, the mature TGFβ family polypeptide, or the fragment or derivative thereof, may bind a Transforming Growth Factor β Receptor (TGFβR) upon dissociation or release of the mature TGFβ family polypeptide, or the fragment or derivative thereof, from the LAP, or the fragment or derivative thereof. In some embodiments, the mature TGFβ family polypeptide, or the fragment or derivative thereof, may induce Smad2/3 signaling in the target cell or a cell adjacent to the target cell upon dissociation or release of the mature TGFβ family polypeptide, or the fragment or derivative thereof, from the LAP, or the fragment or derivative thereof.

In some embodiments, the fusion polypeptide may comprise any of various linker(s) described herein. As an example, without limitation, linkers that may be used include any of SEQ ID NOs: 19, 46-79, and/or 96. In some embodiments, the linker comprises the amino acid sequence set forth in any of SEQ ID NOs: 19, 46-79, and/or 96, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NOs: 19, 46-79, and/or 96.

In some embodiments, the linker may comprise the sequence (GGGGS)n (SEQ ID NO: 46), wherein n=1-10, or n is 1, 2, 3, 4, 5, 6, 7, 8, 9, or 10. In some embodiments, the linker may consist of the sequence (GGGGS)n (SEQ ID NO: 46). As a non-limiting example, the linker may comprise the sequence GGGGS (SEQ ID NO: 47); GGGGSGGGGS ((G₄S)₂; SEQ ID NO: 48); GGGGSGGGGSGGGGS ((G₄S)₃; SEQ ID NO: 49); or GGGGSGGGGSGGGGSGGGGS ((G₄S)₄; SEQ ID NO: 50).

In some embodiments, the linker may comprise the sequence (GGGS)n (SEQ ID NO: 51) wherein n=1-10, or n is 1, 2, 3, 4, 5, 6, 7, 8, 9, or 10. In some embodiments, the linker may consist of the sequence (GGGS)n (SEQ ID NO: 51). In some embodiments, the linker may comprise the sequence GGGSGGGSGGGS (SEQ ID NO: 19). In some embodiments, the linker consists of the sequence GGGSGGGSGGGS (SEQ ID NO: 19).

In some embodiments, the linker may comprise the sequence GSGESGGGSG (SEQ ID NO: 96). In some embodiments, the linker consists of the sequence GSGESGGGSG (SEQ ID NO: 96).

In some embodiments, the linker may be located between the target-binding polypeptide and the LAP, or the fragment or derivative thereof.

In some embodiments, the fusion polypeptide comprises, from N-terminus to C-terminus, (i) a target-binding polypeptide described herein, (ii) a linker described herein, (iii) a LAP, or a fragment or derivative thereof described herein, and (iv) a mature TGFβ family polypeptide, or a fragment or derivative thereof described herein.

In some embodiments, the fusion polypeptide comprises, from N-terminus to C-terminus, (i) a mature TGFβ family polypeptide, or a fragment or derivative thereof described herein, (ii) a LAP, or a fragment or derivative thereof described herein, (iii) a linker described herein, and (iv) a target-binding polypeptide described herein.

In some embodiments, the mature TGFβ family polypeptide, or the fragment or derivative thereof, and the LAP, or the fragment or derivative thereof, may be separated by a protease cleavage site. The protease cleavage site may be any of various protease cleavage sites of the present disclosure such as but not limited to a furin cleavage site. In some embodiments, the furin cleavage site may comprise the sequence RXXR (SEQ ID NO: 35). In some embodiments, the protease cleavage site can be a furin cleavage site. In some embodiments, the furin cleavage site comprises the sequence RXXR (SEQ ID NO: 35). In some embodiments, the furin cleavage site consists of the sequence RXXR (SEQ ID NO: 35). In some embodiment, the furin cleavage site is RHRR (SEQ ID NO: 85), RRKR (SEQ ID NO: 86), or RKKR (SEQ ID NO: 87).

In various embodiments, the fusion polypeptide of the present disclosure may further comprise a signal peptide, e.g., at the N-terminus of the fusion polypeptide. Without wishing to be bound by theory, a signal peptide may comprise a leader sequence at the amino-terminus (N-terminus) of a nascent polypeptide, e.g., a fusion polypeptide described herein, which co-translationally or post-translationally directs the nascent protein to the endoplasmic reticulum and/or subsequent surface expression or secretion. Any of various signal peptides known in the art, or fragments, derivatives, or combinations thereof, may be used in the practice of the present disclosure, for example, such as any of those described in signalpeptide.com/index.php?m=listspdb_mammalia, incorporated by reference for all intended purposes.

In some embodiments, the signal peptide may comprise an mROR signal peptide. In some embodiments, the signal peptide is an mROR signal peptide.

In some embodiments, the mROR signal peptide comprises the amino acid sequence of SEQ ID NO: 1, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 1. In certain embodiments, the nucleotide sequence that encodes the mROR signal peptide comprises the nucleotide sequence that encodes the amino acid sequence of SEQ ID NO: 1, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 1. In certain embodiments, the nucleotide sequence that encodes the mROR signal peptide comprises the nucleotide sequence of SEQ ID NO: 2, or a nucleotide sequence having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 2. In certain embodiments mROR signal peptide comprises the amino acid sequence of SEQ ID NO: 1. In certain embodiments, the nucleotide sequence that encodes the mROR signal peptide comprises the nucleotide sequence of SEQ ID NO: 2.

In some embodiments, the mROR signal peptide comprises the sequence MHRPRRRGTRPPPLALLAALLLAARGADA (SEQ ID NO: 1). In some embodiments, the mROR signal peptide consists of the sequence MHRPRRRGTRPPPLALLAALLLAARGADA (SEQ ID NO: 1).

In some embodiments, the mature TGFβ family polypeptide, or the fragment or derivative thereof, induces signaling via a Smad-dependent canonical signaling pathway in a target cell or a cell adjacent to the target cell upon activation of the mature TGFβ family polypeptide. In some embodiments, the mature TGFβ family polypeptide, or the fragment or derivative thereof, induces Smad2/3 signaling in a target cell or a cell adjacent to the target cell upon activation of the mature TGFβ family polypeptide. In some embodiments, the mature TGFβ family polypeptide, or the fragment or derivative thereof, induces signaling via a non-canonical signaling pathway disclosed herein in a target cell or a cell adjacent to the target cell upon activation of the mature TGFβ family polypeptide. Activation of mature TGFβ family polypeptide may occur by way of any mechanism disclosed herein.

In some embodiments, a TGFβ family polypeptide, or a fragment or derivative thereof described herein, may be an autoactive TGFβ family polypeptide, i.e., a TGFβ family polypeptide that does not require activation (such as by way of proteolytic or mechanical activation) to induce TGFβ signaling (e.g., Smad2/3 signaling and/or ERK kinase signaling, or the like). In some embodiments, the TGFβ family polypeptide, or the fragment or derivative thereof, may comprise one or more mutations to produce an autoactive form of TGF-β. The one or more mutations which produce the autoactive form of TGF-β may comprise any of various mutations described herein. By way of a non-limiting example, a TGF-β family polypeptide described herein, e.g., a TGF-β1 polypeptide, may comprise one or more of a cysteine (C) residue(s) in the pro region (e.g., the LAP) of the TGF-β precursor which has been substituted with one or more of a serine (S) residue(s). In certain embodiments, the TGF-β family polypeptide may comprise a C223S mutation, and position 223 is in relation to the sequence of SEQ ID NO: 82. In certain embodiments, the TGF-β family polypeptide may comprise a C225S mutation, and position 225 is in relation to the sequence of SEQ ID NO: 82. In certain embodiments, the TGF-β family polypeptide may comprise a C223S and/or a C225S mutation thereby rendering the TGF-β family polypeptide described herein autoactive.

In some embodiments, the LAP, or the fragment or derivative thereof, may comprise an integrin binding motif disclosed herein. As a non-limiting example, the integrin-binding motif may comprise a sequence RGD. In some embodiments, the integrin binding motif may consist of a sequence RGD. In some embodiments, the LAP, or the fragment or derivative thereof, does not comprise an integrin binding motif. Non-limiting examples of integrins include αvβ6 integrin, αvβ8 integrin, and αvβ1 integrin.

In some embodiments, the LAP, or the fragment or derivative thereof, may interact with a milieu molecule(s) disclosed herein. Non-limiting example of a milieu molecule include a latency associated binding protein (LTBP), a glycoprotein-A repetition predominant protein (GARP), a leucine rich repeat containing protein 32 (LRRC32), and a leucine rich repeat containing protein 33 (LRRC33/NRROS).

In some embodiments, the fusion polypeptide disclosed herein may comprise a target-binding polypeptide comprising an antigen-binding polypeptide or antigen-binding fragment thereof disclosed herein. Antigens that may be targeted by the antigen binding polypeptide or antigen-binding fragments disclosed herein may be any of various antigens disclosed herein such as, but not limited to, PD-1, EDB-FN, CD63, CD4, CD90, Epcam, Madcam, a4b7, CCR9, Clec9a, Xcr1, and Olfm4. In some embodiments, the antigen targeted by the antigen-binding polypeptide or antigen-binding fragment thereof may comprise HLA-A2:INS. In some embodiments, the antigen targeted by the antigen-binding polypeptide or antigen-binding fragment thereof may comprise fibronectin. In some embodiments, the antigen targeted by the antigen-binding polypeptide or antigen-binding fragment thereof may comprise collagen-10.

In some embodiments, the target-binding polypeptide is not internalizing.

In some embodiments, the target-binding peptide is capable of internalization.

In various embodiments, the antigen-binding polypeptide may comprise any of various antibodies or antigen-binding fragments thereof described herein. The antibody or antigen-binding fragment thereof may comprise a heavy chain variable region disclosed herein. The antibody or antigen-binding fragment thereof may comprise a light chain variable region disclosed herein. The antibody or antigen-binding fragment thereof may comprise an immunoglobulin heavy chain constant domain disclosed herein. Non-limiting examples of an immunoglobulin heavy chain constant domain are an IgG1 domain and an IgG4 domain.

In some embodiments, the mature TGFβ family polypeptide may be a mature TGFβ polypeptide disclosed herein. In some embodiments, the mature TGFβ family polypeptide may be a mature TGFβ polypeptide, or the fragment of derivative thereof disclosed herein. In some embodiments, the mature TGFβ polypeptide may be a mature TGFβ1 polypeptide, or the fragment of derivative thereof disclosed herein. In some embodiments, the mature TGFβ1 polypeptide comprises the sequence of SEQ ID NO: 23. In some embodiments, the mature TGFβ1 polypeptide consists of the sequence of SEQ ID NO: 23. In some embodiments, the mature TGFβ1 polypeptide comprises the sequence of SEQ ID NO: 90. In some embodiments, the mature TGFβ1 polypeptide consists of the sequence of SEQ ID NO: 90. In some embodiments, the mature TGFβ polypeptide may be a mature TGFβ2 polypeptide, or the fragment of derivative thereof disclosed herein. In some embodiments, the mature TGFβ2 comprises the sequence of SEQ ID NO: 27. In some embodiments, the mature TGFβ2 consists of the sequence of SEQ ID NO: 27.

In some embodiments, the mature TGFβ family polypeptide may be a mature Growth Differentiation Factor 8 (GDF8) or a mature Growth Differentiation Factor 11 (GDF11) polypeptide. In some embodiments, the mature TGFβ family polypeptide may be a mature Growth Differentiation Factor 8 (GDF8) or a mature Bone Morphogenetic Protein 4 (BMP4).

In some embodiments, the LAP, or the fragment or derivative thereof, may be any of various LAPs or fragments or derivatives thereof described herein. In some embodiments, the LAP may comprise the sequence of SEQ ID NO: 29. In some embodiments the LAP may consist of the sequence of SEQ ID NO: 29. In some embodiments, the LAP may comprise the sequence of positions 30-274 of the sequence of SEQ ID NO: 82. In some embodiments the LAP may consist of the sequence of positions 30-274 of the sequence of SEQ ID NO: 82. In some embodiments, the LAP may comprise the sequence of SEQ ID NO: 118. In some embodiments the LAP may consist of the sequence of SEQ ID NO: 118. In some embodiments, the LAP may comprise the sequence of positions 21-298 of the sequence of SEQ ID NO: 116. In some embodiments the LAP may consist of the sequence of positions 21-298 of the sequence of SEQ ID NO: 116. In some embodiments, the LAP may comprise the sequence of SEQ ID NO: 31. In some embodiments the LAP may consist of the sequence of SEQ ID NO: 31. In some embodiments, the LAP may comprise the sequence of SEQ ID NO: 80. In some embodiments the LAP may consist of the sequence of SEQ ID NO: 80. In some embodiments, the LAP may comprise the sequence of SEQ ID NO: 88. In some embodiments the LAP may consist of the sequence of SEQ ID NO: 88. In some embodiments, the LAP may comprise the sequence of SEQ ID NO: 94. In some embodiments the LAP may consist of the sequence of SEQ ID NO: 94. In some embodiments, the LAP, or the fragment or derivative thereof, may be heterologous to the mature TGFβ family polypeptide, or the fragment or derivative thereof.

In some embodiments, the LAP, or the fragment or derivative thereof, may comprise one or more mutations. The one or more mutations in the LAP may comprise any of various mutations described herein, e.g., a C24S mutation, and position 24 is in relation to SEQ ID NO: 116; a C33S mutation, and position 33 is in relation to the sequence of SEQ ID NO: 82; a C223S mutation, and position 223 is in relation to the sequence of SEQ ID NO: 82; a C225S, and position 225 is in relation to the sequence of SEQ ID NO: 82. In some embodiments, the LAP, or the fragment or derivative thereof, comprises one or more mutations which make the LAP specific to proteolytic activation. In some embodiments, the LAP, or the fragment of derivative thereof, comprises one or more mutations that allow for proteolytic activation of the LAP. In some embodiments, the LAP, or the fragment or derivative thereof, comprises one or more mutations which allow for mechanical activation of the LAP. In some embodiments, the LAP, or the fragment or derivative thereof, comprises one or more mutations which facilitate mechanical activation of the LAP. In some embodiments, the LAP, or the fragment or derivative thereof, comprises one or more mutations which inhibit or block mechanical activation of the LAP. In some embodiments, the LAP, or the fragment or derivative thereof, comprises one or more mutations which eliminate one or more protease cleavage sites within the LAP, or the fragment or derivative thereof. In some embodiments, the LAP, or the fragment or derivative thereof, comprises one or more mutations that decrease binding of the LAP to a latency associated binding protein (LTBP).

In various embodiments, the LAP, or the fragment or derivative thereof described herein, may comprise one more mutations described herein. In some embodiments, the one or more mutations may allow for proteolytic activation of a mature TGFβ family polypeptide, or a fragment or derivative thereof, described herein. In some embodiments, the one or more mutations may allow for mechanical activation of a mature TGFβ family polypeptide, or a fragment or derivative thereof, described herein.

In various embodiments, the LAP, or the fragment or derivative thereof, comprises one or more mutations which make the TGFβ family polypeptide described herein autoactive. The autoactive form of the TGFβ family polypeptide does not require activation (such as by way of proteolytic or mechanical activation) to induce TGFβ signaling (e.g., Smad2/3 signaling and/or ERK signaling, or the like).

In some embodiments, the LAP or the fragment or derivative thereof may comprise one or more mutations which may, for example, eliminate one or more protease cleavage sites within the LAP, or the fragment or derivative thereof. In some embodiments, the one or more mutations may, for example, decrease binding of the LAP, or the fragment or derivative thereof, to a latency associated binding protein (LTBP).

In some embodiments, the LAP or the fragment or derivative thereof may comprise one or more mutations which may, for example, introduce one or more protease cleavage sites into the LAP, or the fragment or derivative thereof such as via insertion of a protease cleavage site described herein. In some embodiments, the insertion may comprise a PLGL insertion such as that which may be useful for MMP2 cleavage. In some embodiments, the insertion may comprise a PLGI insertion such as that which may be useful for MMP3, MMP7 and/or MMP8 cleavage.

In some embodiments, the LAP, or the fragment or derivative thereof comprises an amino acid sequence that has at least 80% sequence identity to the sequence of positions 30-274 of the sequence of SEQ ID NO: 82. In some embodiments, the LAP, or the fragment or derivative thereof, comprises the sequence of positions 30-274 of the sequence of SEQ ID NO: 82. In some embodiments, the LAP consists of the sequence of positions 30-274 of the sequence of SEQ ID NO: 82.

In some embodiments, the LAP, or the fragment or derivative thereof comprises an amino acid sequence that has at least 80% sequence identity to SEQ ID NO: 29. In some embodiments, the LAP, or the fragment or derivative thereof comprises the amino acid sequence of SEQ ID NO: 29. In some embodiments, the LAP consists of the amino acid sequence of SEQ ID NO: 29.

In some embodiments, the LAP, or the fragment or derivative thereof comprises an amino acid sequence that has at least 80% sequence identity to the sequence of positions 21-298 of the sequence of SEQ ID NO: 116. In some embodiments, the LAP, or the fragment or derivative thereof, comprises the sequence of positions 21-298 of the sequence of SEQ ID NO: 116. In some embodiments, the LAP consists of the sequence of positions 21-298 of the sequence of SEQ ID NO: 116.

In some embodiments, the LAP, or the fragment or derivative thereof comprises an amino acid sequence that has at least 80% sequence identity to SEQ ID NO: 118. In some embodiments, the LAP, or the fragment or derivative thereof, comprises the amino acid sequence of SEQ ID NO: 118. In some embodiments, the LAP consists of the amino acid sequence of SEQ ID NO: 118.

In some embodiments, the LAP, or the fragment or derivative thereof comprises an amino acid sequence that has at least 80% sequence identity to SEQ ID NO: 31. In some embodiments, the LAP, or the fragment or derivative thereof, comprises the amino acid sequence of SEQ ID NO: 31. In some embodiments, the LAP consists of the amino acid sequence of SEQ ID NO: 31.

In some embodiments, the LAP, or the fragment or derivative thereof comprises an amino acid sequence that has at least 80% sequence identity to SEQ ID NO: 80. In some embodiments, the LAP, or the fragment or derivative thereof, comprises the amino acid sequence of SEQ ID NO: 80. In some embodiments, the LAP consists of the amino acid sequence of SEQ ID NO: 80.

In some embodiments, the LAP, or the fragment or derivative thereof comprises an amino acid sequence that has at least 80% sequence identity to SEQ ID NO: 88. In some embodiments, the LAP, or the fragment or derivative thereof, comprises the amino acid sequence of SEQ ID NO: 88. In some embodiments, the LAP consists of the amino acid sequence of SEQ ID NO: 88.

In some embodiments, the LAP, or the fragment or derivative thereof comprises an amino acid sequence that has at least 80% sequence identity to SEQ ID NO: 94. In some embodiments, the LAP, or the fragment or derivative thereof, comprises the amino acid sequence of SEQ ID NO: 94. In some embodiments, the LAP consists of the amino acid sequence of SEQ ID NO: 94.

### Non-Limiting Examples of Fusion Polypeptide Sequences

In various embodiments, the fusion polypeptide comprises the amino acid sequence of SEQ ID NO: 36, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 36. In certain embodiments, the nucleotide sequence that encodes the fusion polypeptide comprises the nucleotide sequence that encodes the amino acid sequence of SEQ ID NO: 36, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 36. In certain embodiments, the nucleotide sequence that encodes the fusion polypeptide comprises the nucleotide sequence of SEQ ID NO: 37, or a nucleotide sequence having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 37. In certain embodiments the fusion polypeptide comprises the amino acid sequence of SEQ ID NO: 36. In certain embodiments, the nucleotide sequence that encodes the fusion polypeptide comprises the nucleotide sequence of SEQ ID NO: 37.

In various embodiments, the fusion polypeptide comprises the amino acid sequence of SEQ ID NO: 38, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 38. In certain embodiments, the nucleotide sequence that encodes the fusion polypeptide comprises the nucleotide sequence that encodes the amino acid sequence of SEQ ID NO: 38, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 38. In certain embodiments, the nucleotide sequence that encodes the fusion polypeptide comprises the nucleotide sequence of SEQ ID NO: 39, or a nucleotide sequence having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 39. In certain embodiments the fusion polypeptide comprises the amino acid sequence of SEQ ID NO: 38. In certain embodiments, the nucleotide sequence that encodes the fusion polypeptide comprises the nucleotide sequence of SEQ ID NO: 39.

In various embodiments, the fusion polypeptide comprises the amino acid sequence of SEQ ID NO: 40, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 40. In certain embodiments, the nucleotide sequence that encodes the fusion polypeptide comprises the nucleotide sequence that encodes the amino acid sequence of SEQ ID NO: 40, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 40. In certain embodiments, the nucleotide sequence that encodes the fusion polypeptide comprises the nucleotide sequence of SEQ ID NO: 41, or a nucleotide sequence having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 41. In certain embodiments the fusion polypeptide comprises the amino acid sequence of SEQ ID NO: 40. In certain embodiments, the nucleotide sequence that encodes the fusion polypeptide comprises the nucleotide sequence of SEQ ID NO: 41.

In various embodiments, the fusion polypeptide comprises the amino acid sequence of SEQ ID NO: 42, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 42. In certain embodiments, the nucleotide sequence that encodes the fusion polypeptide comprises the nucleotide sequence that encodes the amino acid sequence of SEQ ID NO: 42, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 42. In certain embodiments, the nucleotide sequence that encodes the fusion polypeptide comprises the nucleotide sequence of SEQ ID NO: 43, or a nucleotide sequence having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 43. In certain embodiments the fusion polypeptide comprises the amino acid sequence of SEQ ID NO: 42. In certain embodiments, the nucleotide sequence that encodes the fusion polypeptide comprises the nucleotide sequence of SEQ ID NO: 43.

In various embodiments, the fusion polypeptide comprises the amino acid sequence of SEQ ID NO: 44, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 44. In certain embodiments, the nucleotide sequence that encodes the fusion polypeptide comprises the nucleotide sequence that encodes the amino acid sequence of SEQ ID NO: 44, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 44. In certain embodiments, the nucleotide sequence that encodes the fusion polypeptide comprises the nucleotide sequence of SEQ ID NO: 45, or a nucleotide sequence having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 45. In certain embodiments the fusion polypeptide comprises the amino acid sequence of SEQ ID NO: 44. In certain embodiments, the nucleotide sequence that encodes the fusion polypeptide comprises the nucleotide sequence of SEQ ID NO: 45.

In various embodiments, the fusion polypeptide comprises the amino acid sequence of SEQ ID NO: 110, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 110. In certain embodiments, the nucleotide sequence that encodes the fusion polypeptide comprises the nucleotide sequence that encodes the amino acid sequence of SEQ ID NO: 110, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 110. In certain embodiments, the nucleotide sequence that encodes the fusion polypeptide comprises the nucleotide sequence of SEQ ID NO: 111, or a nucleotide sequence having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 111. In certain embodiments the fusion polypeptide comprises the amino acid sequence of SEQ ID NO: 110. In certain embodiments, the nucleotide sequence that encodes the fusion polypeptide comprises the nucleotide sequence of SEQ ID NO: 111.

In various embodiments, the fusion polypeptide comprises the amino acid sequence of SEQ ID NO: 112, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 112. In certain embodiments, the nucleotide sequence that encodes the fusion polypeptide comprises the nucleotide sequence that encodes the amino acid sequence of SEQ ID NO: 112, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 112. In certain embodiments, the nucleotide sequence that encodes the fusion polypeptide comprises the nucleotide sequence of SEQ ID NO: 113, or a nucleotide sequence having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 113. In certain embodiments the fusion polypeptide comprises the amino acid sequence of SEQ ID NO: 112. In certain embodiments, the nucleotide sequence that encodes the fusion polypeptide comprises the nucleotide sequence of SEQ ID NO: 113.

In various embodiments, the fusion polypeptide comprises the amino acid sequence of SEQ ID NO: 114, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 114. In certain embodiments, the nucleotide sequence that encodes the fusion polypeptide comprises the nucleotide sequence that encodes the amino acid sequence of SEQ ID NO: 114, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 114. In certain embodiments, the nucleotide sequence that encodes the fusion polypeptide comprises the nucleotide sequence of SEQ ID NO: 115, or a nucleotide sequence having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 115. In certain embodiments the fusion polypeptide comprises the amino acid sequence of SEQ ID NO: 114. In certain embodiments, the nucleotide sequence that encodes the fusion polypeptide comprises the nucleotide sequence of SEQ ID NO: 115.

In various embodiments, the fusion polypeptide comprises the amino acid sequence of SEQ ID NO: 120, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 120. In certain embodiments, the nucleotide sequence that encodes the fusion polypeptide comprises the nucleotide sequence that encodes the amino acid sequence of SEQ ID NO: 120, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 120. In certain embodiments, the nucleotide sequence that encodes the fusion polypeptide comprises the nucleotide sequence of SEQ ID NO: 121, or a nucleotide sequence having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 121. In certain embodiments the fusion polypeptide comprises the amino acid sequence of SEQ ID NO: 120. In certain embodiments, the nucleotide sequence that encodes the fusion polypeptide comprises the nucleotide sequence of SEQ ID NO: 121.

In various embodiments, the fusion polypeptide comprises the amino acid sequence of SEQ ID NO: 122, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 122. In certain embodiments, the nucleotide sequence that encodes the fusion polypeptide comprises the nucleotide sequence that encodes the amino acid sequence of SEQ ID NO: 122, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 122. In certain embodiments, the nucleotide sequence that encodes the fusion polypeptide comprises the nucleotide sequence of SEQ ID NO: 123, or a nucleotide sequence having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 123. In certain embodiments the fusion polypeptide comprises the amino acid sequence of SEQ ID NO: 122. In certain embodiments, the nucleotide sequence that encodes the fusion polypeptide comprises the nucleotide sequence of SEQ ID NO: 123.

In various embodiments, the fusion polypeptide comprises the amino acid sequence of SEQ ID NO: 134, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 134. In certain embodiments, the nucleotide sequence that encodes the fusion polypeptide comprises the nucleotide sequence that encodes the amino acid sequence of SEQ ID NO: 134, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 134. In certain embodiments, the nucleotide sequence that encodes the fusion polypeptide comprises the nucleotide sequence of SEQ ID NO: 135, or a nucleotide sequence having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 135. In certain embodiments the fusion polypeptide comprises the amino acid sequence of SEQ ID NO: 134. In certain embodiments, the nucleotide sequence that encodes the fusion polypeptide comprises the nucleotide sequence of SEQ ID NO: 135.

In various embodiments, the fusion polypeptide comprises the amino acid sequence of SEQ ID NO: 136, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 136. In certain embodiments, the nucleotide sequence that encodes the fusion polypeptide comprises the nucleotide sequence that encodes the amino acid sequence of SEQ ID NO: 136, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 136. In certain embodiments, the nucleotide sequence that encodes the fusion polypeptide comprises the nucleotide sequence of SEQ ID NO: 137, or a nucleotide sequence having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 137. In certain embodiments the fusion polypeptide comprises the amino acid sequence of SEQ ID NO: 136. In certain embodiments, the nucleotide sequence that encodes the fusion polypeptide comprises the nucleotide sequence of SEQ ID NO: 137.

### Polynucleotides and Vectors

In certain aspects, the present disclosure provides polynucleotides encoding one or more of the above-described polypeptides. In one aspect, the present disclosure provides polynucleotides encoding a polypeptide complex disclosed herein. In one aspect, the present disclosure provides polynucleotides encoding fusion polypeptides disclosed herein. In some embodiments, the polynucleotide is DNA. In some embodiments, the polynucleotide is RNA.

In certain embodiments, the polynucleotide encoding the polypeptides disclosed herein may comprise one or more regulatory elements. The regulatory element may be capable of modulating expression of the polypeptides. Non-limiting examples of regulatory elements are, promoters, initiation sites, polyadenylation (polyA) tails, IRES elements, enhancers, response elements, and termination signals.

In some embodiments, the polynucleotides sequence that encodes the polypeptides (e.g., fusion polypeptides) described herein may be operatively linked to a promoter for expression. In some embodiments, when the sequence encoding a polypeptide(s) described herein is operably linked to a promoter, the promoter may mediate the expression of the polypeptide(s). A “promoter” is a regulatory region of DNA usually comprising a TATA box capable of directing RNA polymerase II to initiate RNA synthesis at the appropriate transcription initiation site for a particular polynucleotide sequence. A promoter may additionally comprise other regions which influence the transcription initiation rate. As used herein, the term “promoter” encompasses enhancers. The promoter sequences disclosed herein modulate transcription of an operably linked polynucleotide. A promoter can be active in one or more of the cell types disclosed herein (e.g., a eukaryotic cell, a non-human mammalian cell, a human cell, a rodent cell, a pluripotent cell, a one-cell stage embryo, a differentiated cell, or a combination thereof). A promoter can be, for example, a constitutively active promoter, a conditional promoter, an inducible promoter, a temporally restricted promoter (e.g., a developmentally regulated promoter), or a spatially restricted promoter (e.g., a cell-specific or tissue-specific promoter).

Examples of constitutive promoters include, but are not limited to, cytomegalovirus (CMV) promoter, EF1a, SV40, PGK1 (human or mouse), Ubc, human beta actin, CAG, Ac5, Polyhedrin, TEF1, GDS, CaMV35S, Ubi, H1, and U6 promoters.

In some embodiments, the promoter can be a CMV promoter. In some embodiments, the promoter can be a CMV/EF1 hybrid promoter.

Inducible promoters can include, for example, chemically regulated promoters and physically-regulated promoters. Chemically regulated promoters include, for example, alcohol-regulated promoters (e.g., an alcohol dehydrogenase (alcA) gene promoter), tetracycline-regulated promoters (e.g., a tetracycline-responsive promoter, a tetracycline operator sequence (tetO), a tet-On promoter, or a tet-Off promoter), steroid regulated promoters (e.g., a rat glucocorticoid receptor, a promoter of an estrogen receptor, or a promoter of an ecdysone receptor), or metal-regulated promoters (e.g., a metalloprotein promoter). Physically regulated promoters include, for example temperature-regulated promoters (e.g., a heat shock promoter such as Hsp70- and Hsp90-derived promoters) and light-regulated promoters (e.g., a light-inducible promoter or a light-repressible promoter). Other inducible promoters include lac, sp6, and an T7 promotor.

Tissue-specific promoters can be, for example, neuron-specific promoters, glia-specific promoters, muscle cell-specific promoters, heart cell-specific promoters, kidney cell-specific promoters, bone cell-specific promoters, endothelial cell-specific promoters, or immune cell-specific promoters (e.g., a B cell promoter or a T cell promoter).

Developmentally regulated promoters include, for example, promoters active only during an embryonic stage of development, or only in an adult cell.

Other non-limiting examples of promoters useful in the nucleic acid molecules of the present disclosure include a CB7/CAG promoter and associated upstream regulatory sequences, EF-1 alpha promoter, mU1a promoter, UB6 promoter, chicken beta-actin (CBA) promoter, and liver-specific promoters, such as TBG (Thyroxine-binding Globulin) promoter, APOA2 promoter, SERPINA1 (hAAT) promoter, ApoE.hAAT, or muscle-specific promoters, such as a human desmin promoter, CK8 promoter or Pitx3 promoter, inducible promoters, such as a hypoxia-inducible promoter or a rapamycin-inducible promoter, or a combination thereof.

In some embodiments, nucleic acid molecules of the present disclosure may include one promoter. In some embodiments, nucleic acid molecules of the present disclosure may include more than one (e.g., 2, 3, 4, or more) promoter.

In a further aspect, the present disclosure provides a vector comprising any of the above-described polynucleotides. Such vectors may comprise polynucleotides encoding the polypeptides disclosed above. The vector can be a viral vector or non-viral vector.

In some embodiments, the vector can be a viral vector. Non-limiting examples of viral vectors include adenovirus, adeno-associated virus (AAV, e.g., AAV8, AAV9, AAVrh10, AAVS3), lentivirus, helper-dependent adenovirus, herpes simplex virus, poxvirus, hemagglutinin virus of Japan (HVJ), alphavirus (e.g., semliki forest virus (SFV), sindbis virus (SIN)), vaccinia virus, baculovirus vectors, and retrovirus vectors (e.g., murine leukemia virus (MLV), human immunodeficiency virus (HIV)).

In some embodiments, the viral vectors described herein are recombinant viral vectors. In some embodiments, the viral vectors described herein are altered such that they are replication-deficient in humans. In some embodiments, the viral vectors are hybrid vectors, e.g., an AAV vector placed into a “helpless” adenoviral vector. In some embodiments, viral vectors comprise a viral capsid from a first virus and viral envelope proteins from a second virus, e.g., VSV-G protein from vesicular stomatitis virus (VSV).

In some embodiments, the viral vectors described herein are AAV based viral vectors. In some embodiments, the AAV-based vectors described herein do not encode the AAV rep gene (required for replication) and/or the AAV cap gene (required for synthesis of the capsid proteins) (the rep and cap proteins may be provided by the packaging cells in trans). Multiple AAV serotypes have been identified. In some embodiments, AAV based vectors described herein comprise capsid components from one or more of AAV1, AAV2, AAV3, AAV4, AAV5, AAV6, AAV7, AAV8, AAV9, AAV10, AAV11, AAV12, AAV13, AAV14, AAV15, AAV16, AAVS3, AAV.rh8, AAV.rh10, AAV.rh20, AAV.rh39, AAV.rh46, AAV.rh73, AAV.Rh74, AAV.RHM4-1, AAV.hu37, AAV.Anc80, AAV.Anc80L65, AAV.7m8, AAV.PHP.B, AAV.PHP.eB, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, or AAV.HSC16 or other rAAV particles, or combinations of two or more thereof. In some embodiments, AAV-based vectors provided herein comprise components from one or more serotypes of AAV. In some embodiments, AAV-based vectors described herein comprise components from one or more serotypes of AAV with tropism to desired tissues (e.g., liver, muscle, heart, kidney, neuron).

In some embodiments, the viral vectors described herein are lentivirus-based viral vectors. In some embodiments, lentiviral vectors described herein are derived from human lentiviruses. In some embodiments, lentiviral vectors described herein are derived from non-human lentiviruses. In some embodiments, lentiviral vectors described herein are packaged into a lentiviral capsid. In some embodiments, lentiviral vectors described herein comprise one or more of the following elements: long terminal repeats, a primer binding site, a polypurine tract, att sites, and an encapsidation site.

In some embodiments, the viral vectors described herein are HIV-based viral vectors. In some embodiments, HIV-based vectors described herein comprise at least two polynucleotides, wherein the gag and pol genes are from an HIV genome and the env gene is from another virus.

In some embodiments, the viral vectors described herein are herpes simplex virus-based viral vectors. In some embodiments, herpes simplex virus-based vectors described herein are modified such that they do not comprise one or more immediately early (IE) genes, rendering them non-cytotoxic.

In some embodiments, the viral vectors provided herein are MLV based viral vectors. In some embodiments, MLV-based vectors provided herein comprise up to 8 kb of heterologous DNA in place of the viral genes.

In some embodiments, the viral vectors provided herein are alphavirus-based viral vectors. In some embodiments, alphavirus vectors provided herein are recombinant, replication defective alphaviruses. In some embodiments, alphavirus replicons in the alphavirus vectors provided herein are targeted to specific cell types by displaying a functional heterologous ligand on their virion surface.

In some embodiments, the vector can be a non-viral vector. Non-limiting examples of non-viral vectors include a plasmid (e.g., minicircle plasmid), a Sleeping Beauty transposon, a piggyBac transposon, or a single- or double-stranded DNA molecule that is used as a template for homology directed repair (HDR) based gene editing.

### Cells and Production Methods

In one aspect, the present disclosure provides a cell, e.g., a host cell, comprising a polynucleotide and/or a recombinant vector described herein. In some embodiments, the polynucleotide may encode, for example, a fusion polypeptide described herein. In some embodiments, the vector may comprise a polynucleotide described herein. The term “host cell” refers to any cell that comprises a heterologous nucleic acid. By way of a non-limiting example, the heterologous nucleic acid may be a vector disclosed herein. A host cell, for example, without limitation, may be a cell from any organism that is used, manipulated, modified, selected, transformed, or grown, for the production of a substance by the cell, e.g., the expression by the cell of, an RNA or DNA sequence, a gene, a protein, or an enzyme. An appropriate host may be determined.

In some embodiments, the host cell may be selected based on the vector backbone. In some embodiments, a cosmid or plasmid or may be introduced into a prokaryote host cell for replication of several types of vectors. Bacterial cells including, may be used as host cells for vector replication and/or expression or for phage viruses. Eukaryotic cells that can be used as host cells include, but are not limited to mammals, insects and yeast. Non-limiting examples of mammalian eukaryotic host cells are PC12, NIH3T3, HeLa, COS, Jurkat, 293, CHO (Chinese hamster ovary), ExpiCHO-S, FreedomCHO-S, and Saos.

Packaging cells useful for production of the polynucleotides and/or recombinant vectors described herein include, e.g., animal cells permissive for the vector, e.g., a viral vector, or cells modified to be permissive for the vector; or the packaging cell construct, for example, with the use of a transformation agent such as calcium phosphate. Non-limiting examples of packaging cell lines useful production methods described herein include, e.g., human embryonic kidney 293 (HEK-293) cells (e.g., American Type Culture Collection [ATCC] No. CRL-1573), HEK-293 cells that contain the SV40 Large T-antigen (HEK-293T or 293T), HEK293T/17 cells, human sarcoma cell line HT-1080 (CCL-121), lymphoblast-like cell line Raj i (CCL-86), glioblastoma-astrocytoma epithelial-like cell line U87-MG (HTB-14), T-lymphoma cell line HuT78 (TIB-161), NIH/3T3 cells, Chinese Hamster Ovary cells (CHO) (e.g., ATCC Nos. CRL9618, CCL61, CRL9096), HeLa cells (e.g., ATCC No. CCL-2), Vero cells, NIH 3T3 cells (e.g., ATCC No. CRL-1658), Huh-7 cells, BHK cells (e.g., ATCC No. CCL10), PC12 cells (ATCC No. CRL1721), COS cells, COS-7 cells (ATCC No. CRL1651), RATI cells, mouse L cells (ATCC No. CCLI.3), HLHepG2 cells, CAP cells, CAP-T cells, and the like.

Further non-limiting examples of packaging cells and/or systems that may be useful for the production methods described herein include, for example, L929 cells, the FLY viral packaging cell system outlined in Cosset et al (1995) J Virol 69, 7430-7436, NSO (murine myeloma) cells, human amniocytic cells (e.g., CAP, CAP-T), yeast cells (including, but not limited to, S. cerevisiae, Pichia pastoris), plant cells (including, but not limited to, Tobacco NT1, BY-2), insect cells (including but not limited to SF9, S2, SF21, Tni (e.g. High 5)) or bacterial cells (including, but not limited to, E. coli).

Additional packaging cells and systems, packaging techniques and vectors for packaging the nucleic acids genome into a vector may include method steps comprising, e.g., construction of structural protein expression cassettes comprising plasmids for vector-inducible expression of virus structural proteins, and incorporation of any additional elements by polymerase chain reaction (PCR) amplification or by using synthetic oligonucleotides. By way of a non-limiting example, for selection, screening, and/or characterization of packaging cell lines, cells transfected with expression cassette constructs may be selected with, e.g., G418 or hygromycin. Pooled foci of drug-resistant cells may be cloned by limiting dilution, and individual clones may be screened for packaging activity, e.g., by transfection with a vector using, e.g., Lipofection or electroporation. Those clones with the highest levels of activity may be expanded for further use. Northern and Western blot analysis of vector-specific or structural protein-specific RNA and proteins expressed in packaging cells may be performed. The titer of replication-incompetent vector particles in clarified packaging cell line culture supernatants may be determined, e.g., by infection of naïve monolayers with serial dilutions, X-gal staining and counting the total number of stained cells per well at the appropriate dilution. Vector titer may be designated as infectious units (IU)/ml. Contaminating replication-competent virus in culture supernatants may detected by standard plaque assay (plaque-forming units or PFU/ml) and by serial undiluted passages in naïve cells. Methods of packaging include using packaging cells that permanently express the viral components, or by transiently transfecting cells with plasmids.

In some embodiments, the present disclosure provides a cell (e.g., a FreedomCHO-S cell or an ExpiCHO cell) comprising a polypeptide complex or a fusion polypeptide disclosed herein. In a related aspect, the present disclosure provides a cell comprising a polynucleotide disclosed herein. In yet another related aspect, the present disclosure provides a cell comprising a vector disclosed herein. By way of an example, without limitation, any of the above-described cells may comprising the polypeptide complex(es), fusion polypeptide(s), polynucleotide(s), and/or vectors disclosed herein. In some embodiments, the cell may be a FreedomCHO-S cell.

In some embodiments, the present disclosure provides a method of making a polypeptide complex or a fusion polypeptide disclosure herein. The method may comprise incubating the cell comprising the polynucleotide disclosure herein, and/or the vector disclosed herein, under conditions allowing for production of the polypeptide complex or fusion polypeptide.

Isolation or purification of the polypeptide complex or the fusion polypeptide disclosed herein, e.g., from a virus or virus extract may include, without limitation, techniques and/or method steps comprising any of freeze/thaw cycles, microfluidization, filtration, e.g., nanofiltration and crossflow filtration, osmotic shock, nuclease, detergents and/or protease treatments, cell lysis and DNA digestion, clarification (including filtration and centrifugation), ultracentrifugation, precipitation, e.g., precipitation with crowding reagents, crossflow filtration, affinity purification, nanoscale flow cytometry, CsCl density gradient, iodixanol gradient centrifugation, chromatography, e.g., column chromatography, including application of various resins such as, but not limited to, e.g., ion-exchange, anion- and cation-exchange, affinity including antibody affinity, chromate-focusing, desalting and buffer exchange, hydrophobic interaction, immunoprecipitation, multi-modal, mixed modal, reverse-phase, and size-exclusion, heparinized support matrix chromatography, DEAE Sepharose Fast Flow (FF), POROS 50 D, Fractogel® EMD DEAE (M), Macro-Prep DEAE Support, DEAE Ceramic HyperD® 20, and Toyopearl DEAE-650M, and/or use of various medias, e.g., ceramic hydroxyapatite, ceramic fluorapatite, ceramic hydroxyfluoroapatite, cellufine sulfate media analytical chromatographic methods, including, e.g., analytical ion-exchange high-performance liquid chromatography (HPLC), reversed-phase HPLC and sodium dodecyl-sulfate polyacrylamide gel electrophoresis (SDS-PAGE) analysis, or combinations thereof.

In some embodiments, the method of making the polypeptide complex or the fusion polypeptide may comprise collecting cell culture medium and isolating the produced polypeptide complex or a fusion polypeptide by a process comprising affinity chromatography. In some embodiments, the affinity chromatography may comprise, e.g., Protein A column or beads or a Protein G column or beads.

### Pharmaceutical Compositions

In a further aspect, the disclosure provides a composition (e.g., a pharmaceutical composition) comprising a polypeptide complex, a fusion polypeptide, a polynucleotide, a vector, and/or a cell disclosed herein, for example, together with a pharmaceutically acceptable carrier and/or diluent. The pharmaceutical compositions of the disclosure may be in any suitable form depending upon the desired method of administering to a subject.

The pharmaceutical compositions may comprise the polypeptide complexes or polypeptide complex-based molecules and/or fusion polypeptides or fusion polypeptide-based molecules of the disclosure either in the free form or in the form of a pharmaceutically acceptable salt. The term “pharmaceutically acceptable salt” as used herein refers to a derivative of the disclosed polypeptide complex(s) or fusion polypeptide(s) wherein the polypeptide(s) or complexes thereof are modified by making acid or base salts of the agent. For example, acid salts are prepared from the free base (typically wherein the neutral form of the drug has a neutral —NH₂ group) involving reaction with a suitable acid. Suitable acids for preparing acid salts include both organic acids, e.g., acetic acid, benzoic acid, citric acid, propionic acid, glycolic acid, trifluoroacetic acid, pyruvic acid, oxalic acid, malic acid, malonic acid, maleic acid, succinic acid, fumaric acid, tartaric acid, cinnamic acid, mandelic acid, methanesulfonic acid, ethanesulfonic acid, p-toluenesulfonic acid, salicylic acid, and the like, as well as inorganic acids, e.g., hydrochloric acid, hydrobromic acid, sulfuric acid, nitric acid phosphoric acid and the like. Conversely, preparation of basic salts of acid moieties which may be present on polypeptide are prepared using a pharmaceutically acceptable base such as sodium hydroxide, potassium hydroxide, ammonium hydroxide, calcium hydroxide, trimethylamine or the like.

Compositions of the disclosure may comprise multiple polypeptide complex(s) and/or fusion polypeptide(s), e.g., 2 to 50, 2 to 40, 2 to 30, 5 to 25, 5 to 20, or 10 to 15 polypeptide complex(s) and/or fusion polypeptide(s) as described herein. In some embodiment, the compositions of the disclosure may comprise 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, or 54 polypeptide complex(s) and/or fusion polypeptide(s), or a pharmaceutically acceptable salt thereof.

In some embodiments, the polypeptide complexes or polypeptide complex-based molecules and/or fusion polypeptides or fusion polypeptide-based molecules described herein may be present in a solution at a concentration of about 1 g/mL to 50 mg/mL, for example, about 0.1 mg/mL to 10 mg/mL, about 0.2 mg/mL to 5 mg/mL, about 0.5 mg/mL to 8 mg/mL, about 0.8 mg/mL to 12 mg/mL, about 1 mg/mL to 15 mg/mL, about 2 mg/mL to 20 mg/mL, or about 5 mg/mL to 25 mg/mL, or about 0.1 mg/mL, 0.2 mg/mL, 0.3 mg/mL, 0.4 mg/mL, 0.5 mg/mL, 0.6 mg/mL, 0.7 mg/mL, 0.8 mg/mL, 0.9 mg/mL, 1 mg/mL, 1.25 mg/mL, 1.5 mg/mL, 1.75 mg/mL, 2 mg/mL, 2.25 mg/mL, 2.5 mg/mL, 2.75 mg/mL, 3 mg/mL, 3.25 mg/mL, 3.5 mg/mL, 3.75 mg/mL, 4 mg/mL, 5 mg/mL, 6 mg/mL, 7 mg/mL, 8 mg/mL, 9 mg/mL, 10 mg/mL, 11 mg/mL, 12 mg/mL, 13 mg/mL, 14 mg/mL, 15 mg/mL or 20 mg/mL.

The pharmaceutical composition may be adapted for administration by any appropriate route such as, e.g., parenteral (including intraperitoneal, subcutaneous, intramuscular, or intravenous), enteral (including oral or rectal), inhalation, or intranasal routes.

Such compositions may be prepared, for example, by mixing the active ingredient with the carrier(s) or excipient(s) under sterile conditions.

In addition, disclosed herein are pharmaceutical dosage forms comprising the polypeptide complexes or polypeptide complex-based molecules and/or fusion polypeptides or fusion polypeptide-based molecules, or conjugates comprising the polypeptide complexes or polypeptide complex-based molecules and/or fusion polypeptides or fusion polypeptide-based molecules, nucleic acid molecules, vectors, cells, or target-binding polypeptides of the disclosure.

Pharmaceutical compositions based on the polypeptide complexes or polypeptide complex-based molecules and/or fusion polypeptides or fusion polypeptide-based molecules, or conjugates comprising the polypeptide complexes or polypeptide complex-based molecules and/or fusion polypeptides or fusion polypeptide-based molecules, nucleic acid molecules, vectors, cells, or target-binding moieties disclosed herein can be formulated in any conventional manner using one or more physiologically acceptable carriers and/or excipients. The polypeptide complexes or polypeptide complex-based molecules and/or fusion polypeptides or fusion polypeptide-based molecules, or conjugates comprising the polypeptide complexes or polypeptide complex-based molecules and/or fusion polypeptides or fusion polypeptide-based molecules, nucleic acid molecules, vectors, cells, or target-binding moieties may be formulated for administration by, for example, injection, inhalation, or insulation (either through the mouth or the nose) or by oral, buccal, parenteral or rectal administration, or by administration directly to an organ or tissue.

The pharmaceutical compositions can be formulated for a variety of modes of administration, including systemic, topical, or localized administration. Techniques and formulations can be found in, for example, Remington's Pharmaceutical Sciences, Meade Publishing Co., Easton, Pa. For systemic administration, injection is preferred, including intramuscular, intravenous, intraperitoneal, and subcutaneous. For the purposes of injection, the pharmaceutical compositions can be formulated in liquid solutions, preferably in physiologically compatible buffers, such as Hank's solution or Ringer's solution. In addition, the pharmaceutical compositions may be formulated in solid form and redissolved or suspended immediately prior to use. Lyophilized forms of the pharmaceutical composition are also suitable.

In some embodiments, the pharmaceutical compositions of the present disclosure may be lyophilized. As a non-limiting example, the obtained lyophilizate can be reconstituted into a hydrous composition by adding a hydrous solvent. In some embodiments, the hydrous composition may be able to be directly administered parenterally to a patient. Therefore, in a further embodiment of the present disclosure, the pharmaceutical composition can be a hydrous pharmaceutical composition, obtainable via reconstitution of the lyophilizate with a hydrous solvent.

In some embodiments, the pharmaceutical composition disclosed herein may comprise a lyophilized formulation. As a non-limiting example, the lyophilization formulation may comprise polypeptide complexes or polypeptide complex-based molecules and/or fusion polypeptides or fusion polypeptide-based molecules of the disclosure, mannitol, and/or TWEEN 80®. As another non-limiting example, the lyophilization formulation may comprise the polypeptide complexes or polypeptide complex-based molecules and/or fusion polypeptides or fusion polypeptide-based molecules disclosed herein, mannitol and poloxamer 188. In some embodiments, the pharmaceutical composition may comprise a lyophilization formulation comprising a reconstituted-liquid composition.

In some embodiments, pharmaceutical compositions of the present disclosure may provide a formulation with an enhanced solubility and/or moistening of the lyophilizate over previously known compositions. As a non-limiting example, enhanced solubility and/or moistening of the lyophilizate may be achieved using an appropriate composition of excipients. In this way, pharmaceutical compositions of the present disclosure comprising polypeptide complexes or polypeptide complex-based molecules and/or fusion polypeptides or fusion polypeptide-based molecules variants thereof may be developed to show a desired shelf stability at (e.g., at −20° C., +5° C., or +25° C.) and can be easily resolubilized such that the lyophilizate can be completely dissolved through the use of a buffer or other excipients from seconds up to two or more minutes, with or without the use of an of ultrasonic homogenizer. Furthermore, the composition can be easily provided to a patient in need of treatment via any appropriate delivery route disclosed herein, e.g., parenteral (including intraperitoneal, subcutaneous, intramuscular, or intravenous), enteral (including oral or rectal), inhalation, or intranasal routes. As a non-limiting example, the pH-value of the resulting solution may be between pH 2.7 and pH 9.0.

For oral administration, the pharmaceutical compositions may take the form of, for example, tablets or capsules prepared by conventional means with pharmaceutically acceptable excipients such as binding agents (e.g. pregelatinized maize starch, polyvinylpyrrolidone or hydroxypropyl methylcellulose); fillers (e.g. lactose, microcrystalline cellulose or calcium hydrogen phosphate); lubricants (e.g. magnesium stearate, talc or silica); disintegrants (e.g. potato starch or sodium starch glycolate); or wetting agents (e.g. sodium lauryl sulfate). The tablets can also be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., ationd oil, oily esters, ethyl alcohol or fractionated vegetable oils); and preservatives (e.g., methyl or propyl-p-hydroxybenzoates or sorbic acid). The preparations can also contain buffer salts, flavoring, coloring and sweetening agents as appropriate.

The pharmaceutical compositions can be formulated for parenteral administration by injection, e.g., by bolus injection or continuous infusion. Formulations for injection can be presented in a unit dosage form, e.g., in ampoules or in multi-dose containers, with an optionally added preservative. The pharmaceutical compositions can further be formulated as suspensions, solutions or emulsions in oily or aqueous vehicles, and may contain other agents including suspending, stabilizing and/or dispersing agents.

Additionally, the pharmaceutical compositions can also be formulated as a depot preparation. These long-acting formulations can be administered by implantation (e.g., subcutaneously or intramuscularly) or by intramuscular injection. Thus, for example, the compounds may be formulated with suitable polymeric or hydrophobic materials (e.g., as an emulsion in an acceptable oil) or ion exchange resins, or as sparingly soluble derivatives, for example, as a sparingly soluble salt. Other suitable delivery systems include microspheres, which offer the possibility of local noninvasive delivery of drugs over an extended period of time. This technology can include microspheres having a precapillary size, which can be injected via a coronary catheter into any selected part of an organ without causing inflammation or ischemia. The administered therapeutic is then slowly released from the microspheres and absorbed by the surrounding cells present in the selected tissue.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, bile salts, and fusidic acid derivatives. In addition, detergents may be used to facilitate permeation. Transmucosal administration can occur using nasal sprays or suppositories. For topical administration, the vector particles described herein can be formulated into ointments, salves, gels, or creams as generally known in the art. A wash solution can also be used locally to treat an injury or inflammation in order to accelerate healing.

Pharmaceutical forms suitable for injectable use can include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid. It must be stable under the conditions of manufacture and certain storage parameters (e.g., refrigeration and freezing) and must be preserved against the contaminating action of microorganisms, such as bacteria and fungi.

If formulations disclosed herein are used as a therapeutic to boost an immune response in a subject, a therapeutic agent can be formulated into a composition in a neutral or salt form. Pharmaceutically acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

A carrier can also be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents known in the art. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions can be prepared by incorporating the active compounds or constructs in the required amount in the appropriate solvent with various of the other ingredients enumerated above, as required, followed by filtered sterilization.

Upon formulation, solutions can be administered in a manner compatible with the dosage formulation and in such amount as is therapeutically effective. The formulations are easily administered in a variety of dosage forms, such as the type of injectable solutions described above, but slow-release capsules or microparticles and microspheres and the like can also be employed.

For parenteral administration in an aqueous solution, for example, the solution should be suitably buffered if necessary and the liquid diluent first rendered isotonic with sufficient saline or glucose. These particular aqueous solutions are especially suitable for intravenous, intratumorally, intramuscular, subcutaneous and intraperitoneal administration. In this context, sterile aqueous media that can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion.

The person responsible for administration will, in any event, determine the appropriate dose for the individual subject. For example, a subject may be administered the polypeptide complexes or polypeptide complex-based molecules and/or fusion polypeptides or fusion polypeptide-based molecules, or conjugates comprising the polypeptide complexes or polypeptide complex-based molecules and/or fusion polypeptides or fusion polypeptide-based molecules, nucleic acid molecules, vectors, cells, or target-binding moieties described herein on a daily or weekly basis for a time period or on a monthly, bi-yearly or yearly basis depending on need or a condition in the subject (e.g. cancer).

In addition to the compounds formulated for parenteral administration, such as intravenous, intratumorally, intradermal or intramuscular injection, other pharmaceutically acceptable forms include, e.g., tablets or other solids for oral administration; liposomal formulations; time release capsules; biodegradable and any other form currently used.

One may also use intranasal or inhalable solutions or sprays, aerosols or inhalants. Nasal solutions can be aqueous solutions designed to be administered to the nasal passages in drops or sprays. Nasal solutions can be prepared so that they are similar in many respects to nasal secretions. Thus, the aqueous nasal solutions usually are isotonic and slightly buffered to maintain a pH of 5.5 to 7.5. In addition, antimicrobial preservatives, similar to those used in ophthalmic preparations, and appropriate drug stabilizers, if required, may be included in the formulation. Various commercial nasal preparations are known and can include, for example, antibiotics and antihistamines and are used for asthma prophylaxis.

Oral formulations can include excipients as, for example, pharmaceutical grades of mannitol, lactose, starch, magnesium stearate, sodium saccharine, cellulose, magnesium carbonate and the like. These compositions take the form of solutions, suspensions, tablets, pills, capsules, sustained release formulations or powders. In certain defined embodiments, oral pharmaceutical compositions will include an inert diluent or assimilable edible carrier, or they may be enclosed in hard or soft-shell gelatin capsule, or they may be compressed into tablets, or they may be incorporated directly with the food of the diet. For oral therapeutic administration, the active compounds may be incorporated with excipients and used in the form of ingestible tablets, buccal tablets, troches, capsules, elixirs, suspensions, syrups, wafers, and the like.

The tablets, troches, pills, capsules and the like may also contain the following: a binder, as gum tragacanth, acacia, cornstarch, or gelatin; excipients, such as dicalcium phosphate; a disintegrating agent, such as corn starch, potato starch, alginic acid and the like; a lubricant, such as magnesium stearate; and a sweetening agent, such as sucrose, lactose or saccharin may be added or a flavoring agent, such as peppermint, oil of wintergreen, or cherry flavoring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar, or both. A syrup of elixir may contain the active compounds sucrose as a sweetening agent methyl and propylparabens as preservatives, a dye and flavoring, such as cherry or orange flavor.

Further embodiments disclosed herein can concern kits for use with methods and compositions. Kits can also include a suitable container, for example, vials, tubes, mini- or microfuge tubes, test tube, flask, bottle, syringe or other container. Where an additional component or agent is provided, the kit can contain one or more additional containers into which this agent or component may be placed. Kits herein will also typically include a means for containing the polypeptide complexes or polypeptide complex-based molecules and/or fusion polypeptides or fusion polypeptide-based molecules, or conjugates comprising the polypeptide complexes or polypeptide complex-based molecules and/or fusion polypeptides or fusion polypeptide-based molecules, nucleic acid molecules, vectors, cells, or target-binding moieties and any other reagent containers in close confinement for commercial sale. Such containers may include injection or blow-molded plastic containers into which the desired vials are retained. Optionally, one or more additional active agents may be needed for compositions described.

Dose ranges and frequency of administration can vary depending on the nature of the composition and the medical condition as well as parameters of a specific patient and the route of administration used. A dose can also depend on the subject in which it is being administered. For example, a lower dose may be required if the subject is juvenile, and a higher dose may be required if the subject is an adult human subject. In certain embodiments, a more accurate dose can depend on the weight of the subject. A suitable, non-limiting example of a dosage of a pharmaceutical composition containing the same disclosed herein may vary depending upon the age and the size of a subject to be administered, target disease, the purpose of the treatment, conditions, route of administration, and the like. Non-limiting examples of suitable dosages include, e.g., 0.01 to about 20 mg/kg body weight, more preferably about 0.02 to about 7, about 0.03 to about 5, or about 0.05 to about 3 mg/kg body weight. Depending on the severity of the condition, the frequency and the duration of the treatment can be adjusted. In certain embodiments, the initial dose may be followed by administration of a second or a plurality of subsequent doses in an amount that can be approximately the same or less than that of the initial dose, wherein the subsequent doses are separated by at least 1 day to 3 days; at least one week, at least 2 weeks; at least 3 weeks; at least 4 weeks; at least 5 weeks; at least 6 weeks; at least 7 weeks; at least 8 weeks; at least 9 weeks; at least 10 weeks; at least 12 weeks; or at least 14 weeks.

Compositions may include administration to a subject intravenously, intratumorally, intradermally, intraarterially, intraperitoneally, intralesionally, intracranially, intraarticularly, intraprostaticaly, intrapleurally, intratracheally, intranasally, intravitreally, intravaginally, intrarectally, topically, intratumorally, intramuscularly, intrathecally, subcutaneously, subconjunctival, intravesicularlly, mucosally, intrapericardially, intraumbilically, intraocularly, orally, locally, by inhalation, by injection, by infusion, by continuous infusion, by localized perfusion, via a catheter, via a lavage, in a cream, or in a lipid composition.

Certain additional agents used in the combination therapies can be formulated and administered by any means known in the art.

Compositions as disclosed herein can also include adjuvants such as aluminum salts and other mineral adjuvants, tensoactive agents, bacterial derivatives, vehicles and cytokines. Adjuvants can also have antagonizing immunomodulating properties. Compositions and methods as disclosed herein can also include adjuvant therapy.

The pharmaceutical compositions of the disclosure may be administered directly into the patient, into the affected organ or systemically i.d., i.m., s.c., i.p. and i.v., or applied ex vivo to cells derived from the patient or a human cell line which are subsequently administered to the patient or used in vitro to select a subpopulation of immune cells derived from the patient, which are then re-administered to the patient. If the nucleic acid is administered to cells in vitro, it may be useful for the cells to be transfected so as to co-express immune-stimulating cytokines, such as interleukin-2. The peptide or peptide-based molecule may be substantially pure or combined with an immune-stimulating adjuvant or used in combination with immune-stimulatory cytokines, or be administered with a suitable delivery system, e.g., liposomes, viral particles, VLPs. The peptide or peptide-based molecule may also be conjugated to a suitable carrier such as keyhole limpet haemocyanin (KLH) or mannan (see, e.g., WO 95/18145 and Longenecker et al., 1993).

Methods for introducing polypeptide or polynucleotides of the present disclosure into a cell or subject can include, for example, vector delivery, particle-mediated delivery, exosome-mediated delivery, lipid-nanoparticle-mediated delivery, cell-penetrating-peptide-mediated delivery, or implantable-device-mediated delivery. In some embodiments, a nucleic acid or protein can be introduced into a cell or subject in a carrier such as a poly(lactic acid) (PLA) microsphere, a poly(D,L-lactic-coglycolic-acid) (PLGA) microsphere, a liposome, a micelle, an inverse micelle, a lipid cochleate, or a lipid microtubule.

The use of nanoparticles to deliver the polypeptide or polynucleotides compositions of the disclosure is contemplated herein. Exemplary nanoparticles include, but are not limited to, polymeric nanoparticles, inorganic nanoparticles, liposomes, lipid nanoparticles (LNP), an immune stimulating complex (ISCOM), a virus-like particle (VLP), or a self-assembling protein. The nanoparticles may be calcium phosphate nanoparticles, silicon nanoparticles or gold nanoparticles. For examples, the polymeric nanoparticles may comprise one or more synthetic polymers, such as poly(d,l-lactide-co-glycolide) (PLG), poly(d,l-lactic-coglycolic acid) (PLGA), poly(g-glutamic acid) (g-PGA), poly(ethylene glycol) (PEG), or polystyrene or one or more natural polymers such as a polysaccharide, for example pullulan, alginate, inulin, and chitosan. The use of a polymeric nanoparticles may be advantageous due to the properties of the polymers that may be include in the nanoparticle. For instance, the natural and synthetic polymers recited above may have good biocompatibility and biodegradability, a non-toxic nature and/or the ability to be manipulated into desired shapes and sizes. The polymeric nanoparticle may also form hydrogel nanoparticles, hydrophilic three-dimensional polymer networks with favorable properties including flexible mesh size, large surface area for multivalent conjugation, high water content, and high loading capacity for antigens. Polymers such as Poly(L-lactic acid) (PLA), PLGA, PEG, and polysaccharides are suitable for forming hydrogel nanoparticles. Inorganic nanoparticles typically have a rigid structure and comprise a shell in which an antigen is encapsulated or a core to which the antigen may be covalently attached. The core may comprise one or more atoms such as gold (Au), silver (Ag), copper (Cu) atoms, Au/Ag, Au/Cu, Au/Ag/Cu, Au/Pt, Au/Pd or Au/Ag/Cu/Pd or calcium phosphate (CaP).

Other molecules suitable for complexing with the polypeptide or polynucleotides of the disclosure include cationic molecules, such as, polyamidoamine, dendritic polylysine, polyethylene irinine or polypropylene imine, polylysine, chitosan, DNA-gelatin coarcervates, DEAE dextran, dendrimers, or polyethylenimine (PEI).

In some embodiments, compositions of the present disclosure can be conjugated to nanoparticles. Nanoparticles that may be used for conjugation with antibodies of the present disclosure include but not are limited to PEGylated liposomes, poly(d,l-lactide-co-glycolide)/montmorillonite nanoparticles (PLGA/MMT NPs), poly(lactide-co-glycolide) (PLGA) nanoparticles, poly-(malic acid)-based nanoparticles, chitosan-shelled nanoparticles, carbon nanotubes, and other inorganic nanoparticles (such as nanoparticles made of magnesium-aluminum layered double hydroxides with disuccinimidyl carbonate (DSC), and TiO₂ nanoparticles). Nanoparticles can be developed and conjugated to an antibody contained in a pharmaceutical composition for targeting virus-infected cells.

### Methods for Delivering Mature TGFβ Family Polypeptides

In various embodiments, the present disclosure provides methods for delivering a mature TGFβ family polypeptide, or the fragment or derivative thereof, to a target cell disclosed herein within a subject in need thereof. The method comprises administering to the subject a polypeptide complex disclosed herein, a pharmaceutical composition disclosed herein, a polynucleotide disclosed herein, and/or a vector disclosed herein. In certain embodiments, a target-binding polypeptide (e.g., an antigen-binding polypeptide) within the polypeptide complex a molecule on the target cell. In some embodiments, the mature TGFβ family polypeptide, or the fragment or derivative thereof, is capable of binding a TGFβR on the target cell. In some embodiments, the mature TGFβ family polypeptide, or the fragment or derivative thereof, may bind a TGFβR on the target cell. In some embodiments, the mature TGFβ family polypeptide, or the fragment or derivative thereof, is capable of inducing Smad2/3 signaling in the target cell.

The polypeptide complex(es), pharmaceutical composition(s), polynucleotide(s), and/or a vector(s) for delivery of the mature TGFβ family polypeptide, or the fragment(s) or derivative(s) thereof, may be administered via any of various delivery routes described herein, and in accordance with any of the dosages and/or frequencies of administration described herein. The polypeptide complex(es), pharmaceutical composition(s), polynucleotide(s), and/or a vector(s) for delivery of the mature TGFβ family polypeptide, or the fragment(s) or derivative(s) thereof, may be prepared in accordance with any of the preparation techniques described herein. By way of a non-limiting example, the polypeptide complex(es), pharmaceutical composition(s), polynucleotide(s), and/or a vector(s) for delivery of the mature TGFβ family polypeptide, or the fragment(s) or derivative(s) thereof may be formulated into any of the formulations described herein. Kits comprising polypeptide complex(es), pharmaceutical composition(s), polynucleotide(s), and/or vector(s) for delivery of the mature TGFβ family polypeptide, or the fragment(s) or derivative(s) thereof are also contemplated herein

### Treatment Methods

The pharmaceutical compositions comprising, e.g., a polypeptide complex, a fusion polypeptide, a polynucleotide, a vector, and/or a cell disclosed herein and a carrier and/or excipient disclosed herein may be used for various therapeutic applications (in vivo and ex vivo) and as research tools. In one aspect, described herein is a method for treating a disease in a subject in need thereof, comprising administering to the subject a therapeutically effective amount of a polypeptide complex, a fusion polypeptide, a polynucleotide, a vector, and/or a cell, and/or pharmaceutical compositions thereof, disclosed herein. In some embodiments, the subject can be a human.

In certain aspects, the present disclosure provides methods for treating a TGFβ dysregulation disorder in a subject in need thereof. The method comprises administering to the subject a therapeutically effective amount of, e.g., a polypeptide complex, a fusion polypeptide, a polynucleotide, a vector, and/or a cell, and/or pharmaceutical compositions thereof, disclosed herein. A TGFβ dysregulation disorder disclosed herein may comprise a state, disorder, disease, or condition associated with dysregulation of TGFβ, including, e.g., low TGFβ expression and expression of variant forms of TGFβ. Non-limiting examples of TGFβ dysregulation disorders include Type 1 diabetes mellitus, inflammatory bowel disease (IBD), colitis, Marfan syndrome (MFS), aortic dilation and rupture (aortic aneurysm), an autoimmune disorder, an arthritis, lupus (e.g., systemic lupus), and a wound healing disorder.

In certain aspects, the present disclosure provides methods for promoting wound healing in a subject in need thereof, comprising administering to the subject a therapeutically effective amount of, e.g., a polypeptide complex, a fusion polypeptide, a polynucleotide, a vector, and/or a cell, and/or pharmaceutical compositions thereof, disclosed herein. In some embodiments, the subject has a wound. In some embodiments, the polypeptide complex, the fusion polypeptide, the polynucleotide, the vector, and/or the cell, and/or the pharmaceutical composition may be administered to the wound of the subject.

In some embodiments, a polypeptide complex, fusion polypeptide, polynucleotide, vector, and/or a cell, and/or pharmaceutical composition thereof, disclosed herein may be used to treat different types of autoimmune disorders such as, but not limited to, e.g., autoimmune gastritis, vasculitis, Wegener's granulomatosis, Hashimoto's thyroiditis, psoriasis Graves' disease, Guillain-Barre syndrome, chronic inflammatory demyelinating polyneuropathy Crohn's disease, ulcerative colitis, Rheumatoid arthritis (RA), multiple sclerosis (MS), Sjögren's syndrome, sarcoidosis, Systemic lupus erythematosus, Type 1 diabetes mellitus, insulin dependent diabetes mellitus (IDDM), autoimmune thyroiditis, reactive arthritis, Myasthenia gravis, ankylosing spondylitis, scleroderma, polymyositis, or dermatomyositis.

In various embodiments, the administration of the polypeptide complex(es), fusion polypeptide(s), polynucleotide(s), vector(s), and/or a cell(s), and/or pharmaceutical compositions thereof, may be administered at an amount effective to achieve, for example, decreased joint swelling, inflammatory cell infiltration, white blood cell (WBC), total IgG production, kidney inflammation and/or disease incidence.

In various embodiments, the administration of the polypeptide complex(es), fusion polypeptide(s), polynucleotide(s), vector(s), and/or a cell(s), or pharmaceutical compositions thereof, may be administered at an amount effective to increase survival.

In certain embodiments, the polypeptide complex(es), fusion polypeptide(s), polynucleotide(s), vector(s), and/or a cell(s), or pharmaceutical compositions thereof, may be administered, for example, via injection. In some embodiments, the injection is intraperitoneal, intravenous, subcutaneous, intramuscular, or transdermal.

In certain embodiments, the polypeptide complex(es), fusion polypeptide(s), polynucleotide(s), vector(s), and/or a cell(s), or pharmaceutical compositions thereof, may be administered intranasally, orally, or mucosally.

In certain embodiments, the polypeptide complex(es), fusion polypeptide(s), polynucleotide(s), vector(s), and/or a cell(s), or pharmaceutical compositions thereof, may be administered via hydrodynamic delivery (HDD).

In certain embodiments, the polypeptide complex(es), fusion polypeptide(s), polynucleotide(s), vector(s), and/or a cell(s), or pharmaceutical compositions thereof, may be administered to the liver of a subject in need thereof.

In certain embodiments, a polypeptide complex, fusion polypeptide, polynucleotide, vector, and/or a cell, and/or pharmaceutical composition thereof, may be administered, for example, intraperitoneally, intravenously, subcutaneously, intramuscularly, transdermally, intranasally, orally, or mucosally.

In some embodiments, a polypeptide complex, fusion polypeptide, polynucleotide, vector, and/or a cell, and/or pharmaceutical composition thereof can be administered via systemic delivery. In some embodiments, the recombinant virus or composition is administered via parenteral delivery. Non-limiting examples of parenteral delivery include subcutaneous, intraperitoneal, intradermal, intramuscular, or intravenous delivery. In some embodiments, the parental delivery is intravenous delivery.

It is contemplated that when used to treat various diseases, the compositions and methods disclosed herein can be combined with additional therapeutic agents such as therapeutic agents which may be suitable for the same or similar diseases, for example, a second anti-autoimmune disease or IBD treatment. Also, two or more embodiments described herein may be also co-administered to generate additive or synergistic effects. When co-administered with an additional therapeutic agent, the embodiment described herein, and the additional therapeutic agent may be administered simultaneously or sequentially (in any order). Suitable therapeutically effective dosages for each agent may be lowered due to the additive action or synergy.

### EXAMPLES

The following examples are provided to further describe some of the embodiments disclosed herein. The examples are intended to illustrate, not to limit, the disclosed embodiments.

### Example 1. Generation of Targeted Antibody-TGFβ Fusion Proteins

The goal of the present experiment was to generate targeted antibody-TGFβ fusion proteins to deliver latent TGFβ to specific cell types (FIG. 3A). The objective of the present Example was to find an expression system to obtain furin-processed antibody-TGFβ SLC proteins. As a proof-of-concept study, anti-hCD63-TGFβ1 fusion constructs were generated (see, e.g., FIG. 3B).

To generate anti-hCD63-TGFβ constructs, mROR1 signal peptide (SP) was fused to a human CD63 antibody heavy chain (hIgG4) and linked to human full-length TGFβ1 (Uniprot ID P01137) via a 12-amino acid linker. The anti-hCD63 clone H5C6 was used to generate anti-hCD63-TGFβ constructs. A separate construct was used to express the human CD63 antibody light chain. Additionally, an anti-hCD63-TGFβ2 (Uniprot ID: P61812) construct was generated to use as a negative control in the mechanical activation assays because TGFβ2 does not have the canonical tripeptide Arg-Gly-Asp (Arginine, Glycine, and Aspartate) ‘RGD’ binding motif required for integrin-mediated mechanical activation. A positive control construct (which included hIgG1 instead of hIgG4) was used to check the expression and secretion of TGFβ1 under the present experimental conditions. All tested constructs contained a C33S mutation (cysteine-to-serine mutation at position 33) in the LAP domain. Since LAP is covalently linked to cell surface-bound milieu molecules at C33, the C33S mutation prevents disulfide bonding to milieu molecules during secretion, which prevents incorporation of milieu molecules into TGFβ SLC.

Anti-hCD63-TGFβ1 and anti-hCD63-TGFβ2 heavy chain constructs were cloned in a pRGN4631 vector, the anti-hCD63 light chain construct was cloned in a pRGN4641 vector, and the TGFβ1 positive control vector was cloned in a pRG1174 vector. Anti-hCD63-TGFβ1, anti-hCD63-TGFβ2, the positive control vector, and the enhanced green fluorescent protein (EGFP) were expressed in ExpiCHO (Chinese hamster ovary)-S cells.

### Transient Expression in ExpiCHO-S Cells:

1. ExpiCHO-S cells were passaged three times below 5×10⁶ cells/mL in ExpiCHO expression medium before transfections. The cells were maintained at 37° C., 80% humidity, 8% CO₂, and 120 rpm orbital shaker (25 mm shaking diameter).

2. One day before transfections, the cells were centrifuged at 100 g for 10 min, the cell pellet was resuspended in fresh media, and passed through a 40 μm cell strainer. After cell counting, the cells were seeded at 3×10⁶ cells/mL (30 mL total volume) in 125 mL vented flasks.

3. On the day of transfection, the cells were diluted to 6×10⁶ cells/mL (total volume 25 mL). In one tube, 25 μg plasmid DNA was diluted in 1 mL OptiProSFM. In another tube, 80 μL ExpiFectamine CHO transfection reagent was diluted in 920 μL OptiProSFM. Diluted ExpiFectamine CHO transfection reagent was added to a diluted plasmid DNA tube slowly and incubated at room temperature for 5 min. This DNA-transfection reagent complex was added to the cells slowly by swirling the flask.

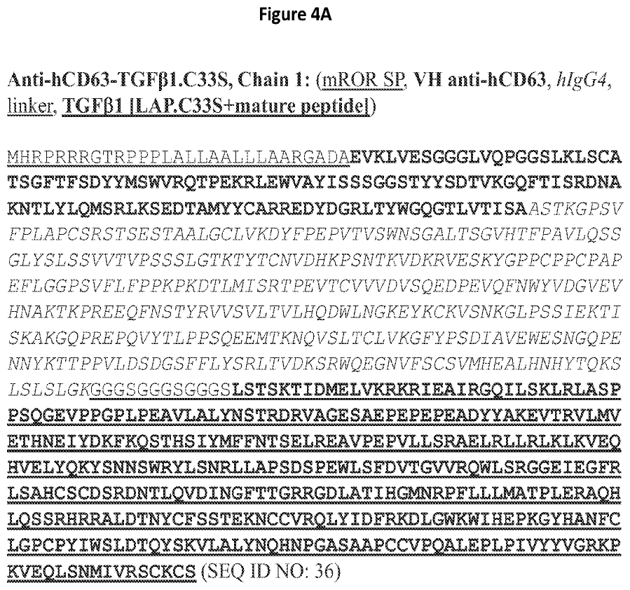

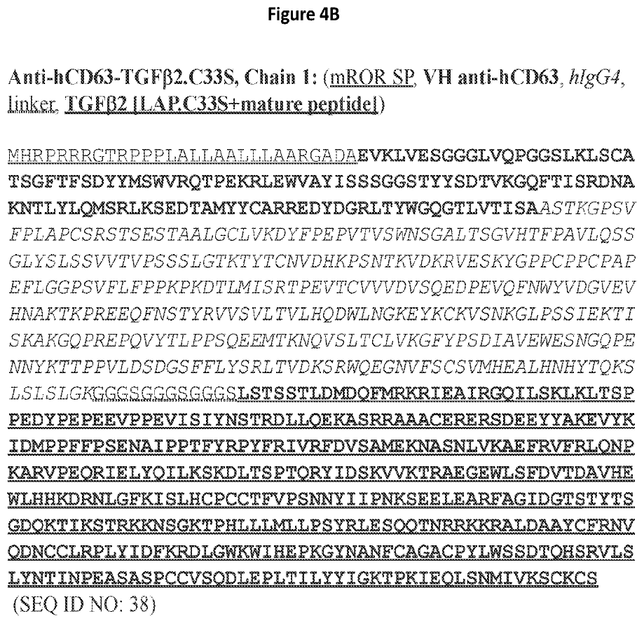

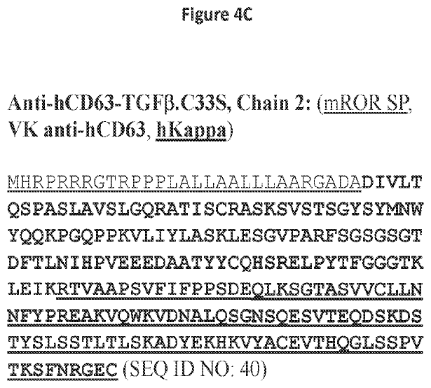

4. For antiCD63-TGFβ1 and antiCD63-TGFβ2 transfections, 8 μg heavy chain and 16 μg light chain constructs were combined (heavy chain:light chain ratio=1:2). Heavy and light chain amino acid sequences of antiCD63-TGFβ1 and antiCD63-TGFβ2 are shown in FIGS. 4A-4C.

5. 20 hours after transfection, 150 μL ExpiCHO enhancer and 6 mL ExpiCHO Feed were combined and added to the transfected cells.

6. 4 days after transfections, transfected ExpiCHO cultures were transferred to a 50 mL falcon tube and centrifuged at 4000 g for 10 min. The supernatant was filtered through a 0.2 μm filter and a protease inhibitor cocktail was added (1× final concentration). Both ExpiCHO supernatants and pellets were stored at −80° C.

To find a better expression system yielding less aggregation and more processing, antiCD63-TGFβ1 was expressed in FreedomCHO-S cells.

### Transient Expression in FreedomCHO-S Cells:

1. FreedomCHO-S cells were passaged three times below 2×10⁶ cells/mL in CD FortiCHO expression medium supplemented with 8 mM L-Glutamine before the transfections. The cells were maintained at 37° C., 80% humidity, 8% CO₂, and 125 rpm orbital shaker (25 mm shaking diameter).

2. One day before transfections, the cells were centrifuged at 100 g for 10 min, the cell pellet was resuspended in fresh complete media, and passed through a 40 μm cell. After cell counting, the cells were seeded at 5×10⁵ cells/mL (30 mL total volume) in 125 mL vented flasks.

3. On the day of transfection, the cells were diluted to 1×10⁶ cells/mL (total volume 30 mL). In one tube, 50 μg plasmid DNA was diluted in 1.5 mL OptiProSFM. In another tube, 50 μL FreeStyle Max transfection reagent was diluted in 1.45 mL OptiProSFM. Diluted FreeStyle Max transfection reagent was added to diluted plasmid DNA tube slowly and incubated at room temperature for 10 min. The DNA-transfection reagent complex was added to the cells slowly by swirling the flask.

4. For anti-hCD63-TGFβ1 and anti-hCD63-TGFβ2 transfections, 16 μg heavy chain and 32 μg light chain constructs were combined (heavy chain:light chain ratio=1:2). Heavy and light chain sequences of antiCD63-TGFβ1 and antiCD63-TGFβ2 are shown in FIGS. 4A-4C.

5. Two days after transfections, transfected FreedomCHO cultures were transferred to a 50 mL falcon tube and centrifuged at 4000 g for 10 min. The supernatant was filtered through a 0.2 μm filter and a protease inhibitor cocktail was added (1× final concentration). Both FreedomCHO supernatants and pellets were stored at −80° C.

To analyze expression, secretion, and processing of anti-hCD63-TGFβ fusion proteins, immunoblots were run. Both cell pellet and conditioned media supernatant samples were used. Cell pellets were resuspended in 1× Ripa buffer supplemented with protease inhibitor cocktail (1× final concentration) and incubated on a rotating platform at 4° C. for 10 min. After lysis, samples were centrifuged at 12,000 rpm at 4° C. for 15 min. Supernatants from the cell lysates were collected. Both conditioned media supernatant and cell lysate supernatant samples corresponding to the same volume of culture were run under reducing and non-reducing conditions on 4-12% Novex SDS-PAGE and transferred to PVDF (polyvinylidene fluoride) membranes. Membranes were blocked in Superblock buffer for 3 hours at room temperature and incubated with primary anti-TGFβ1/2 antibody at a 1:2000 dilution or with primary anti-hIgG4 antibody at a 1:2000 dilution overnight at 4° C., followed by incubation with horseradish peroxidase (HRP)-conjugated secondary antibody at a 1:5000 dilution for 1 hour at room temperature. Western Bright enhanced chemiluminescence (ECL) HRP substrate was used for detection.

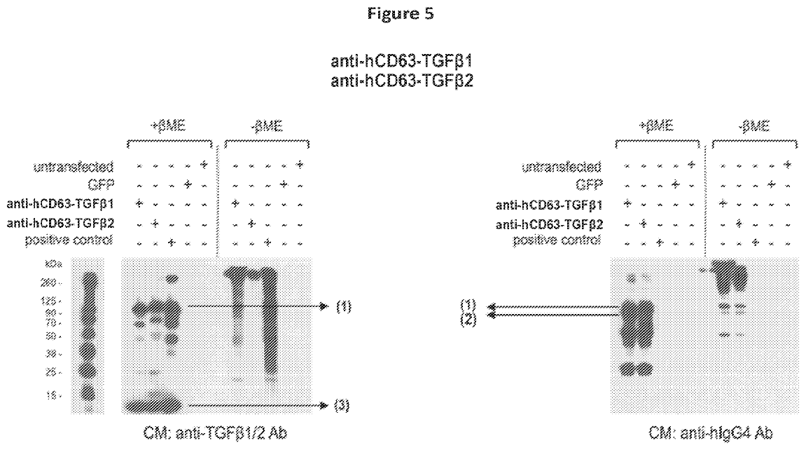

As shown in FIG. 5, anti-hCD63-TGFβ1, anti-hCD63-TGFβ2, gLuciFc-TGFβ1 (positive control), and EGFP were expressed in ExpiCHO-S cells. Conditioned media (CM) samples were run on western blots under reducing (with β-mercaptoethanol, +BME) and non-reducing (without β-mercaptoethanol, -BME) conditions. FIG. 5 shows that both unprocessed anti-hCD63-TGFβ fusion protein (labeled as form 1) and processed anti-hCD63-TGFβ SLC (form 2 and 3 are noncovalently associated) are secreted in ExpiCHO CM. Form 1 is unprocessed anti-hCD63-TGFβ (LAP+mature domain) fusion protein (detected with both anti-TGFβ1/2 and anti-IgG4 antibodies). Form 3 is mature TGFβ (12 kDa) (detected with only anti-TGFβ1/2 antibody) and form 2 is anti-hCD63-LAP (90 kDa) (detected only with anti-IgG4 antibody).

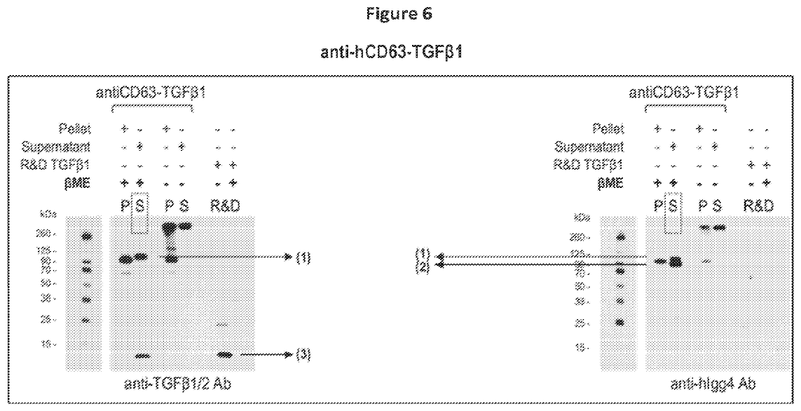

Because of the disulfide bonds, the molecular weight of unprocessed and processed anti-hCD63-TGFβ constructs double under non-reducing conditions. However, several aggregation bands were also seen under non-reducing conditions on the western blots run with CM samples (FIG. 5). To identify an improved expression system yielding less aggregation and more furin processing, anti-hCD63-TGFβ1 was expressed in FreedomCHO cells. Both CM (labelled as ‘S’) and cell lysate (labelled as ‘P’) samples were run on western blots under reducing (+BME) and non-reducing (-BME) conditions (FIG. 6). FIG. 6 shows that both unprocessed anti-hCD63-TGFβ1 fusion protein (form 1) and processed anti-hCD63-TGFβ SLC (forms 2 and 3 are noncovalently associated) are secreted in the FreedomCHO cells. R&D TGFβ1 was used as a control to detect mature TGFβ1 under reducing and non-reducing conditions. No aggregation band was observed in FreedomCHO CM samples. Based on the density of form 1 and form 2 on anti-IgG4 western, 60% processing of anti-hCD63-TGFβ is obtained with endogenous Furin in FreedomCHO cells.

Based on these results, FreedomCHO cells will be used to express target antibody-TGFβ fusion constructs in the future. Anti-hCD63-TGFβ was used for proof-of-concept (POC) studies and anti-hCD63 could be swapped out with alternative antibodies.

### Example 2. Processed Anti-hCD63-TGFβ1 SLC can be Activated Chemically, Proteolytically, and Mechanically

The objective of this Example was to determine whether the anti-hCD63-TGFβ1 SLC fusion protein expressed in ExpiCHO cells can be activated in vitro to induce Smad2/3 signaling. Both chemical and mechanical activation approaches were tested to activate anti-hCD63-TGFβ1 SLC fusions.

In chemical activation, anti-hCD63-TGFβ1 transfected ExpiCHO conditioned media (CM) samples were either treated with acid or heat to dissociate the LAP domain and release mature TGFβ1. For heat treatment, CM samples were incubated at 80° C. for 10 min. For acid treatment, CM samples were acidified to pH 4 with HCl (hydrochloric acid), incubated on ice for 1 hr, and neutralized to pH 7 with NaOH (sodium hydroxide). Non-activated samples were kept as a control in the chemical activation assay.

To test the activity of non-activated, heat-activated, and acid-activated samples, a dose-response signaling assay was performed in Hek293 cells carrying Smad2/3 responsive luciferase reporter vector. In this assay:

1. Day 1: 10,000 Hek293-CAGA (cells carrying Smad2/3 responsive luciferase vector) were plated in Poly-D-Lysine (PDL) coated 96-well luciferase plates in 100 μL/well complete DMEM media supplemented with 10% FBS, 2 mM L-Glutamine, 100 units/mL P/S, and 500 μg/mL G418. Cells were maintained at 37° C., 5% CO₂.

2. Day 2: 100 μL media was removed from each well and fresh 100 μL serum-free media (DMEM+2 mM L-Glutamine+100 units/mL P/S+500 μg/mL G418) supplemented with 0.1% BSA was added to each well.

3. Day 3: Non-activated, heat-activated, and acid-activated samples were titrated in serum free media (DMEM+2 mM L-Glutamine+100 units/mL P/S) supplemented with 0.1% BSA. 100 μL media was removed from each well and 100 μL of titration sample was added to each well.

4. After 16 hrs, the plate was incubated at RT for 10 min. Then, 100 L/well BrightGlo luciferase reagent was added and incubated at RT for 5 min. After the incubation, luminescence measurement was taken, and the results were analyzed with Prism software.

For mechanical activation, CD63 expressing cells, integrin αvβ6 expressing cells, and reporter cells were co-cultured in a PDL-coated 96-well luciferase plate. 2,500 cells/well hCD63 overexpressing Hek293 cells, hCD63.Y235A overexpressing Hek293 cells (made using construct pNSc0199) or CD63 KO Hek293 cells were co-cultured with 2,500 cells/well hIntegrin αvβ6 overexpressing CHO-K1 (or regular CHO-K1 cells in complete media (1:1 mix of Ham's F12 media:DMEM media supplemented with 10% FBS+2 mM L-Glutamine+100 units/mL P/S)). On the second day, the media was removed from each well and 2,500 Hek293-CAGA reporter cells were added to each well in complete media. On the third day, media was removed from each well and starvation was performed for 3 hrs with serum free media (1:1 mix of Ham's F12 media:DMEM) supplemented with 2 mM L-Glutamine+100 units/mL P/S+0.1% BSA. Anti-hCD63-TGFβ1 transfected ExpiCHO samples were titrated in the same serum free media. After a 3 hr starvation period, media was replaced with anti-hCD63-TGFβ1 CM-containing titration samples. After 16 hrs of incubation with titration samples, the plate was incubated at RT for 10 min. Then, 100 μL/well BrightGlo luciferase reagent was added to the samples and incubated at RT for 5 min. After incubation, luminescence was measured, and the results were analyzed with Prism software.

Anti-hCD63-TGFβ1 was expressed in ExpiCHO cells to show activation and Smad2/3 signaling induction. Both unprocessed full-length and processed anti-hCD63-TGFβ1 SLC were secreted in conditioned media. Only processed anti-hCD63-TGFβ1 SLC can be activated chemically, proteolytically, and mechanically. Unprocessed anti-hCD63-TGFβ1 full-length form is inactive and does not induce signaling.

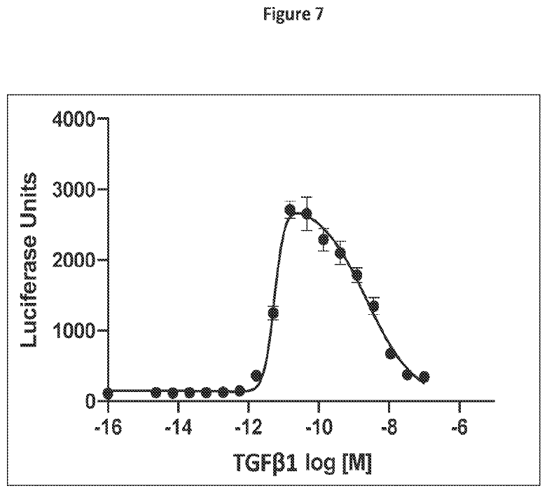

To determine the amount of mature TGFβ1 present after activation in CM of anti-hCD63-TGFβ1 transfected ExpiCHO cells, commercially available TGFβ1 was titrated in Hek293-CAGA reporter cells, and Smad2/3 was measured (FIG. 7). TGFβ1 dose-response signaling data in FIG. 7 was used as a standard curve to calculate how much mature TGFβ1 was released after heat and acid activation of anti-hCD63-TGFβ1 SLC. Based on this calculation, approximately 936.6 ng/mL mature TGFβ1 was present in the heat-activated sample, and approximately 311.2 ng/mL mature TGFβ1 was present in the acid-activated sample. Raw data of TGFβ1 dose-response signaling activation corresponding to FIG. 7 are shown in Table 1. For Table 1, LU indicates luciferase units, and SEM indicates Standard Error of Mean. For each condition, quadruplicate samples were run.

TABLE 1
Raw data of TGFβ1 dose-response signaling activation

| | Mean | |
|---|---|---|
| TGFβ1 [M] | LU | SEM |
| 1e−007 | 340 | 33.9 |
| 3.3333e−008 | 376 | 39.9 |
| 1.1111e−008 | 672 | 52.2 |
| 3.7037e−009 | 1341 | 122.3 |
| 1.2346e−009 | 1781 | 101.4 |
| 4.1152e−010 | 2102 | 164.1 |
| 1.3717e−010 | 2293 | 162.5 |
| 4.5725e−011 | 2656 | 236.2 |
| 1.5242e−011 | 2712 | 120.8 |
| 5.0805e−012 | 1246 | 97.3 |
| 1.6935e−012 | 361 | 15.1 |
| 5.645e−013 | 144 | 2.2 |
| 1.8817e−013 | 124 | 4.2 |
| 6.2723e−014 | 121 | 3.4 |
| 2.0908e−014 | 117 | 5.9 |
| 6.9692e−015 | 115 | 9.0 |
| 2.3231e−015 | 122 | 7.7 |
| 1e−016 | 109 | 3.4 |

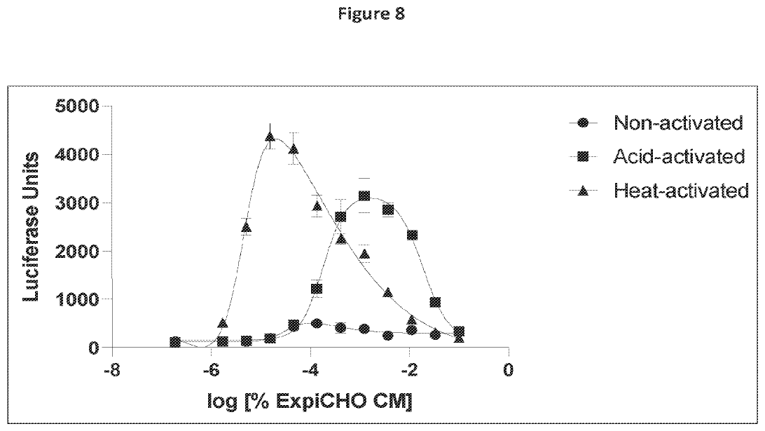

As shown in FIG. 8, both acid and heat activation released mature TGFβ1 from processed anti-hCD63-TGFβ1 SLC, and mature TGFβ1 induced Smad2/3 signaling in Hek293-CAGA reporter cells. Heat-activated samples induced Smad2/3 signaling at lower doses compared to acid-activated samples, showing that heat activation was more efficient at releasing mature TGFβ in this system. Raw data of the chemical activation of anti-hCD63-TGFβ1 corresponding to FIG. 8 are shown in Table 2. For Table 2, LU indicates luciferase units, SEM indicates Standard Error of Mean, and CM indicates Condition Media. For each condition, quadruplicate samples were run.

TABLE 2
Raw data of the chemical activation of anti-hCD63-TGFβ1

| | Non-activated CM | | Acid-activated CM | | Heat-activated CM | |
|---|---|---|---|---|---|---|
| CM (%) | Mean LU | SEM | Mean LU | SEM | Mean LU | SEM |
| 0.1 | 323.0 | 14.9 | 336.0 | 26.0 | 205.0 | 9.0 |
| 0.03333 | 266.2 | 9.5 | 941.0 | 31.0 | 315.0 | 22.0 |
| 0.01111 | 361.2 | 30.2 | 2334.0 | 68.0 | 583.0 | 22.0 |
| 0.00370 | 248.7 | 7.1 | 2858.0 | 149.0 | 1144.0 | 23.0 |
| 0.00123 | 389.8 | 59.6 | 3144.0 | 353.0 | 1951.0 | 178.0 |
| 0.00041 | 412.0 | 109.9 | 2710.0 | 352.0 | 2266.0 | 128.0 |
| 0.00013 | 503.0 | 56.9 | 1218.0 | 188.0 | 2934.0 | 231.0 |
| 4.6E−05 | 432.0 | 66.1 | 482.0 | 40.0 | 4117.0 | 327.0 |
| 1.5E−05 | 198.0 | 32.2 | 192.0 | 35.0 | 4374.0 | 271.0 |
| 5.1E−06 | 123.0 | 9.8 | 142.0 | 8.0 | 2508.0 | 172.0 |
| 1.7E−06 | 122.0 | 9.5 | 130.0 | 5.0 | 520.0 | 20.0 |
| 1.9E−07 | 128.0 | 9.0 | 113.0 | 10.0 | 132.0 | 8.0 |

To determine whether anti-hCD63-TGFβ1 SLC can be mechanically activated, a co-culture assay of target protein-expressing cells, integrin αvβ6 expressing cells, and Smad2/3 responsive reporter cells was performed. Human CD63 wild-type (WT) expressing Hek293 cells were used to anchor anti-hCD63-TGFβ1 SLC fusion proteins. Human integrin αvβ6 expressing CHO-K1 cells were used to provide mechanical activation, and Hek293-CAGA reporter cells were used to measure Smad2/3 signaling. It is known that CD63 internalizes in a relatively short time. If WT CD63 internalizes faster than anti-hCD63-TGFβ1 SLC activation, Smad2/3 may not be induced. Therefore, in addition to hCD63 WT expressing cells, hCD63.Y235A non-internalizing mutant expressing cells were also used in the mechanical activation assay.

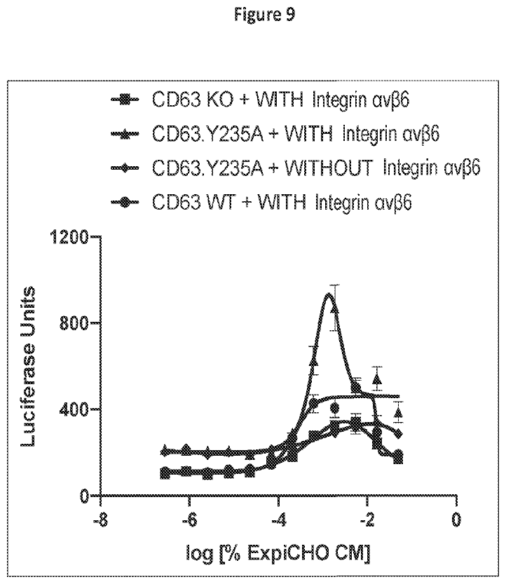

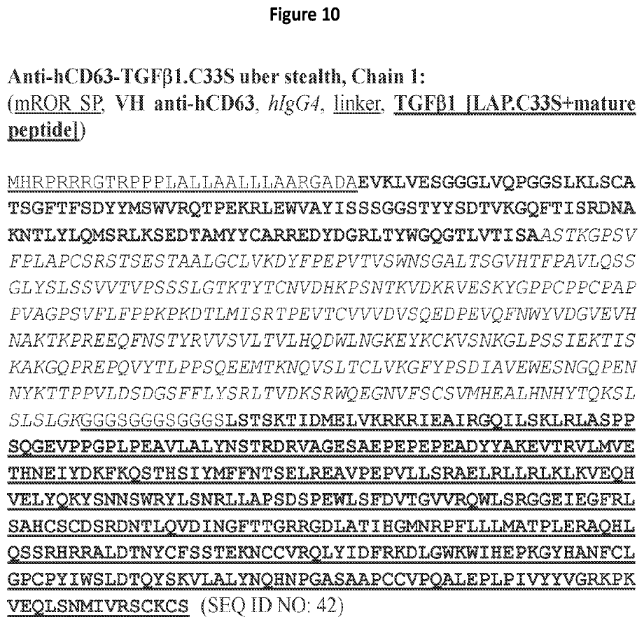

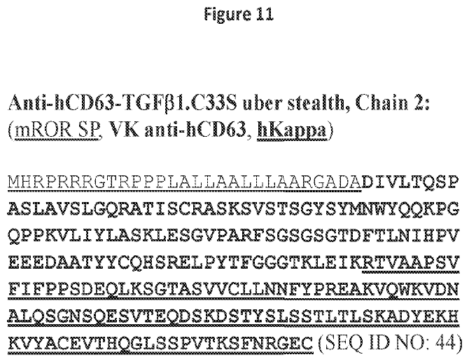

As shown in FIG. 9, anti-hCD63-TGFβ1 SLC was activated mostly in CD63.Y235A expressing cells co-cultured with integrin αvβ6 expressing cells. Without integrin αvβ6 expressing cells, CD63.Y235A binding alone did not activate anti-hCD63-TGFβ1 SLC. When CD63 knockout (KO) cells were co-cultured with integrin αvβ6 expressing cells, Smad2/3 activation was significantly less. This slight activation in CD63 KO may be due to the presence of endogenous ligands in ExpiCHO CM (including TGFβ SLC) or endogenous CD63 expression in CHO-K1 and Hek293-CAGA reporter cells. These results show that both integrin αvβ6 and CD63 binding is required for activation of anti-hCD63-TGFβ1 SLC. Interestingly, anti-hCD63-TGFβ1 SLC was activated significantly less in the presence of WT CD63 expressing cells compared to the non-internalizing CD63.Y235A mutant expressing cells, suggesting that rapid internalization of the target protein decreases activation of anti-hCD63-TGFβ SLC fusion proteins. Raw data of the mechanical activation of anti-hCD63-TGFβ1 corresponding to FIG. 9 are shown in Table 3. For Table 3, LU indicates luciferase units, SEM indicates Standard Error of Mean, and CM indicates Condition Media. For each condition, quadruplicate samples were run.

TABLE 3
Raw data of the mechanical activation of anti-hCD63-TGFβ1

| | CD63 WT + | | CD63 KO + | | CD63.Y235A + | | CD63.Y235A + | |
|---|---|---|---|---|---|---|---|---|
| | with Integrin | | with Integrin | | with Integrin | | without | |
| | αvβ6 | | αvβ6 | | αvβ6 | | Integrin αvβ6 | |
| | Mean | | Mean | | Mean | | Mean | |
| CM (%) | LU | SEM | LU | SEM | LU | SEM | LU | SEM |
| 0.05 | 192 | 19.1 | 170 | 14.0 | 387 | 49.6 | 286 | 17.2 |
| 0.016666667 | 293 | 14.3 | 243 | 25.8 | 543 | 54.2 | 337 | 36.0 |
| 0.005555556 | 502 | 44.9 | 341 | 41 | 498 | 36.9 | 316 | 29.9 |
| 0.001851852 | 406 | 44.9 | 322 | 25.3 | 870 | 106.2 | 291 | 18.7 |
| 0.000617284 | 427 | 42.2 | 279 | 4.8 | 625 | 66.3 | 279 | 7.5 |
| 0.000205761 | 266 | 24.7 | 182 | 14.3 | 289 | 15.7 | 219 | 4.4 |
| 0.000068587 | 144 | 14 | 157 | 21.3 | 197 | 7 | 212 | 15.7 |
| 0.000022862 | 123 | 12.1 | 111 | 10.1 | 192 | 7.8 | 189 | 11.1 |
| 0.000007621 | 120 | 5.9 | 106 | 5.5 | 208 | 5.2 | 202 | 4.7 |
| 0.000002540 | 108 | 4.7 | 100 | 4.5 | 208 | 16.2 | 191 | 19.6 |
| 8.46754e−07 | 114 | 9.2 | 114 | 8.7 | 211 | 14.4 | 218 | 13.5 |
| 2.82251e−07 | 112 | 3.8 | 103 | 3.1 | 215 | 10.6 | 204 | 11 |
| | | | | | | | | 316 |

In conclusion, processed anti-hCD63-TGFβ1 SLC fusion proteins can be chemically (via heat or acid treatment) or mechanically activated. Activated anti-hCD63-TGFβ1 SLC fusion proteins induce Smad2/3 signaling. In this study, anti-hCD63 was used for proof-of-concept. If anti-hCD63 is swapped out for alternative targeting arms, then TGFβ1 SLC may be delivered to target protein-expressing cells where it can be activated locally (by endogenous mechanisms) to induce Smad2/3 signaling in diseases exacerbated by decreased TGFβ signaling.

### Example 3. Anti-hCD63-TGFβ SLC Fusion Protein Purification

Heavy and light chains of anti-hCD63-TGFβ SLC constructs were cloned into a double promoter pCHO vector. A stable FreedomCHO-S cell line was generated after transfecting the cells with the anti-hCD63-TGFβ pCHO vector. After completing the first phase of selection (Puromycin 10 μg/mL), cryo stocks of the stable cell lines were made.

To express anti-hCD63-TGFβ, one cryovial of the stable cell line was added to 75 mL CD-FortiCHO media supplemented with 8 mM L-Glutamine. Once the culture reached high enough density, 1 liter CD-Forti was seeded with anti-hCD63-TGFβ expressing cells at 3×10⁵ cells/mL. The growing culture was fed with 500 g/liter glucose on days 3, 5, and 7. The culture was harvested on day 10 by centrifuging the culture at 20,000 rpm for 20 min. 1× protease inhibitor cocktail was added to the supernatant and filter-sterilized using a 0.2 μm filter.

One liter CM was loaded to the Protein A column. 20 mM sodium phosphate, 0.15M NaCl, pH 7.2 was used as a binding buffer. Two different purification conditions were tested: low pH elution (0.1 M sodium citrate, pH 3.0) and salt elution (3.5M MgCl₂). Low pH eluted fractions were immediately neutralized with 1M Tris, pH 9.0. After each purification cycle, the column was cleaned with 1M NaOH.

The purified fractions were pooled and dialyzed immediately in 1×PBS (phosphate-buffered saline) or 1×TBS (tris-buffered saline) overnight at 4° C. After dialysis, the purified samples were filter-sterilized using a 0.45 μm polyethersulfone (PES) filter. The concentration of purified samples was determined using the bicinchoninic acid (BCA) kit. To test if there was any aggregation, 500 μg purified anti-hCD63-TGFβ from each elution was loaded to the Superdex 200 SEC.

The activity of the purified samples was compared with the one of RD latent TGFβ1 as described (see, e.g., Example 4).

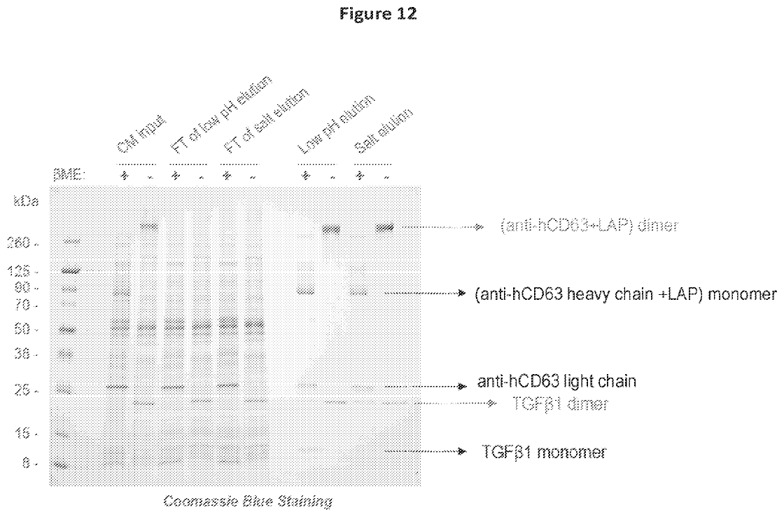

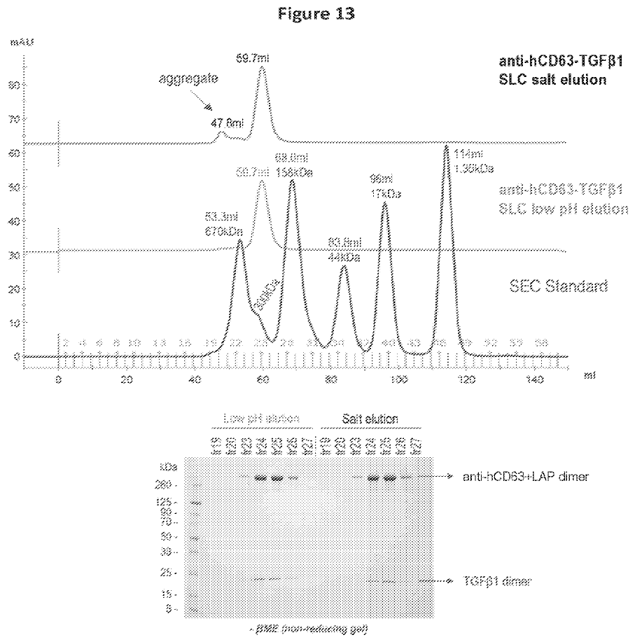

FIG. 12 shows the SDS-PAGE analysis of purified antiCD63-TGFβ1. Anti-hCD63-TGFβ1 expressed in FreedomCHO cells was completely processed. Under non-reducing conditions, there were only two bands: one was the antiCD63-LAP dimer (above 260 kDa) and the other one was the TGFβ1 dimer (around 24 kDa). The corresponding SEC chromatograms and accompanying SDS-PAGE analysis of eluates are shown in FIG. 13 (top and bottom panels, respectively). A small aggregation peak was observed in the MgCl₂ elution indicating that a low pH elution may be more suitable for purification of the fusion construct. In particular, there was a minor aggregation in the anti-CD63-TGFβ1 salt elution sample and no aggregation in the anti-CD63-TGFβ1 low pH elution sample.

### Example 4. Anti-hCD63-TGFβ1 SLC Fusion Protein Specifically Binds to hCD63-Expressing Cells

The objective of this Example was to test whether the anti-hCD63-TGFβ1 SLC fusion protein expressed in ExpiCHO cells can specifically bind to CD63-expressing cells. CD63 is localized on the cell surface and binding to the cell surface target is essential for the activation of anti-hCD63-TGFβ1 SLC fusion proteins. The binding of anti-hCD63-TGFβ1 SLC to cell surface target(s) via the antibody (e.g., anti-hCD63 antibody) and binding to integrin via LAP creates a bidirectional pulling force. This force opens up the LAP domain and releases mature TGFβ1. Mature TGFβ1 binds TGFβR1 and TGFβR2 to induce Smad2/3 signaling.

To test whether the anti-hCD63-TGFβ1 SLC fusion protein specifically binds CD63 expressing cells, HEK293 cells expressing a human CD63.Y235A non-internalizing mutant and human CD63 knockout (KO) Hek293 cells were treated with anti-hCD63-TGFβ1. The CD63.Y235A non-internalizing mutant remained on the cell surface for an extended timeframe, whereas CD63 WT (wild-type) was internalized faster by comparison. Therefore, the CD63.Y235A mutant expressing Hek293 cells were chosen to anchor anti-hCD63-TGFβ1 SLC instead of hCD63 WT expressing Hek293 cells. In this assay:

1. 30,000 Hek293 cells expressing hCD63.Y235A or hCD63 KO Hek293 cells were added to a 24-well black tissue culture (TC) treated plate in complete Dulbecco's Modified Eagle Medium (DMEM media) supplemented with 10% fetal bovine serum (FBS), 2 mM L-Glutamine, 100 units/mL P/S (Penicillin/Streptomycin). Cells were maintained at 37° C., 5% CO₂.

2. On the following day, the complete media (CM) was removed and subsequently replaced with starvation media comprising serum-free DMEM media supplemented with 2 mM L-Glutamine and 100 units/mL P/S for a 1 hr starvation period.

3. During the starvation, purified anti-hCD63-TGFβ1 was diluted in the serum-free DMEM media to a final concentration of 10 nM. After the 1 hr starvation period, the starvation media was removed, the diluted anti-hCD63-TGFβ1 CM was added to the cells, and the cells were incubated with 10 nM anti-hCD63-TGFβ1 for 1 hr.

4. After the anti-hCD63-TGFβ1 treatment, the cells were washed with 1×PBS (one time) and then fixed with 4% PFA for 15 min at room temperature (RT).

5. The cells were permeabilized and blocked in 10% BSA, 0.3% TritonX-100 buffer for 45 minutes at RT.

6. Then, the cells were incubated in human CD63 antibody diluted in antibody dilution buffer to a final concentration of 5 μg/mL overnight at 4° C. On the following day, human LAP-TGFβ1 antibody conjugated to Alexa488 and anti-rat secondary antibody conjugated to Alexa647 were diluted in antibody dilution buffer to a final concentration of 5 μg/mL. The human CD63 antibody was then removed from the cells, the cells were washed 3 times with 1×PBS, and the diluted antibody mixture was added to the cells and incubated for 3 hr at room temperature (RT) during which time the plate was covered with aluminum foil to maintain darkness.

7. The cells were then washed three times with 1×PBS and then covered with an anti-fade reagent containing 4′,6-diamidino-2-phenylindole (DAPI). Confocal imaging was then performed to visualize the cells.

Both hCD63.Y235A overexpressing Hek293 cells and hCD63 KO Hek293 cells were treated with CM of anti-hCD63-TGFβ1 transfected ExpiCHO cells. After staining the cells with CD63 antibody (Alexa647), LAP(TGFβ1)-Alexa488 antibody, and nuclear stain (DAPI), confocal imaging was performed.

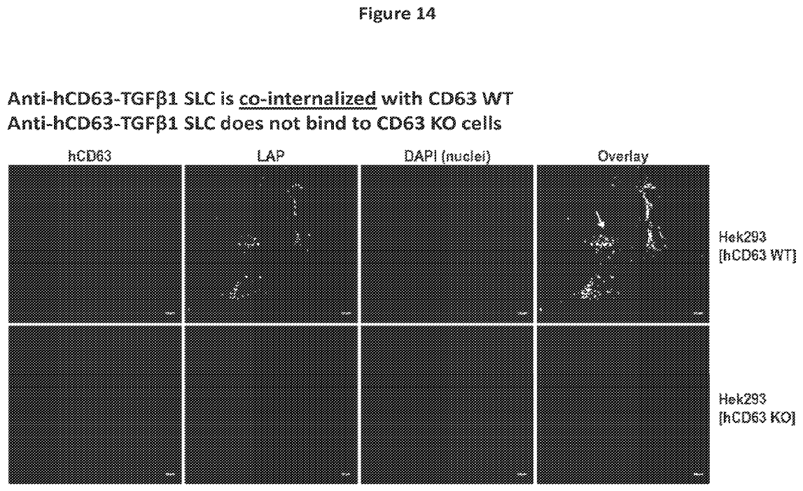

In FIG. 14, ‘red’ (left most panel) represents CD63-Alexa647 antibody binding, ‘green’ (left middle panel) represents LAP(TGFβ1)-Alexa488 antibody binding, and ‘blue’ (right middle panel) represents the nuclear stain. An overlay of the three colors (red, green, and blue) is also depicted (right most panel). As shown in FIG. 14, LAP(TGFβ1)-Alexa488 antibody only bound to anti-hCD63-TGFβ1 treated Hek293 cells overexpressing hCD63.Y235A, but not anti-hCD63-TGFβ1 treated hCD63 KO Hek293 cells. LAP(TGFβ1)-Alexa488 antibody staining overlapped with CD63 staining showing that LAP(TGFβ1)-Alexa488 antibody bound to LAP domain of anti-hCD63-TGFβ1 bound to hCD63.Y235A expressing cells.

These data show that anti-hCD63-TGFβ1 binds specifically to hCD63-expressing cells, which was visualized by LAP(TGFβ1)-Alexa488 antibody binding.

### Example 5. Determination of the Internalization Rate of Anti-hCD63-TGFβ1 Using Confocal Imaging and Flow Cytometry Approaches

The present Example is designed to determine the internalization rate of anti-hCD63-TGFβ1 using confocal and flow cytometry approaches. For confocal experiments, hCD63 wild type (WT), hCD63.Y235A non-internalizing mutant and non-transfected cells are placed in a 24-well plate in the complete media. After 2 days of incubation in complete media, the cells are starved for 1 hr and treated with various concentrations of Zenon pHrodo iFL green (Thermoscientific, catalog number Z25611) labeled anti-hCD63-TGFβ1. Then, the treated cells are fixed at different time points. The fixed cells are washed with PBS and covered with the anti-fade reagent containing DAPI. The confocal images are acquired to determine the internalization of anti-hCD63-TGFβ1. Zenon pHrodo dye labels the Fc portion of the antibody, and as such, does not affect the binding properties of the antibody. Zenon pHrodo-labeled antibody increases fluorescence only in the acidic environment, e.g., within the late endosome or lysosome. Therefore, levels of detected fluorescence are completely correlated with internalization.

For flow cytometry experiments, hCD63 wild type (WT), hCD63.Y235A non-internalizing mutant and non-transfected cells are seeded at 100,000 cells/well to a 96-well plate in Flow Cytometry Staining Buffer (FSB). The cells are maintained on ice. Fc blocking is performed for 15 min on ice. After blocking, the cells are treated with various concentrations of anti-hCD63-TGFβ1, and the plate is returned to the incubator for 1 hour at 37° C./5% CO₂. Cells are then washed with FSB buffer and stained with Alexa488 conjugated FAB secondary antibody for 30 min on ice. The cells are washed again, fixed, and divided into two equal parts. Half of the cells are resuspended in FSB and the other half in Alexa488 quencher. The cells are analyzed in the FACS instrument on the following day. Cells resuspended in FSB buffer (unquenched) represent the total staining (both intracellular and membrane). The 488 quencher blocks the 488 fluorescence on the membrane, so only the intracellular fluorescence is detected. The difference between unquenched and quenched cells shows the internalization rate.

### Example 6. In Vitro Chemical and Heat Activation of Processed Anti-hCD63-TGFβ1 SLC Induces Smad2/3 in NBL7 Cells

The objective of this Example was to show that anti-hCD63-TGFβ1 SLC fusion protein expressed in ExpiCHO cells can be activated in vitro to induce Smad2/3 signaling in NBL7 cells (American Mink lung epithelial cells). Both chemical and mechanical activation approaches were tested to activate anti-hCD63-TGFβ1 SLC fusions.

In chemical activation, purified anti-hCD63-TGFβ1 diluted in 1×PBS containing 50% glycerol was treated with heat to dissociate the LAP domain and release mature TGFβ1. For heat treatment, the samples were incubated at 80° C. for 10 minutes. Non-activated samples were kept as a control in the chemical activation assay.

To test the activity of non-activated and heat-activated a dose-response signaling assay was performed in NBL7 cells carrying Smad2/3 responsive luciferase reporter vector. In this assay:

1. Day 1: 10,000 NBL7-Smadluc cl.6 cells (cells carrying Smad2/3 responsive luciferase vector) were plated in Poly-D-Lysine (PDL) coated 96-well luciferase plates in 100 μL/well complete DMEM media supplemented with 10% FBS, 2 mM L-Glutamine, 100 units/mL P/S, and 500 μg/mL G418. Cells were maintained at 37° C., 5% CO₂.

2. Day 2: 100 μL media was removed from each well and fresh 100 μL serum-free media (DMEM+2 mM L-Glutamine+100 units/mL P/S+500 μg/mL G418) supplemented with 0.1% BSA was added to each well.

3. Day 3: Non-activated and heat-activated samples were titrated in serum free media (DMEM+2 mM L-Glutamine+100 units/mL P/S) supplemented with 0.1% BSA. 100 μL media was removed from each well and 100 μL of titration sample was added to each well.

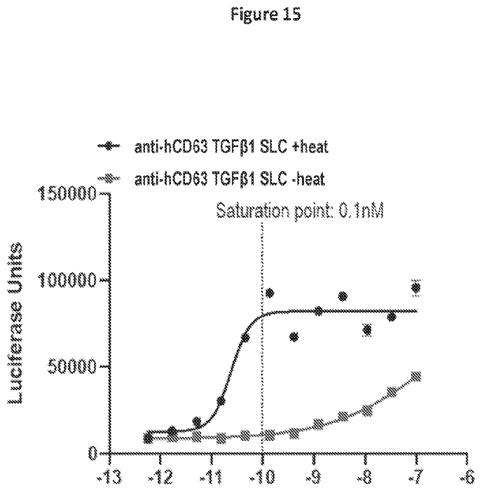

4. After 16 hrs, the plate was incubated at RT for 10 min. Then, 100 L/well BrightGlo luciferase reagent was added and incubated at RT for 5 min. After the incubation, luminescence measurement was taken, and the results were analyzed with Prism software. Heat activation data of purified antiCD63-TGFβ1 in NBL7 cells carrying the Smad2/3 reporter vector are displayed in FIG. 15.

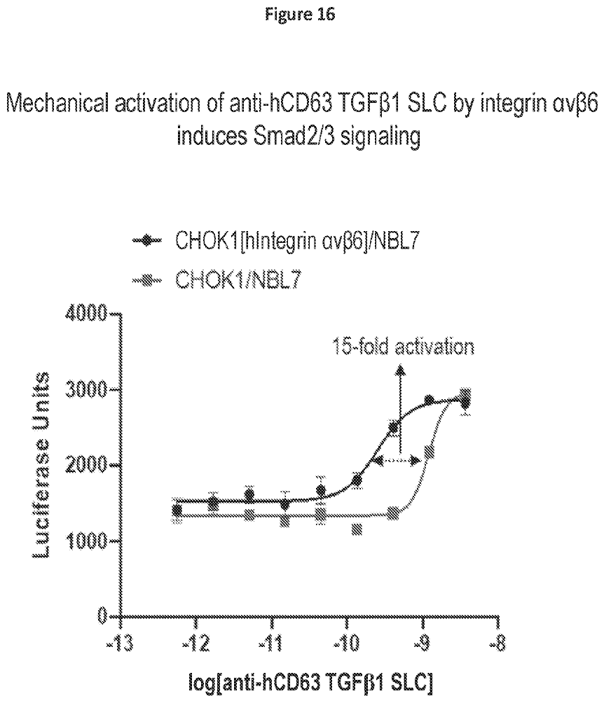

For mechanical activation, hIntegrin αvβ6-expressing CHOK1 cells and NBL7-Smadluc cl.6 cells reporter cells were co-cultured in a PDL-coated 96-well luciferase plate (3,000 cells/well) in complete media (1:1 mix of Ham's F12 media:DMEM media supplemented with 10% FBS+2 mM L-Glutamine+100 units/mL P/S) for 48 hours. On the second day, 50p Ni beads (washed two times with PBS) were incubated with 20 μl hCD63 EC2 loop-mmh (7.76 mg/ml)+10 μL processed antiCD63-TGFβ1 (2.7 mg/ml) resuspended in 500 μl PBS for 3 hours at 4° C. After incubation, the beads were washed two times with PBS and then resuspended in 1 ml with starvation media (1:1 mix of Ham's F12 media:DMEM) supplemented with 2 mM L-Glutamine+100 units/mL P/S+0.1% BSA). Three-fold dilutions of the starvation media were performed and the titrated samples were added to the cells. A magnet was placed under the plate and the plate and magnet were incubated overnight at 37° C. After 20 hours of incubation, 100 μL/well BrightGlo luciferase reagent was added to the samples and incubated at room temperature (RT) for 5 min. After incubation, luminescence was measured, and the results were analyzed with Prism software. FIG. 16 confirms mechanical activation of antiCD63-TGFβ1 SLC by integrin αvβ6 induces Smad2/3 signaling in NBL7 cells.

### Example 7. Cell-Based Assay Testing REGN14660 Fused to SLC TGFβ1 and TGFβ2

GAGA Luc reporter cell line expressing Luciferase under the control of a TGFβ SMAD3-dependent promoter were maintained in DMEM media (ThermoFisherScientific, 10313021) supplemented with 10% FBS (ThermoFisherScientific, A3840001), 2 mM L-Glutamine (ThermoFisherScientific, 25030081), 100 units/mL P/S (ThermoFisherScientific, 15140122), and 500 mg/mL G418 (ThermoFisherScientific, 10131035).

The day of the experiment, cells were seeded in 96-well plates coated with poly-D-Lysine (50 g/ml ThermoFisherScientific, A3890401) at 90,000 cells/cm². Once attached to the surface (>4 h), media was replaced with DMEM containing 0.1% BSA (Sigma A9576) 2 mM L-Glutamine, 100 units/mL P/S until the day after. Progressive dilutions of each REGN14660-SLC antibody (from 625 pg/μl to 4.8 pg/μl) in DMEM containing 0.1% BSA were prepared in duplicate. One of the duplicates was heated at 80° C. for ten minutes (heat activation, HA) to release TGFβ from the LAP. Both dilutions were incubated on top of the reporter cells for >16 h.

The day after the luciferase assay was performed, the plates were incubated at room temperature (RT) for 10 min. Then, 100 mL/well BrightGlo luciferase reagent (Promega, E2620) was added and incubated at RT for 5 minutes then luminescence measurements were taken. Reads were taken using 2030 Multilabel reader VictorX5 (PerkinElmer).

In another set of experiments, REGN14660-TGFβ1 was tested for fibronectin (FN) binding and integrin-dependent TGFβ activation from the SLC. In these experiments, 96-well plates were coated with 1 μg/cm² of either human recombinant FN (containing extra domain B (EDB); R&D system 4305-FBN) or human plasma FN (not containing EDB; Sigma F2006) in PBS for 30 min. Wells were then washed with PBS and dried for 45 min. All the wells were blocked with DMEM/BSA 0.1% for 30 minutes then dilutions of REGN14660-TGFβ1 in DMEM/BSA 0.1% were added to the wells and incubated for 1 h at 37° C. The antibody was removed and wells were washed three times with DMEM/BSA 0.1% (10 min each). Then 90,000 cells/cm² of CagaLux together with 90,000 cells/cm² of CHO cells or αvβ6-expressing CHO cells were added in DMEM/BSA 0.1% in each well.

A luciferase assay was performed the day after as described above.

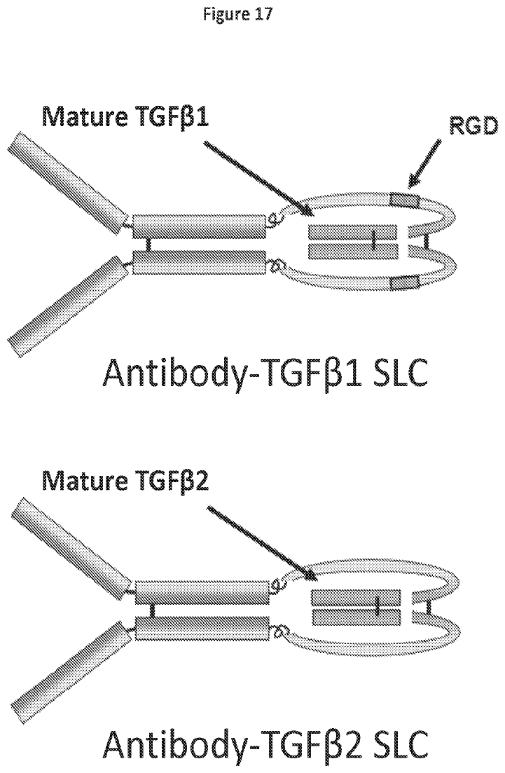

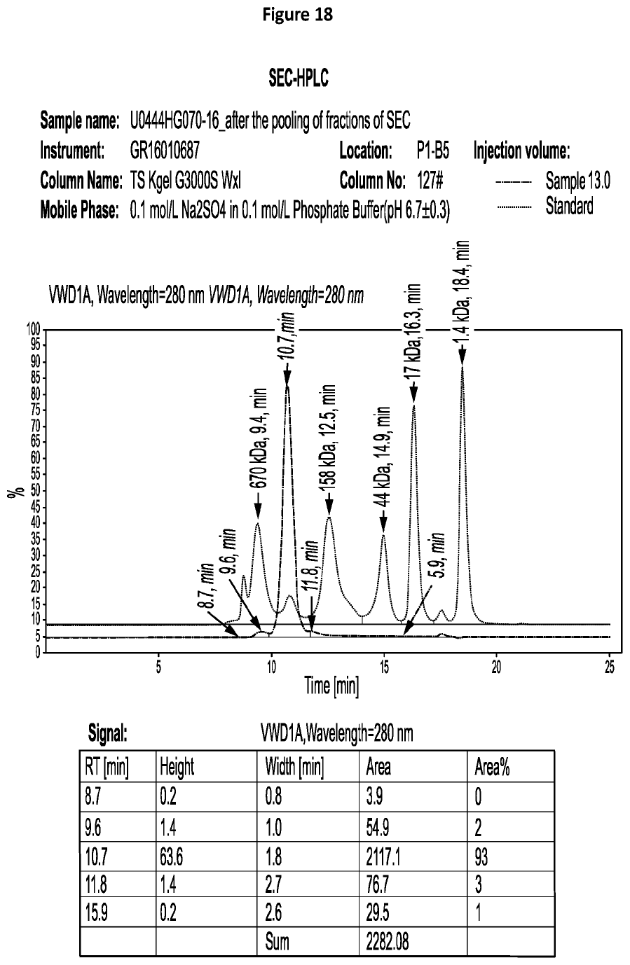

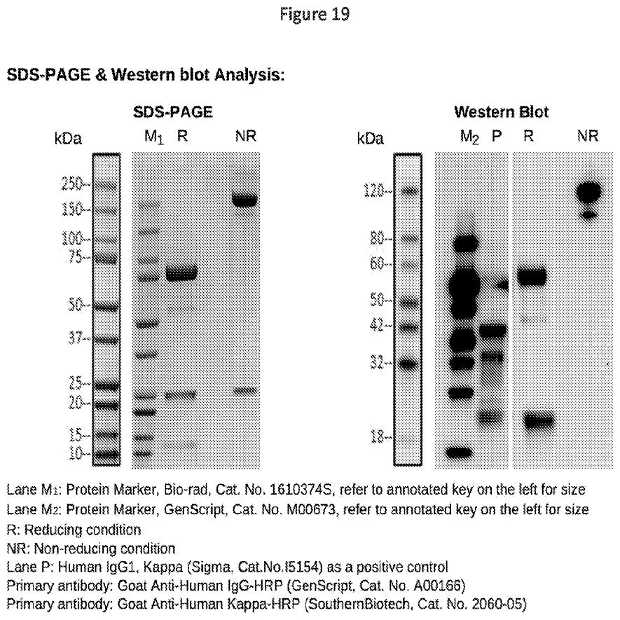

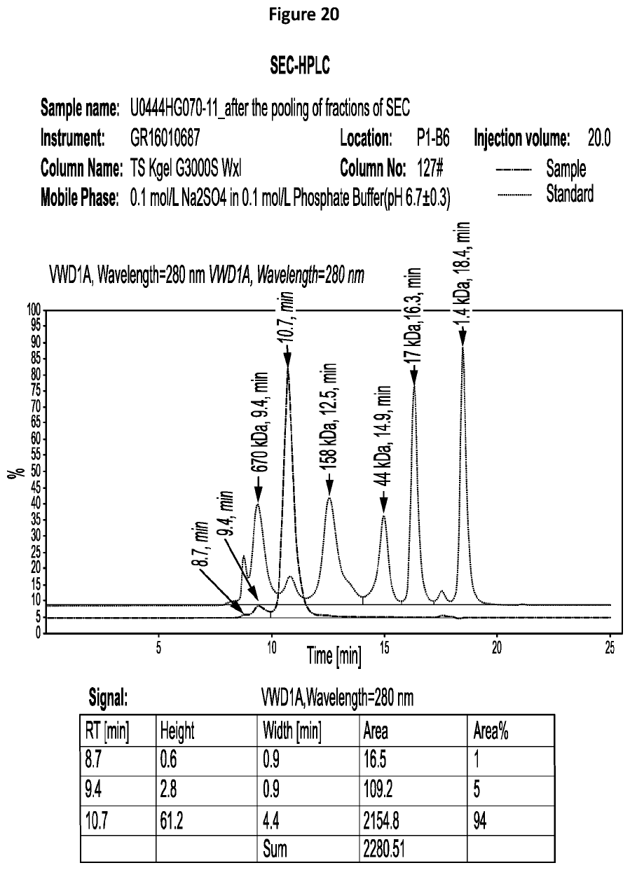

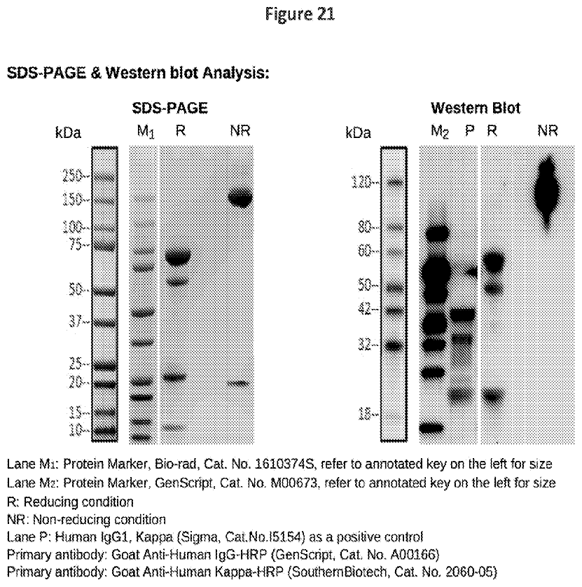

REGN14660-SLC (REGN14660-TGFβ1 and REGN14660-TGFβ2) were manufactured by GenScript (see, e.g., FIGS. 17-21).

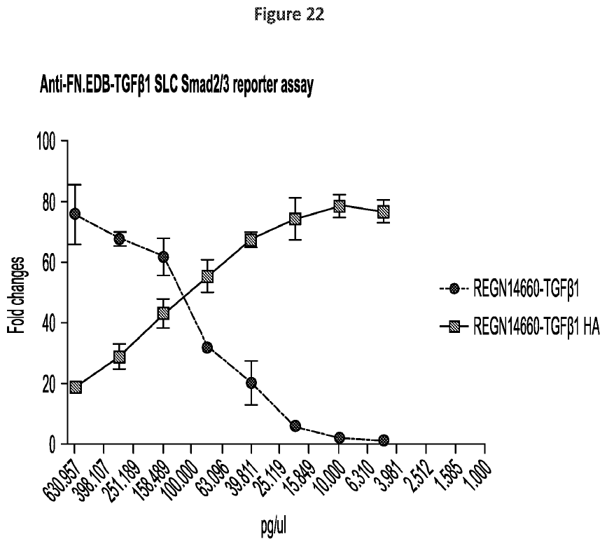

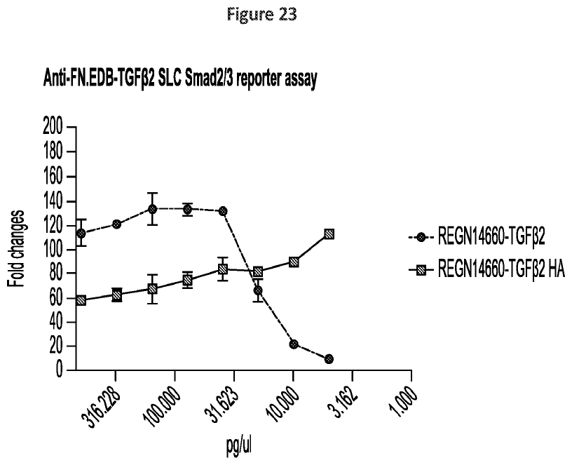

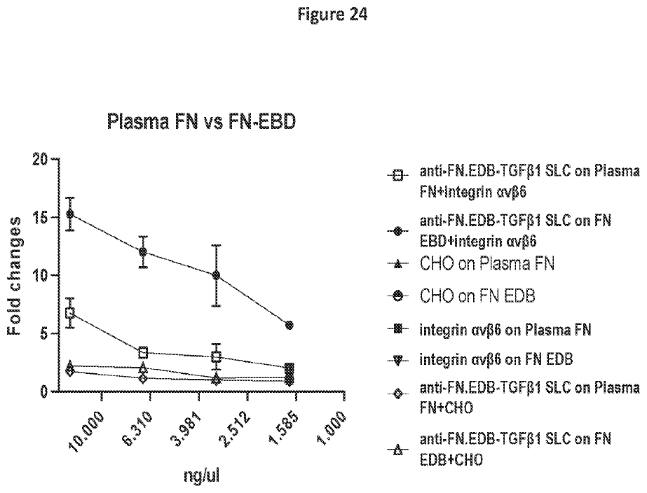

The present Example showed that REGN14660-TGFβ1 and REGN14660-TGFβ2 fusions retained TGFβ in a latent form. REGN14660-TGFβ1 was bound to extra domain B of fibronectin (EDB-FN; extracellular matrix FN) with higher affinity compared to plasma FN (soluble FN). TGFβ1 could be activated/released from LAP by integrins in vitro when REGN14660-TGFβ1 was incubated with αvβ6-expressing CHO cells (FIGS. 22-24).

### Example 8. Immunostaining of Mouse Tissues

Aortas were isolated from P14 wild-type (WT) and Fibrillin 1 knockout (Fbn1KO) mice (MAID 9419), fixed in 4% paraformaldehyde and processed for paraffin embedding. 4 μm tissue sections were cut, de-paraffinized, and subject to antigen retrieval by treating tissue sections with 6M guanidine hydrochloric acid (HCl)-20 mM tris(hydroxymethyl)aminomethane (Tris)-50 mM Dithiothreitol (DTT), pH 8.0, for 15 minutes. After washing with 20 mM Tris pH 8.0, the slides are treated with 100 mM iodoacetamide in the dark for 15 minutes, washed, blocked in Tris-buffered saline (TBS) 1× containing 3% bovine serum albumin (BSA) and 10% fetal bovine serum (FBS) for 1 hour and stained with anti-EDB-FN antibody (REGN14660) at 10 μg/ml or with anti-cluster of differentiation 31 (anti-CD31; Invitrogen clone SP38) over-night in TBS 1× containing 3% BSA. The next day, slides were stained with anti-human or anti-rabbit secondary antibody Alexa 594 (ThermoFisher) diluted 1:400 in TBS 1× containing 3% BSA for 1 hour. Autofluorescence was quenched with RedyProbes™ (Invitrogen) and stained with 4′,6-diamidino-2-phenylindole (DAPI; Nucblue Invitrogen) following the manufacturer's instructions. Images were captured using Zeiss Axiobserver connected to Axiocam 506 color. Alternatively, tissue sections were stained with the same antibody by using an automated staining instrument (BOND RX Fully Automated Research Stainer; Leica BioSystems) and following the manufacturer's instructions.

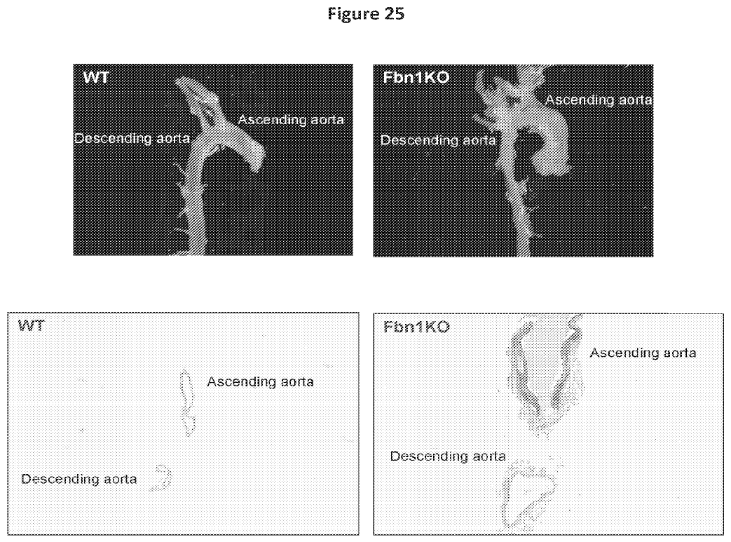

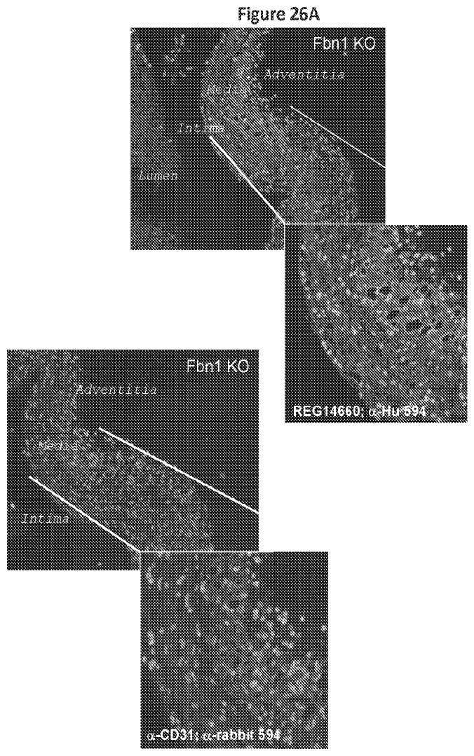

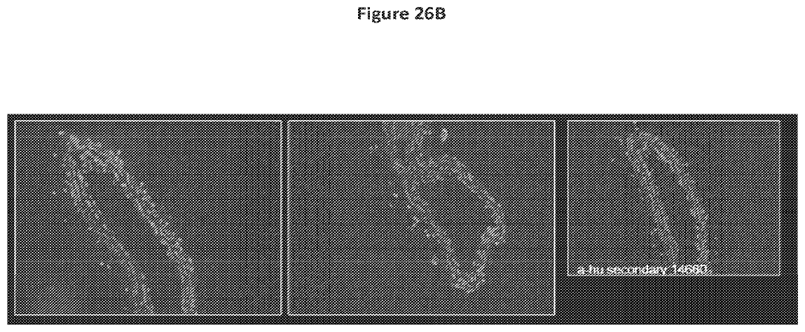

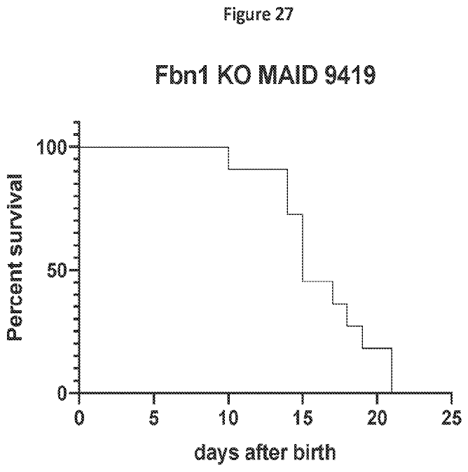

The results showed that EDB-FN was detected by the REG14660 antibody in Fbn1KO aortic lesions from P15 mice but not in normal aorta (FIG. 26B). EDB-FN was associated with abnormal microvessels formed within the aortic wall of Fbn1KO mice (FIGS. 25-26). A survival curve plot showing Fibrillin 1 knockout (Fbn1KO) mice (MAID 9419) have median survival of P15 (after birth) FIG. 27. None of the Fbn1KO survive beyond about day 21.

### Example 9. Single Dose PK Analysis of Mice Injected with Antibody-TGFβ1 SLC Fusions

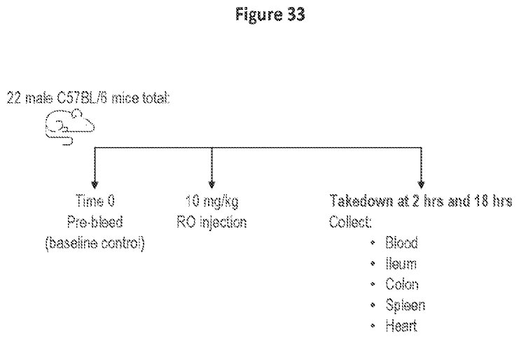

The objective of this Example was to ascertain whether antibody-TGFβ1 SLC fusions could be delivered to specific tissues and subsequently activate downstream Smad2/3 signaling. C57BL/6 male mice were administered anti-mEpcam-TGFβ1 SLC, anti-mClec9a (REGN6550)-TGFβ1 SLC, and anti-mIgG1 isotype control (REGN2390)-TGFβ1 SLC (10 mg/kg retroorbital [RO] injection). Following this administration, serum and tissue samples were collected at two intervals: 2 hours and 18 hours post-injection (FIG. 33).

Serum samples were analyzed to determine the circulating levels of antibody-TGFβ1 SLC fusions and whether the antibody-TGFβ1 SLC fusions stayed latent in circulation.

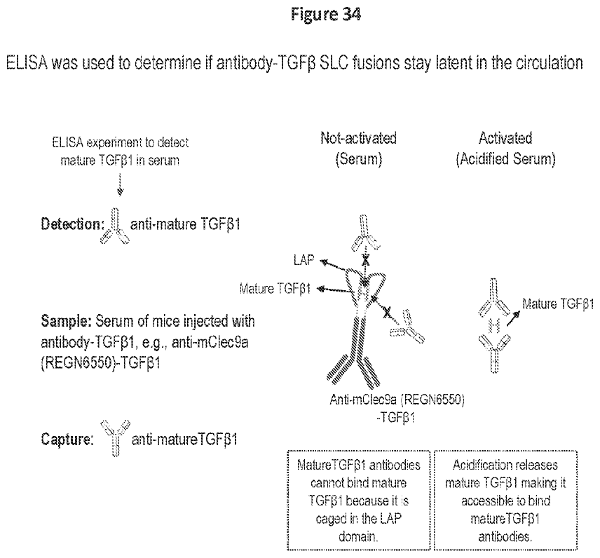

In this experiment:
1. The R&D hTGFβ1 DuoSet ELISA kit (DY240-05) was used to determine the mature TGFβ1 levels in the serum samples (FIG. 34). Briefly, 96-well plates were coated with the capture antibody and incubated overnight at 4° C. The following day, a serial dilution of acid-activated or non-activated serum samples were prepared and added to the plate. After a 2-hour incubation period at room temperature, a secondary antibody was introduced, followed by an incubation with streptavidin-HRP. In the final step, 3,3′,5,5′-Tetramethylbenzidine (TMB) substrate was added and the reaction was halted by 2N H₂SO₄ after a 20-minute incubation. The optical density (OD) 450 nm reading was taken immediately after stopping the reaction.
2. To measure the circulating levels of the antibody-TGFβ1 SLC fusions, an ELISA assay was used. Briefly:
   a. 96-well plates were coated with the 1 μg/ml capture antigen overnight at 4° C. For example, to detect anti-mEpcam-TGFβ1 SLC, the plate was coated with mEpcam.
   b. On the second day, a serial dilution of the non-acid-activated serum samples was prepared and added to the plates. A purified antibody-TGFβ1 SLC fusion was used as a standard.
   c. Samples were incubated at room temperature (RT) for 2 hours. Next, plates were washed, secondary antibody horseradish peroxidase (HRP)-conjugated anti-mIgG antibody (Jackson, 115-035-071) was added to the plates, and the plates were incubated for 2 hours at RT.
   d. Following the secondary antibody incubation, the plates were washed again. Then, TMB substrate was added (R&D, DY999B) and incubated at RT for 20 minutes.
   e. The reaction was stopped with 2N H₂SO₄, and the OD 450 nM reading was measured immediately.

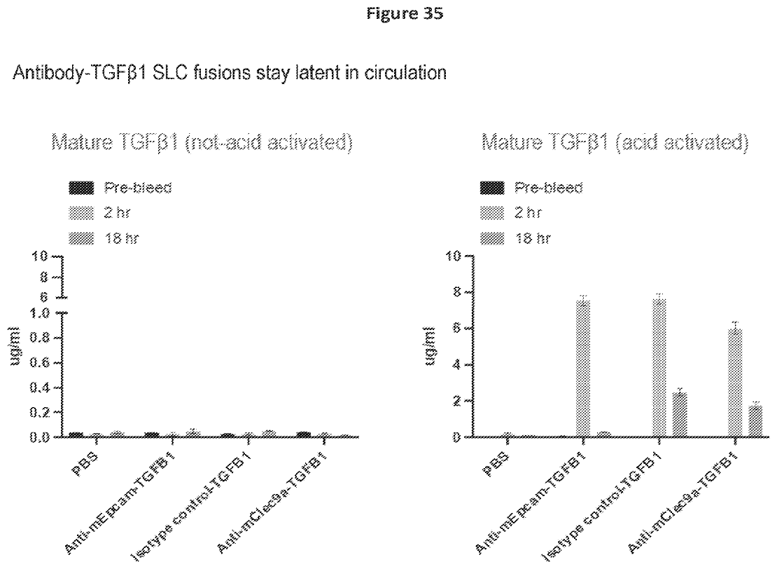

As depicted in Table 4, Table 5, and FIG. 35, no mature TGFβ1 was detected in the non-acid-activated serum samples. Mature TGFβ1 was only identified post-acid activation, which removes the LAP domain and enables mature TGFβ1 to bind to the capture/detection antibodies. The absence of mature TGFβ1 in the non-acid-activated serum samples suggests that antibody-TGF1 SLCs remain latent in circulation. Comparable levels of mature TGFβ1 were detected post-acid activation across all treatment groups. The highest level of mature TGFβ1 was detected 2 hours post-injection, with a significant decrease noted after 18 hours, indicating rapid clearance from circulation.

TABLE 4
Mature TGFβ1 levels in circulation after acid activation (μg/ml)

| | Pre-bleed | | | | | |
|---|---|---|---|---|---|---|
| PBS | 0.05425635 | 0.06929833 | 0.06725416 | 0.04357474 | — | — |
| Anti- | 0.10143524 | 0.08016844 | 0.05219502 | 0.06213451 | 0.07881246 | 0.05185119 |
| mEpcam- | | | | | | |
| TGFβ1 | | | | | | |
| Isotype | 0.0720208 | 0.04392061 | 0.07881246 | 0.03872231 | 0.03280121 | 0.03210228 |
| control- | | | | | | |
| TGFβ1 | | | | | | |
| Anti- | 0.03698431 | 0.04944224 | 0.06179271 | 0.03767984 | 0.03559187 | 0.03698431 |
| mClec9a | | | | | | |
| (REGN6550) | | | | | | |
| -TGFβ1 | | | | | | |
| | 2 hr | | | | | |
| PBS | 0.17973207 | 0.21733252 | — | — | — | — |
| Anti- | 7.1207778 | 7.41853625 | 8.07891755 | | | |
| mEpcam- | | | | | | |
| TGFβ1 | | | | | | |
| Isotype | 8.07320123 | 7.14691715 | 7.73337133 | — | — | — |
| control- | | | | | | |
| TGFβ1 | | | | | | |
| Anti- | 5.56610077 | 6.68612863 | 5.73743178 | — | — | — |
| mClec9a | | | | | | |
| (REGN6550) | | | | | | |
| -TGFβ1 | | | | | | |
| | 18 hr | | | | | |
| PBS | 0.14457493 | 0.14856629 | | — | — | — |
| Anti- | 0.31883929 | 0.26819658 | 0.32894677 | — | — | — |
| mEpcam- | | | | | | |
| TGFβ1 | | | | | | |
| Isotype | 2.61890271 | 2.77916833 | 2.07149033 | — | — | — |
| control- | | | | | | |
| TGFβ1 | | | | | | |
| Anti- | 1.8726939 | 1.35931682 | 1.99271583 | — | — | — |
| mClec9a | | | | | | |
| (REGN6550) | | | | | | |
| -TGFβ1 | | | | | | |

TABLE 5
Mature TGFβ1 levels in circulation (μg/ml)

| | Pre-bleed | | | | | |
|---|---|---|---|---|---|---|
| PBS | 0.04405823 | 0.03329981 | 0.02612888 | 0.03707045 | — | — |
| Anti- | 0.02757496 | 0.03800804 | 0.04080959 | 0.02901474 | 0.02516113 | 0.0491243 |
| mEpcam- | | | | | | |
| TGFβ1 | | | | | | |
| Isotype | 0.02419026 | 0.01829001 | 0.02076534 | 0.01222174 | 0.03753949 | 0.03471774 |
| control- | | | | | | |
| TGFβ1 | | | | | | |
| Anti- | 0.04313212 | 0.03847612 | 0.03566036 | 0.05232575 | 0.04682713 | 0.03518931 |
| mClec9a | | | | | | |
| (REGN6550) | | | | | | |
| -TGFβ1 | | | | | | |
| | 2 hr | | | | | |
| PBS | 0.03092528 | 0.02223854 | — | — | — | — |
| Anti- | 0.02612888 | 0.04820651 | 0.00587241 | — | — | — |
| mEpcam- | | | | | | |
| TGFβ1 | | | | | | |
| Isotype | 0.02805557 | 0.0403438 | 0.01477537 | — | — | — |
| control- | | | | | | |
| TGFβ1 | | | | | | |
| Anti- | 0.04313212 | 0.02321612 | 0.02853549 | — | — | — |
| mClec9a | | | | | | |
| (REGN6550) | | | | | | |
| -TGFβ1 | | | | | | |
| | 18 hr | | | | | |
| PBS | 0.05323748 | 0.03092528 | — | — | — | — |
| Anti- | 0.02419026 | 0.02997128 | 0.08405145 | — | — | — |
| mEpcam- | | | | | | |
| TGFβ1 | | | | | | |
| Isotype | 0.0546027 | 0.04728729 | 0.05596516 | — | — | — |
| control- | | | | | | |
| TGFβ1 | | | | | | |
| Anti- | 0.01375878 | 0.02076534 | 0.01878721 | — | — | — |
| mClec9a | | | | | | |
| (REGN6550) | | | | | | |
| -TGFβ1 | | | | | | |

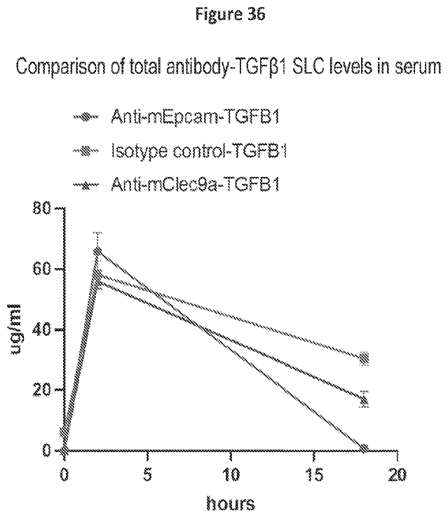

Table 6 and FIG. 36 present the quantity of antibody-TGFβ SLC fusion proteins in circulation. Consistent with FIG. 35, the highest and similar amount of antibody-TGFβ31 SLC fusion was detected 2 hours post-injection. The anti-mEpcam-TGFβ1 SLC was cleared more rapidly than the anti-mClec9a (REGN6550)-TGFβ11 SLC, likely due to the higher abundance of the target of anti-Epcam-TGFβ11 SLC (epithelial cell marker).

TABLE 6
The amount of antibody-TGFβ1 SLC fusions in circulation (μg/ml)

| Hours | | | | | | |
|---|---|---|---|---|---|---|
| | Isotype control-TGFβ1 | | | | | |
| 0 | ND | 3.18500262 | ND | 6.3764391 | 8.70520256 | 6.00113288 |
| 2 | 66.9928843 | 55.784946 | 51.7057046 | — | — | — |
| 18 | 26.2543568 | 33.4445586 | 31.6852949 | — | — | — |
| | Anti-m Clec9a (REGN6550)-TGFβ1 | | | | | |
| 0 | ND | ND | ND | 0.11863649 | 0.11221178 | ND |
| 2 | 51.8378951 | 60.6789419 | 55.389198 | — | — | — |
| 18 | 15.1859041 | 13.9388587 | 22.1010981 | — | — | — |
| | Anti-m Epcam-TGFβ1 | | | | | |
| 0 | ND | ND | ND | ND | ND | ND |
| 2 | 54.2347292 | 67.9061023 | 75.3443743 | — | — | — |
| 18 | 0.66609501 | 0.50316143 | 1.16674354 | — | — | — |

### Example 10. Tissue Delivery of Antibody-TGFβ1 SLC Fusions

The objective of this Example was to ascertain whether antibody-TGFβ1 SLC fusions could be delivered to specific tissues and subsequently activate downstream Smad2/3 signaling. C57BL/6 male mice were administered anti-mEpcam-TGFβ1 SLC, anti-mClec9a (REGN6550)-TGFβ1 SLC, and mIgG1 isotype control (REGN2390)-TGFβ1 SLC (10 mg/kg RO injection). Following this, serum and tissue samples were collected at two intervals: 2 hours and 18 hours post-injection (see, e.g., FIG. 33).

Tissue samples were analyzed to determine the amount of antibody-TGFβ1 SLC delivered to the target tissue and if the delivered antibody-TGFβ1 SLC fusions activated Smad2/3 signaling.

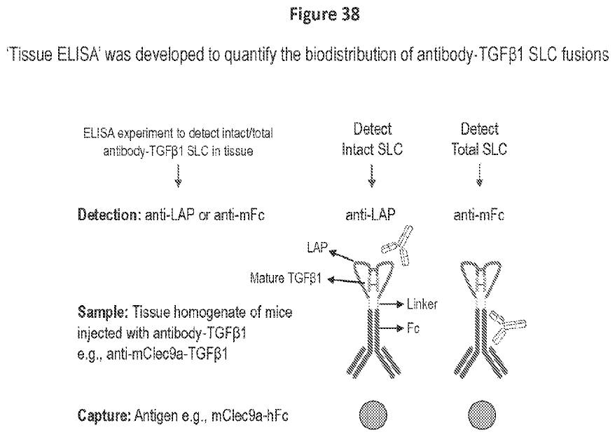

In this experiment:
1. To measure the delivered amount of the antibody-TGFβ1 SLC fusions in the tissue, an ELISA assay was used (FIG. 38). The process is briefly outlined as follows:
   a. Frozen tissue samples were homogenized in tissue extraction buffer (Thermoscientific, 78510) containing 2× protease phosphatase inhibitor (ThermoScientific, 78447).
   b. The total protein concentration of the tissue homogenate was measured using the Bicinchoninic acid (BCA) assay (ThermoScientific, 23227).
   c. 96-well plates were coated with the 1 μg/ml capture antigen overnight at 4° C. For example, to detect anti-mEpcam-TGFβ1 SLC, the plate was coated with mEpcam.
   d. On the second day, tissue homogenates were diluted in the tissue extraction buffer containing 1× protease phosphatase inhibitor to obtain 100 mg/well and 10 mg/well concentrations. Diluted tissue samples were added to the plates. A purified antibody-TGFβ1 SLC fusion was used as a standard.
   e. Samples were incubated at RT for 2 hours. Subsequently, plates were washed, and secondary antibody HRP-conjugated anti-mIgG antibody (Jackson, 115-035-071) was added to the plates and incubated for 2 hours at RT.
   f. After the secondary antibody incubation, the plates were washed again. Then, TMB substrate was added (R&D, DY999B) and incubated at RT for 20 minutes.
   g. The reaction was stopped with 2N H₂SO₄, and the OD 450 nM reading was measured immediately.
2. To determine whether the delivered antibody-TGFβ1 SLC fusions activated Smad2/3 signaling in the target tissue, immunoblots were run with the tissue samples. The process is briefly outlined as follows:
   a. Tissue homogenates were prepared as outlined in step 1.
   b. 10 μg of tissue homogenate was run on 4-20 kDa SDS-PAGE.
   c. Gels were transferred to the polyvinylidene difluoride (PVDF) membrane.
   d. Membranes were blotted with the phospho-Smad2 antibody (Cell Signaling, 138D4) and total-Smad2 antibody (Cell Signaling, D43B4).

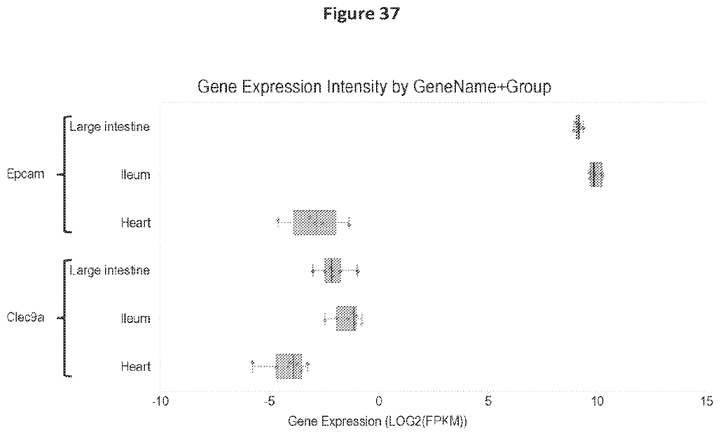

Samples were collected from the ileum, colon, and heart at two intervals: 2 hours and 18 hours post-injection of the antibody-TGFβ1 SLC fusions. Epcam, an epithelial cell marker, has broad expression, while Clec9a, a dendritic cell marker, is expressed in a small population of dendritic cells. The tissue expression levels of Epcam and Clec9a are depicted in FIG. 37.

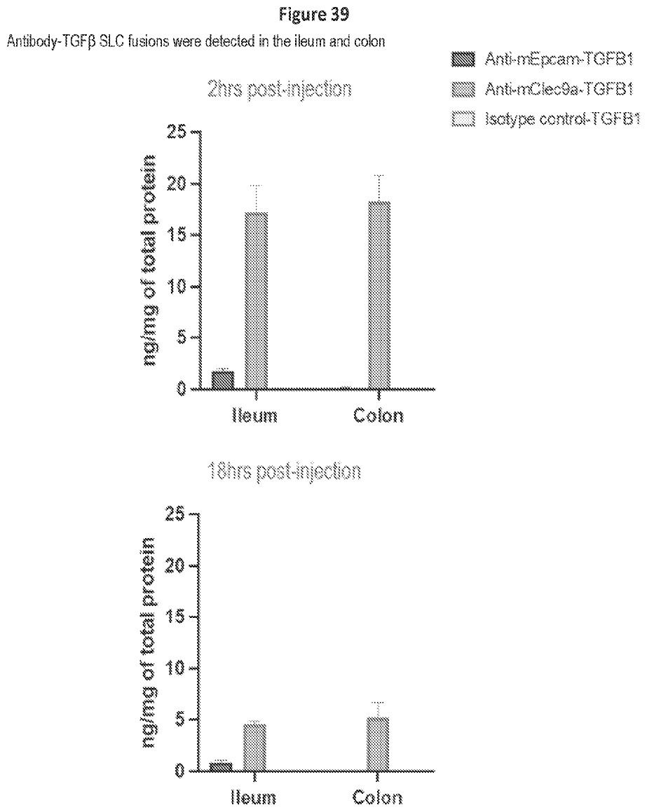

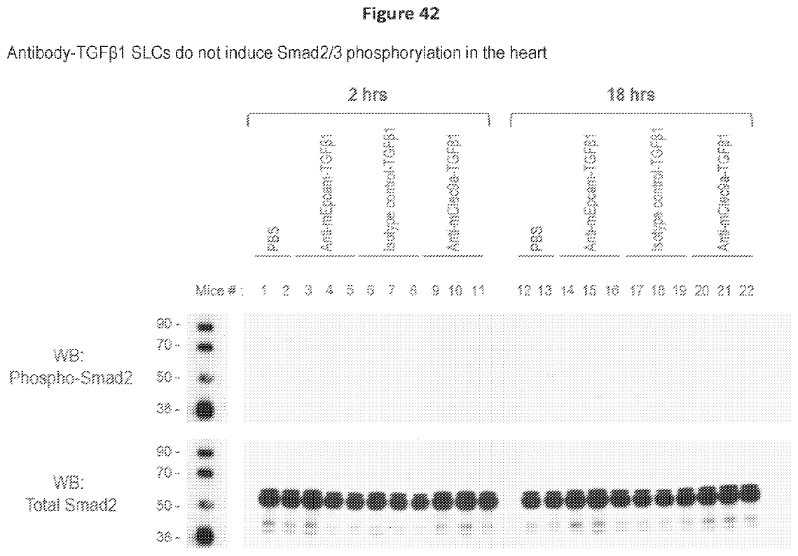

The ileum and colon were selected for sampling as these are two tissues where significant inflammation is observed in inflammatory bowel disease (IBD). Hence, the objective of the present experiment was to deliver the antibody-TGFβ1 SLC fusions to the gut. The heart was chosen as a negative control due to the absence of Epcam and Clec9a expression in the heart. FIG. 39 demonstrates that the anti-mEpcam-TGFβ1 SLC and the anti-mClec9a (REGN6550)-TGFβ1 SLC were delivered to the ileum and colon. No isotype control-TGFβ1 SLC was detected in these tissues. Furthermore, none of the tested antibody-TGFβ1 SLC fusions were detected in the heart (FIG. 42).

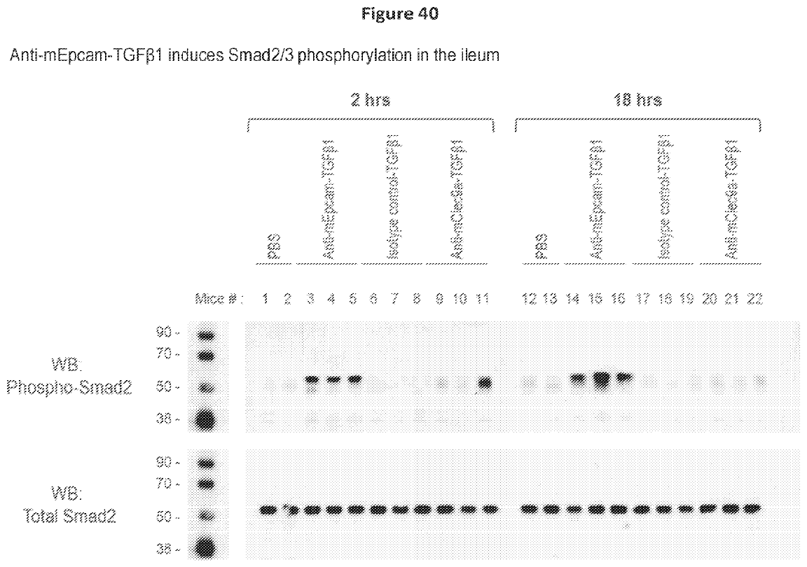

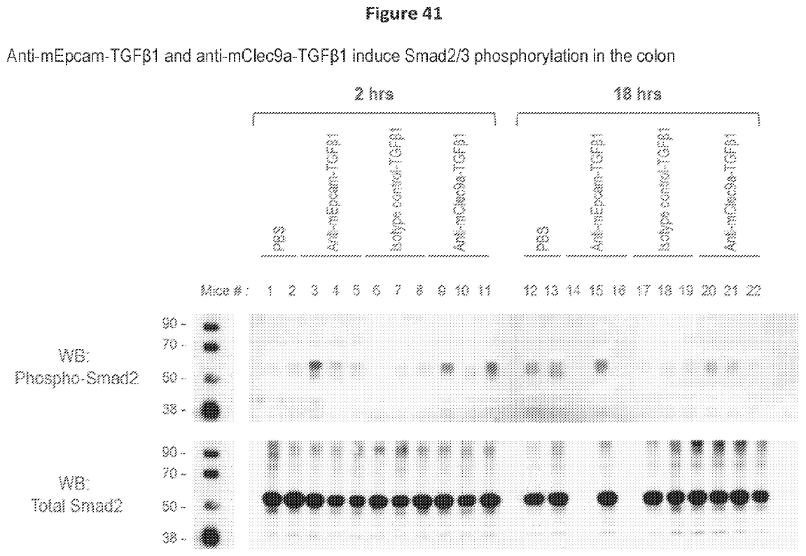

FIG. 40 illustrates that the delivered anti-mEpcam-TGFβ1 SLC strongly induced phosphorylated (P)-Smad2 in the ileum, with this activation persisting even 18 hours post-injection. FIG. 41 shows that the delivered anti-mClec9a (REGN6550)-TGFβ1 SLC induced phosphorylation of Smad2, i.e., pSmad2, in the colon 2 hours post-injection. No pSmad2 induction was detected in the heart, as shown in FIG. 42.

This tissue analysis described herein demonstrates that antibody-TGFβ1 SLC fusions were specifically delivered to the target-expressing tissue and successfully activated the downstream Smad2/3 signaling pathway.

### Example 11. In Vivo Tracing of Antibody-TGFβ1 SLC Fusions

The objective of this Example was to ascertain whether antibody-TGFβ1 SLC fusions could be delivered to specific tissues and subsequently activate downstream Smad2/3 signaling. C57BL/6 male mice were administered anti-mEpcam-TGFβ1 SLC, anti-mClec9a (REGN6550)-TGFβ1 SLC, and mIgG1 isotype control (REGN2390)-TGFβ1 SLC (10 mg/kg RO injection). Following this administration, serum and tissue samples were collected at two intervals: 2 hours and 18 hours post-injection (see, e.g., FIG. 33).

Immunofluorescence (IF) staining of tissues was performed to determine whether antibody-TGFβ1 SLC was delivered to the target tissue and whether the delivered antibody-TGFβ1 SLC fusions activated Smad2/3 signaling.

In this experiment:
1. To determine whether the antibody-TGFβ1 SLC fusions are delivered to the target tissue, tissue staining was performed using the anti-human LAP antibody. The procedure is briefly summarized below:
   a. Sections were sliced from OCT-embedded tissues.
   b. These sections underwent fixation in 4% PFA for 30 minutes, followed by permeabilization in 0.3% TritonX-100 for another 30 minutes.
   c. Subsequently, the sections were stained with PE-conjugated anti-hLAP antibody (Miltenyi, 30-123-409) at 4° C. overnight.
   d. The next day, the slides were rinsed and covered with mounting media containing DAPI.
   e. Images were taken using the Axioscan imager.
2. To verify whether the delivered antibody-TGFβ1 SLC fusions activated Smad2/3 signaling within the target tissue, the tissues were stained with the anti-pSmad2/3 antibody.

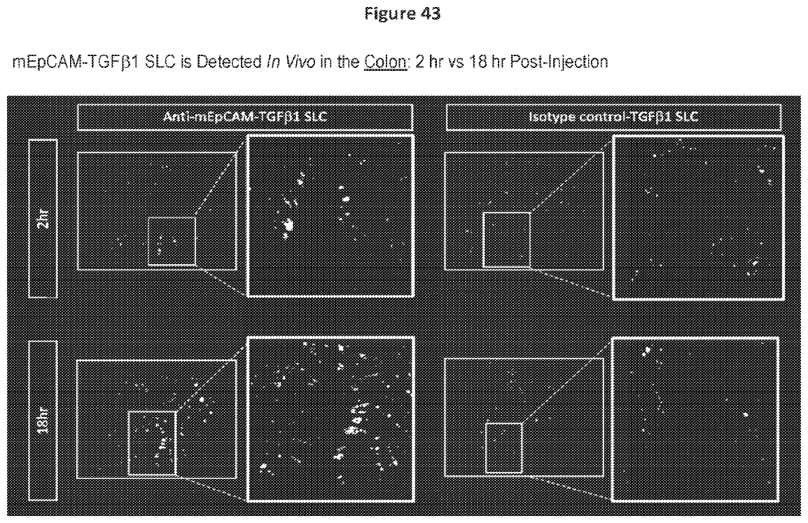

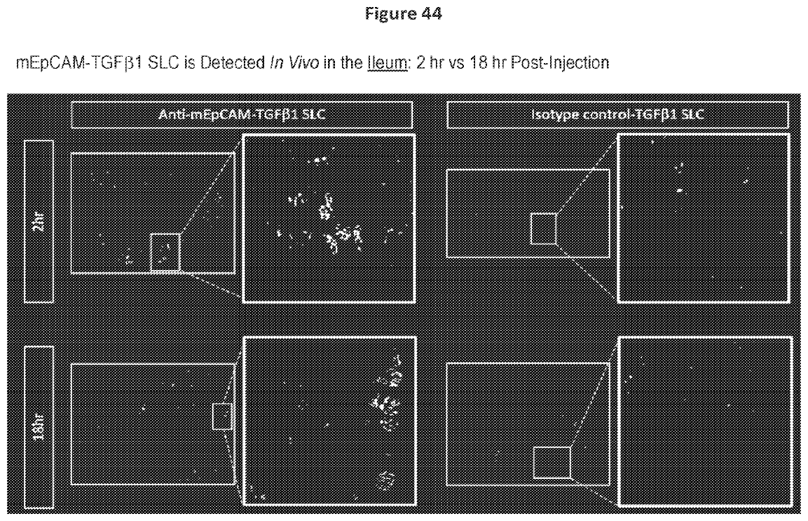

Immunofluorescence (IF) staining of the ileum (FIG. 44) and colon (FIG. 43) was performed using the anti-hLAP antibody to ascertain whether the antibody-TGFβ1 SLC fusions were delivered to the target tissues. The presence of anti-hLAP staining (indicated by ‘red’ staining, appearing as ‘white’ staining in the black and white versions of the images, as provided, and by comparison to isotype control background levels, see e.g., FIG. 44 and FIG. 43, left panels versus right panels) shows the presence of intact antibody-TGFβ1 SLC fusion in the tissue. FIG. 44 and FIG. 43 illustrate that the anti-mEpcam-TGFβ1 SLC is delivered to the ileum and colon, respectively, and is detected in both tissues 2 hours (top, left panels) and 18 hours (bottom, left panels) post-injection. However, no anti-hLAP staining was observed in the ileum and colon samples collected from mice injected with the isotype control-TGFβ1 SLC fusion (see, e.g., 2 hours post-injection, top right panels; 18 hours post-injection, bottom right panels).

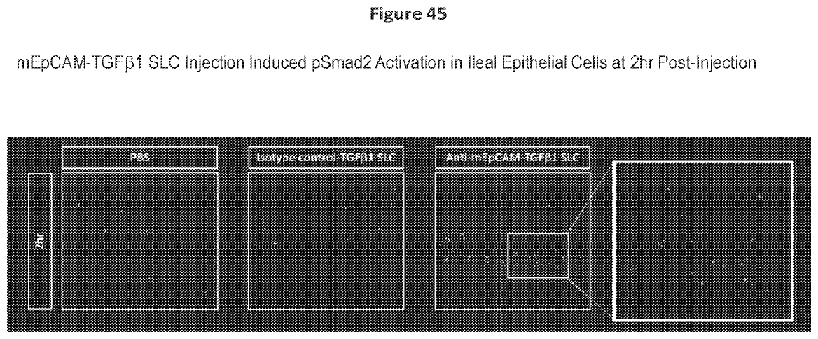

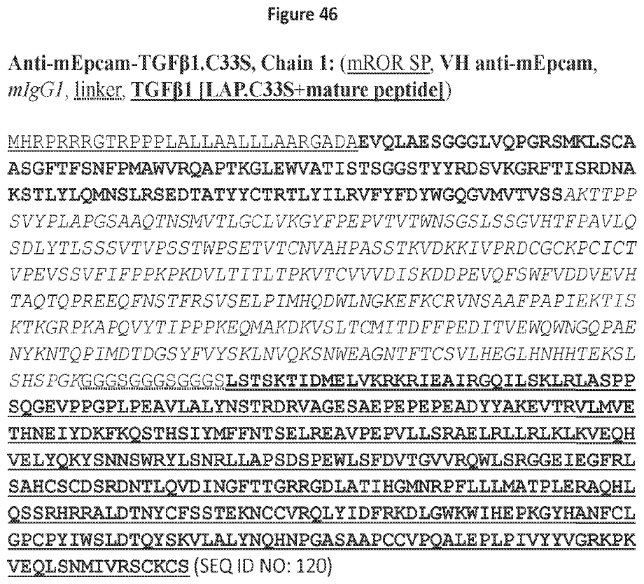

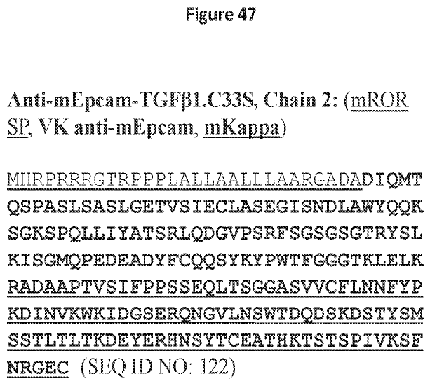

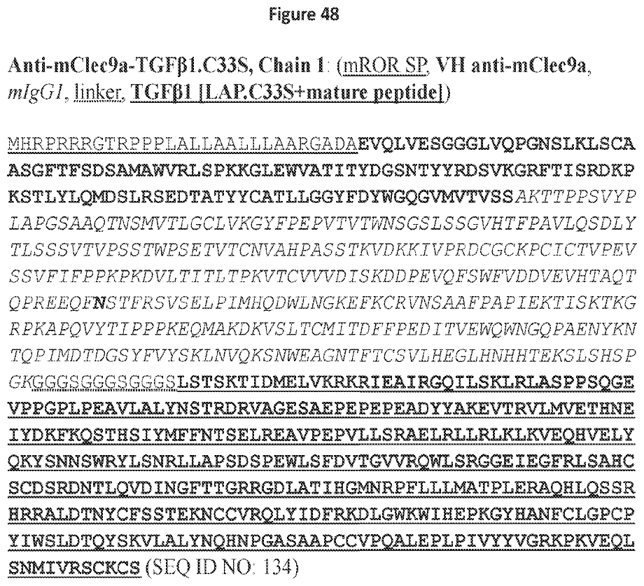

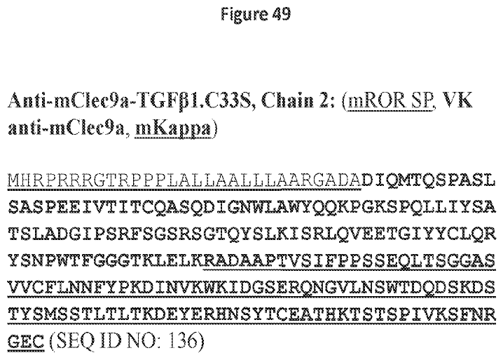

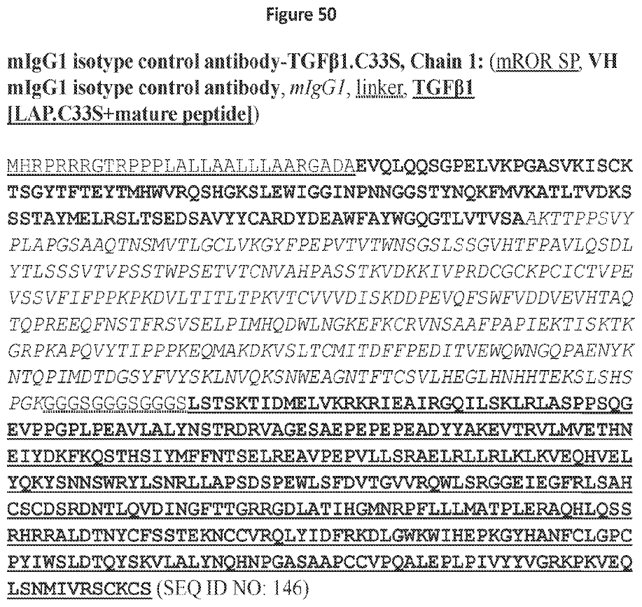

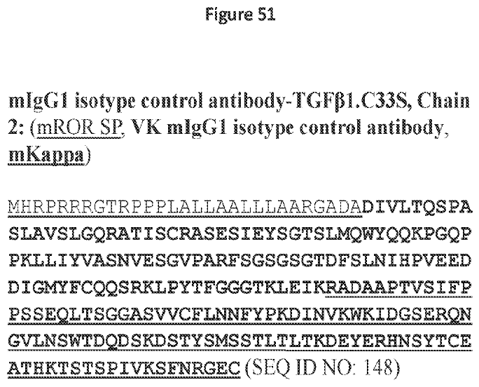

FIG. 45 demonstrates that the anti-mEpcam-TGFβ1 SLC delivered to the ileum induces phosphorylation of Smad2, i.e., pSmad2 (as detected by pSmad2/3 (Thr8) polyclonal antibody, Thermo PA5-99378), in the crypt region of the ileum, where Epcam is also expressed. No pSmad2 signal was detected in the ileum samples collected from mice injected with either PBS or the isotype control-TGFβ1 SLC fusion.

IF staining of the ileum and colon with the antibody against human LAP confirms that anti-mEpcam-TGFβ1 SLC fusion is specifically delivered to the target-expressing tissues. This specificity is driven by the targeting arm, as the isotype control antibody-TGFβ1 SLC fusion was not detected in the tested tissues. IF staining of the ileum with the pSmad2/3 antibody indicates that the delivered anti-mEpcam-TGFβ1 SLC fusion successfully activates the downstream Smad2/3 signaling pathway.

### Example 12. Summary of In Vitro and In Vivo Testing

### In Vitro Testing

In vitro integrin-mediated mechanical and chemical activation assays similar to or the same as those described herein (see, e.g., Example 2, Example 6, and Example 7, above) were performed for additional antibody-TGFβ1 SLC fusions, including, e.g., anti-CD4-TGFβ1 SLC, anti-Clec9a-TGFβ1 SLC, anti-Epcam-TGFβ1 SLC, anti-Olfm4-TGFβ1 SLC, anti-fibronectin-TGFβ1 SLC, anti-HLA:A2-TGFB1 SLC, and anti-Collagen X-TGFβ1 SLC. All tested antibody-TGFβ1 SLC fusions induced Smad2/3 signaling in both the integrin-mediated mechanical activation and chemical activation assays. In the chemical activation assays, the LAP domain was degraded upon exposure to heat or acid. In the integrin-mediated mechanical activation assays, both target and integrin binding were needed to remove the LAP domain, which simulates the biological activation process. Without the removal the LAP domain, antibody-TGFβ1 SLC fusions failed to activate Smad2/3 signaling, thereby demonstrating the latency of the antibody-TGFβ1 SLC fusions.

### In Vivo Testing

The delivery of anti-CD4-TGFβ1 SLC for T-cell targeting, anti-Clec9a-TGFβ1 SLC for dendritic cell targeting, anti-Epcam-TGFβ1 SLC for epithelial cell targeting, anti-Olfm4-TGFβ1 SLC for epithelial cell targeting, and anti-fibronectin-TGFβ1 SLC for matrix targeting were tested in vivo using mice. Utilizing techniques such as immunofluorescence (IF), immunoblotting, and tissue ELISA (see, e.g., Example 10 and Example 11), all antibody-TGFβ1 SLC fusions tested were successfully delivered to their respective cell types in target-expressing tissues. Delivered antibody-TGFβ1 SLC fusions subsequently activated downstream signaling, which was contingent on the availability of activating partners, e.g., integrin αvβ6. For example, an anti-Epcam-TGFβ1 SLC was delivered to the epithelial cells in the ileum, which then activated Smad2/3 signaling (see, e.g., Example 11). None of the antibody-TGFβ1 fusions tested were delivered to non-target expressing tissues. Isotype control antibody-TGFβ1 SLC fusions were not detected in the tested tissues.

The present disclosure is not to be limited in scope by the specific embodiments described herein. Indeed, various modifications of the disclosure in addition to those described herein will become apparent to those skilled in the art from the foregoing description. Such modifications are intended to fall within the scope of the appended claims.

All patents, applications, publications, test methods, literature, and other materials cited herein are hereby incorporated by reference in their entirety as if physically present in this specification.

1. A polypeptide complex comprising:
a. a target-binding polypeptide that binds a molecule on a target cell or a molecule in an extracellular matrix (ECM); and
b. a small latent complex (SLC) comprising:
i. a dimeric latency associated polypeptide (LAP), or a fragment or derivative thereof; and
ii. a dimeric mature Transforming Growth Factor 3 (TGFβ) family polypeptide, or a fragment or derivative thereof, wherein the mature TGFβ family polypeptide, or the fragment or derivative thereof, is inactive as a result of an interaction with the dimeric LAP, or the fragment or derivative thereof.

2. The polypeptide complex of claim 1, wherein the LAP, or the fragment or derivative thereof, is covalently attached to the target-binding polypeptide.

3. The polypeptide complex of claim 2, wherein the LAP, or the fragment or derivative thereof, is covalently attached to the target-binding polypeptide via a linker.

4. The polypeptide complex of claim 3, wherein the linker comprises the sequence (GGGGS)n (SEQ ID NO: 46), (GGGS)n (SEQ ID NO: 51), or GSGESGGGSG (SEQ ID NO: 96).

5. The polypeptide complex of claim 4, wherein the linker consists of the sequence (GGGGS)n (SEQ ID NO: 46), (GGGS)n (SEQ ID NO: 51), or GSGESGGGSG (SEQ ID NO: 96).

6. The polypeptide complex of claim 3, wherein the linker comprises the sequence GGGSGGGSGGGS (SEQ ID NO: 19).

7. The polypeptide complex of claim 6, wherein the linker consists of the sequence GGGSGGGSGGGS (SEQ ID NO: 19).

8. The polypeptide complex of claim 3, wherein the linker comprises the sequence GSGESGGGSG (SEQ ID NO: 96).

9. The polypeptide complex of claim 8, wherein the linker consists of the sequence GSGESGGGSG (SEQ ID NO: 96).

10. The polypeptide complex of claim 7 or claim 9, wherein the target-binding polypeptide binds both the LAP, or the fragment or derivative thereof, and the molecule on the target cell or the molecule in the ECM.

11. The polypeptide complex of claim 10, wherein the target-binding polypeptide is an antibody or a fragment or derivative thereof.

12. The polypeptide complex of any one of claims 1-11, wherein the mature TGFβ family polypeptide, or the fragment or derivative thereof, and the LAP, or the fragment or derivative thereof, are associated via a noncovalent interaction.

13. The polypeptide complex of any one of claims 1-11, wherein the mature TGFβ family polypeptide, or the fragment or derivative thereof, and the LAP, or the fragment or derivative thereof, are separated by a protease cleavage site.

14. The polypeptide complex of claim 13, wherein the protease cleavage site is a furin cleavage site.

15. The polypeptide complex of claim 14, wherein the furin cleavage site comprises the sequence RXXR (SEQ ID NO: 35).

16. The polypeptide complex of claim 15, wherein the furin cleavage site consists of the sequence RXXR (SEQ ID NO: 35).

17. The polypeptide complex of claim 16, wherein the RXXR (SEQ ID NO: 35) is RHRR (SEQ ID NO: 85), RRKR (SEQ ID NO: 86), or RKKR (SEQ ID NO: 87).

18. The polypeptide complex of any one of claims 1-17, wherein the mature TGFβ family polypeptide, or the fragment or derivative thereof, binds a Transforming Growth Factor R Receptor (TGFβR) upon release of the mature TGFβ family polypeptide, or the fragment or derivative thereof, from the SLC.

19. The polypeptide complex of any one of claims 1-18, wherein the mature TGFβ family polypeptide, or the fragment or derivative thereof, induces Smad2/3 signaling in the target cell or a cell adjacent to the target cell upon release from the SLC.

20. The polypeptide complex of any one of claims 1-19, wherein the LAP, or the fragment or derivative thereof, comprises an integrin binding motif.

21. The polypeptide complex of claim 20, wherein the integrin binding motif comprises the sequence RGD.

22. The polypeptide complex of claim 20, wherein the integrin is αvβ6 integrin or αvβ8 integrin.

23. The polypeptide complex of any one of claims 1-19, wherein the LAP, or the fragment or derivative thereof, does not comprise an integrin binding motif.

24. The polypeptide complex of any one of claims 1-23, wherein the target-binding polypeptide is an antigen-binding polypeptide or antigen-binding fragment thereof.

25. The polypeptide complex of claim 24, wherein the antigen-binding polypeptide is an antibody or antigen-binding fragment thereof.

26. The polypeptide complex of claim 25, wherein the antibody or antigen-binding fragment thereof comprises a heavy chain variable region.

27. The polypeptide complex of claim 25 or claim 26, wherein the antibody or antigen-binding fragment thereof comprises a light chain variable region.

28. The polypeptide complex of any one of claims 25-27, wherein the antibody or antigen-binding fragment thereof comprises an immunoglobulin heavy chain constant domain.

29. The polypeptide complex of claim 28, wherein the immunoglobulin heavy chain constant domain is an IgG1 domain.

30. The polypeptide complex of claim 28, wherein the immunoglobulin heavy chain constant domain is an IgG4 domain.

31. The polypeptide complex of any one of claims 25-30, wherein the antigen-binding polypeptide or the antigen-binding fragment thereof binds to CD63.

32. The polypeptide complex of any one of claims 25-30, wherein the antigen-binding polypeptide or the antigen-binding fragment thereof binds to extra domain B of fibronectin (EDB-FN).

33. The polypeptide complex of any one of claims 25-30, wherein the antigen-binding polypeptide or the antigen-binding fragment thereof binds to epithelial cell adhesion molecule (Epcam).

34. The polypeptide complex of any one of claims 25-30, wherein the antigen-binding polypeptide or the antigen-binding fragment thereof binds to C-type lectin domain family 9 member A (Clec9a).

35. The polypeptide complex of any one of claims 1-34, wherein the target-binding polypeptide is not internalizing.

36. The polypeptide complex of any one of claims 1-35, wherein the mature TGFβ family polypeptide is a mature TGFβ polypeptide.

37. The polypeptide complex of claim 36, wherein the mature TGFβ polypeptide is a mature TGFβ1 polypeptide.

38. The polypeptide complex of claim 37, wherein the mature TGFβ1 polypeptide comprises the sequence of SEQ ID NO: 23.

39. The polypeptide complex of claim 38, wherein the mature TGFβ1 polypeptide consists of the sequence of SEQ ID NO: 23.

40. The polypeptide complex of claim 37, wherein the mature TGFβ1 polypeptide comprises the sequence of SEQ ID NO: 90.

41. The polypeptide complex of claim 40, wherein the mature TGFβ1 polypeptide consists of the sequence of SEQ ID NO: 90.

42. The polypeptide complex of claim 36, wherein the mature TGFβ polypeptide is a mature TGFβ2 polypeptide.

43. The polypeptide complex of claim 42, wherein the mature TGFβ2 polypeptide comprises the sequence of SEQ ID NO: 27.

44. The polypeptide complex of claim 43, wherein the mature TGFβ2 polypeptide consists of the sequence of SEQ ID NO: 27.

45. The polypeptide complex of any one of claims 1-35, wherein the mature TGFβ family polypeptide is a mature Growth Differentiation Factor 8 (GDF8), a mature Growth Differentiation Factor 11 (GDF11) polypeptide, or a mature Bone Morphogenetic Protein 4 (BMP4).

46. The polypeptide complex of any one of claims 1-44, wherein the LAP comprises the sequence of positions 30-274 of the sequence of SEQ ID NO: 82.

47. The polypeptide complex of claim 46, wherein the LAP consists of the sequence of positions 30-274 of the sequence of SEQ ID NO: 82.

48. The polypeptide complex of any one of claims 1-44, wherein the LAP comprises the sequence of positions 21-298 of the sequence of SEQ ID NO: 116.

49. The polypeptide complex of claim 48, wherein the LAP consists of the sequence of positions 21-298 of the sequence of SEQ ID NO: 116.

50. The polypeptide complex of any one of claims 1-49, wherein the LAP, or the fragment or derivative thereof, is heterologous to the mature TGFβ family polypeptide, or the fragment or derivative thereof.

51. The polypeptide complex of any one of claims 1-50, wherein the LAP, or the fragment or derivative thereof, comprises one or more mutations, wherein the one or more mutations allow for proteolytic activation of the mature TGFβ family polypeptide, or the fragment or derivative thereof.

52. The polypeptide complex of any one of claims 1-51, wherein the LAP, or the fragment or derivative thereof, comprises one or more mutations, wherein the one or more mutations allow for mechanical activation of the mature TGFβ family polypeptide, or the fragment or derivative thereof.

53. The polypeptide complex of any one of claims 1-52, wherein the LAP, or the fragment or derivative thereof, comprises one or more mutations, wherein the one or more mutations eliminate one or more protease cleavage sites within the LAP, or the fragment or derivative thereof.

54. The polypeptide complex of any one of claims 1-53, wherein the LAP, or the fragment or derivative thereof, comprises one or more mutations, wherein the one or more mutations decrease binding of the LAP, or the fragment or derivative thereof, to a latency associated binding protein (LTBP).

55. The polypeptide complex of claim 54, wherein the one or more mutations comprise a C33S mutation, wherein position 33 is in relation to the sequence of SEQ ID NO: 82.

56. The polypeptide complex of claim 54, wherein the one or more mutations comprise a C24S mutation, wherein position 24 is in relation to SEQ ID NO: 116.

57. The polypeptide complex of any one of claims 1-44 and 54-55, wherein the LAP comprises the sequence of SEQ ID NO: 31.

58. The polypeptide complex of claim 57, wherein the LAP consists of the sequence of SEQ ID NO: 31.

59. The polypeptide complex of any one of claims 1-44, 54, and 56, wherein the LAP comprises the sequence of SEQ ID NO: 94.

60. The polypeptide complex of claim 59, wherein the LAP consists of the sequence of SEQ ID NO: 94.

61. The polypeptide complex of any one of claims 1-60, wherein the mature TGFβ family polypeptide, or the fragment or derivative thereof, is chemically dissociated from the LAP, or the fragment or derivative thereof, and released in an active form from the SLC.

62. The polypeptide complex of claim 61, wherein the chemical dissociation comprises a protease treatment, a temperature treatment, an acid treatment, or any combination thereof.

63. The polypeptide complex of any one of claims 1-60, wherein the mature TGFβ family polypeptide, or the fragment or derivative thereof, is mechanically dissociated from the LAP, or the fragment or derivative thereof, and released in an active form from the SLC.

64. The polypeptide complex of claim 63, wherein the mechanical dissociation occurs a result of an interaction between the LAP, or the fragment or derivative thereof, and an integrin polypeptide.

65. A pharmaceutical composition comprising the polypeptide complex of any one of claims 1-64.

66. The pharmaceutical composition of claim 65, further comprising a pharmaceutically acceptable carrier or diluent.

67. A fusion polypeptide comprising:
a. a target-binding polypeptide that binds a molecule on a target cell or a molecule in an extracellular matrix (ECM);
b. a latency associated polypeptide (LAP), or a fragment or derivative thereof, and
c. a mature Transforming Growth Factor β (TGFβ) family polypeptide, or a fragment or derivative thereof.

68. The fusion polypeptide of claim 67, wherein the mature TGFβ family polypeptide, or the fragment or derivative thereof, is inactive as a result of an interaction with the LAP, or the fragment or derivative thereof.

69. The fusion polypeptide of claim 67 or claim 68, wherein the mature TGFβ family polypeptide, or the fragment or derivative thereof, binds a Transforming Growth Factor R Receptor (TGFβR) upon release of the mature TGFβ family polypeptide, or the fragment or derivative thereof, from the LAP, or the fragment or derivative thereof.

70. The fusion polypeptide of any one of claims 67-69, wherein the mature TGFβ family polypeptide, or the fragment or derivative thereof, induces Smad2/3 signaling in the target cell or a cell adjacent to the target cell upon release of the mature TGFβ family polypeptide, or the fragment or derivative thereof, from the LAP, or the fragment or derivative thereof.

71. The fusion polypeptide of any one of claims 67-70, wherein the fusion polypeptide comprises a linker.

72. The fusion polypeptide of claim 71, wherein the linker is located between the target-binding polypeptide and the LAP, or the fragment or derivative thereof.

73. The fusion polypeptide of claim 72, wherein the fusion polypeptide comprises, from N-terminus to C-terminus, (i) the target-binding polypeptide, (ii) the linker, (iii) the LAP, or the fragment or derivative thereof, and (iv) the mature TGFβ family polypeptide, or the fragment or derivative thereof.

74. The fusion polypeptide of claim 72, wherein the fusion polypeptide comprises, from N-terminus to C-terminus, (i) the mature TGFβ family polypeptide, or the fragment or derivative thereof, (ii) the LAP, or the fragment or derivative thereof, (iii) the linker, and (iv) the target-binding polypeptide.

75. The fusion polypeptide of any one of claims 71-74, wherein the linker comprises the sequence (GGGGS)n (SEQ ID NO: 46), (GGGS)n (SEQ ID NO: 51), or GSGESGGGSG (SEQ ID NO: 96).

76. The fusion polypeptide of claim 75, wherein the linker consists of the sequence (GGGGS)n (SEQ ID NO: 46), (GGGS)n (SEQ ID NO: 51), or GSGESGGGSG (SEQ ID NO: 96).

77. The fusion polypeptide of claim 75, wherein the linker comprises the sequence GGGSGGGSGGGS (SEQ ID NO: 19).

78. The fusion polypeptide of claim 77, wherein the linker consists of the sequence GGGSGGGSGGGS (SEQ ID NO: 19).

79. The fusion polypeptide complex of claim 75, wherein the linker comprises the sequence GSGESGGGSG (SEQ ID NO: 96).

80. The fusion polypeptide complex of claim 79, wherein the linker consists of the sequence GSGESGGGSG (SEQ ID NO: 96).

81. The fusion polypeptide of any one of claims 67-80, wherein the mature TGFβ family polypeptide, or the fragment or derivative thereof, and the LAP, or the fragment or derivative thereof, are separated by a protease cleavage site.

82. The fusion polypeptide of claim 81, wherein the protease cleavage site is a furin cleavage site.

83. The fusion polypeptide of claim 82, wherein the furin cleavage site comprises the sequence RXXR (SEQ ID NO: 35).

84. The fusion polypeptide of claim 83, wherein the furin cleavage site consists of the sequence RXXR (SEQ ID NO: 35).

85. The fusion polypeptide of claim 84, wherein the RXXR (SEQ ID NO: 35) is RHRR (SEQ ID NO: 85), RRKR (SEQ ID NO: 86), or RKKR (SEQ ID NO: 87).

86. The fusion polypeptide of any one of claims 67-84, further comprising a signal peptide.

87. The fusion polypeptide of claim 86, wherein the signal peptide is mROR signal peptide.

88. The fusion polypeptide of claim 87, wherein the mROR signal peptide comprises the sequence MHRPRRRGTRPPPLALLAALLLAARGADA (SEQ ID NO: 1).

89. The fusion polypeptide of claim 88, wherein the mROR signal peptide consists of the sequence MHRPRRRGTRPPPLALLAALLLAARGADA (SEQ ID NO: 1).

90. The fusion polypeptide of any one of claims 67-89, wherein the LAP, or the fragment or derivative thereof, comprises an integrin binding motif.

91. The fusion polypeptide of claim 90, wherein the integrin binding motif comprises the sequence RGD.

92. The fusion polypeptide of claim 90, wherein the integrin is αvβ6 integrin or αvβ8 integrin.

93. The fusion polypeptide of any one of claims 67-89, wherein the LAP, or the fragment or derivative thereof, does not comprise an integrin binding motif.

94. The fusion polypeptide of any one of claims 67-93, wherein the target-binding polypeptide is an antigen-binding polypeptide or antigen-binding fragment thereof.

95. The fusion polypeptide of claim 94, wherein the antigen-binding polypeptide is an antibody or antigen-binding fragment thereof.

96. The fusion polypeptide of claim 95, wherein the antibody or antigen-binding fragment thereof comprises a heavy chain variable region.

97. The fusion polypeptide of claim 95 or claim 96, wherein the antibody or antigen-binding fragment thereof comprises a light chain variable region.

98. The fusion polypeptide of any one of claims 95-97, wherein the antibody or antigen-binding fragment thereof comprises an immunoglobulin heavy chain constant domain.

99. The fusion polypeptide of claim 98, wherein the immunoglobulin heavy chain constant domain is an IgG1 domain.

100. The fusion polypeptide of claim 98, wherein the immunoglobulin heavy chain constant domain is an IgG4 domain.

101. The fusion polypeptide of any one of claims 94-100, wherein the antigen-binding polypeptide or the antigen-binding fragment thereof binds to CD63.

102. The fusion polypeptide of any one of claims 94-100, wherein the antigen binding polypeptide of the antigen-binding fragment thereof binds to extra domain B of fibronectin (EDB-FN).

103. The fusion polypeptide of any one of claims 94-100, wherein the antigen-binding polypeptide or the antigen-binding fragment thereof binds to epithelial cell adhesion molecule (Epcam).

104. The fusion polypeptide of any one of claims 94-100, wherein the antigen-binding polypeptide or the antigen-binding fragment thereof binds to C-type lectin domain family 9 member A (Clec9a).

105. The fusion polypeptide of any one of claims 67-104, wherein the target-binding polypeptide is not internalizing.

106. The fusion polypeptide of any one of claims 67-105, wherein the mature TGFβ family polypeptide is a mature TGFβ polypeptide.

107. The fusion polypeptide of claim 106, wherein the mature TGFβ polypeptide is a mature TGFβ1 polypeptide.

108. The fusion polypeptide of claim 107, wherein the mature TGFβ1 polypeptide comprises the sequence of SEQ ID NO: 23.

109. The fusion polypeptide of claim 108, wherein the mature TGFβ1 polypeptide consists of the sequence of SEQ ID NO: 23.

110. The fusion polypeptide of claim 107, wherein the mature TGFβ1 polypeptide comprises the sequence of SEQ ID NO: 90.

111. The fusion polypeptide of claim 110, wherein the mature TGFβ1 polypeptide comprises the sequence of SEQ ID NO: 90.

112. The fusion polypeptide of claim 106, wherein the mature TGFβ polypeptide is a mature TGFβ2 polypeptide.

113. The fusion polypeptide of claim 112, wherein the mature TGFβ2 polypeptide comprises the sequence of SEQ ID NO: 27.

114. The fusion polypeptide of claim 113, wherein the mature TGFβ2 polypeptide consists of the sequence of SEQ ID NO: 27.

115. The fusion polypeptide of any one of claims 67-105, wherein the mature TGFβ family polypeptide is a mature Growth Differentiation Factor 8 (GDF8), a mature Growth Differentiation Factor 11 (GDF11) polypeptide, or a mature Bone Morphogenetic Protein 4 (BMP4).

116. The fusion polypeptide of any one of claims 67-114, wherein the LAP comprises the sequence of positions 30-274 of the sequence of SEQ ID NO: 82.

117. The fusion polypeptide of claim 116, wherein the LAP consists of the sequence of positions 30-274 of the sequence of SEQ ID NO: 82.

118. The fusion polypeptide of any one of claims 67-114, wherein the LAP comprises the sequence of positions 21-298 of the sequence of SEQ ID NO: 116.

119. The fusion polypeptide of claim 118, wherein the LAP consists of the sequence of positions 21-298 of the sequence of SEQ ID NO: 116.

120. The fusion polypeptide of any one of claims 67-119, wherein the LAP, or the fragment or derivative thereof, is heterologous to the mature TGFβ family polypeptide, or the fragment or derivative thereof.

121. The fusion polypeptide of any one of claims 67-120, wherein the LAP, or the fragment or derivative thereof, comprises one or more mutations, wherein the one or more mutations allow for proteolytic activation of the mature TGFβ family polypeptide, or the fragment or derivative thereof.

122. The fusion polypeptide of any one of claims 67-121, wherein the LAP, or the fragment or derivative thereof, comprises one or more mutations, wherein the one or more mutations allow for mechanical activation of the mature TGFβ family polypeptide, or the fragment or derivative thereof.

123. The fusion polypeptide of any one of claims 67-122, wherein the LAP, or the fragment or derivative thereof, comprises one or more mutations, wherein the one or more mutations eliminate one or more protease cleavage sites within the LAP, or the fragment or derivative thereof.

124. The fusion polypeptide of any one of claims 67-123, wherein the LAP, or the fragment or derivative thereof, comprises one or more mutations, wherein the one or more mutations decrease binding of the LAP, or the fragment or derivative thereof, to a latency associated binding protein (LTBP).

125. The fusion polypeptide of claim 124, wherein the one or more mutations comprise a C33S mutation, wherein position 33 is in relation to the sequence of SEQ ID NO: 82.

126. The fusion polypeptide of claim 124, wherein the one or more mutations comprise a C24S mutation, wherein position 24 is in relation to SEQ ID NO: 116

127. The fusion polypeptide of any one of claims 67-114 and 124-125, wherein the LAP comprises the sequence of SEQ ID NO: 31.

128. The fusion polypeptide of claim 127, wherein the LAP consists of the sequence of SEQ ID NO: 31.

129. The fusion polypeptide of any one of claims 67-114, 124, and 126 wherein the LAP comprises the sequence of SEQ ID NO: 94.

130. The fusion polypeptide of claim 129, wherein the LAP consists of the sequence of SEQ ID NO: 94.

131. A polynucleotide encoding the fusion polypeptide of any one of claims 67-128.

132. A vector comprising the polynucleotide of claim 131.

133. The vector of claim 132, wherein the sequence encoding the fusion polypeptide is operably linked to a promoter, wherein the promoter mediates expression of the fusion polypeptide.

134. The vector of claim 132 or claim 133, wherein the vector is a viral vector.

135. The vector of claim 134, wherein the viral vector is an adeno-associated virus (AAV) vector.

136. A cell comprising the polypeptide complex of any one of claims 1-64, the fusion polypeptide of any one of claims 67-130, the polynucleotide of claim 131, or the vector of any one of claims 132-135.

137. A method of making the polypeptide complex of any one of claims 1-64, comprising incubating the cell comprising the polynucleotide of claim 131, or the vector of any one of claims 132-135, under conditions allowing for production of the polypeptide complex.

138. The method of claim 137, further comprising collecting the cell culture medium and isolating the produced polypeptide complex by a process comprising affinity chromatography.

139. The method of claim 138, wherein the affinity chromatography comprises a Protein A or a Protein G column or beads.

140. A method for treating a TGFβ dysregulation disorder in a subject in need thereof, comprising administering to the subject a therapeutically effective amount of the polypeptide complex of any one of claims 1-64, the pharmaceutical composition of claim 65 or claim 66, the polynucleotide of claim 131, or the vector of any one of claims 132-135.

141. The method of claim 140, wherein the polypeptide complex, pharmaceutical composition, polynucleotide, or vector is administered via injection.

142. The method of claim 141, wherein the injection is intravenous, intramuscular, subcutaneous, or intraperitoneal.

143. The method of any one of claims 140-142, wherein the polypeptide complex, pharmaceutical composition, polynucleotide, or vector is administered via hydrodynamic delivery (HDD).

144. The method of any one of claims 140-143, wherein the polypeptide complex, pharmaceutical composition, polynucleotide, or vector is administered to the liver of the subject.

145. The method of any one of claims 140-144, wherein the polypeptide complex, pharmaceutical composition, polynucleotide, or vector is administered in combination with an additional therapeutic agent.

146. The method of any one of claims 140-145, wherein the TGFβ dysregulation disorder is an inflammatory bowel disease (IBD).

147. The method of any one of claims 140-145, wherein the TGFβ dysregulation disorder is Marfan syndrome.

148. The method of any one of claims 140-145, wherein the TGFβ dysregulation disorder is an autoimmune disorder.

149. The method of any one of claims 140-145, wherein the TGFβ dysregulation disorder is a wound healing disorder.

150. A method for promoting wound healing in a subject in need thereof, comprising administering to the subject a therapeutically effective amount of the polypeptide complex of any one of claims 1-64, the pharmaceutical composition of claim 65 or claim 66, the polynucleotide of claim 131, or the vector of any one of claims 132-135.

151. The method of claim 150, wherein the polypeptide complex, pharmaceutical composition, polynucleotide, or vector is administered to the wound of the subject.

152. The method of any one of claims 140-151, wherein the subject is human.
